(19) United States
(12) Patent Application Publication
(10) Pub. No.: US 2026/0284243 A1
(43) Pub. Date: Sep. 24, 2026

(54) GASTRIN-RELEASING PEPTIDE RECEPTOR (GRPR)-TARGETED COMPOUNDS AND USES THEREOF

(71) Applicant: Alpha-9 Oncology Inc., Vancouver (CA)

(72) Inventors: Jutta WANNER, Salt Spring Island (CA); Chengcheng ZHANG, Richmond (CA); Kaveh MATINKHOO, Vancouver (CA); Stephen BARIGYE, Vancouver (CA); Hsiou-Ting KUO, Vancouver (CA)

(21) Appl. No.: 19/680,120

(22) Filed: May 18, 2026

Related U.S. Application Data
(63) Continuation of application No. 19/334,526, filed on Sep. 19, 2025.
Application No. 19/334,526 is a continuation of application No. PCT/CA2025/051213, filed on Sep. 12, 2025.
(60) Provisional application No. 63/694,257, filed on Sep. 13, 2024.

(51) Int. Cl. A61K 51/08; A61K 51/04; A61P 35/00; C07K 1/107

(52) U.S. Cl. CPC A61K 51/088; A61K 51/0482; A61P 35/00; C07K 1/1077; A61K 2121/00; A61K 2123/00

(57) ABSTRACT

The present invention relates to gastrin-releasing peptide receptor (GRPR)-targeted compounds and their use for imaging and treatment of diseases or conditions characterized by expression of the gastrin-releasing peptide receptor.

### CROSS-REFERENCE TO RELATED APPLICATIONS

This application is a continuation of U.S. application Ser. No. 19/334,526, filed on Sep. 19, 2025, which is a continuation of International Patent Application No. PCT/CA2025/051213, filed on Sep. 12, 2025, which claims priority to U.S. Provisional Patent Application No. 63/694,257, filed on Sep. 13, 2024, the contents of each of which is hereby incorporated by reference in its entirety for all purposes.

### REFERENCE TO AN ELECTRONIC SEQUENCE LISTING

The contents of the electronic sequence listing (A9TH_025_02US_SeqList_ST26.xml; Size: 218,099 bytes; and Date of Creation: May 18, 2026) are herein incorporated by reference in its entirety.

### FIELD

The present invention relates to gastrin-releasing peptide receptor (GRPR)-targeted compounds and their use for imaging and treatment of diseases or conditions characterized by expression of the gastrin-releasing peptide receptor.

### BACKGROUND

Gastrin-releasing peptide receptor (GRPR) is a G protein-coupled receptor of the bombesin (BBN) receptor family (Roesler & Schwartsmann. 2012. Front Endocrinol (Lausanne) 3:159; Bitar & Zhu. 1993. Gastroenterology. 105:1672-1680; Weber. 2009. Curr Opin Endocrinol Diabetes Obes. 16:66-71). Together with its endogenous ligand, gastrin-releasing peptide (GRP), GRPR is involved in synaptic plasticity, emotional and feeding behavior, hormone secretion, smooth muscle contraction, and cell proliferation (ibid.). In normal conditions, the expression of GRPR is restricted to the central nervous system, pancreas, adrenal cortex and gastrointestinal tract (Jensen, et al. 2008. Pharmacol Rev. 60:1-42). GRPR is also implicated in neoplastic progression, with overexpression of GRPR having been reported in many cancer subtypes including lung, head and neck, colon, kidney, ovarian, breast and prostate cancers (Cornelio, et al. Ann Oncol. 2007, 18:1457-1466). This ectopic expression in cancers makes it an attractive target for personalized therapies.

BBN is a 14 amino acid GRPR binding peptide (Lin, et al. 2004. Bioconjugate Chemistry. Vol 15. American Chemical Society pages 1416-1423; Inkster, et al. 2013 Bioorganic Med Chem Lett. 23:3920-3926; Mansi, et al. 2016 J Nucl Med. 57:67S-72S; Bodei, et al. 2007. ¹⁷⁷Lu-AMBA Bombesin analogue in hormone refractory prostate cancer patients: a phase I escalation study with single-cycle administrations. In: JOINT EANM-EORTC Symposium; Sah, et al. 2015 J Nucl Med. 56:372-378; Zang, et al. 2018 Clin Nucl Med. 43:663-669; Nock, et al. 2017 J Nucl Med. 58:75-80; Maina, et al. 2016 Eur J Nucl Med Mol Imaging 43:964-973). BBN derivatives have been radiolabeled for imaging with single photon emission computed tomography (SPECT), positron emission tomography (PET), and have also been radiolabeled for therapy with beta and alpha emitters (Maina, et al. PET Clin. 2017; 12:297-309; Lin, et al. 2004. Bioconjugate Chemistry. Vol 15. American Chemical Society pages 1416-1423; Inkster, et al. 2013 Bioorganic Med Chem Lett. 23:3920-3926). Often, a radiolabelled group is appended directly onto the structure or via a linker at the N-terminus, while modifications at the C-terminus dictate agonist/antagonist properties. For targeting GRPR, antagonists are preferred since agonists have been shown to induce gastrointestinal adverse events (Bodei, et al. 2007. ¹⁷⁷Lu-AMBA Bombesin analogue in hormone refractory prostate cancer patients: a phase I escalation study with single-cycle administrations. In: JOINT EANM-EORTC Symposium). Examples of GRPR antagonists evaluated in the clinic include: ⁶⁸Ga-RM2, ⁶⁸Ga—SB3, ⁶⁸Ga-NeoBOMB1, ⁶⁸Ga-RM26, ¹⁸F-BAY-864367, and ⁶⁴Cu—CB-TE2A-AR06 (Mansi, et al. 2016 J Nucl Med. 57:67S-72S; Sah, et al. 2015 J Nucl Med. 56:372-378; Zang, et al. 2018 Clin Nucl Med. 43:663-669; Nock, et al. 2017 J Nucl Med. 58:75-80; Maina, et al. 2016 Eur J Nucl Med Mol Imaging 43:964-973; Kahkonen, et al. Clin Cancer Res. 2013; 19:5434-5443, Kahkonen, et al. Clin Cancer Res. 2013; 19:5434-5443; Baum, et al. 2007 Journal of Nuclear Medicine 48, 79P-79P). However, high pancreas uptake remains the major limitation of currently reported GRPR-targeting radioligands.

In a study, the high pancreas uptake of ⁶⁸Ga-labeled AMBA was up to 54.9 SUV (SUV: standard uptake value) (Baum, et al. 2007 Journal of Nuclear Medicine 48, 79P-79P). In addition, ⁶⁸Ga-labeled RM2 was also reported to show high uptake in pancreas (Kurth, et al. 2020. European journal of nuclear medicine and molecular imaging 47,123-135; Minamimoto, et al. 2016 J Nucl Med. 57:557-562). It has also been reported that radiolabeled NeoBOMB1 showed high pancreas uptake in both PC-3 tumor-bearing mice and prostate cancer patients (Nock, et al. 2017 J Nucl Med. 58:75-80).

Another limitation for most of the reported GRPR-targeting ligands is their in vivo metabolic instability (Bakker, et al. 2018 Molecular imaging and biology 20, 973-983; Rousseau, et al. 2020 Journal of Labelled Compounds and Radiopharmaceuticals 63, 56-64) due to enzymatic degradation by neutral endopeptidase (NEP) (Nock, et al. 2014 J Nucl Med. 55:121-127). His¹²-Leu¹³, Trp⁸-Ala⁹ and Gln⁷-Trp⁸ were reported to be the main cleavage sites within the AMBA's sequence, and Trp⁸-Ala⁹, Ala⁹-Val¹⁰ and Gln⁷-Trp⁸ were considered to be the cleavage sites of RM2 (Kahkonen, et al. 2013 Clin Cancer Res. 19:5434-5443; Linder et al. 2009 Bioconjugate chemistry 20, 1171-1178).

A series of bombesin analogs modifying its C-terminal Leu¹³-Met¹⁴ have been developed. See, Rousseau, E. et al. Journal of Labelled Compounds and Radiopharmaceuticals 63, 56-64 (2020), Lau, J. et al. ACS omega 4, 1470-1478 (2019), and Bratanovic, I. J. et al. J. of Nuclear Medicine July 2021, jnumed.120.257758, each of which is hereby incorporated by reference in its entirety for all purposes. However, there still remain a need in the field for improved tracers for the non-invasive in-vivo imaging of the GRPR. Such tracers are useful for the diagnosis of disorders related to aberrant/ectopic expression of GRPR, including but not limited to cancer (e.g., prostate cancer). There also remains an unmet need for improved radiotherapeutic agents for treatment of diseases/disorders related to aberrant/ectopic expression of GRPR, including but not limited to cancer (e.g., prostate cancer). In particular, there is a need for GRPR-targeting radioligands (for imaging and/or therapy) with lower pancreas uptake, and useful stability in vivo.

No admission is necessarily intended, nor should it be construed, that any of the preceding information constitutes prior art against the present invention.

### SUMMARY

In one aspect, this disclosure provides a peptidic compound of Formula (A), or a salt or solvate thereof:

(A)
R^{rad}ₙ₆-R^{L}-[linker]-R^{L}-Xaa¹-Xaa²-Xaa³-Xaa⁴-Xaa⁵-
Xaa⁶-Xaa⁷-Xaa⁸-[ψ-Xaa⁹-(Xaa¹⁰)ₘ₂]ₘ₁-R

wherein:
Xaa¹ is an N-terminal amino acid residue selected from D-Phe, 4-chlorophenylalanine (Cpa), D-Cpa, 3-(1-naphthyl)alanine (Nal), D-Nal, 3-(2-naphthyl)alanine (2-Nal), D-2-Nal, 2,3,4,9-tetrahydro-1H-pyrido[3,4-b]indol-3-carboxylic acid (Tpi), or D-Tpi;
Xaa² is Gly, Arg, Asp, Asn, Gln, Glu, homoserine (Hse), citrulline (Cit), 2-amino-4-ureidobutanoic acid (Aub), 2-amino-4-guanidinobutyric acid (Agb), or His;
Xaa³ is Trp, β-(3-benzothienyl)alanine (Bta), Trp(Me), Trp(7-Me), Trp(6-Me), Trp(5-Me), Trp(4-Me), Trp(2-Me), Trp(7-F), Trp(6-F), Trp(5-F), Trp(4-F), Trp(7-Cl), Trp(6-Cl), Trp(5-Cl), Trp(4-Cl), Trp(7-Br), Trp(6-Br), Trp(5-Br), Trp(4-Br), Trp(7-1), Trp(6-1), Trp(5-1), Trp(4-1), Trp(5-OH), αMe-Trp, Tpi, 7-azatryptophan (7-Aza), Nal, 2-pyridylalanine (2-Pal), 3-pyridylalanine (3-Pal), 4-pyridylalanine (4-Pal), 2-quinoylalanine, or 3-quinoylalanine;
Xaa⁴ is Ala, D-Ala, or Ser;
Xaa⁵ is Val, 2,3-dehydro-Val, cyclopentylglycine (Cpg), L-cyclopropylglycine, L-cyclobutylglycine, or tert-leucine (Tle);
Xaa⁶ is Gly or D-Ala;
Xaa⁷ is His, Trp, Trp(Me), Trp(7-Me), Trp(6-Me), Trp(5-Me), Trp(4-Me), Trp(2-Me), Trp(7-F), Trp(6-F), Trp(5-F), Trp(4-F), Trp(7-Cl), Trp(6-Cl), Trp(5-Cl), Trp(4-Cl), Trp(7-Br), Trp(6-Br), Trp(5-Br), Trp(4-Br), Trp(7-1), Trp(6-1), Trp(5-1), Trp(4-1), Trp(5-OH), αMe-Trp, Nal, 2-pyridylalanine (2-Pal), 3-pyridylalanine (3-Pal), 4-pyridylalanine (4-Pal), 2-quinoylalanine, or 3-quinoylalanine;
Xaa⁸ is statine (Sta), Leu, D-Pro, Phe, cis-4-NHR^{a}-D-Pro or cis-4-OR^{a}-D-Pro, wherein R^{a} is alkyl, cycloalkyl, or aryl;
R is —NH₂ or —NHOH;
m1 is 0 or 1;
m2 is 0 or 1;
   when m1 is 1 and m2 is 0, Xaa⁹-R is a C-terminally amidated (R is —NH₂) or a C-terminally hydroxylamidated (R is —NHOH) amino acid residue selected from Pro, Phe, oxazolidine-4-carboxylic acid (4-oxa-L-Pro), 4,4-difluoroproline (difluoroPro), cis-4-NHR^{a}-D-Pro, cis-4-OR^{a}-D-Pro, Leu, 5,5-dimethyl-1,3-thiazolidine-4-carboxylic acid (Me₂Thz), or thiazoline-4-carboxylic acid (Thz), wherein R^{a} is alkyl, cycloalkyl, or aryl;
   when m1 is 1 and m2 is 1, Xaa⁹ is Pro, Phe, oxazolidine-4-carboxylic acid (4-oxa-L-Pro), 4,4-difluoroproline (difluoroPro), Leu, Me₂Thz (5,5-dimethyl-1,3-thiazolidine-4-carboxylic acid), or thiazoline-4-carboxylic acid (Thz); and Xaa¹⁰-R is a C-terminally amidated (R is —NH₂) or a C-terminally hydroxylamidated (R is —NHOH) amino acid residue selected from Val, Leu, Tle, Ile, Nle, Ala, Gly, or Met;
ψ represents a peptide bond or reduced peptide bond joining Xaa⁸ to Xaa⁹;
each R^{L} is independently absent, —(C₁₋₆ alkyl)-, —(C₁₋₆ alkyl)-C(O)—, —C(O)—, —NH—C(O)—, or —NH—C(S)—;
the linker is a linear or branched chain of n1 units of -Xaa¹¹-, and/or -L¹R¹— and/or -(L¹)₂R¹—, wherein:
   n1 is 1-20;
   each Xaa¹¹ is independently an amino acid;
   each R¹ is, independently, a linear, branched, and/or cyclic C₁₋₂₀ alkylenyl, alkenylenyl and/or alkynylenyl, wherein any carbon bonded to two other carbons is optionally independently replaced by N, S, or O, and carbons are optionally independently substituted with oxo, hydroxyl, sulfhydryl, —SeH, halogen, guanidino, amine, amide, urea, carboxylic acid, sulfonic acid, sulfinic acid, or phosphoric acid;
   L¹ bonds to carbon, wherein each L¹ is independently —S—, —N(R²)C(O)—, —C(O)N(R²)—, —NH—C(O)—NH—, —NH—C(S)—NH—,

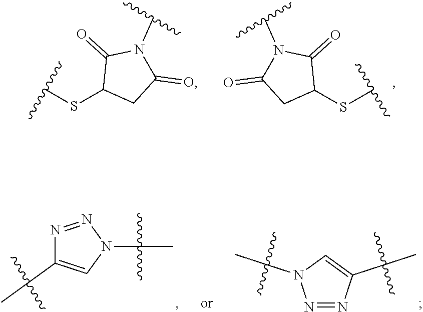

and
R² is H, methyl or ethyl;
optionally, the linker comprises an albumin binder (R^{alb}), wherein the albumin binder is:
   —(CH₂)ₙ₃—CH₃ wherein n3 is 8-20;
   —(CH₂)ₙ₄—C(O)OH wherein n4 is 8-20;

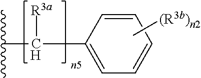

wherein n2 is 1-5, n5 is 1-4, R^{3a} is H or methyl, and R^{3b} is I, Br, F, Cl, H, OH, OCH₃, NH₂, NO₂ or C₁-C₆ alkyl;

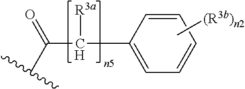

wherein n2 is 1-5, n5 is 1-4, R^{3a} is H or methyl, and R^{3b} is I, Br, F, Cl, H, OH, OCH₃, NH₂, NO₂ or C₁-C₆ alkyl; or

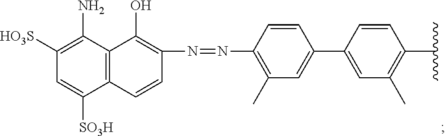 [SMILES: *c1ccc(-c2ccc(N=Nc3ccc4c(S(=O)(=O)O)cc(S(=O)(=O)O)c(N)c4c3O)c(C)c2)cc1C]

n6 is 1-5;
each R^{rad} is a radiolabeling group independently selected from a radiometal chelator; an aryl or heteroaryl substituted with at least one radiohalogen; a prosthetic group containing a trifluoroborate; an aryl or heteroaryl substituted with at least one fluorine precursor leaving group; a prosthetic group containing a silicon-fluorine-acceptor moiety; or a prosthetic group containing a fluorophosphate, fluorosulfate, sulfonyl fluoride, or a combination thereof; wherein:
(i) Xaa³ is αMe-Trp, provided that the compound is not AMTG, AMTG2, and/or LW01186 (DOTA-Pip-D-Phe-Gln-αMe-Trp-Ala-Tle-Gly-His-Leu-ψ-Thz-NH₂ (SEQ ID NO: 1));
(ii) Xaa⁷ is Trp, Trp(Me), Trp(7-Me), Trp(6-Me), Trp(5-Me), Trp(4-Me), Trp(2-Me), Trp(7-F), Trp(6-F), Trp(5-F), Trp(4-F), Trp(7-Cl), Trp(6-Cl), Trp(5-Cl), Trp(4-Cl), Trp(7-Br), Trp(6-Br), Trp(5-Br), Trp(4-Br), Trp(7-1), Trp(6-1), Trp(5-1), Trp(4-1), Trp(5-OH), αMe-Trp, Nal, 2-pyridylalanine (2-Pal), 3-pyridylalanine (3-Pal), 4-pyridylalanine (4-Pal), 2-quinoylalanine (2-Quin-Ala), or 3-quinoylalanine (3-Quin-Ala);
(iii) Xaa² is Gly, Arg, Asp, Glu, citrulline (Cit), 2-amino-4-ureidobutanoic acid (Aub), or 2-amino-4-guanidinobutyric acid (Agb);
(iv) m2 is 1;
(v) at least one R^{rad} is CROWN, CROWNGA, TCMC, PSC, a prosthetic group containing a trifluoroborate, an aryl or heteroaryl substituted with at least one radiohalogen, or an aryl or heteroaryl substituted with at least one fluorine precursor leaving group;
(vi) m1 is 0; or
(vii) any combinations of (i)-(vi); and
   wherein Xaa¹, Xaa², Xaa³, Xaa⁴, Xaa⁵, Xaa⁶, Xaa⁷, Xaa⁸, Xaa⁹ or Xaa¹⁰ is each optionally N-methylated.

In another aspect, this disclosure provides a peptidic compound of Formula (B-1), or a salt or solvate thereof:

(B-1)
R^{rad}-[linker]-Xaa¹³-[Xaa¹²-Xaa¹-Xaa²-Xaa³-Xaa⁴-
Xaa⁵-Xaa⁶-Xaa⁷-Xaa⁸-ψ-Xaa⁹-(Xaa¹⁰)ₘ₂-R]₂

wherein:
Xaa¹² is an N-terminal amino acid residue selected from p-aminomethylaniline-diglycolic acid (pABzA-DIG), 4-amino-(1-carboxymethyl)piperidine (Pip), 9-amino-4,7-dioxanonanoic acid (dPEG2), or 4-(2-aminoethyl)-1-carboxymethyl-piperazine (Acp);
Xaa¹ is D-Phe, 4-chlorophenylalanine (Cpa), D-Cpa, 3-(1-naphthyl)alanine (Nal), D-Nal, 3-(2-naphthyl)alanine (2-Nal), D-2-Nal, 2,3,4,9-tetrahydro-1H-pyrido[3,4-b]indol-3-carboxylic acid (Tpi), or D-Tpi;
Xaa² is Gly, Arg, Asp, Asn, Gln, Glu, homoserine (Hse), citrulline (Cit), 2-amino-4-ureidobutanoic acid (Aub), 2-amino-4-guanidinobutyric acid (Agb), or His;
Xaa³ is Trp, β-(3-benzothienyl)alanine (Bta), Trp(Me), Trp(7-Me), Trp(6-Me), Trp(5-Me), Trp(4-Me), Trp(2-Me), Trp(7-F), Trp(6-F), Trp(5-F), Trp(4-F), Trp(7-Cl), Trp(6-Cl), Trp(5-Cl), Trp(4-Cl), Trp(7-Br), Trp(6-Br), Trp(5-Br), Trp(4-Br), Trp(7-1), Trp(6-1), Trp(5-1), Trp(4-1), Trp(5-OH), αMe-Trp, Tpi, 7-azatryptophan (7-Aza), Nal, 2-pyridylalanine (2-Pal), 3-pyridylalanine (3-Pal), 4-pyridylalanine (4-Pal), 2-quinoylalanine (2-Quin-Ala), or 3-quinoylalanine (3-Quin-Ala);
Xaa⁴ is Ala, D-Ala, or Ser;
Xaa⁵ is Val, 2,3-dehydro-Val, cyclopentylglycine (Cpg), L-cyclopropylglycine, L-cyclobutylglycine, or tert-leucine (Tle);
Xaa⁶ is Gly or D-Ala;
Xaa⁷ is His, Trp, Trp(Me), Trp(7-Me), Trp(6-Me), Trp(5-Me), Trp(4-Me), Trp(2-Me), Trp(7-F), Trp(6-F), Trp(5-F), Trp(4-F), Trp(7-Cl), Trp(6-Cl), Trp(5-Cl), Trp(4-Cl), Trp(7-Br), Trp(6-Br), Trp(5-Br), Trp(4-Br), Trp(7-1), Trp(6-1), Trp(5-1), Trp(4-1), Trp(5-OH), αMe-Trp, Nal, 2-pyridylalanine (2-Pal), 3-pyridylalanine (3-Pal), 4-pyridylalanine (4-Pal), 2-quinoylalanine (2-Quin-Ala), or 3-quinoylalanine (3-Quin-Ala);
Xaa⁸ is statine (Sta), Leu, D-Pro, or Phe;
R is —NH₂ or —NHOH;
m2 is 0 or 1;
   when m2 is 0, Xaa⁹-R is a C-terminally amidated (R is —NH₂) or a C-terminally hydroxylamidated (R is —NHOH) amino acid residue selected from Pro, Phe, oxazolidine-4-carboxylic acid (4-oxa-L-Pro), 4,4-difluoroproline (difluoroPro), cis-4-NHR^{a}-D-Pro, cis-4-OR^{a}-D-Pro, Leu, 5,5-dimethyl-1,3-thiazolidine-4-carboxylic acid (Me₂Thz), or thiazoline-4-carboxylic acid (Thz), wherein R^{a} is alkyl, cycloalkyl, or aryl; when m2 is 1, Xaa⁹ is Pro, Phe, oxazolidine-4-carboxylic acid (4-oxa-L-Pro), 4,4-difluoroproline (difluoroPro), Leu, 5,5-dimethyl-1,3-thiazolidine-4-carboxylic acid (Me₂Thz), or thiazoline-4-carboxylic acid (Thz); and Xaa¹⁰-R is a C-terminally amidated (R is —NH₂) or a C-terminally hydroxylamidated (R is —NHOH) amino acid residue selected from Val, Leu, Tle, Ile, Nle, Ala, Gly, or Met;
Xaa¹³ is an amino acid with a sidechain comprising a carboxylic acid, wherein one Xaa¹² is bonded to the C-terminus of Xaa¹³ and the other Xaa¹² is bonded to the sidechain of Xaa¹³;
ψ represents a peptide bond or reduced peptide bond joining Xaa⁸ to Xaa⁹;
the linker absent or is a linear or branched chain of n1 units of -Xaa¹¹-, wherein:
   n1 is 1-6;
   each Xaa¹¹ is independently an amino acid;
R^{rad} is a radiolabeling group independently selected from a radiometal chelator; an aryl or heteroaryl substituted with at least one radiohalogen; a prosthetic group containing a trifluoroborate; an aryl or heteroaryl substituted with at least one fluorine precursor leaving group; a prosthetic group containing a silicon-fluorine-acceptor moiety; or a prosthetic group containing a fluorophosphate, fluorosulfate, sulfonyl fluoride, or a combination thereof; and wherein Xaa¹, Xaa², Xaa³, Xaa⁴, Xaa⁵, Xaa⁶, Xaa⁷, Xaa⁸, Xaa⁹ or Xaa¹⁰ is each optionally N-methylated.

In still another aspect, the disclosure provides a composition comprising any one of the peptidic compounds as described herein and a pharmaceutically acceptable carrier or excipient.

In a further aspect, the disclosure provides a method of imaging gastrin-releasing peptide receptor (GRPR)-expressing tissue in a subject, the method comprising administering to the subject any one of the peptidic compounds described herein or compositions thereof, and imaging tissue of the subject.

In an additional aspect, the disclosure provides a method of treating cancer in a subject comprising, administering to the subject in need thereof a therapeutically effective amount of any one of the peptidic compounds described herein or compositions thereof.

### DETAILED DESCRIPTION

As used herein, the terms “comprising,” “having”, “including” and “containing,” and grammatical variations thereof, are inclusive or open-ended and do not exclude additional, unrecited elements and/or method steps, even if a feature/component defined as a part thereof consists or consists essentially of specified feature(s)/component(s). The term “consisting essentially of” if used herein in connection with a compound, composition, use or method, denotes that additional elements and/or method steps may be present, but that these additions do not materially affect the manner in which the recited compound, composition, method or use functions. The term “consisting of” if used herein in connection with a feature of a compound, composition, use or method, excludes the presence of additional elements and/or method steps in that feature. A compound, composition, use or method described herein as comprising certain elements and/or steps may also, in certain embodiments consist essentially of those elements and/or steps, and in other embodiments consist of those elements and/or steps, whether or not these embodiments are specifically referred to. A use or method described herein as comprising certain elements and/or steps may also, in certain embodiments consist essentially of those elements and/or steps, and in other embodiments consist of those elements and/or steps, whether or not these embodiments are specifically referred to.

A reference to an element by the indefinite article “a” does not exclude the possibility that more than one of the elements is present, unless the context clearly requires that there be one and only one of the elements. The singular forms “a”, “an”, and “the” include plural referents unless the context clearly dictates otherwise. The use of the word “a” or “an” when used herein in conjunction with the term “comprising” may mean “one,” but it is also consistent with the meaning of “one or more,” “at least one” and “one or more than one.”

The term “about” can mean within an acceptable error range for the particular value as determined by one of ordinary skill in the art, which will depend in part on how the value is measured or determined, i.e., the limitations of the measurement system. For example, “about” can refer to a range of ±20%, ±15%, ±10%, ±5%, or ±1% of a given value.

In this disclosure, the recitation of numerical ranges by endpoints includes all numbers subsumed within that range including all whole numbers, all integers and, where suitable, all fractional intermediates (e.g., 1 to 5 may include 1, 1.5, 2, 2.75, 3, 3.80, 4, and 5 etc.).

Unless otherwise specified, “certain embodiments”, “various embodiments”, “an embodiment” and similar terms includes the particular feature(s) described for that embodiment either alone or in combination with any other embodiment or embodiments described herein, whether or not the other embodiments are directly or indirectly referenced and regardless of whether the feature or embodiment is described in the context of a method, product, use, composition, compound, etcetera.

As used herein, the terms “treat”, “treatment”, “therapeutic” and the like includes ameliorating symptoms, reducing disease progression, improving prognosis and reducing recurrence.

As used herein, the term “diagnostic agent” includes an “imaging agent”. As such, a “diagnostic radionuclide” includes radionuclides that are suitable for use in imaging agents.

The term “subject” refers to an animal (e.g., a mammal or a non-mammal animal). The subject may be a human or a non-human primate. The subject may be a laboratory mammal (e.g., mouse, rat, rabbit, hamster and the like). The subject may be an agricultural animal (e.g., equine, ovine, bovine, porcine, camelid and the like) or a domestic animal (e.g., canine, feline and the like). In some embodiments, the subject is a human.

The compounds disclosed herein may also include base-free forms, solvates, salts or pharmaceutically acceptable salts thereof. Unless otherwise specified or indicated, the compounds claimed and described herein are meant to include all racemic mixtures and all individual enantiomers or combinations thereof, whether or not they are explicitly represented herein.

The compounds disclosed herein may be shown as having one or more charged groups, may be shown with ionizable groups in an uncharged (e.g., protonated) state or may be shown without specifying formal charges. As will be appreciated by a person of skill in the art, the ionization state of certain groups within a compound (e.g., without limitation, COOH, and the like) is dependent, inter alia, on the pKa of that group and the pH at that location. For example, but without limitation, a carboxylic acid group (i.e. COOH) would be understood to usually be deprotonated (and negatively charged) at neutral pH and at most physiological pH values, unless the protonated state is stabilized.

As used herein, the terms “salt” and “solvate” have their usual meaning in chemistry. As such, when the compound is a salt or solvate, it is associated with a suitable counter-ion. It is well known in the art how to prepare salts or to exchange counter-ions. Generally, such salts can be prepared by reacting free acid forms of these compounds with a stoichiometric amount of a suitable base (e.g., without limitation, Na, Ca, Mg, or K hydroxide, carbonate, bicarbonate, or the like), or by reacting free base forms of these compounds with a stoichiometric amount of a suitable acid. Such reactions are generally carried out in water or in an organic solvent, or in a mixture of the two. Counter-ions may be changed, for example, by ion-exchange techniques such as ion-exchange chromatography. All zwitterions, salts, solvates and counter-ions are intended, unless a particular form is specifically indicated.

In certain embodiments, the salt or counter-ion may be pharmaceutically acceptable, for administration to a subject. As used herein, “pharmaceutically acceptable” means suitable for in vivo use in a subject, and is not necessarily restricted to therapeutic use, but also includes diagnostic use. More generally, with respect to any pharmaceutical composition disclosed herein, non-limiting examples of suitable excipients include any suitable buffers, stabilizing agents, salts, antioxidants, complexing agents, tonicity agents, cryoprotectants, lyoprotectants, suspending agents, emulsifying agents, antimicrobial agents, preservatives, chelating agents, binding agents, surfactants, wetting agents, non-aqueous vehicles such as fixed oils, or polymers for sustained or controlled release. See, for example, Berge et al. 1977. (J. Pharm Sci. 66:1-19), or Remington—The Science and Practice of Pharmacy, 21st edition (Gennaro et al editors. Lippincott Williams & Wilkins Philadelphia), each of which is incorporated by reference in its entirety.

As used herein, the expression “Cₙ” where n is an integer (e.g., 1, 2, 3, 4, 5, 6, 7, 8, 9, 10, 11, 12, 13, 14, 15, 16, 17, 18, 19, 20, and the like) or where n is defined as a range of integers (e.g., 1-20, 1-18, 2-15, 3-20, and the like) refers to the number of carbons in a compound, R-group, L-group, or substituent, or refers to the number of carbons plus heteroatoms in a compound, R-group, L-group, or substituent. A range of integers includes all integers in the range; e.g., the range 1-20 includes the integers 1, 2, 3, 4, 5, 6, 7, 8, 9, 10, 11, 12, 13, 14, 15, 16, 17, 18, 19, and 20. Unless otherwise defined, heteroatoms may include any, some or all possible heteroatoms. For example, in some embodiments, the heteroatoms may be selected from N, O, S, P and Se. In some embodiments, the heteroatoms are selected from N, S, or O. Such embodiments are non-limiting. The alternative expression “Cy-Cz”, where y and z are integers (e.g., C₃-C₁₅ and the like), is equivalent to “Cₙ” where n is a range of integers from y to z.

The terms “alkyl”, “alkylenyl”, “alkenylenyl”, and “alkynylenyl” have their usual meanings in organic chemistry. For example, an “alkyenylenyl” has at least one carbon-carbon double bond, and may have any number of carbon-carbon single bonds. Similarly, an “alkynylenyl” has at least one carbon-carbon triple bond, and may have any number of carbon-carbon single bonds. The expressions “alkylenyl, alkenylenyl and/or alkynylenyl” and “alkylenyl, alkenylenyl or alkynylenyl” are intended to be equivalent and each includes hydrocarbon chains that can have any reasonable number or combination of carbon-carbon single bonds, double bonds, and triple bonds. These hydrocarbon chains can be linear, branched, cyclic, or any combination of linear and branched, linear and cyclic, cyclic and branched, branched and cyclic, or linear, branched and cyclic. Cyclic hydrocarbons may be nonaromatic, partially aromatic, or aromatic. Unless otherwise specified, the term “cyclic” includes single rings, multiple non-fused rings, fused rings, bridged rings, and combinations thereof.

The expression “wherein any carbon . . . is optionally independently replaced by N, S, or O” and other similar expressions means that the defined hydrocarbon (e.g., “alkyl”, “alkylenyl”, “alkenylenyl”, or “alkynylenyl”) includes zero, one, more than one, or any reasonable combination of two or more heteroatoms selected from N, S, and O. The above expression therefore expands the defined hydrocarbon to additionally encompass heteroalkyls, heteroalkylenyls, heteroalkenylenyls, and heteroalkynylenyls, etc. A person of skill in the art would understand that various combinations of different heteroatoms may be used. The expression “wherein any carbon bonded to two other carbons is optionally independently replaced by N, S, or O” and other similar expressions means that any carbon in the defined hydrocarbon bonded to two other carbons (e.g., the underlined carbon in —C—C—C—), whether those bonds are single, double, or triple bonds, may be a heteroatom, but excludes heteroatoms bonded to other heteroatoms (e.g., excludes —C—N—S—, —S—S—N—, —N—S—C—, and the like).

Various R-groups (e.g., R¹, R², R³, etc.) and L-groups (e.g., L¹, L², L³, etc.) are defined in this disclosure. Some groups are divalent (e.g., —S—, —NH—C(O)—, —C(O)—NH—, —N(alkyl)-C(O)—, —C(O)—N(alkyl)-, —NH—C(O)—NH—, —NH—C(S)—NH—,

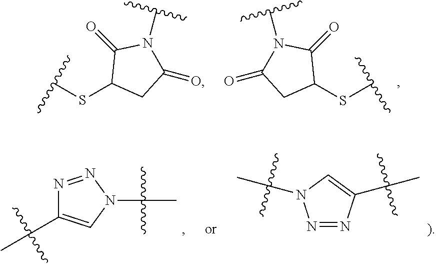

If unspecified, the size of an R-group or L-group is what would be considered reasonable to a person of skill in the art. For example, but without limitation, if unspecified, the size of an alkyl may be 1, 2, 3, 4, 5, 6, 7, 8, 9, 10, 11, 12, 13, 14, 15, 16, 17, 18, 19, 20 or more than 20 carbons in length, subject to the common general knowledge of the person of skill in the art. Further, but without limitation, if unspecified, the size of a heteroalkyl may be 1, 2, 3, 4, 5, 6, 7, 8, 9, 10, 11, 12, 13, 14, 15, 16, 17, 18, 19, 20 or more than 20 carbons and heteroatoms in length, subject to the common general knowledge of the person of skill in the art. In the context of the expression “alkyl, alkenyl or alkynyl” and similar expressions, the “alkyl” would be understood to be a saturated alkyl, and the “alkenyl” and the “alkynyl” would be understood to be unsaturated.

As used herein, in the context of an alkyl/heteroalkyl group of a compound, the term “linear” may be used as it is normally understood to a person of skill in the art and generally refers to a chemical entity that comprises a skeleton or main chain that does not split off into more than one contiguous chain. Non-limiting examples of linear alkyls include methyl, ethyl, n-propyl, and n-butyl.

As used herein, the term “branched” may be used as it is normally understood to a person of skill in the art and generally refers to a chemical entity that comprises a skeleton or main chain that splits off into more than one contiguous chain. The portions of the skeleton or main chain that split off in more than one direction may be linear, cyclic or any combination thereof. Non-limiting examples of a branched alkyl group include tert-butyl and isopropyl.

The term “alkylenyl” refers to a divalent analog of an alkyl group. In the context of the expression “alkylenyl, alkenylenyl and/or alkynylenyl”, and similar expressions, the “alkylenyl” would be understood to be a saturated alkylenyl, and the “alkenylenyl” and the “alkynylenyl” would be understood to be unsaturated. The term “heteroalkylenyl” refers to a divalent analog of a heteroalkyl group. The term “heteroalkenylenyl” refers to a divalent analog of a heteroalkenyl group. The term “heteroalkynylenyl” refers to a divalent analog of a heteroalkynyl group.

As used herein, the term “saturated” when referring to a chemical entity may be used as it is normally understood to a person of skill in the art and generally refers to a chemical entity that comprises only single bonds, and may include linear, branched, and/or cyclic groups. Non-limiting examples of a saturated C₁-C₂₀ alkyl group may include methyl, ethyl, n-propyl, i-propyl, sec-propyl, n-butyl, i-butyl, sec-butyl, t-butyl, n-pentyl, i-pentyl, sec-pentyl, t-pentyl, n-hexyl, i-hexyl, 1,2-dimethylpropyl, 2-ethylpropyl, 1-methyl-2-ethylpropyl, I-ethyl-2-methylpropyl, 1,1,2-trimethylpropyl, 1,1,2-triethylpropyl, 1,1-dimethylbutyl, 2,2-dimethylbutyl, 2-ethylbutyl, 1,3-dimethylbutyl, 2-methylpentyl, 3-methylpentyl, sec-hexyl, t-hexyl, n-heptyl, i-heptyl, sec-heptyl, t-heptyl, n-octyl, i-octyl, sec-octyl, t-octyl, n-nonyl, i-nonyl, sec-nonyl, t-nonyl, n-decyl, i-decyl, sec-decyl, t-decyl, cyclopropanyl, cyclobutanyl, cyclopentanyl, cyclohexanyl, cycloheptanyl, cyclooctanyl, cyclononanyl, cyclodecanyl, and the like. Unless otherwise specified, a C₁-C₂₀ alkylenyl therefore encompasses, without limitation, all divalent analogs of the above-listed saturated alkyl groups.

As used herein, the term “unsaturated” when referring to a chemical entity may be used as it is normally understood to a person of skill in the art and generally refers to a chemical entity that comprises at least one double or triple bond, and may include linear, branched, and/or cyclic groups. Non-limiting examples of a C₂-C₂₀ alkenyl group may include vinyl, allyl, isopropenyl, I-propene-2-yl, 1-butene-1-yl, I-butene-2-yl, I-butene-3-yl, 2-butene-1-yl, 2-butene-2-yl, octenyl, decenyl, cyclopropenyl, cyclobutenyl, cyclopentenyl, cyclohexenyl, cycloheptenyl, cyclooctenyl, cyclononanenyl, cyclodecanenyl, and the like. Unless otherwise specified, a C₁-C₂₀ alkenylenyl therefore encompasses, without limitation, all divalent analogs of the above-listed alkenyl groups. Non-limiting examples of a C₂-C₂₀ alkynyl group may include ethynyl, propynyl, butynyl, pentynyl, hexynyl, heptynyl, octynyl, nonynyl, decynyl, and the like. Unless otherwise specified, a C₁-C₂₀ alkynylenyl therefore encompasses, without limitation, all divalent analogs of the above-listed alkynyl groups.

Non-limiting examples of non-aromatic cyclic groups include cylcopropyl, cyclobutyl, cyclopentyl, cyclohexyl, and the like. Non-limiting examples of non-aromatic heterocyclic groups include aziridinyl, azetidinyl, diazetidinyl, pyrrolidinyl, pyrrolinyl, piperidinyl, piperazinyl, imidazolinyl, pyrazolidinyl, imidazolydinyl, phthalimidyl, succinimidyl, oxiranyl, tetrahydropyranyl, oxetanyl, dioxanyl, thietanyl, thiepinyl, morpholinyl, oxathiolanyl, and the like.

Unless further specified, an “aryl” group includes both single aromatic rings as well as fused rings containing at least one aromatic ring. non-limiting examples of C₃-C₂₀ aryl groups include phenyl (Ph), pentalenyl, indenyl, naphthyl and azulenyl. Non-limiting examples of aromatic heterocyclic groups of similar size include pyrrolyl, imidazolyl, pyrazolyl, pyridinyl, pyridazinyl, pyrimidinyl, pirazinyl, quinolinyl, isoquinolinyl, acridinyl, indolyl, isoindolyl, indolizinyl, purinyl, carbazolyl, indazolyl, phthalazinyl, naphthyridinyl, quinoxalinyl, quinazolinyl, cinnolinyl, pteridinyl, phenanthridinyl, phenazinyl, phenanthrolinyl, perimidinyl, furyl, dibenzofuryl, xanthenyl, benzofuryl, thiophenyl, thianthrenyl, benzothiophenyl, phosphorinyl, phosphinolinyl, phosphindolyl, thiazolyl, oxazolyl, isoxazolyl, and the like.

As used herein, the term “substituted” is used as it would normally be understood to a person of skill in the art and generally refers to a compound or chemical entity that has one chemical group replaced with a different chemical group. Unless otherwise specified, a substituted alkyl, alkylenyl, alkenylenyl, or alkynylenyl has one or more hydrogen atom(s) independently replaced with an atom that is not hydrogen. For example, chloromethyl is a non-limiting example of a substituted alkyl, more particularly an example of a substituted methyl. Aminoethyl is another non-limiting example of a substituted alkyl, more particularly an example of a substituted ethyl. Unless otherwise specified, a substituted compound or group (e.g., R-group or L-group) may be substituted with any chemical group reasonable to a person of skill in the art. For example, but without limitation, a hydrogen bonded to a carbon or heteroatom (e.g., N) may be substituted with halide (e.g., F, I, Br, Cl), amine, amide, oxo, hydroxyl, thiol, phosphate, phosphonate, sulfate, SO₂H, SO₃H, alkyls, heteroalkyls, aryl, heteroaryl, ketones, carboxaldehyde, carboxylates, carboxamides, nitriles, monohalomethyl, dihalomethyl or trihalomethyl. In some embodiments, each carbon may be independently substituted or unsubstituted with oxo, hydroxyl, sulfhydryl, amine, amide, urea, halogen, guanidino, carboxylic acid, sulfonic acid, sulfinic acid, or phosphoric acid. In some embodiments, the amide substituent is —C(O)—NH₂.

As used herein, the term “unsubstituted” is used as it would normally be understood to a person of skill in the art. Non-limiting examples of unsubstituted alkyls include methyl, ethyl, tert-butyl, pentyl and the like. The expression “optionally substituted” is used interchangeably with the expression “unsubstituted or substituted”. The expression “optionally independently substituted” means that each location may be substituted or may not be substituted, and when substituted each substituent may be the same or different.

In the structures provided herein, hydrogen may or may not be shown. In some embodiments, hydrogens (whether shown or implicit) may be protium (i.e. ¹H), deuterium (i.e. ²H) or combinations of ¹H and ²H. Methods for exchanging ¹H with ²H are well known in the art. For solvent-exchangeable hydrogens, the exchange of ¹H with ²H occurs readily in the presence of a suitable deuterium source, without any catalyst. The use of acid, base or metal catalysts, coupled with conditions of increased temperature and pressure, can facilitate the exchange of non-exchangeable hydrogen atoms, generally resulting in the exchange of all ¹H to ²H in a molecule.

The term “Xaa” refers to an amino acid residue in a peptide chain or an amino acid that is otherwise part of a compound. Amino acids have both an amino group and a carboxylic acid group, either or both of which can be used for covalent attachment. In attaching to the remainder of the compound, the amino group and/or the carboxylic acid group may be converted to an amide or other structure; e.g., a carboxylic acid group of a first amino acid is converted to an amide (i.e. a peptide bond) when bonded to the amino group of a second amino acid. As such, Xaa may have the formula —N(R^{a})R^{b}C(O)—, where R^{a} and R^{b} are R-groups. R^{a} will typically be hydrogen or alkyl (e.g., methyl) or R^{a} and R^{b} may form a cyclic structure. The amino acid residues of a peptide may comprise typical peptide (amide) bonds and may further comprise bonds between side chain functional groups and the side chain or main chain functional group of another amino acid. For example, the side chain carboxylate of one amino acid residue in the peptide (e.g., Asp, Glu, etc.) may be bonded to and the amine of another amino acid residue in the peptide (e.g., Dap, Dab, Orn, Lys). Further details are provided below. Unless otherwise indicated, “Xaa” may be any amino acid, including a proteinogenic or nonproteinogenic amino acid. Non-limiting examples of nonproteinogenic amino acids are shown in Table A and include: D-amino acids (including without limitation any D-form of the following amino acids), ornithine (Orn), 3-(1-naphtyl)alanine (Nal), 3-(2-naphtyl)alanine (2-Nal), a-aminobutyric acid, norvaline, norleucine (Nle), homonorleucine, beta-(1,2,3-triazol-4-yl)-L-alanine, 1,2,4-triazole-3-alanine, Phe(4-F), Phe(4-Cl), Phe(4-Br), Phe(4-1), Phe(4-NH₂), Phe(4-NO₂), homoarginine (hArg), 2-amino-4-guanidinobutyric acid (Agb), 2-amino-3-guanidinopropionic acid (Agp), B-alanine, 4-aminobutyric acid, 5-aminovaleric acid, 6-aminohexanoic acid, 7-aminoheptanoic acid, 8-aminooctanoic acid, 9-aminononanoic acid, 10-aminodecanoic acid, 2-aminooctanoic acid, 2-amino-3-(anthracen-2-yl)propanoic acid, 2-amino-3-(anthracen-9-yl)propanoic acid, 2-amino-3-(pyren-1-yl)propanoic acid, Trp(5-Br), Trp(5-OCH₃), Trp(6-F), Trp(5-OH) or Trp(CHO), 2-aminoadipic acid (2-Aad), 3-aminoadipic acid (3-Aad), propargylglycine (Pra), homopropargylglycine (Hpg), beta-homopropargylglycine (Bpg), 2,3-diaminopropionic acid (Dap), 2,4-diaminobutyric acid (Dab), azidolysine (Lys(N₃)), azido-ornithine (Orn(N₃)), 2-amino-4-azidobutanoic acid Dab(N₃), Dap(N₃), 2-(5′-azidopentyl)alanine, 2-(6′-azidohexyl)alanine, 4-amino-1-carboxymethyl-piperidine (Pip), 4-(2-aminoethyl)-1-carboxymethyl-piperazine (Acp), tranexamic acid, tert-leucine (Tle), 4-chlorophenylalanine (Cpa), thiazoline-4-carboxylic acid (Thz), αMe-Trp, p-aminomethylaniline-diglycolic acid (pABzA-DIG), 4-amino-1-carboxymethyl-piperidine (Pip), NH₂(CH₂)₂O(CH₂)₂C(O)OH, NH₂(CH₂)₂[O(CH₂)₂]₂C(O)OH (dPEG2), NH₂(CH₂)₂[O(CH₂)₂]₃C(O)OH, NH₂(CH₂)₂[O(CH₂)₂]₄C(O)OH, NH₂(CH₂)₂[O(CH₂)₂]₅C(O)OH, NH₂(CH₂)₂[O(CH₂)₂]₆C(O)OH, oxazolidine-4-carboxylic acid (4-oxa-L-Pro), 4,4-difluoroproline (diFPro), statine (Sta), β-(3-benzothienyl)alanine (Bta), citrulline (Cit), Trp(Me), Trp (7-Me), Trp(6-Me), Trp(5-Me), Trp(4-Me), Trp(2-Me), Trp(7-F), Trp(5-F), Trp(4-F) or cyclopentylglycine (Cpa). If not specified as an L- or D-amino acid, then the amino acid includes both L- and D-amino acid. If an unspecified (with respect to L- or D-) amino acid is listed together with its corresponding D-amino acid (e.g., Pro and D-Pro), then the unspecified amino acid is understood to be an L-amino acid.

TABLE A
List of non-limiting examples of non-proteinogenic amino acids.

| | |
|---|---|
| p-aminomethylaniline-diglycolic acid (pABzA-DIG) | 2-amino-3-(anthracen-2-yl)propanoic acid |
| ornithine (Orn) | 2-amino-3-(anthracen-9-yl)propanoic acid |
| 3-(1-naphtyl)alanine (Nal) | 2-amino-3-(pyren-1-yl)propanoic acid |
| 3-(2-naphtyl)alanine (2-Nal) | Trp(5-Br) |
| α-aminobutyric acid | Trp(5-OCH₃) |
| norvaline | Trp(6-F) |
| norleucine (Nle) | Trp(5-OH) |
| homonorleucine | Trp(CHO) |
| beta-(1,2,3-triazol-4-yl)-L-alanine | N^{ε},N^{ε},N^{ε}-trimethyl-lysine |
| 1,2,4-triazole-3-alanine | cysteic acid |
| Phe(4-F), Phe(2-F), Phe(3-F), | 2-aminoadipic acid (2-Aad) |
| Phe(4-Cl), Phe(2-Cl), Phe(3-Cl), | 3-aminoadipic acid (3-Aad) |
| Phe(4-Br), Phe(2-Br), Phe(3-Br), | propargylglycine (Pra) |
| Phe(4-I), Phe(2-I), Phe(2-I), | homopropargylglycine (Hpg) |
| Phe(4-NH₂), Phe(2-NH₂), Phe(3-NH₂), | beta-homopropargylglycine (Bpg) |
| Phe(4-NO₂), Phe(2-NO₂), Phe(2-NO₂), | 2,3-diaminopropionic acid (Dap) |
| homoarginine (hArg) | 2,4-diaminobutyric acid (Dab) |
| 4-(2-aminoethyl)-1-carboxymethyl-piperazine (Acp) | azidolysine (Lys(N₃)) |
| 2-(5′-azidopentyl)alanine, 2-(6′-azidohexyl)alanine | azido-ornithine (Orn(N₃)) |
| 2-amino-4-guanidinobutyric acid (Agb) | amino-4-azidobutanoic acid (Dab(N₃)) |
| 2-amino-3-guanidinopropionic acid (Agp) | tranexamic acid |
| β-alanine | 4-amino-1-carboxymethyl-piperidine (Pip) |
| 4-aminobutyric acid | NH₂(CH₂)₂O(CH₂)₂C(O)OH |
| 5-aminovaleric acid | NH₂(CH₂)₂[O(CH₂)₂]₂C(O)OH (dPEG2) |
| 6-aminohexanoic acid | NH₂(CH₂)₂[O(CH₂)₂]₃C(O)OH |
| 7-aminoheptanoic acid | NH₂(CH₂)₂[O(CH₂)₂]₄C(O)OH |
| 8-aminooctanoic acid | NH₂(CH₂)₂[O(CH₂)₂]₅C(O)OH |
| 9-aminononanoic acid | NH₂(CH₂)₂[O(CH₂)₂]₆C(O)OH |
| 10-aminodecanoic acid | citrulline (Cit), norcitrulline (nCit) |
| 2-aminooctanoic acid | β-(3-benzothienyl)alanine (Bta) |
| tert-leucine (Tle) | oxazolidine-4-carboxylic acid (4-oxa-L-Pro) |
| 4-chlorophenylalanine (Cpa) | cyclopentylglycine (Cpg) |
| thiazoline-4-carboxylic acid (Thz) | αMe-Trp |
| any N-methylated version of a proteinogenic amino | any D-amino acid of a proteinogenic amino |
| acid or non-proteinogenic amino acid in this Table | acid or any D-amino acid of a |
| | non-proteinogenic amino acid in this Table |
| Trp(Me), Trp(2-Me) | Trp(7-F), Trp(6-F), Trp(5-F), Trp(4-F) |
| Trp(7-Me), Trp(6-Me), Trp(5-Me), Trp(4-Me), | Trp(7-Cl), Trp(6-Cl), Trp(5-Cl), Trp(4-Cl) |
| 2-Pyridylalanine (2-Pal), 3-Pyridylalanine (3-Pal) | Trp(7-Br), Trp(6-Br), Trp(5-Br), Trp(4-Br) |
| 4-Pyridylalanine (4-Pal) | Trp(7-I), Trp(6-I), Trp(5-I), Trp(4-I) |
| 2-Quinoylalanine, 3-Quinoylalanine (3-Quin-Ala) | cyclobutylglycine |
| 2,3,4,9-tetrahydro-1H-pyrido[3,4-b]indol-3-carboxylic | 5,5-dimethyl-1,3-thiazolidine-4-carboxylic |
| acid (Tpi) | acid (Me₂Thz) |
| 7-Aza (7-azatryptophan) | 2,3-dehyrdo-Val |
| 4,4-difluoroproline (diFPro) | cyclopropylglycine |
| cis-4-cyclohexyloxy-D-Pro (cis-4-OCy-D-Pro) | statine (Sta) |
| cis-4-cyclohexylamino-D-Pro (cis-4-NHCy-D-Pro) | 2-amino-4-ureidobutanoic acid (Aub) |

The wavy line “ [symbol] ” symbol shown through or at the end of a bond in a chemical formula (e.g., in the definitions L¹ or R^{A} of Formula I) is intended to define the group on one side of the wavy line, without modifying the definition of the structure on the opposite side of the wavy line. Where an R-group or L-group is bonded on two or more sides, any atoms shown outside the wavy lines are intended to clarify orientation of the defined group. As such, only the atoms between the two wavy lines constitute the definition of the R-group or L-group. When atoms are not shown outside the wavy lines (e.g., L¹), or for a chemical group shown without wavy lines but does have bonds on multiple sides (e.g., —C(O)NH—, and the like), the chemical group should be read from left to right matching the orientation in the formula that the group relates to; e.g., for formula —R^{a}—R^{b}—R^{c}—, the definition of R^{b} as —C(O)NH— would be incorporated into the formula as —R^{a}—C(O)NH—R^{c}— not as —R^{a}—NHC(O)—R^{c}—.

The term “therapeutically effective amount” as used herein refers to an amount effective at the dosage and duration necessary to achieve the desired therapeutic result. A therapeutically effective amount of the composition may vary depending on factors such as the individual's condition, age, sex, and weight, and the ability of the protein to elicit the desired response of the individual. A therapeutically effective amount can also be an amount that exceeds any toxic or deleterious effect of the composition that would have a beneficial effect on the treatment.

### Compounds

Previously, a series of bombesin analogs modifying its C-terminal Leu¹³-Met¹⁴ have been developed. See, Rousseau, E. et al. Journal of Labelled Compounds and Radiopharmaceuticals 63, 56-64 (2020), Lau, J. et al. ACS omega 4, 1470-1478 (2019), and Bratanovic, I. J. et al. J. of Nuclear Medicine July 2021, jnumed.120.257758, each of which is hereby incorporated by reference in its entirety for all purposes.

In one aspect, this disclosure provides a peptidic compound of Formula (A), (I), (IA), (IA-1), (IB), (IB-1), (IC), (IC-1), (ID), (ID-1), (IE), (IE-1), (IF), (IF-1), (II), (IIA), (B), or (B-1) (each as defined below), or a salt or solvate thereof.

In some embodiments, the present disclosure relates to a peptidic compound of Formula (A), or a salt or solvate of Formula (A),

(A)
R^{rad}ₙ₆-R^{L}-[linker]-R^{L}-Xaa¹-Xaa²-Xaa³-Xaa⁴-Xaa⁵-
Xaa⁶-Xaa⁷-Xaa⁸-[ψ-Xaa⁹-(Xaa¹⁰)ₘ₂]ₘ₁-R

wherein:
Xaa¹ is an N-terminal amino acid residue selected from D-Phe, 4-chlorophenylalanine (Cpa), D-Cpa, 3-(1-naphthyl)alanine (Nal), D-Nal, 3-(2-naphthyl)alanine (2-Nal), D-2-Nal, 2,3,4,9-tetrahydro-1H-pyrido[3,4-b]indol-3-carboxylic acid (Tpi), or D-Tpi;
Xaa² is Gly, Arg, Asp, Asn, Gln, Glu, homoserine (Hse), citrulline (Cit), 2-amino-4-ureidobutanoic acid (Aub), 2-amino-4-guanidinobutyric acid (Agb), or His;
Xaa³ is Trp, β-(3-benzothienyl)alanine (Bta), Trp(Me), Trp(7-Me), Trp(6-Me), Trp(5-Me), Trp(4-Me), Trp(2-Me), Trp(7-F), Trp(6-F), Trp(5-F), Trp(4-F), Trp(7-Cl), Trp(6-Cl), Trp(5-Cl), Trp(4-Cl), Trp(7-Br), Trp(6-Br), Trp(5-Br), Trp(4-Br), Trp(7-1), Trp(6-1), Trp(5-1), Trp(4-1), Trp(5-OH), αMe-Trp, Tpi, 7-azatryptophan (7-Aza), Nal, 2-pyridylalanine (2-Pal), 3-pyridylalanine (3-Pal), 4-pyridylalanine (4-Pal), 2-quinoylalanine (2-Quin-Ala), or 3-quinoylalanine (3-Quin-Ala);
Xaa⁴ is Ala, D-Ala, or Ser;
Xaa⁵ is Val, 2,3-dehydro-Val, cyclopentylglycine (Cpg), L-cyclopropylglycine, L-cyclobutylglycine, or tert-leucine (Tle);
Xaa⁶ is Gly or D-Ala;
Xaa⁷ is His, Trp, Trp(Me), Trp(7-Me), Trp(6-Me), Trp(5-Me), Trp(4-Me), Trp(2-Me), Trp(7-F), Trp(6-F), Trp(5-F), Trp(4-F), Trp(7-Cl), Trp(6-Cl), Trp(5-Cl), Trp(4-Cl), Trp(7-Br), Trp(6-Br), Trp(5-Br), Trp(4-Br), Trp(7-1), Trp(6-1), Trp(5-1), Trp(4-1), Trp(5-OH), αMe-Trp, Nal, 2-pyridylalanine (2-Pal), 3-pyridylalanine (3-Pal), 4-pyridylalanine (4-Pal), 2-quinoylalanine (2-Quin-Ala), or 3-quinoylalanine (3-Quin-Ala);
Xaa⁸ is statine (Sta), Leu, D-Pro, Phe, cis-4-NHR^{a}-D-Pro or cis-4-OR^{a}-D-Pro, wherein R^{a} is alkyl, cycloalkyl, or aryl;
R is —NH₂ or —NHOH;
m1 is 0 or 1;
m2 is 0 or 1;
   when m1 is 1 and m2 is 0, Xaa⁹-R is a C-terminally amidated (R is —NH₂) or a C-terminally hydroxylamidated (R is —NHOH) amino acid residue selected from Pro, Phe, oxazolidine-4-carboxylic acid (4-oxa-L-Pro), 4,4-difluoroproline (difluoroPro), cis-4-NHR^{a}-D-Pro, cis-4-OR^{a}-D-Pro, Leu, 5,5-dimethyl-1,3-thiazolidine-4-carboxylic acid (Me₂Thz), or thiazoline-4-carboxylic acid (Thz), wherein R^{a} is alkyl, cycloalkyl, or aryl;
   when m1 is 1 and m2 is 1, Xaa⁹ is Pro, Phe, oxazolidine-4-carboxylic acid (4-oxa-L-Pro), 4,4-difluoroproline (difluoroPro), Leu, 5,5-dimethyl-1,3-thiazolidine-4-carboxylic acid (Me₂Thz), or thiazoline-4-carboxylic acid (Thz); and Xaa¹⁰-R is a C-terminally amidated (R is —NH₂) or a C-terminally hydroxylamidated (R is —NHOH) amino acid residue selected from Val, Leu, Tle, Ile, Nle, Ala, Gly, or Met;
ψ represents a peptide bond or reduced peptide bond joining Xaa⁸ to Xaa⁹;
each R^{L} is independently absent, —(C₁₋₆ alkyl)-, —(C₁₋₆ alkyl)-C(O)—, —C(O)—, —NH—C(O)—, or —NH—C(S)—;
the linker is a linear or branched chain of n1 units of -Xaa¹¹-, and/or -L¹R¹— and/or -(L¹)₂R¹—, wherein:
   n1 is 1-20;
   each Xaa¹¹ is independently an amino acid;
   each R¹ is, independently, a linear, branched, and/or cyclic C₁₋₂₀ alkylenyl, alkenylenyl and/or alkynylenyl, wherein any carbon bonded to two other carbons is optionally independently replaced by N, S, or O, and carbons are optionally independently substituted with oxo, hydroxyl, sulfhydryl, —SeH, halogen, guanidino, amine, amide, urea, carboxylic acid, sulfonic acid, sulfinic acid, or phosphoric acid;
   L¹ bonds to carbon, wherein each L¹ is independently —S—, —N(R²)C(O)—, —C(O)N(R²)—, —NH—C(O)—NH—, —NH—C(S)—NH—

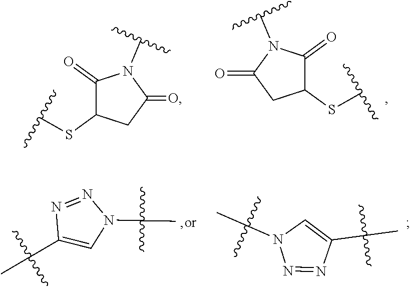

and
R² is H, methyl or ethyl;
optionally, the linker comprises an albumin binder (R^{alb}), wherein the albumin binder is:
   —(CH₂)ₙ₃—CH₃ wherein n3 is 8-20;
   —(CH₂)ₙ₄—C(O)OH wherein n4 is 8-20;

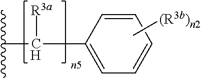

wherein n2 is 1-5, n5 is 1-4, R^{3a} is H or methyl, and R^{3b} is I, Br, F, Cl, H, OH, OCH₃, NH₂, NO₂ or C₁-C₆ alkyl;

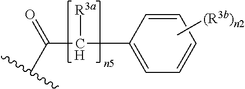

wherein n2 is 1-5, n5 is 1-4, R^{3a} is H or methyl, and R^{3b} is I, Br, F, Cl, H, OH, OCH₃, NH₂, NO₂ or C₁-C₆ alkyl; or

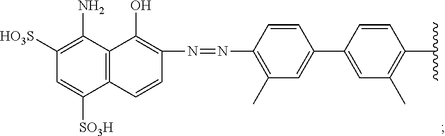 [SMILES: *c1ccc(-c2ccc(N=Nc3ccc4c(S(=O)(=O)O)cc(S(=O)(=O)O)c(N)c4c3O)c(C)c2)cc1C]

n6 is 1-5;
each R^{rad} is a radiolabeling group independently selected from a radiometal chelator; an aryl or heteroaryl substituted with at least one radiohalogen; a prosthetic group containing a trifluoroborate; an aryl or heteroaryl substituted with at least one fluorine precursor leaving group; a prosthetic group containing a silicon-fluorine-acceptor moiety; or a prosthetic group containing a fluorophosphate, fluorosulfate, sulfonyl fluoride, or a combination thereof; and
wherein Xaa¹, Xaa², Xaa³, Xaa⁴, Xaa⁵, Xaa⁶, Xaa⁷, Xaa⁸, Xaa⁹ or Xaa¹⁰ is each optionally N-methylated.

In some embodiments, the present disclosure relates to a peptidic compound of Formula I or a salt or solvate of Formula I

(I)
R^{rad}ₙ₆-[linker]-Xaa¹-Xaa²-Xaa³-Xaa⁴-Xaa⁵-Xaa⁶-
Xaa⁷-Xaa⁸-[ψ-Xaa⁹-(Xaa¹⁰)ₘ₂]ₘ₁-R

wherein:
Xaa¹ is an N-terminal amino acid residue selected from D-Phe, 4-chlorophenylalanine (Cpa), D-Cpa, 3-(1-naphthyl)alanine (Nal), D-Nal, 3-(2-naphthyl)alanine (2-Nal), D-2-Nal, 2,3,4,9-tetrahydro-1H-pyrido[3,4-b]indol-3-carboxylic acid (Tpi), or D-Tpi;
Xaa² is Gly, Arg, Asp, Asn, Gln, Glu, homoserine (Hse), citrulline (Cit), 2-amino-4-ureidobutanoic acid (Aub), 2-amino-4-guanidinobutyric acid (Agb), or His;
Xaa³ is Trp, β-(3-benzothienyl)alanine (Bta), Trp(Me), Trp(7-Me), Trp(6-Me), Trp(5-Me), Trp(4-Me), Trp(2-Me), Trp(7-F), Trp(6-F), Trp(5-F), Trp(4-F), Trp(7-Cl), Trp(6-Cl), Trp(5-Cl), Trp(4-Cl), Trp(7-Br), Trp(6-Br), Trp(5-Br), Trp(4-Br), Trp(7-1), Trp(6-1), Trp(5-1), Trp(4-1), Trp(5-OH), αMe-Trp, Tpi, 7-azatryptophan (7-Aza), Nal, 2-pyridylalanine (2-Pal), 3-pyridylalanine (3-Pal), 4-pyridylalanine (4-Pal), 2-quinoylalanine, or 3-quinoylalanine;
Xaa⁴ is Ala, D-Ala, or Ser;
Xaa⁵ is Val, 2,3-dehydro-Val, cyclopentylglycine (Cpg), L-cyclopropylglycine, L-cyclobutylglycine, or tert-leucine (Tle);
Xaa⁶ is Gly or D-Ala;
Xaa⁷ is His, Trp, Trp(Me), Trp(7-Me), Trp(6-Me), Trp(5-Me), Trp(4-Me), Trp(2-Me), Trp(7-F), Trp(6-F), Trp(5-F), Trp(4-F), Trp(7-Cl), Trp(6-Cl), Trp(5-Cl), Trp(4-Cl), Trp(7-Br), Trp(6-Br), Trp(5-Br), Trp(4-Br), Trp(7-1), Trp(6-1), Trp(5-1), Trp(4-1), Trp(5-OH), αMe-Trp, Nal, 2-pyridylalanine (2-Pal), 3-pyridylalanine (3-Pal), 4-pyridylalanine (4-Pal), 2-quinoylalanine, or 3-quinoylalanine;
Xaa⁸ is statine (Sta), Leu, D-Pro, Phe, cis-4-NHR^{a}-D-Pro or cis-4-OR^{a}-D-Pro, wherein R^{a} is alkyl, cycloalkyl, or aryl;
R is —NH₂ or —NHOH;
m1 is 0 or 1;
m2 is 0 or 1;
   when m1 is 1 and m2 is 0, Xaa⁹-R is a C-terminally amidated (R is —NH₂) or a C-terminally hydroxylamidated (R is —NHOH) amino acid residue selected from Pro, Phe, oxazolidine-4-carboxylic acid (4-oxa-L-Pro), 4,4-difluoroproline (difluoroPro), cis-4-NHR^{a}-D-Pro, cis-4-OR^{a}-D-Pro, Leu, 5,5-dimethyl-1,3-thiazolidine-4-carboxylic acid (Me₂Thz), or thiazoline-4-carboxylic acid (Thz), wherein R^{a} is alkyl, cycloalkyl, or aryl;
   when m1 is 1 and m2 is 1, Xaa⁹ is Pro, Phe, oxazolidine-4-carboxylic acid (4-oxa-L-Pro), 4,4-difluoroproline (difluoroPro), Leu, Me₂Thz (5,5-dimethyl-1,3-thiazolidine-4-carboxylic acid), or thiazoline-4-carboxylic acid (Thz); and
Xaa¹⁰-R is a C-terminally amidated (R is —NH₂) or a C-terminally hydroxylamidated (R is —NHOH) amino acid residue selected from Val, Leu, Tle, Ile, Nle, Ala, Gly, or Met;
ψ represents a peptide bond or reduced peptide bond joining Xaa⁸ to Xaa⁹;
the linker is a linear or branched chain of n1 units of -Xaa¹¹-;
   n1 is 1-20;
   each Xaa¹¹ is independently an amino acid;
   optionally, the linker comprises an albumin binder (R^{alb}), wherein the albumin binder is:
   —(CH₂)ₙ₃—CH₃ wherein n3 is 8-20;
   —(CH₂)ₙ₄—C(O)OH wherein n4 is 8-20;

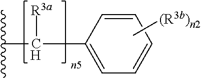

wherein n2 is 1-5, n5 is 1-4, R^{3a} is H or methyl, and R^{3b} is I, Br, F, Cl, H, OH, OCH₃, NH₂, NO₂ or C₁-C₆ alkyl;

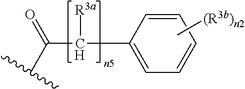

wherein n2 is 1-5, n5 is 1-4, R^{3a} is H or methyl, and R^{3b} is I, Br, F, Cl, H, OH, OCH₃, NH₂, NO₂ or C₁-C₆ alkyl; or

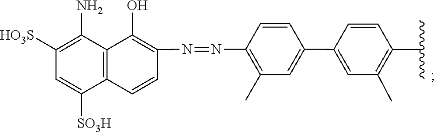 [SMILES: *c1ccc(-c2ccc(N=Nc3ccc4c(S(=O)(=O)O)cc(S(=O)(=O)O)c(N)c4c3O)c(C)c2)cc1C]

n6 is 1-5;
each R^{rad} is a radiolabeling group independently selected from a radiometal chelator; an aryl or heteroaryl substituted with at least one radiohalogen; a prosthetic group containing a trifluoroborate; an aryl or heteroaryl substituted with at least one fluorine precursor leaving group; a prosthetic group containing a silicon-fluorine-acceptor moiety; or a prosthetic group containing a fluorophosphate, fluorosulfate, sulfonyl fluoride, or a combination thereof; and
wherein Xaa¹, Xaa², Xaa³, Xaa⁴, Xaa⁵, Xaa⁶, Xaa⁷, Xaa^{B}, Xaa⁹ or Xaa¹⁰ is each optionally N-methylated.

In some embodiments of the compound of formula (A) or (I), or a salt or solvate of formula (A) or (I):
(i) Xaa³ is αMe-Trp, provided that the compound is not AMTG, AMTG2, and/or LW01186;
(ii) Xaa⁷ is Trp, Trp(Me), Trp(7-Me), Trp(6-Me), Trp(5-Me), Trp(4-Me), Trp(2-Me), Trp(7-F), Trp(6-F), Trp(5-F), Trp(4-F), Trp(7-Cl), Trp(6-Cl), Trp(5-Cl), Trp(4-Cl), Trp(7-Br), Trp(6-Br), Trp(5-Br), Trp(4-Br), Trp(7-1), Trp(6-1), Trp(5-1), Trp(4-1), Trp(5-OH), αMe-Trp, Nal, 2-pyridylalanine (2-Pal), 3-pyridylalanine (3-Pal), 4-pyridylalanine (4-Pal), 2-quinoylalanine (2-Quin-Ala), or 3-quinoylalanine (3-Quin-Ala);
(iii) Xaa² is Gly, Arg, Asp, Glu, citrulline (Cit), 2-amino-4-ureidobutanoic acid (Aub), or 2-amino-4-guanidinobutyric acid (Agb);
(iv) m2 is 1;
(v) at least one R^{rad} is CROWN, CROWNGA, TCMC, PSC, a prosthetic group containing a trifluoroborate, an aryl or heteroaryl substituted with at least one radiohalogen, or an aryl or heteroaryl substituted with at least one fluorine precursor leaving group;
(vi) m1 is 0; or
(vii) any combinations of (i)-(vi).

In some embodiments of the compound of formula (A) or (I), or a salt or solvate of formula (A) or (I), at least one R^{rad} is CROWN, CROWNGA, TCMC, PSC, a prosthetic group containing a trifluoroborate, an aryl or heteroaryl substituted with at least one radiohalogen, or an aryl or heteroaryl substituted with at least one fluorine precursor leaving group. In some embodiments of the compound of formula (A) or (I), or a salt or solvate of formula (A) or (I), at least one R^{rad} is CROWN, TCMC, PSC, a prosthetic group containing a trifluoroborate, an aryl or heteroaryl substituted with at least one radiohalogen, or an aryl or heteroaryl substituted with at least one fluorine precursor leaving group.

In some embodiments, Xaa¹ being an N-terminal amino acid residue D-Phe indicates Xaa¹ having the structure

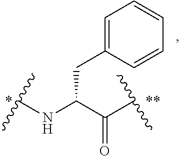 [SMILES: *N[C@H](Cc1ccccc1)C(*)=O]

where * indicates the connectivity to the -[linker]- or to the -[linker]-R^{L}— and ** indicates the connectivity to Xaa². In embodiments, similar configuration is understood with other Xaa¹ amino acids.

In some embodiments of the compound of formula (A) or (I), or salt or solvate of formula (A) or (I), each Xaa¹¹ is independently selected from any proteinogenic amino acid or nonproteinogenic amino acid disclosed in Table A.

In some embodiments, the present disclosure relates to a peptidic compound of Formula (IA), or a salt or solvate of Formula (IA),

(IA)
R^{rad}ₙ₆-[linker]-Xaa¹-Xaa²-Xaa³-Xaa⁴-Xaa⁵-Xaa⁶-
Xaa⁷-Xaa⁸-[ψ-Xaa⁹-(Xaa¹⁰)ₘ₂]ₘ₁-R

wherein:
Xaa¹ is an N-terminal amino acid residue selected from D-Phe, 4-chlorophenylalanine (Cpa), D-Cpa, 3-(1-naphthyl)alanine (Nal), D-Nal, 3-(2-naphthyl)alanine (2-Nal), D-2-Nal, 2,3,4,9-tetrahydro-1H-pyrido[3,4-b]indol-3-carboxylic acid (Tpi), or D-Tpi;
Xaa² is Gly, Arg, Asp, Asn, Gln, Glu, homoserine (Hse), citrulline (Cit), 2-amino-4-ureidobutanoic acid (Aub), 2-amino-4-guanidinobutyric acid (Agb), or His;
Xaa³ is αMe-Trp;
Xaa⁴ is Ala, D-Ala, or Ser;
Xaa⁵ is Val or tert-leucine (Tle);
Xaa⁶ is Gly or D-Ala;
Xaa⁷ is His, Trp, Trp(Me), Trp(7-Me), Trp(6-Me), Trp(5-Me), Trp(4-Me), Trp(2-Me), Trp(7-F), Trp(6-F), Trp(5-F), Trp(4-F), Trp(7-Cl), Trp(6-Cl), Trp(5-Cl), Trp(4-Cl), Trp(7-Br), Trp(6-Br), Trp(5-Br), Trp(4-Br), Trp(7-1), Trp(6-1), Trp(5-1), Trp(4-1), Trp(5-OH), αMe-Trp, Nal, 2-pyridylalanine (2-Pal), 3-pyridylalanine (3-Pal), 4-pyridylalanine (4-Pal), 2-quinoylalanine (2-Quin-Ala), or 3-quinoylalanine (3-Quin-Ala);
Xaa⁸ is statine (Sta), Leu, D-Pro, Phe, cis-4-NHR^{a}-D-Pro or cis-4-OR^{a}-D-Pro, wherein R^{a} is alkyl, cycloalkyl, or aryl;
R is —NH₂ or —NHOH;
m1 is 0 or 1;
when m1 is 1 and m2 is 0, Xaa⁹-R is a C-terminally amidated (R is —NH₂) or a C-terminally hydroxylamidated (R is —NHOH) amino acid residue selected from Pro, Phe, oxazolidine-4-carboxylic acid (4-oxa-L-Pro), 4,4-difluoroproline (difluoroPro), cis-4-NHR^{a}-D-Pro, cis-4-OR^{a}-D-Pro, or Leu, wherein R^{a} is alkyl, cycloalkyl, or aryl;
when m1 is 1 and m2 is 1, Xaa⁹ is Pro, Phe, oxazolidine-4-carboxylic acid (4-oxa-L-Pro), 4,4-difluoroproline (difluoroPro), Leu, 5,5-dimethyl-1,3-thiazolidine-4-carboxylic acid (Me₂Thz), or thiazoline-4-carboxylic acid (Thz); and Xaa¹⁰-R is a C-terminally amidated (R is —NH₂) or a C-terminally hydroxylamidated (R is —NHOH) amino acid residue selected from Val, Leu, Tle, Ile, Nle, Ala, Gly, or Met;
ψ represents a peptide bond or reduced peptide bond joining Xaa⁸ to Xaa⁹;
R^{L} is absent, —(C₁₋₆ alkyl)-, —(C₁₋₆ alkyl)-C(O)—, —C(O)—, —NH—C(O)—, or —NH—C(S)—;
the linker is a linear or branched chain of n1 units of -Xaa¹¹-:
n1 is 1-20;
each Xaa¹¹ is independently an amino acid;
optionally, the linker is bonded to an albumin binder (R^{ab}), wherein R^{alb} is:
—(CH₂)ₙ₃—CH₃ wherein n3 is 8-20;
—(CH₂)ₙ₄—C(O)OH wherein n4 is 8-20;

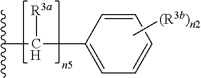

wherein n2 is 1-5, n5 is 1-4, each R^{3a} is independently H or methyl, and each R^{3b} is independently I, Br, F, Cl, H, OH, OCH₃, NH₂, NO₂ or C₁-C₆ alkyl;

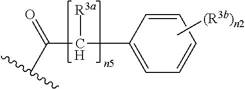

wherein n2 is 1-5, n5 is 1-4, each R^{3a} is independently H or methyl, and each R^{3b} is independently I, Br, F, Cl, H, OH, OCH₃, NH₂, NO₂ or C₁-C₆ alkyl; or

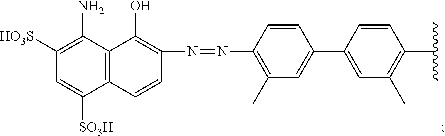 [SMILES: *c1ccc(-c2ccc(N=Nc3ccc4c(S(=O)(=O)O)cc(S(=O)(=O)O)c(N)c4c3O)c(C)c2)cc1C]

n6 is 1-5;
each R^{rad} is a radiolabeling group independently selected from a radiometal chelator; an aryl or heteroaryl substituted with at least one radiohalogen; a prosthetic group containing a trifluoroborate; an aryl or heteroaryl substituted with at least one fluorine precursor leaving group; a prosthetic group containing a silicon-fluorine-acceptor moiety; or a prosthetic group containing a fluorophosphate, fluorosulfate, sulfonyl fluoride, or a combination thereof;
wherein when Xaa⁵ is Val, R^{L} is absent, and the linker is 4-amino-(1-carboxymethyl)piperidine (Pip), then R^{rad} is not DOTA or DOTAGA; and
wherein Xaa¹, Xaa², Xaa³, Xaa⁴, Xaa⁵, Xaa⁶, Xaa⁷, Xaa⁸, Xaa⁹ or Xaa¹⁰ is each optionally N-methylated.

In some embodiments of the compounds of Formula (A), (I) or (IA), the linker and R^{L} together form a p-aminomethylaniline-diglycolic acid (pABzA-DIG) linker, a 4-amino-(1-carboxymethyl)piperidine (Pip) linker, a 9-amino-4,7-dioxanonanoic acid (dPEG2) linker, or a 4-(2-aminoethyl)-1-carboxymethyl-piperazine (Acp) linker.

In some embodiments, the present disclosure relates to a peptidic compound of Formula (IA-1), or a salt or solvate of Formula (IA-1),

(IA-1)
(SEQ ID NO: 2)
R^{rad}-[linker]-Xaa¹-Xaa²-Xaa³-Xaa⁴-Xaa⁵-Xaa⁶-
Xaa⁷-Xaa⁸-ψ-Xaa⁹-(Xaa¹⁰)ₘ₂-R

wherein:
Xaa¹ is an N-terminal amino acid residue of D-Phe;
Xaa² is Gln;
Xaa³ is αMe-Trp;
Xaa⁴ is Ala;
Xaa⁵ is Val or Tle;
Xaa⁶ is Gly;
Xaa⁷ is His;
Xaa⁸ is Sta or Leu;
R is —NH₂ or —NHOH;
when m2 is 0, Xaa⁹-R is a C-terminally amidated (R is —NH₂) or a C-terminally hydroxylamidated (R is —NHOH) amino acid residue selected from Pro or Leu;
when m2 is 1, Xaa⁹ is Pro or Leu; and Xaa¹⁰-R is a C-terminally amidated (R is —NH₂) or a C-terminally hydroxylamidated (R is —NHOH) amino acid residue selected from Val or Tle;
ψ represents a peptide bond or reduced peptide bond joining Xaa⁸ to Xaa⁹;
the linker is

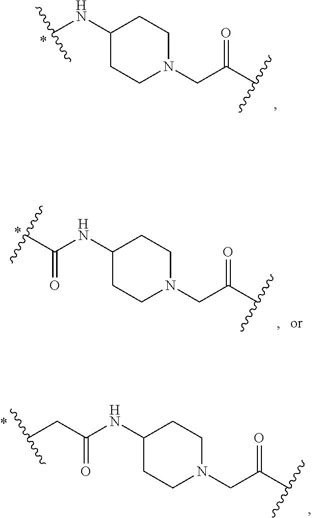 [SMILES: *NC1CCN(CC(*)=O)CC1 ; *C(=O)CN1CCC(NC(*)=O)CC1 ; *CC(=O)NC1CCN(CC(*)=O)CC1]

wherein * indicates the connectivity to R^{rad};
R^{rad} is a radiometal chelator;
wherein when Xaa⁵ is Val, then R^{rad} is not DOTA or DOTAGA; and
wherein Xaa¹, Xaa², Xaa³, Xaa⁴, Xaa⁵, Xaa⁶, Xaa⁷, Xaa⁸, Xaa⁹ or Xaa¹⁰ is each optionally N-methylated.

In some embodiments, the present disclosure relates to a peptidic compound of Formula (IB), or a salt or solvate of Formula (IB),

(IB)
R^{rad}ₙ₆-[linker]-R^{L}-Xaa¹-Xaa²-Xaa³-Xaa⁴-Xaa⁵-
Xaa⁶-Xaa⁷-Xaa⁸-]ψ-Xaa⁹-(Xaa¹⁰)ₘ₂]ₘ₁-R

wherein:
Xaa¹ is an N-terminal amino acid residue selected from D-Phe, 4-chlorophenylalanine (Cpa), D-Cpa, 3-(1-naphthyl)alanine (Nal), D-Nal, 3-(2-naphthyl)alanine (2-Nal), D-2-Nal, 2,3,4,9-tetrahydro-1H-pyrido[3,4-b]indol-3-carboxylic acid (Tpi), or D-Tpi;
Xaa² is Gly, Arg, Asp, Asn, Gln, Glu, homoserine (Hse), citrulline (Cit), 2-amino-4-ureidobutanoic acid (Aub), 2-amino-4-guanidinobutyric acid (Agb), or His;
Xaa³ is Trp, β-(3-benzothienyl)alanine (Bta), Trp(Me), Trp(7-Me), Trp(6-Me), Trp(5-Me), Trp(4-Me), Trp(2-Me), Trp(7-F), Trp(6-F), Trp(5-F), Trp(4-F), Trp(7-Cl), Trp(6-Cl), Trp(5-Cl), Trp(4-Cl), Trp(7-Br), Trp(6-Br), Trp(5-Br), Trp(4-Br), Trp(7-1), Trp(6-1), Trp(5-1), Trp(4-1), Trp(5-OH), αMe-Trp, Tpi, 7-azatryptophan (7-Aza), Nal, 2-pyridylalanine (2-Pal), 3-pyridylalanine (3-Pal), 4-pyridylalanine (4-Pal), 2-quinoylalanine (2-Quin-Ala), or 3-quinoylalanine (3-Quin-Ala);
Xaa⁴ is Ala, D-Ala, or Ser;
Xaa⁵ is Val, 2,3-dehydro-Val, cyclopentylglycine (Cpg), L-cyclopropylglycine, L-cyclobutylglycine, or tert-leucine (Tle);
Xaa⁶ is Gly or D-Ala;
Xaa⁷ is Trp, Trp(Me), Trp(7-Me), Trp(6-Me), Trp(5-Me), Trp(4-Me), Trp(2-Me), Trp(7-F), Trp(6-F), Trp(5-F), Trp(4-F), Trp(7-Cl), Trp(6-Cl), Trp(5-Cl), Trp(4-Cl), Trp(7-Br), Trp(6-Br), Trp(5-Br), Trp(4-Br), Trp(7-1), Trp(6-1), Trp(5-1), Trp(4-1), Trp(5-OH), αMe-Trp, Nal, 2-pyridylalanine (2-Pal), 3-pyridylalanine (3-Pal), 4-pyridylalanine (4-Pal), 2-quinoylalanine (2-Quin-Ala), or 3-quinoylalanine (3-Quin-Ala);
Xaa⁸ is statine (Sta), Leu, D-Pro, Phe, cis-4-NHR^{a}-D-Pro or cis-4-OR^{a}-D-Pro, wherein R^{a} is alkyl, cycloalkyl, or aryl
R is —NH₂ or —NHOH;
m1 is 0 or 1;
m2 is 0 or 1;
   when m1 is 1 and m2 is 0, Xaa⁹-R is a C-terminally amidated (R is —NH₂) or a C-terminally hydroxylamidated (R is —NHOH) amino acid residue selected from Pro, Phe, oxazolidine-4-carboxylic acid (4-oxa-L-Pro), 4,4-difluoroproline (difluoroPro), cis-4-NHR^{a}-D-Pro, cis-4-OR^{a}-D-Pro, Leu, 5,5-dimethyl-1,3-thiazolidine-4-carboxylic acid (Me₂Thz), or thiazoline-4-carboxylic acid (Thz);
   when m1 is 1 and m2 is 1, Xaa⁹ is Pro, Phe, oxazolidine-4-carboxylic acid (4-oxa-L-Pro), 4,4-difluoroproline (difluoroPro), Leu, 5,5-dimethyl-1,3-thiazolidine-4-carboxylic acid (Me₂Thz), or thiazoline-4-carboxylic acid (Thz); and Xaa¹⁰-R is a C-terminally amidated (R is —NH₂) or a C-terminally hydroxylamidated (R is —NHOH) amino acid residue selected from Val, Leu, Tle, Ile, Nle, Ala, Gly, or Met;
      ψ represents a peptide bond or reduced peptide bond joining Xaa⁸ to Xaa⁹;
      R^{L} is absent, —C(O)—, —NH—C(O)—, or —NH—C(S)—;
the linker is a linear or branched chain of n1 units of -Xaa¹¹-, and/or -L¹R¹— and/or -(L¹)₂R¹—, wherein:
   n1 is 1-20;
   each Xaa¹¹ is independently an amino acid;
   each R¹ is, independently, a linear, branched, and/or cyclic C₁₋₂₀ alkylenyl, C₂₋₂₀ alkenylenyl and/or C₂₋₂₀ alkynylenyl, wherein any carbon bonded to two other carbons is optionally independently replaced by N, S, or O, and carbons are optionally independently substituted with oxo, hydroxyl, sulfhydryl, —SeH, halogen, guanidino, amine, amide, urea, carboxylic acid, sulfonic acid, sulfinic acid, or phosphoric acid;
   L¹ bonds to carbon, wherein each L¹ is independently —S—, —N(R²)C(O)—, —C(O)N(R²)—, —NH—C(O)—NH—, —NH—C(S)—NH—,

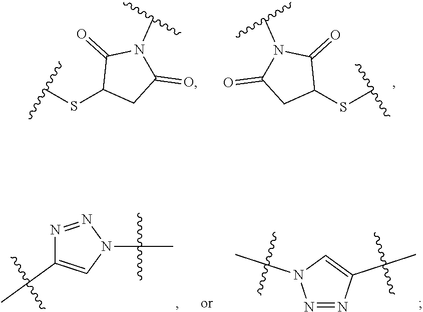

and
R² is H, methyl or ethyl;
optionally, the linker is bonded to an albumin binder (R^{alb}), wherein R^{alb} is:
   —(CH₂)ₙ₃—CH₃ wherein n3 is 8-20;
   —(CH₂)ₙ₄—C(O)OH wherein n4 is 8-20;

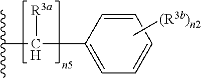

wherein n2 is 1-5, n5 is 1-4, R^{3a} is H or methyl, and R^{3b} is I, Br, F, Cl, H, OH, OCH₃, NH₂, NO₂ or C₁-C₆ alkyl;

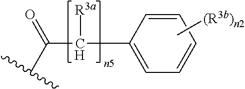

wherein n2 is 1-5, n5 is 1-4, R^{3a} is H or methyl, and R^{3b} is I, Br, F, Cl, H, OH, OCH₃, NH₂, NO₂ or C₁-C₆ alkyl; or

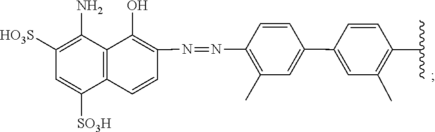 [SMILES: *c1ccc(-c2ccc(N=Nc3ccc4c(S(=O)(=O)O)cc(S(=O)(=O)O)c(N)c4c3O)c(C)c2)cc1C]

n6 is 1-5;
each R^{rad} is a radiolabeling group independently selected from a radiometal chelator; an aryl or heteroaryl substituted with at least one radiohalogen; a prosthetic group containing a trifluoroborate; an aryl or heteroaryl substituted with at least one fluorine precursor leaving group; a prosthetic group containing a silicon-fluorine-acceptor moiety; or a prosthetic group containing a fluorophosphate, fluorosulfate, sulfonyl fluoride, or a combination thereof; and
wherein Xaa¹, Xaa², Xaa³, Xaa⁴, Xaa⁵, Xaa⁶, Xaa⁷, Xaa^{B}, Xaa⁹ or Xaa¹⁰ is each optionally N-methylated.

In some embodiments, the present disclosure relates to a peptidic compound of Formula (IB-1), or a salt or solvate of Formula (IB-1),

(IB-1)
R^{rad}-[linker]-R^{L}-Xaa¹-Xaa²-Xaa³-Xaa⁴-Xaa⁵-Xaa⁶-Xaa⁷-
Xaa⁸-ψ-Xaa⁹-(Xaa¹⁰)ₘ₂-R

wherein:
Xaa¹ is an N-terminal amino acid residue selected from D-Phe, 4-chlorophenylalanine (Cpa), D-Cpa, 3-(1-naphthyl)alanine (Nal), D-Nal, 3-(2-naphthyl)alanine (2-Nal), D-2-Nal, 2,3,4,9-tetrahydro-1H-pyrido[3,4-b]indol-3-carboxylic acid (Tpi), or D-Tpi;
Xaa² is Gly, Arg, Asp, Asn, Gln, Glu, homoserine (Hse), citrulline (Cit), 2-amino-4-ureidobutanoic acid (Aub), 2-amino-4-guanidinobutyric acid (Agb), or His;
Xaa³ is Trp, β-(3-benzothienyl)alanine (Bta), Trp(Me), Trp(7-Me), Trp(6-Me), Trp(5-Me), Trp(4-Me), Trp(2-Me), Trp(7-F), Trp(6-F), Trp(5-F), Trp(4-F), Trp(7-Cl), Trp(6-Cl), Trp(5-Cl), Trp(4-Cl), Trp(7-Br), Trp(6-Br), Trp(5-Br), Trp(4-Br), Trp(7-1), Trp(6-1), Trp(5-1), Trp(4-1), Trp(5-OH), αMe-Trp, Tpi, 7-azatryptophan (7-Aza), Nal, 2-pyridylalanine (2-Pal), 3-pyridylalanine (3-Pal), 4-pyridylalanine (4-Pal), 2-quinoylalanine (2-Quin-Ala), or 3-quinoylalanine (3-Quin-Ala);
Xaa⁴ is Ala, D-Ala, or Ser;
Xaa⁵ is Val, 2,3-dehydro-Val, cyclopentylglycine (Cpg), L-cyclopropylglycine, L-cyclobutylglycine, or tert-leucine (Tle);
Xaa⁶ is Gly or D-Ala;
Xaa⁷ is Trp, Trp(Me), Trp(7-Me), Trp(6-Me), Trp(5-Me), Trp(4-Me), Trp(2-Me), Trp(7-F), Trp(6-F), Trp(5-F), Trp(4-F), Trp(7-Cl), Trp(6-Cl), Trp(5-Cl), Trp(4-Cl), Trp(7-Br), Trp(6-Br), Trp(5-Br), Trp(4-Br), Trp(7-1), Trp(6-1), Trp(5-1), Trp(4-1), Trp(5-OH), αMe-Trp, Nal, 2-pyridylalanine (2-Pal), 3-pyridylalanine (3-Pal), 4-pyridylalanine (4-Pal), 2-quinoylalanine (2-Quin-Ala), or 3-quinoylalanine (3-Quin-Ala);
Xaa⁸ is statine (Sta), Leu, D-Pro, or Phe;
R is —NH₂ or —NHOH;
m2 is 0 or 1;
   when m2 is 0, Xaa⁹-R is a C-terminally amidated (R is —NH₂) or a C-terminally hydroxylamidated (R is —NHOH) amino acid residue selected from Pro, Phe, oxazolidine-4-carboxylic acid (4-oxa-L-Pro), 4,4-difluoroproline (difluoroPro), cis-4-NHR^{a}-D-Pro, cis-4-OR^{a}-D-Pro, Leu, 5,5-dimethyl-1,3-thiazolidine-4-carboxylic acid (Me₂Thz), or thiazoline-4-carboxylic acid (Thz), wherein R^{a} is alkyl, cycloalkyl, or aryl;
   when m2 is 1, Xaa⁹ is Pro, Phe, oxazolidine-4-carboxylic acid (4-oxa-L-Pro), 4,4-difluoroproline (difluoroPro), Leu, Me₂Thz (5,5-dimethyl-1,3-thiazolidine-4-carboxylic acid), or thiazoline-4-carboxylic acid (Thz); and Xaa¹⁰-R is a C-terminally amidated (R is —NH₂) or a C-terminally hydroxylamidated (R is —NHOH) amino acid residue selected from Val, Leu, Tle, Ile, Nle, Ala, Gly, or Met;
ψ represents a peptide bond or reduced peptide bond joining Xaa⁸ to Xaa⁹;
the linker and R^{L} together form a p-aminomethylaniline-diglycolic acid (pABzA-DIG) linker, a 4-amino-(1-carboxymethyl)piperidine (Pip) linker, a 9-amino-4,7-dioxanonanoic acid (dPEG2) linker, or a 4-(2-aminoethyl)-1-carboxymethyl-piperazine (Acp) linker;
R^{rad} is a radiometal chelator; and
wherein Xaa¹, Xaa², Xaa³, Xaa⁴, Xaa⁵, Xaa⁶, Xaa⁷, Xaa⁸, Xaa⁹ or Xaa¹⁰ is each optionally N-methylated.

In some embodiments of the compounds of formula (IB) or (IB-1), Xaa⁷ is Trp, Trp(7-F), Trp(6-F), Trp(5-F), Trp(4-F), 3-Quin-Ala, or 2-Quin-Ala. In some embodiments, Xaa⁷ is Trp, Trp(5-F), or 3-Quin-Ala.

In some embodiments of the compounds of formula (IB) or (IB-1), Xaa¹ is an N-terminal amino acid residue D-Phe. In some embodiments, Xaa² is Gln or Cit. In some embodiments, Xaa² is Gln. In some embodiments, Xaa³ is Trp or αMe-Trp. In some embodiments, Xaa⁴ is Ala. In some embodiments, Xaa⁵ is Val or Tle. In some embodiments, Xaa⁶ is Gly or NMe-Gly. In some embodiments, Xaa⁸ is Sta or Leu. In some embodiments, m2 is 0 and Xaa⁹-R is a C-terminally amidated (R is —NH₂) amino acid residue Pro or Leu. In some embodiments, m2 is 1 and Xaa⁹ is Pro or Leu.

In some embodiments of the compounds of formula (IB) or (IB-1), Xaa¹ is an N-terminal amino acid residue D-Phe and Xaa² is Gln or Cit. In some embodiments, Xaa¹ is an N-terminal amino acid residue D-Phe; Xaa² is Gln or Cit; and Xaa³ is Trp or αMe-Trp. In some embodiments, Xaa¹ is an N-terminal amino acid residue D-Phe; Xaa² is Gln or Cit; Xaa³ is Trp or αMe-Trp; and Xaa⁴ is Ala. In some embodiments, Xaa¹ is an N-terminal amino acid residue D-Phe; Xaa² is Gln or Cit; Xaa³ is Trp or αMe-Trp; Xaa⁴ is Ala; and Xaa⁵ is Val or Tle. In some embodiments, Xaa¹ is an N-terminal amino acid residue D-Phe; Xaa² is Gln or Cit; Xaa³ is Trp or αMe-Trp; Xaa⁴ is Ala; Xaa⁵ is Val or Tle; and Xaa⁶ is Gly or NMe-Gly. In some embodiments, Xaa¹ is an N-terminal amino acid residue D-Phe; Xaa² is Gln or Cit; Xaa³ is Trp or αMe-Trp; Xaa⁴ is Ala; Xaa⁵ is Val or Tle; Xaa⁶ is Gly or NMe-Gly; and Xaa⁸ is Sta or Leu. In some embodiments, Xaa¹ is an N-terminal amino acid residue D-Phe; Xaa² is Gln or Cit; Xaa³ is Trp or αMe-Trp; Xaa⁴ is Ala; Xaa⁵ is Val or Tle; Xaa⁶ is Gly or NMe-Gly; Xaa⁷ is Trp, Trp(7-F), Trp(6-F), Trp(5-F), Trp(4-F), 3-Quin-Ala, or 2-Quin-Ala; and Xaa⁸ is Sta or Leu. In some embodiments, Xaa¹ is an N-terminal amino acid residue D-Phe; Xaa² is Gln or Cit; Xaa³ is Trp or αMe-Trp; Xaa⁴ is Ala; Xaa⁵ is Val or Tle; Xaa⁶ is Gly or NMe-Gly; Xaa⁷ is Trp, Trp(5-F), or 3-Quin-Ala; and Xaa⁸ is Sta or Leu.

In some embodiments, the present disclosure relates to a peptidic compound of Formula (I), or a salt or solvate of Formula (I):

(I)
R^{rad}ₙ₆-[linker]-Xaa¹-Xaa²-Xaa³-Xaa⁴-Xaa⁵-Xaa⁶-Xaa⁷-
Xaa⁸-[ψ-Xaa⁹-(Xaa¹⁰)ₘ₂]ₘ₁-R

wherein:
Xaa¹ is an N-terminal amino acid residue selected from D-Phe, D-Cpa, D-Nal, or D-2-Nal;
Xaa² is Asp, Asn, Gln, or Glu;
Xaa³ is Trp, Trp(Me), αMe-Trp, Trp(7-F), Trp(6-F), Trp(5-F), or Trp(4-F);
Xaa⁴ is Ala or D-Ala;
Xaa⁵ is Val or tert-leucine (Tle);
Xaa⁶ is Gly or D-Ala;
Xaa⁷ is Trp, Trp(Me), Trp(7-Me), Trp(6-Me), Trp(5-Me), Trp(4-Me), Trp(2-Me), Trp(7-F), Trp(6-F), Trp(5-F), Trp(4-F), Trp(7-Cl), Trp(6-Cl), Trp(5-Cl), Trp(4-Cl), Trp(7-Br), Trp(6-Br), Trp(5-Br), Trp(4-Br), Trp(7-1), Trp(6-1), Trp(5-1), Trp(4-1), Trp(5-OH), or αMe-Trp;
Xaa⁸ is Leu;
m1 is 1 and m2 is 0;
Xaa⁹ is Pro, oxazolidine-4-carboxylic acid (4-oxa-L-Pro), or 4,4-difluoroproline (difluoroPro);
R is —NH₂;
ψ represents a peptide bond or reduced peptide bond joining Xaa⁸ to Xaa⁹; the linker comprises p-aminomethylaniline-diglycolic acid (pABzA-DIG),4-amino-(1-carboxymethyl)piperidine (Pip, 9-amino-4,7-dioxanonanoic acid (dPEG2), or 4-(2-aminoethyl)-1-carboxymethyl-piperazine (Acp), wherein the linker optionally comprises an albumin binder;
n6 is 1;
R^{rad} is a radiometal chelator; and
wherein Xaa¹, Xaa², Xaa³, Xaa⁴, Xaa⁵, Xaa⁶, Xaa⁷, Xaa⁸, or Xaa⁹ is each optionally N-methylated.

In some embodiments, the compound is selected from the group consisting of:

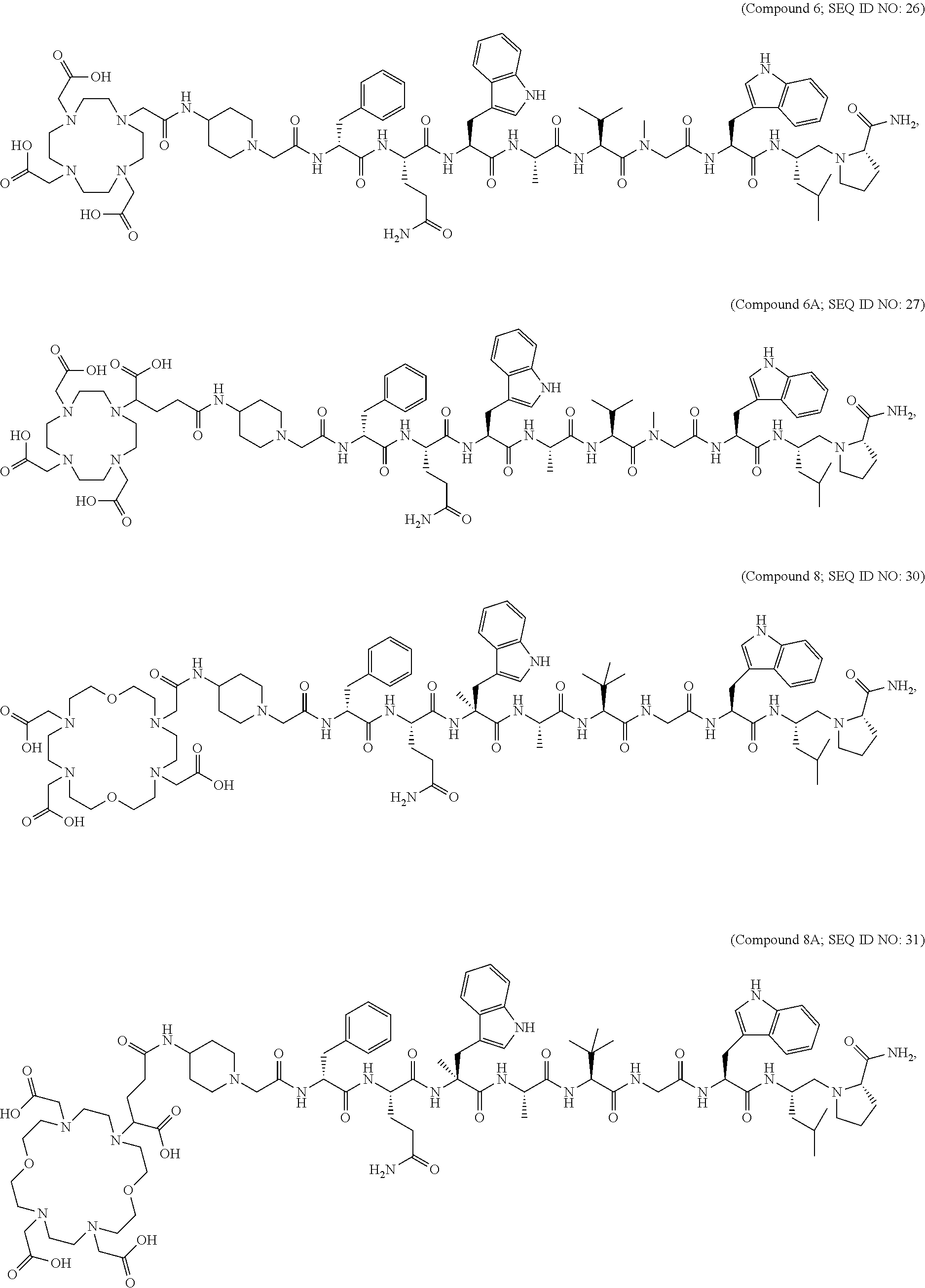

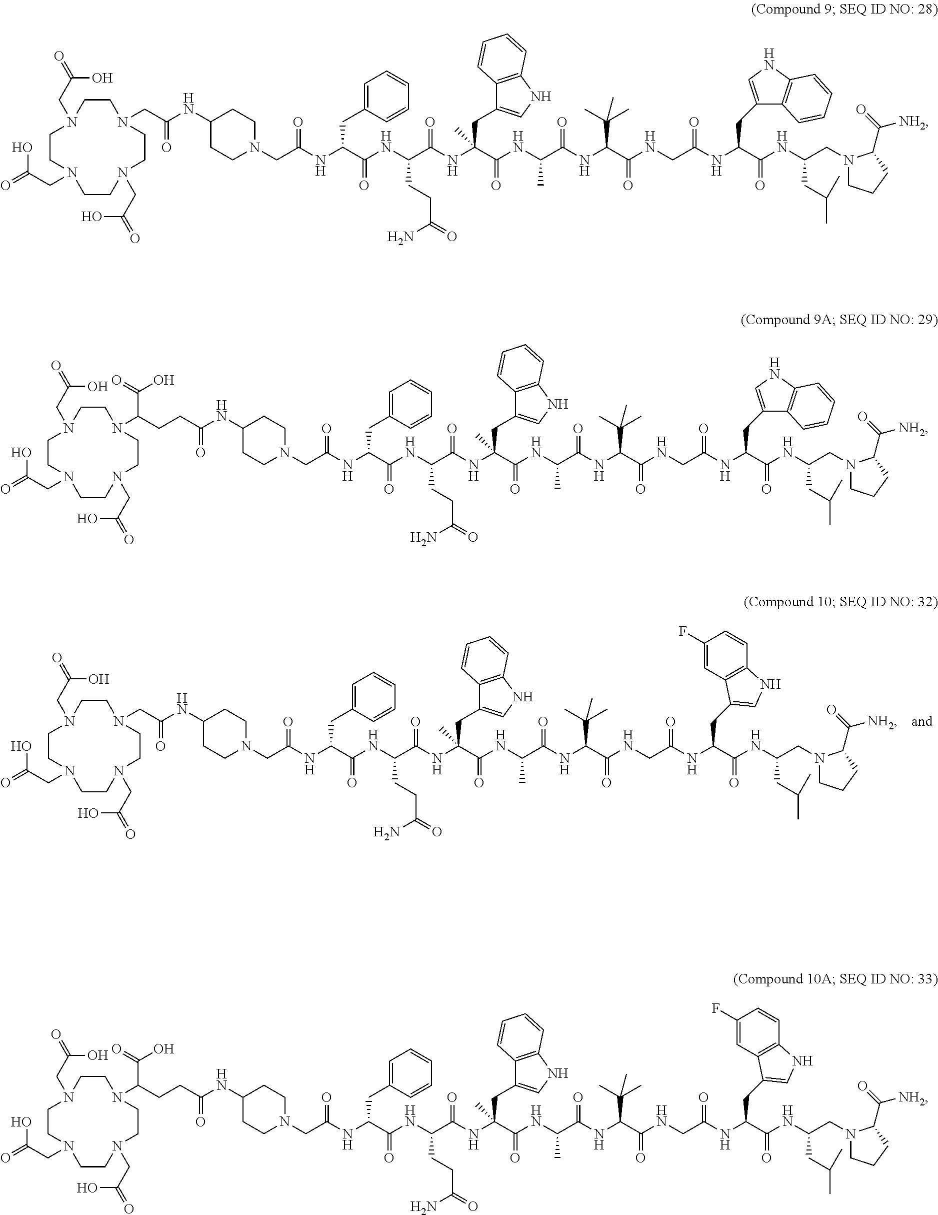

(Compound 10A; SEQ ID NO: 33), or a salt or solvate thereof. In some embodiments, the compound is conjugated by a radiometal. In some embodiments, the radiometal is ⁶⁸Ga, ¹⁷⁷Lu, or ²²⁵Ac.

In some embodiments, the present disclosure relates to a peptidic compound of Formula (IC), or a salt or solvate of Formula (IC),

(IC)
R^{rad}ₙ₆-[linker]-R^{L}-Xaa¹-Xaa²-Xaa³-Xaa⁴-Xaa⁵-Xaa⁶-Xaa⁷-
Xaa⁸-[ψ-Xaa⁹-(Xaa¹⁰)ₘ₂]ₘ₁-R

wherein:
Xaa¹ is an N-terminal amino acid residue selected from D-Phe, 4-chlorophenylalanine (Cpa), D-Cpa, 3-(1-naphthyl)alanine (Nal), D-Nal, 3-(2-naphthyl)alanine (2-Nal), D-2-Nal, 2,3,4,9-tetrahydro-1H-pyrido[3,4-b]indol-3-carboxylic acid (Tpi), or D-Tpi;
Xaa² is Gly, Arg, Asp, Glu, citrulline (Cit), 2-amino-4-ureidobutanoic acid (Aub), or 2-amino-4-guanidinobutyric acid (Agb);
Xaa³ is Trp, β-(3-benzothienyl)alanine (Bta), Trp(Me), Trp(7-Me), Trp(6-Me), Trp(5-Me), Trp(4-Me), Trp(2-Me), Trp(7-F), Trp(6-F), Trp(5-F), Trp(4-F), Trp(7-Cl), Trp(6-Cl), Trp(5-Cl), Trp(4-Cl), Trp(7-Br), Trp(6-Br), Trp(5-Br), Trp(4-Br), Trp(7-1), Trp(6-1), Trp(5-1), Trp(4-1), Trp(5-OH), αMe-Trp, Tpi, 7-azatryptophan (7-Aza), Nal, 2-pyridylalanine (2-Pal), 3-pyridylalanine (3-Pal), 4-pyridylalanine (4-Pal), 2-quinoylalanine (2-Quin-Ala), or 3-quinoylalanine (3-Quin-Ala);
Xaa⁴ is Ala, D-Ala, or Ser;
Xaa⁵ is Val, 2,3-dehydro-Val, cyclopentylglycine (Cpg), L-cyclopropylglycine, L-cyclobutylglycine, or tert-leucine (Tle);
Xaa⁶ is Gly or D-Ala;
Xaa⁷ is His, Trp, Trp(Me), Trp(7-Me), Trp(6-Me), Trp(5-Me), Trp(4-Me), Trp(2-Me), Trp(7-F), Trp(6-F), Trp(5-F), Trp(4-F), Trp(7-Cl), Trp(6-Cl), Trp(5-Cl), Trp(4-Cl), Trp(7-Br), Trp(6-Br), Trp(5-Br), Trp(4-Br), Trp(7-1), Trp(6-1), Trp(5-1), Trp(4-1), Trp(5-OH), αMe-Trp, Nal, 2-pyridylalanine (2-Pal), 3-pyridylalanine (3-Pal), 4-pyridylalanine (4-Pal), 2-quinoylalanine (2-Quin-Ala), or 3-quinoylalanine (3-Quin-Ala);
Xaa⁸ is statine (Sta), Leu, D-Pro, Phe, cis-4-NHR^{a}-D-Pro or cis-4-OR^{a}-D-Pro, wherein R^{a} is alkyl, cycloalkyl, or aryl;
R is —NH₂ or —NHOH;
m1 is 0 or 1;
m2 is 0 or 1;
   when m1 is 1 and m2 is 0, Xaa⁹-R is a C-terminally amidated (R is —NH₂) or a C-terminally hydroxylamidated (R is —NHOH) amino acid residue selected from Pro, Phe, oxazolidine-4-carboxylic acid (4-oxa-L-Pro), 4,4-difluoroproline (difluoroPro), cis-4-NHR^{a}-D-Pro, cis-4-OR^{a}-D-Pro, Leu, 5,5-dimethyl-1,3-thiazolidine-4-carboxylic acid (Me₂Thz), or thiazoline-4-carboxylic acid (Thz), wherein R^{a} is alkyl, cycloalkyl, or aryl;
   when m1 is 1 and m2 is 1, Xaa⁹ is Pro, Phe, oxazolidine-4-carboxylic acid (4-oxa-L-Pro), 4,4-difluoroproline (difluoroPro), Leu, 5,5-dimethyl-1,3-thiazolidine-4-carboxylic acid (Me₂Thz), or thiazoline-4-carboxylic acid (Thz); and Xaa¹⁰-R is a C-terminally amidated (R is —NH₂) or a C-terminally hydroxylamidated (R is —NHOH) amino acid residue selected from Val, Leu, Tle, Ile, Nle, Ala, Gly, or Met;
ψ represents a peptide bond or reduced peptide bond joining Xaa⁸ to Xaa⁹;
R^{L} is —C(O)—, —NH—C(O)—, or —NH—C(S)—;
the linker is a linear or branched chain of n1 units of -Xaa¹¹-, and/or -L¹R¹— and/or -(L¹)₂R¹—, wherein:
   n1 is 1-20;
   each Xaa¹¹ is independently an amino acid;
   each R¹ is, independently, a linear, branched, and/or cyclic C₁₋₂₀ alkylenyl, C₂₋₂₀ alkenylenyl and/or C₂₋₂₀ alkynylenyl, wherein any carbon bonded to two other carbons is optionally independently replaced by N, S, or O, and carbons are optionally independently substituted with oxo, hydroxyl, sulfhydryl, —SeH, halogen, guanidino, amine, amide, urea, carboxylic acid, sulfonic acid, sulfinic acid, or phosphoric acid;
   L¹ bonds to carbon, wherein each L¹ is independently —S—, —N(R²)C(O)—, —C(O) N(R², —NH—C(O)—NH—, —NH—C(S)—NH—,

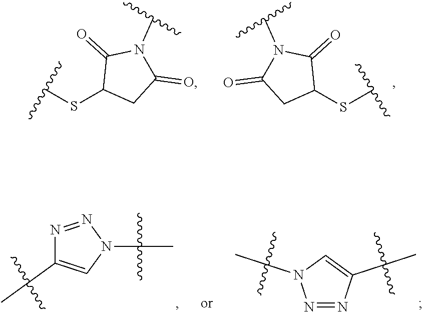

and
R² is H, methyl or ethyl;
optionally, the linker is bonded to an albumin binder (R^{alb}), wherein R^{alb} is:
   —(CH₂)ₙ₃—CH₃ wherein n3 is 8-20;
   —(CH₂)ₙ₄—C(O)OH wherein n4 is 8-20;

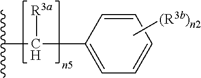

wherein n2 is 1-5, n5 is 1-4, R^{3a} is H or methyl, and R^{3b} is I, Br, F, Cl, H, OH, OCH₃, NH₂, NO₂ or C₁-C₆ alkyl;

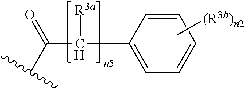

wherein n2 is 1-5, n5 is 1-4, R^{3a} is H or methyl, and R^{3b} is I, Br, F, Cl, H, OH, OCH₃, NH₂, NO₂ or C₁-C₆ alkyl; or

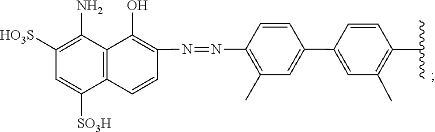 [SMILES: *c1ccc(-c2ccc(N=Nc3ccc4c(S(=O)(=O)O)cc(S(=O)(=O)O)c(N)c4c3O)c(C)c2)cc1C]

n6 is 1-5;
each R^{rad} is a radiolabeling group independently selected from a radiometal chelator; an aryl or heteroaryl substituted with at least one radiohalogen; a prosthetic group containing a trifluoroborate; an aryl or heteroaryl substituted with at least one fluorine precursor leaving group; a prosthetic group containing a silicon-fluorine-acceptor moiety; or a prosthetic group containing a fluorophosphate, fluorosulfate, sulfonyl fluoride, or a combination thereof; and
wherein Xaa¹, Xaa², Xaa³, Xaa⁴, Xaa⁵, Xaa⁶, Xaa⁷, Xaa^{B}, Xaa⁹ or Xaa¹⁰ is each optionally N-methylated.

In some embodiments, the present disclosure relates to a peptidic compound of Formula (IC-1), or a salt or solvate of Formula (IC-1),

(IC-1)
R^{rad}-[linker]-R^{L}-Xaa¹-Xaa²-Xaa³-Xaa⁴-Xaa⁵-Xaa⁶-Xaa⁷-
Xaa⁸-ψ-Xaa⁹-(Xaa¹⁰)ₘ₂-R

wherein:
Xaa¹ is an N-terminal amino acid residue selected from D-Phe, 4-chlorophenylalanine (Cpa), D-Cpa, 3-(1-naphthyl)alanine (Nal), D-Nal, 3-(2-naphthyl)alanine (2-Nal), D-2-Nal, 2,3,4,9-tetrahydro-1H-pyrido[3,4-b]indol-3-carboxylic acid (Tpi), or D-Tpi;
Xaa² is Gly, Arg, Asp, Glu, citrulline (Cit), 2-amino-4-ureidobutanoic acid (Aub), or 2-amino-4-guanidinobutyric acid (Agb);
Xaa³ is Trp, β-(3-benzothienyl)alanine (Bta), Trp(Me), Trp(7-Me), Trp(6-Me), Trp(5-Me), Trp(4-Me), Trp(2-Me), Trp(7-F), Trp(6-F), Trp(5-F), Trp(4-F), Trp(7-Cl), Trp(6-Cl), Trp(5-Cl), Trp(4-Cl), Trp(7-Br), Trp(6-Br), Trp(5-Br), Trp(4-Br), Trp(7-1), Trp(6-1), Trp(5-1), Trp(4-1), Trp(5-OH), αMe-Trp, Tpi, 7-azatryptophan (7-Aza), Nal, 2-pyridylalanine (2-Pal), 3-pyridylalanine (3-Pal), 4-pyridylalanine (4-Pal), 2-quinoylalanine (2-Quin-Ala), or 3-quinoylalanine (3-Quin-Ala);
Xaa⁴ is Ala, D-Ala, or Ser;
Xaa⁵ is Val, 2,3-dehydro-Val, cyclopentylglycine (Cpg), L-cyclopropylglycine, L-cyclobutylglycine, or tert-leucine (Tle);
Xaa⁶ is Gly or D-Ala;
Xaa⁷ is His, Trp, Trp(Me), Trp(7-Me), Trp(6-Me), Trp(5-Me), Trp(4-Me), Trp(2-Me), Trp(7-F), Trp(6-F), Trp(5-F), Trp(4-F), Trp(7-Cl), Trp(6-Cl), Trp(5-Cl), Trp(4-Cl), Trp(7-Br), Trp(6-Br), Trp(5-Br), Trp(4-Br), Trp(7-1), Trp(6-1), Trp(5-1), Trp(4-1), Trp(5-OH), αMe-Trp, Nal, 2-pyridylalanine (2-Pal), 3-pyridylalanine (3-Pal), 4-pyridylalanine (4-Pal), 2-quinoylalanine (2-Quin-Ala), or 3-quinoylalanine (3-Quin-Ala);
Xaa⁸ is statine (Sta), Leu, D-Pro, or Phe;
R is —NH₂ or —NHOH;
m2 is 0 or 1;
   when m2 is 0, Xaa⁹-R is a C-terminally amidated (R is —NH₂) or a C-terminally hydroxylamidated (R is —NHOH) amino acid residue selected from Pro, Phe, oxazolidine-4-carboxylic acid (4-oxa-L-Pro), 4,4-difluoroproline (difluoroPro), cis-4-NHR^{a}-D-Pro, cis-4-OR^{a}-D-Pro, Leu, 5,5-dimethyl-1,3-thiazolidine-4-carboxylic acid (Me₂Thz), or thiazoline-4-carboxylic acid (Thz), wherein R^{a} is alkyl, cycloalkyl, or aryl;
   when m2 is 1, Xaa⁹ is Pro, Phe, oxazolidine-4-carboxylic acid (4-oxa-L-Pro), 4,4-difluoroproline (difluoroPro), Leu, 5,5-dimethyl-1,3-thiazolidine-4-carboxylic acid (Me₂Thz), or thiazoline-4-carboxylic acid (Thz); and Xaa¹⁰-R is a C-terminally amidated (R is —NH₂) or a C-terminally hydroxylamidated (R is —NHOH) amino acid residue selected from Val, Leu, Tle, Ile, Nle, Ala, Gly, or Met;
ψ represents a peptide bond or reduced peptide bond joining Xaa⁸ to Xaa⁹;
the linker and R^{L} together form a p-aminomethylaniline-diglycolic acid (pABzA-DIG) linker, a 4-amino-(1-carboxymethyl)piperidine (Pip) linker, a 9-amino-4,7-dioxanonanoic acid (dPEG2) linker, or a 4-(2-aminoethyl)-1-carboxymethyl-piperazine (Acp) linker;
R^{rad} is a radiometal chelator; and
wherein Xaa¹, Xaa², Xaa³, Xaa⁴, Xaa⁵, Xaa⁶, Xaa⁷, Xaa⁸, Xaa⁹ or Xaa¹⁰ is each optionally N-methylated.

In some embodiments of the compounds of formula (IC) or (IC-1), Xaa² is Cit, Glu, or Agb. In some embodiments, Xaa² is Cit.

In some embodiments of the compounds of formula (IC) or (IC-1), Xaa¹ is an N-terminal amino acid residue D-Phe. In some embodiments, Xaa³ is Trp or αMe-Trp. In some embodiments, Xaa⁴ is Ala. In some embodiments, Xaa⁵ is Val or Tle. In some embodiments, Xaa⁶ is Gly or NMe-Gly. In some embodiments, Xaa⁷ is His, Trp, Trp(5-F), 3-Quin-Ala, or 2-Quin-Ala. In some embodiments, Xaa⁸ is Sta or Leu. In some embodiments, m2 is 0 and Xaa⁹-R is a C-terminally amidated (R is —NH₂) amino acid residue Pro or Leu. In some embodiments, m2 is 1 and Xaa⁹ is Pro or Leu.

In some embodiments of the compounds of formula (IC) or (IC-1), Xaa¹ is an N-terminal amino acid residue D-Phe and Xaa³ is Trp or αMe-Trp. In some embodiments, Xaa¹ is an N-terminal amino acid residue D-Phe; Xaa³ is Trp or αMe-Trp; and Xaa⁴ is Ala. In some embodiments, Xaa¹ is an N-terminal amino acid residue D-Phe; Xaa³ is Trp or αMe-Trp; Xaa⁴ is Ala; and Xaa⁵ is Val or Tle. In some embodiments, Xaa¹ is an N-terminal amino acid residue D-Phe; Xaa³ is Trp or αMe-Trp; Xaa⁴ is Ala; Xaa⁵ is Val or Tle; and Xaa⁶ is Gly or NMe-Gly. In some embodiments, Xaa¹ is an N-terminal amino acid residue D-Phe; Xaa³ is Trp or αMe-Trp; Xaa⁴ is Ala; Xaa⁵ is Val or Tle; Xaa⁶ is Gly or NMe-Gly; and Xaa⁷ is His, Trp, Trp(5-F), 3-Quin-Ala, or 2-Quin-Ala. In some embodiments, Xaa¹ is an N-terminal amino acid residue D-Phe; Xaa³ is Trp or αMe-Trp; Xaa⁴ is Ala; Xaa⁵ is Val or Tle; Xaa⁶ is Gly or NMe-Gly; Xaa⁷ is His, Trp, Trp(5-F), 3-Quin-Ala, or 2-Quin-Ala; and Xaa⁸ is Sta or Leu. In some embodiments, Xaa¹ is an N-terminal amino acid residue D-Phe; Xaa³ is Trp or αMe-Trp; Xaa⁴ is Ala; Xaa⁵ is Val or Tle; Xaa⁶ is Gly or NMe-Gly; Xaa⁷ is His, Trp, Trp(7-F), Trp(6-F), Trp(5-F), Trp(4-F), 3-Quin-Ala, or 2-Quin-Ala; and Xaa⁸ is Sta or Leu. In some embodiments, Xaa¹ is an N-terminal amino acid residue D-Phe; Xaa³ is Trp or αMe-Trp; Xaa⁴ is Ala; Xaa⁵ is Val or Tle; Xaa⁶ is Gly or NMe-Gly; Xaa⁷ is His, Trp, Trp(5-F), or 3-Quin-Ala; and Xaa⁸ is Sta or Leu. In some embodiments, Xaa¹ is an N-terminal amino acid residue D-Phe; Xaa² is Cit, Glu, or Agb; Xaa³ is Trp or αMe-Trp; Xaa⁴ is Ala; Xaa⁵ is Val or Tle; Xaa⁶ is Gly or NMe-Gly; Xaa⁷ is His, Trp, Trp(5-F), or 3-Quin-Ala; and Xaa⁸ is Sta or Leu. In some embodiments, Xaa¹ is an N-terminal amino acid residue D-Phe; Xaa² is Cit; Xaa³ is Trp or αMe-Trp; Xaa⁴ is Ala; Xaa⁵ is Val or Tle; Xaa⁶ is Gly or NMe-Gly; Xaa⁷ is His, Trp, Trp(5-F), or 3-Quin-Ala; and Xaa⁸ is Sta or Leu.

In some embodiments, the present disclosure relates to a peptidic compound of Formula (ID), or a salt or solvate of Formula (ID),

(ID)
R^{rad}ₙ₆-[linker]-R^{L}-Xaa¹-Xaa²-Xaa³-Xaa⁴-Xaa⁵-Xaa⁶-Xaa⁷-
Xaa⁸-ψ-Xaa⁹-(Xaa¹⁰)ₘ₂-R

wherein:
Xaa¹ is an N-terminal amino acid residue selected from D-Phe, 4-chlorophenylalanine (Cpa), D-Cpa, 3-(1-naphthyl)alanine (Nal), D-Nal, 3-(2-naphthyl)alanine (2-Nal), D-2-Nal, 2,3,4,9-tetrahydro-1H-pyrido[3,4-b]indol-3-carboxylic acid (Tpi), or D-Tpi;
Xaa² is Gly, Arg, Asp, Asn, Gln, Glu, homoserine (Hse), citrulline (Cit), 2-amino-4-ureidobutanoic acid (Aub), 2-amino-4-guanidinobutyric acid (Agb), or His;
Xaa³ is Trp, β-(3-benzothienyl)alanine (Bta), Trp(Me), Trp(7-Me), Trp(6-Me), Trp(5-Me), Trp(4-Me), Trp(2-Me), Trp(7-F), Trp(6-F), Trp(5-F), Trp(4-F), Trp(7-Cl), Trp(6-Cl), Trp(5-Cl), Trp(4-Cl), Trp(7-Br), Trp(6-Br), Trp(5-Br), Trp(4-Br), Trp(7-1), Trp(6-1), Trp(5-1), Trp(4-1), Trp(5-OH), αMe-Trp, Tpi, 7-azatryptophan (7-Aza), Nal, 2-pyridylalanine (2-Pal), 3-pyridylalanine (3-Pal), 4-pyridylalanine (4-Pal), 2-quinoylalanine (2-Quin-Ala), or 3-quinoylalanine (3-Quin-Ala);
Xaa⁴ is Ala, D-Ala, or Ser;
Xaa⁵ is Val, 2,3-dehydro-Val, cyclopentylglycine (Cpg), L-cyclopropylglycine, L-cyclobutylglycine, or tert-leucine (Tle);
Xaa⁶ is Gly or D-Ala;
Xaa⁷ is His, Trp, Trp(Me), Trp(7-Me), Trp(6-Me), Trp(5-Me), Trp(4-Me), Trp(2-Me), Trp(7-F), Trp(6-F), Trp(5-F), Trp(4-F), Trp(7-Cl), Trp(6-Cl), Trp(5-Cl), Trp(4-Cl), Trp(7-Br), Trp(6-Br), Trp(5-Br), Trp(4-Br), Trp(7-i), Trp(6-i), Trp(5-i), Trp(4-i), Trp(5-OH), αMe-Trp, Nal, 2-pyridylalanine (2-Pal), 3-pyridylalanine (3-Pal), 4-pyridylalanine (4-Pal), 2-quinoylalanine (2-Quin-Ala), or 3-quinoylalanine (3-Quin-Ala);
Xaa⁸ is statine (Sta), Leu, D-Pro, or Phe;
Xaa⁹ is Pro, Phe, oxazolidine-4-carboxylic acid (4-oxa-L-Pro), 4,4-difluoroproline (difluoroPro), Leu, 5,5-dimethyl-1,3-thiazolidine-4-carboxylic acid (Me₂Thz), or thiazoline-4-carboxylic acid (Thz);
R is —NH₂ or —NHOH;
m2 is 1;
Xaa¹⁰-R is a C-terminally amidated (R is —NH₂) or a C-terminally hydroxylamidated (R is —NHOH) amino acid residue selected from Val, Leu, Tle, Ile, Nle, Ala, Gly, or Met;
ψ represents a peptide bond or reduced peptide bond joining Xaa⁸ to Xaa⁹;
R^{L} is —C(O)—, —NH—C(O)—, or —NH—C(S)—;
the linker is a linear or branched chain of n1 units of -Xaa¹¹-, and/or -L¹R¹— and/or -(L¹)₂R¹—, wherein:
   n1 is 1-20;
   each Xaa¹¹ is independently an amino acid;
   each R¹ is, independently, a linear, branched, and/or cyclic C₁₋₂₀ alkylenyl, C₂₋₂₀ alkenylenyl and/or C₂₋₂₀ alkynylenyl, wherein any carbon bonded to two other carbons is optionally independently replaced by N, S, or O, and carbons are optionally independently substituted with oxo, hydroxyl, sulfhydryl, —SeH, halogen, guanidino, amine, amide, urea, carboxylic acid, sulfonic acid, sulfinic acid, or phosphoric acid;
   L¹ bonds to carbon, wherein each L¹ is independently —S—, —N(R²)C(O)—, —C(O)N(R²)—, —NH—C(O)—NH—, —NH—C(S)—NH—,

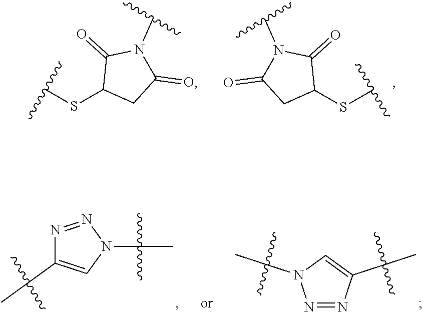

and
R² is H, methyl or ethyl;
optionally, the linker is bonded to an albumin binder (R^{ab}), wherein R^{alb} is:
   —(CH₂)ₙ₃—CH₃ wherein n3 is 8-20;
   —(CH₂)ₙ₄—C(O)OH wherein n4 is 8-20;

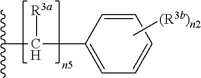

wherein n2 is 1-5, n5 is 1-4, R^{3a} is H or methyl, and R^{3b} is I, Br, F, Cl, H, OH, OCH₃, NH₂, NO₂ or C₁-C₆ alkyl;

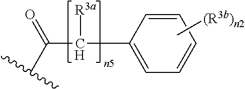

wherein n2 is 1-5, n5 is 1-4, R^{3a} is H or methyl, and R^{3b} is I, Br, F, Cl, H, OH, OCH₃, NH₂, NO₂ or C₁-C₆ alkyl; or

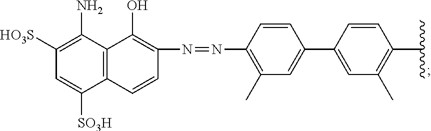 [SMILES: *c1ccc(-c2ccc(N=Nc3ccc4c(S(=O)(=O)O)cc(S(=O)(=O)O)c(N)c4c3O)c(C)c2)cc1C]

n6 is 1-5;
each R^{rad} is a radiolabeling group independently selected from a radiometal chelator; an aryl or heteroaryl substituted with at least one radiohalogen; a prosthetic group containing a trifluoroborate; an aryl or heteroaryl substituted with at least one fluorine precursor leaving group; a prosthetic group containing a silicon-fluorine-acceptor moiety; or a prosthetic group containing a fluorophosphate, fluorosulfate, sulfonyl fluoride, or a combination thereof; and
wherein Xaa¹, Xaa², Xaa³, Xaa⁴, Xaa⁵, Xaa⁶, Xaa⁷, Xaa^{B}, Xaa⁹ or Xaa¹⁰ is each optionally N-methylated.

In some embodiments, the present disclosure relates to a peptidic compound of Formula (ID-1), or a salt or solvate of Formula (ID-1),

(ID-1)
R^{rad}-[linker]-R^{L}-Xaa¹-Xaa²-Xaa³-Xaa⁴-Xaa⁵-Xaa⁶-Xaa⁷-
Xaa⁸-ψ-Xaa⁹-(Xaa¹⁰)ₘ₂-R

wherein:
Xaa¹ is an N-terminal amino acid residue selected from D-Phe, 4-chlorophenylalanine (Cpa), D-Cpa, 3-(1-naphthyl)alanine (Nal), D-Nal, 3-(2-naphthyl)alanine (2-Nal), D-2-Nal, 2,3,4,9-tetrahydro-1H-pyrido[3,4-b]indol-3-carboxylic acid (Tpi), or D-Tpi;
Xaa² is Gly, Arg, Asp, Asn, Gln, Glu, homoserine (Hse), citrulline (Cit), 2-amino-4-ureidobutanoic acid (Aub), 2-amino-4-guanidinobutyric acid (Agb), or His;
Xaa³ is Trp, β-(3-benzothienyl)alanine (Bta), Trp(Me), Trp(7-Me), Trp(6-Me), Trp(5-Me), Trp(4-Me), Trp(2-Me), Trp(7-F), Trp(6-F), Trp(5-F), Trp(4-F), Trp(7-Cl), Trp(6-Cl), Trp(5-Cl), Trp(4-Cl), Trp(7-Br), Trp(6-Br), Trp(5-Br), Trp(4-Br), Trp(7-1), Trp(6-1), Trp(5-1), Trp(4-1), Trp(5-OH), αMe-Trp, Tpi, 7-azatryptophan (7-Aza), Nal, 2-pyridylalanine (2-Pal), 3-pyridylalanine (3-Pal), 4-pyridylalanine (4-Pal), 2-quinoylalanine (2-Quin-Ala), or 3-quinoylalanine (3-Quin-Ala);
Xaa⁴ is Ala, D-Ala, or Ser;
Xaa⁵ is Val, 2,3-dehydro-Val, cyclopentylglycine (Cpg), L-cyclopropylglycine, L-cyclobutylglycine, or tert-leucine (Tle);
Xaa⁶ is Gly or D-Ala;
Xaa⁷ is His, Trp, Trp(Me), Trp(7-Me), Trp(6-Me), Trp(5-Me), Trp(4-Me), Trp(2-Me), Trp(7-F), Trp(6-F), Trp(5-F), Trp(4-F), Trp(7-Cl), Trp(6-Cl), Trp(5-Cl), Trp(4-Cl), Trp(7-Br), Trp(6-Br), Trp(5-Br), Trp(4-Br), Trp(7-1), Trp(6-1), Trp(5-1), Trp(4-1), Trp(5-OH), αMe-Trp, Nal, 2-pyridylalanine (2-Pal), 3-pyridylalanine (3-Pal), 4-pyridylalanine (4-Pal), 2-quinoylalanine (2-Quin-Ala), or 3-quinoylalanine (3-Quin-Ala);
Xaa⁸ is statine (Sta), Leu, D-Pro, or Phe;
Xaa⁹ is Pro, Phe, oxazolidine-4-carboxylic acid (4-oxa-L-Pro), 4,4-difluoroproline (difluoroPro), Leu, 5,5-dimethyl-1,3-thiazolidine-4-carboxylic acid (Me₂Thz), or thiazoline-4-carboxylic acid (Thz);
R is —NH₂ or —NHOH;
m2 is 1;
Xaa¹⁰-R is a C-terminally amidated (R is —NH₂) or a C-terminally hydroxylamidated (R is —NHOH) amino acid residue selected from Val, Leu, Tle, Ile, Nle, Ala, Gly, or Met;
ψ represents a peptide bond or reduced peptide bond joining Xaa⁸ to Xaa⁹;
the linker and R^{L} together form a p-aminomethylaniline-diglycolic acid (pABzA-DIG) linker, a 4-amino-(1-carboxymethyl)piperidine (Pip) linker, a 9-amino-4,7-dioxanonanoic acid (dPEG2) linker, or a 4-(2-aminoethyl)-1-carboxymethyl-piperazine (Acp) linker;
R^{rad} is a radiometal chelator; and
wherein Xaa¹, Xaa², Xaa³, Xaa⁴, Xaa⁵, Xaa⁶, Xaa⁷, Xaa⁸, Xaa⁹ or Xaa¹⁰ is each optionally N-methylated.

In some embodiments of the compounds of formula (ID) or (ID-1), Xaa¹ is an N-terminal amino acid residue D-Phe. In some embodiments, Xaa² is Gln or Cit. In some embodiments, Xaa³ is Trp or αMe-Trp. In some embodiments, Xaa⁴ is Ala. In some embodiments, Xaa⁵ is Val or Tle. In some embodiments, Xaa⁶ is Gly or NMe-Gly. In some embodiments, Xaa⁷ is His, Trp, Trp(5-F), 3-Quin-Ala, or 2-Quin-Ala. In some embodiments, Xaa⁸ is Sta or Leu. In some embodiments, Xaa⁹ is Pro or Leu. In some embodiments, Xaa¹⁰ is Val or Tle.

In some embodiments of the compounds of formula (ID) or (ID-1), Xaa¹ is an N-terminal amino acid residue D-Phe and Xaa² is Gln or Cit. In some embodiments, Xaa¹ is an N-terminal amino acid residue D-Phe; Xaa² is Gln or Cit; and Xaa³ is Trp or αMe-Trp. In some embodiments, Xaa¹ is an N-terminal amino acid residue D-Phe; Xaa² is Gln or Cit; ad Xaa³ is Trp or αMe-Trp; and Xaa⁴ is Ala. In some embodiments, Xaa¹ is an N-terminal amino acid residue D-Phe; Xaa² is Gln or Cit; Xaa³ is Trp or αMe-Trp; Xaa⁴ is Ala; and Xaa⁵ is Val or Tle. In some embodiments, Xaa¹ is an N-terminal amino acid residue D-Phe; Xaa² is Gln or Cit; Xaa³ is Trp or αMe-Trp; Xaa⁴ is Ala; Xaa⁵ is Val or Tle; and Xaa⁶ is Gly or NMe-Gly. In some embodiments, Xaa¹ is an N-terminal amino acid residue D-Phe; Xaa² is Gln or Cit; Xaa³ is Trp or αMe-Trp; Xaa⁴ is Ala; Xaa⁵ is Val or Tle; Xaa⁶ is Gly or NMe-Gly; and Xaa² is His, Trp, Trp(5-F), 3-Quin-Ala, or 2-Quin-Ala. In some embodiments, Xaa¹ is an N-terminal amino acid residue D-Phe; Xaa² is Gln or Cit; Xaa³ is Trp or αMe-Trp; Xaa⁴ is Ala; Xaa⁵ is Val or Tle; Xaa⁶ is Gly or NMe-Gly; Xaa⁷ is His, Trp, Trp(5-F), 3-Quin-Ala, or 2-Quin-Ala; and Xaa⁸ is Sta or Leu. In some embodiments, Xaa¹ is an N-terminal amino acid residue D-Phe; Xaa² is Gln or Cit; Xaa³ is Trp or αMe-Trp; Xaa⁴ is Ala; Xaa⁵ is Val or Tle; Xaa⁶ is Gly or NMe-Gly; Xaa² is His, Trp(5-F), or 3-Quin-Ala; and Xaa⁸ is Sta or Leu. In some embodiments, Xaa¹ is an N-terminal amino acid residue D-Phe; Xaa² is Gln or Cit; Xaa³ is Trp or αMe-Trp; Xaa⁴ is Ala; Xaa⁵ is Val or Tle; Xaa⁶ is Gly or NMe-Gly; Xaa² is His, Trp, Trp(7-F), Trp(6-F), Trp(5-F), Trp(4-F), 3-Quin-Ala, or 2-Quin-Ala; and Xaa⁸ is Sta or Leu. In some embodiments, Xaa¹ is an N-terminal amino acid residue D-Phe; Xaa² is Gln or Cit; Xaa³ is Trp or αMe-Trp; Xaa⁴ is Ala; Xaa⁵ is Val or Tle; Xaa⁶ is Gly or NMe-Gly; Xaa⁷ is His, Trp, Trp(7-F), Trp(6-F), Trp(5-F), Trp(4-F), 3-Quin-Ala, or 2-Quin-Ala; Xaa⁸ is Sta or Leu; and Xaa⁹ is Pro or Leu. In some embodiments, Xaa¹ is an N-terminal amino acid residue D-Phe; Xaa² is Gln or Cit; Xaa³ is Trp or αMe-Trp; Xaa⁴ is Ala; Xaa⁵ is Val or Tle; Xaa⁶ is Gly or NMe-Gly; Xaa⁷ is His, Trp(5-F), or 3-Quin-Ala; and Xaa⁸ is Sta or Leu. In some embodiments, Xaa¹ is an N-terminal amino acid residue D-Phe; Xaa² is Gln or Cit; Xaa³ is Trp or αMe-Trp; Xaa⁴ is Ala; Xaa⁵ is Val or Tle; Xaa⁶ is Gly or NMe-Gly; Xaa² is His, Trp(5-F), or 3-Quin-Ala; Xaa⁸ is Sta or Leu; and Xaa⁹ is Pro or Leu.

In some embodiments, the present disclosure relates to a peptidic compound of Formula (IE), or a salt or solvate of Formula (IE),

(IE)
R^{rad}ₙ₆-[linker]-R^{L}-Xaa¹-Xaa²-Xaa³-Xaa⁴-Xaa⁵-Xaa⁶-Xaa⁷-
Xaa⁸-[ψ-Xaa⁹-(Xaa¹⁰)ₘ₂]ₘ₁-R

wherein:
Xaa¹ is an N-terminal amino acid residue selected from D-Phe, 4-chlorophenylalanine (Cpa), D-Cpa, 3-(1-naphthyl)alanine (Nal), D-Nal, 3-(2-naphthyl)alanine (2-Nal), D-2-Nal, 2,3,4,9-tetrahydro-1H-pyrido[3,4-b]indol-3-carboxylic acid (Tpi), or D-Tpi;
Xaa² is Gly, Arg, Asp, Asn, Gln, Glu, homoserine (Hse), citrulline (Cit), 2-amino-4-ureidobutanoic acid (Aub), 2-amino-4-guanidinobutyric acid (Agb), or His;
Xaa³ is Trp, β-(3-benzothienyl)alanine (Bta), Trp(Me), Trp(7-Me), Trp(6-Me), Trp(5-Me), Trp(4-Me), Trp(2-Me), Trp(7-F), Trp(6-F), Trp(5-F), Trp(4-F), Trp(7-Cl), Trp(6-Cl), Trp(5-Cl), Trp(4-Cl), Trp(7-Br), Trp(6-Br), Trp(5-Br), Trp(4-Br), Trp(7-1), Trp(6-1), Trp(5-1), Trp(4-1), Trp(5-OH), αMe-Trp, Tpi, 7-azatryptophan (7-Aza), Nal, 2-pyridylalanine (2-Pal), 3-pyridylalanine (3-Pal), 4-pyridylalanine (4-Pal), 2-quinoylalanine (2-Quin-Ala), or 3-quinoylalanine (3-Quin-Ala);
Xaa⁴ is Ala, D-Ala, or Ser;
Xaa⁵ is Val, 2,3-dehydro-Val, cyclopentylglycine (Cpg), L-cyclopropylglycine, L-cyclobutylglycine, or tert-leucine (Tle);
Xaa⁶ is Gly or D-Ala;
Xaa⁷ is His, Trp, Trp(Me), Trp(7-Me), Trp(6-Me), Trp(5-Me), Trp(4-Me), Trp(2-Me), Trp(7-F), Trp(6-F), Trp(5-F), Trp(4-F), Trp(7-Cl), Trp(6-Cl), Trp(5-Cl), Trp(4-Cl), Trp(7-Br), Trp(6-Br), Trp(5-Br), Trp(4-Br), Trp(7-1), Trp(6-1), Trp(5-1), Trp(4-1), Trp(5-OH), αMe-Trp, Nal, 2-pyridylalanine (2-Pal), 3-pyridylalanine (3-Pal), 4-pyridylalanine (4-Pal), 2-quinoylalanine (2-Quin-Ala), or 3-quinoylalanine (3-Quin-Ala);
Xaa⁸ is statine (Sta), Leu, D-Pro, Phe, cis-4-NHR^{a}-D-Pro or cis-4-OR^{a}-D-Pro, wherein R^{a} is alkyl, cycloalkyl, or aryl;
R is —NH₂ or —NHOH;
m1 is 0 or 1;
m2 is 0 or 1;
   when m1 is 1 and m2 is 0, Xaa⁹-R is a C-terminally amidated (R is —NH₂) or a C-terminally hydroxylamidated (R is —NHOH) amino acid residue selected from Pro, Phe, oxazolidine-4-carboxylic acid (4-oxa-L-Pro), 4,4-difluoroproline (difluoroPro), cis-4-NHR^{a}-D-Pro, cis-4-OR^{a}-D-Pro, Leu, 5,5-dimethyl-1,3-thiazolidine-4-carboxylic acid (Me₂Thz), or thiazoline-4-carboxylic acid (Thz), wherein R^{a} is alkyl, cycloalkyl, or aryl;
   when m1 is 1 and m2 is 1, Xaa⁹ is Pro, Phe, oxazolidine-4-carboxylic acid (4-oxa-L-Pro), 4,4-difluoroproline (difluoroPro), Leu, 5,5-dimethyl-1,3-thiazolidine-4-carboxylic acid (Me₂Thz), or thiazoline-4-carboxylic acid (Thz); and Xaa¹⁰-R is a C-terminally amidated (R is —NH₂) or a C-terminally hydroxylamidated (R is —NHOH) amino acid residue selected from Val, Leu, Tle, Ile, Nle, Ala, Gly, or Met;
ψ represents a peptide bond or reduced peptide bond joining Xaa⁸ to Xaa⁹;
each R^{L} is independently absent, —(C₁₋₆ alkyl)-, —(C₁₋₆ alkyl)-C(O)—, —C(O)—, —NH—C(O)—, or —NH—C(S)—;
the linker is a linear or branched chain of n1 units of -Xaa¹¹-, and/or -L¹R¹— and/or -(L¹)₂R¹—, wherein:
   n1 is 1-20;
   each Xaa¹¹ is independently an amino acid;
   each R¹ is, independently, a linear, branched, and/or cyclic C₁₋₂₀ alkylenyl, alkenylenyl and/or alkynylenyl, wherein any carbon bonded to two other carbons is optionally independently replaced by N, S, or O, and carbons are optionally independently substituted with oxo, hydroxyl, sulfhydryl, —SeH, halogen, guanidino, amine, amide, urea, carboxylic acid, sulfonic acid, sulfinic acid, or phosphoric acid;
   L¹ bonds to carbon, wherein each L¹ is independently —S—, —N(R²)C(O)—, —C(O)N(R²)—, —NH—C(O)—NH—, —NH—C(S)—NH—,

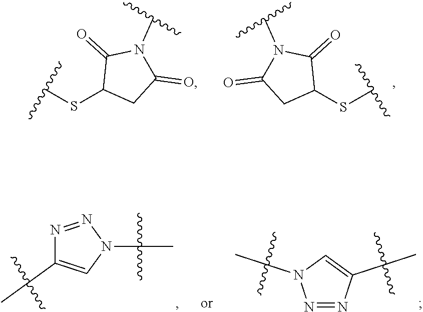

and
R² is H, methyl or ethyl;
optionally, the linker comprises an albumin binder (R^{alb}), wherein the albumin binder is:
   —(CH₂)ₙ₃—CH₃ wherein n3 is 8-20;
   —(CH₂)ₙ₄—C(O)OH wherein n4 is 8-20;

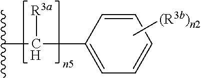

wherein n2 is 1-5, n5 is 1-4, R^{3a} is H or methyl, and R^{3b} is I, Br, F, Cl, H, OH, OCH₃, NH₂, NO₂ or C₁-C₆ alkyl;

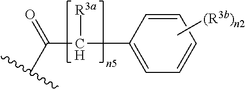

wherein n2 is 1-5, n5 is 1-4, R^{3a} is H or methyl, and R^{3b} is I, Br, F, Cl, H, OH, OCH₃, NH₂, NO₂ or C₁-C₆ alkyl; or

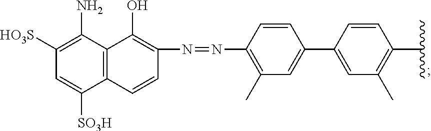 [SMILES: *c1ccc(-c2ccc(N=Nc3ccc4c(S(=O)(=O)O)cc(S(=O)(=O)O)c(N)c4c3O)c(C)c2)cc1C]

n6 is 1-5;
each R^{rad} is a radiolabeling group independently selected from a radiometal chelator; an aryl or heteroaryl substituted with at least one radiohalogen; a prosthetic group containing a trifluoroborate; an aryl or heteroaryl substituted with at least one fluorine precursor leaving group; a prosthetic group containing a silicon-fluorine-acceptor moiety; or a prosthetic group containing a fluorophosphate, fluorosulfate, sulfonyl fluoride, or a combination thereof;
wherein at least one R^{rad} is CROWN, CROWNGA, TCMC, PSC, a prosthetic group containing a trifluoroborate, an aryl or heteroaryl substituted with at least one radiohalogen, or an aryl or heteroaryl substituted with at least one fluorine precursor leaving group; and
wherein Xaa¹, Xaa², Xaa³, Xaa⁴, Xaa⁵, Xaa⁶, Xaa⁷, Xaa^{B}, Xaa⁹ or Xaa¹⁰ is each optionally N-methylated.

In some embodiments, of a compound of Formula (IE), or a salt or solvate of Formula (IE), at least one R^{rad} is CROWN, TCMC, PSC, a prosthetic group containing a trifluoroborate, an aryl or heteroaryl substituted with at least one radiohalogen, or an aryl or heteroaryl substituted with at least one fluorine precursor leaving group.

In some embodiments, the present disclosure relates to a peptidic compound of Formula (IE-1), or a salt or solvate of Formula (IE-1),

(IE-1)
R^{rad}-R^{L}-[linker]-Xaa¹-Xaa²-Xaa³-Xaa⁴-Xaa⁵-Xaa⁶-Xaa⁷-
Xaa⁸-ψ-Xaa⁹-(Xaa¹⁰)ₘ₂-R

wherein:
Xaa¹ is an N-terminal amino acid residue selected from D-Phe, 4-chlorophenylalanine (Cpa), D-Cpa, 3-(1-naphthyl)alanine (Nal), D-Nal, 3-(2-naphthyl)alanine (2-Nal), D-2-Nal, 2,3,4,9-tetrahydro-1H-pyrido[3,4-b]indol-3-carboxylic acid (Tpi), or D-Tpi;
Xaa² is Gly, Arg, Asp, Asn, Gln, Glu, homoserine (Hse), citrulline (Cit), 2-amino-4-ureidobutanoic acid (Aub), 2-amino-4-guanidinobutyric acid (Agb), or His;
Xaa³ is Trp, β-(3-benzothienyl)alanine (Bta), Trp(Me), Trp(7-Me), Trp(6-Me), Trp(5-Me), Trp(4-Me), Trp(2-Me), Trp(7-F), Trp(6-F), Trp(5-F), Trp(4-F), Trp(7-Cl), Trp(6-Cl), Trp(5-Cl), Trp(4-Cl), Trp(7-Br), Trp(6-Br), Trp(5-Br), Trp(4-Br), Trp(7-1), Trp(6-1), Trp(5-1), Trp(4-1), Trp(5-OH), αMe-Trp, Tpi, 7-azatryptophan (7-Aza), Nal, 2-pyridylalanine (2-Pal), 3-pyridylalanine (3-Pal), 4-pyridylalanine (4-Pal), 2-quinoylalanine (2-Quin-Ala), or 3-quinoylalanine (3-Quin-Ala);
Xaa⁴ is Ala, D-Ala, or Ser;
Xaa⁵ is Val, 2,3-dehydro-Val, cyclopentylglycine (Cpg), L-cyclopropylglycine, L-cyclobutylglycine, or tert-leucine (Tle);
Xaa⁶ is Gly or D-Ala;
Xaa⁷ is His, Trp, Trp(Me), Trp(7-Me), Trp(6-Me), Trp(5-Me), Trp(4-Me), Trp(2-Me), Trp(7-F), Trp(6-F), Trp(5-F), Trp(4-F), Trp(7-Cl), Trp(6-Cl), Trp(5-Cl), Trp(4-Cl), Trp(7-Br), Trp(6-Br), Trp(5-Br), Trp(4-Br), Trp(7-1), Trp(6-1), Trp(5-1), Trp(4-1), Trp(5-OH), αMe-Trp, Nal, 2-pyridylalanine (2-Pal), 3-pyridylalanine (3-Pal), 4-pyridylalanine (4-Pal), 2-quinoylalanine (2-Quin-Ala), or 3-quinoylalanine (3-Quin-Ala);
Xaa⁸ is statine (Sta), Leu, D-Pro, or Phe
R is —NH₂ or —NHOH;
m2 is 0 or 1;
   when m2 is 0, Xaa⁹-R is a C-terminally amidated (R is —NH₂) or a C-terminally hydroxylamidated (R is —NHOH) amino acid residue selected from Pro, Phe, oxazolidine-4-carboxylic acid (4-oxa-L-Pro), 4,4-difluoroproline (difluoroPro), cis-4-NHR^{a}-D-Pro, cis-4-OR^{a}-D-Pro, Leu, 5,5-dimethyl-1,3-thiazolidine-4-carboxylic acid (Me₂Thz), or thiazoline-4-carboxylic acid (Thz), wherein R^{a} is alkyl, cycloalkyl, or aryl;
   when m2 is 1, Xaa⁹ is Pro, Phe, oxazolidine-4-carboxylic acid (4-oxa-L-Pro), 4,4-difluoroproline (difluoroPro), Leu, 5,5-dimethyl-1,3-thiazolidine-4-carboxylic acid (Me₂Thz), or thiazoline-4-carboxylic acid (Thz); and Xaa¹⁰-R is a C-terminally amidated (R is —NH₂) or a C-terminally hydroxylamidated (R is —NHOH) amino acid residue selected from Val, Leu, Tle, Ile, Nle, Ala, Gly, or Met;
ψ represents a peptide bond or reduced peptide bond joining Xaa⁸ to Xaa⁹;
R^{L} is independently absent, —(C₁₋₃alkyl)-, —(C₁₋₃ alkyl)-C(O)—, —C(O)—, —NH—C(O)—, or —NH—C(S)—;
the linker is a linear or branched chain of n1 units of -Xaa¹¹-, and/or -L¹R¹— and/or -(L¹)₂R¹—, wherein:
   n1 is 1-6;
   each Xaa¹¹ is independently an amino acid;
   each R¹ is, independently, a linear, branched, and/or cyclic C₁₋₆ alkylenyl, C₂₋₆ alkenylenyl and/or C₂₋₆ alkynylenyl, wherein any carbon bonded to two other carbons is optionally independently replaced by N, S, or O, and carbons are optionally independently substituted with oxo, hydroxyl, sulfhydryl, —SeH, halogen, guanidino, amine, amide, urea, carboxylic acid, sulfonic acid, sulfinic acid, or phosphoric acid;
   L¹ bonds to carbon, wherein each L¹ is independently —S—, —N(R²)C(O)—, —C(O)N(R²)—, —NH—C(O)—NH—, —NH—C(S)—NH—,

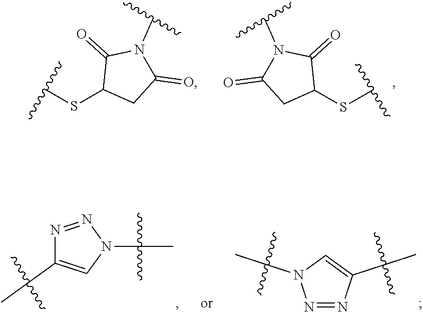

and
R² is H, methyl or ethyl;
R^{rad} is CROWN, CROWNGA, TCMC, PSC, a prosthetic group containing a trifluoroborate, an aryl or heteroaryl substituted with at least one radiohalogen, or an aryl or heteroaryl substituted with at least one fluorine precursor leaving group; and
wherein Xaa¹, Xaa², Xaa³, Xaa⁴, Xaa⁵, Xaa⁶, Xaa⁷, Xaa⁸, Xaa⁹ or Xaa¹⁰ is each optionally N-methylated.

In some embodiments, a compound of Formula (IE-1), or a salt or solvate of Formula (IE-1), at least one R^{rad} is CROWN, TCMC, PSC, a prosthetic group containing a trifluoroborate, an aryl or heteroaryl substituted with at least one radiohalogen, or an aryl or heteroaryl substituted with at least one fluorine precursor leaving group.

In some embodiments of the compounds of formula (IE) or (IE-1), the linker is -Xaa¹¹-. In some embodiments, the linker is -Xaa¹¹- and R^{L} is absent. In some embodiments, the linker is Pip and R^{L} is absent. In some embodiments, the linker is -Xaa¹¹- and R^{L} is —(C₁₋₃ alkyl)-, —(C₁₋₃ alkyl)-C(O)—, or —C(O)—. In some embodiments, the linker is -Xaa¹¹- and R^{L} is —C(O)—. In some embodiments, the linker is Pip and R^{L} is —C(O)—.

In some embodiments of the compounds of formula (IE) or (IE-1), the linker is a linear or branched chain of n1 units of -Xaa¹¹- and/or -L¹R¹—. In some embodiments, n1 is 1-3. In some embodiments, n1 is 1 or 2.

In some embodiments of the compounds of formula (IE) or (IE-1), the linker is -Xaa¹¹-R¹L¹-. In some embodiments, the linker is *-Xaa¹¹-R‘L’-, where * indicates the connectivity to Xaa¹. In some embodiments, the linker is *-Xaa¹¹-C(O)CH₂-L¹-, where * indicates the connectivity to Xaa¹. In some embodiments, the linker is *-Xaa¹¹-C(O)CH₂-L¹-, where * indicates the connectivity to Xaa¹ and L¹ is

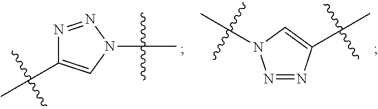 [SMILES: *c1cn(*)nn1 ; *c1cn(*)nn1]

In some embodiments, R^{L} is —(C₁₃ alkyl)-, —(C₁₋₃ alkyl)-C(O)—, or —C(O)—. In some embodiments, R^{L} is —(C₁₋₃ alkyl)-. In some embodiments, R^{L} is —CH₂—. In some embodiments, R^{L} is absent.

In some embodiments of the compounds of formula (IE) or (IE-1), Xaa¹ is an N-terminal amino acid residue D-Phe. In some embodiments, Xaa² is Gln or Cit. In some embodiments, Xaa³ is Trp or αMe-Trp. In some embodiments, Xaa⁴ is Ala. In some embodiments, Xaa⁵ is Val or Tle. In some embodiments, Xaa⁶ is Gly or NMe-Gly. In some embodiments, Xaa⁷ is His, Trp, Trp(5-F), 3-Quin-Ala, or 2-Quin-Ala. In some embodiments, Xaa⁸ is Sta or Leu. In some embodiments, m2 is 0 and Xaa⁹-R is a C-terminally amidated (R is —NH₂) amino acid residue Pro or Leu. In some embodiments, m2 is 1 and Xaa⁹ is Pro or Leu.

In some embodiments of the compounds of formula (IE) or (IE-1), Xaa¹ is an N-terminal amino acid residue D-Phe and Xaa² is Gln or Cit. In some embodiments, Xaa¹ is an N-terminal amino acid residue D-Phe; Xaa² is Gln or Cit; and Xaa³ is Trp or αMe-Trp. In some embodiments, Xaa¹ is an N-terminal amino acid residue D-Phe; Xaa² is Gln or Cit; ad Xaa³ is Trp or αMe-Trp; and Xaa⁴ is Ala. In some embodiments, Xaa¹ is an N-terminal amino acid residue D-Phe; Xaa² is Gln or Cit; Xaa³ is Trp or αMe-Trp; Xaa⁴ is Ala; and Xaa⁵ is Val or Tle. In some embodiments, Xaa¹ is an N-terminal amino acid residue D-Phe; Xaa² is Gln or Cit; Xaa³ is Trp or αMe-Trp; Xaa⁴ is Ala; Xaa⁵ is Val or Tle; and Xaa⁶ is Gly or NMe-Gly. In some embodiments, Xaa¹ is an N-terminal amino acid residue D-Phe; Xaa² is Gln or Cit; Xaa³ is Trp or αMe-Trp; Xaa⁴ is Ala; Xaa⁵ is Val or Tle; Xaa⁶ is Gly or NMe-Gly; and Xaa⁷ is His, Trp, Trp(5-F), 3-Quin-Ala, or 2-Quin-Ala. In some embodiments, Xaa¹ is an N-terminal amino acid residue D-Phe; Xaa² is Gln or Cit; Xaa³ is Trp or αMe-Trp; Xaa⁴ is Ala; Xaa⁵ is Val or Tle; Xaa⁶ is Gly or NMe-Gly; Xaa² is His, Trp, Trp(5-F), 3-Quin-Ala, or 2-Quin-Ala; and Xaa⁸ is Sta or Leu. In some embodiments, Xaa¹ is an N-terminal amino acid residue D-Phe; Xaa² is Gln or Cit; Xaa³ is Trp or αMe-Trp; Xaa⁴ is Ala; Xaa⁵ is Val or Tle; Xaa⁶ is Gly or NMe-Gly; Xaa⁷ is His, Trp, Trp(5-F), or 3-Quin-Ala; and Xaa⁸ is Sta or Leu. In some embodiments, Xaa¹ is an N-terminal amino acid residue D-Phe; Xaa² is Gln or Cit; Xaa³ is Trp or αMe-Trp; Xaa⁴ is Ala; Xaa⁵ is Val or Tle; Xaa⁶ is Gly or NMe-Gly; Xaa⁷ is His, Trp, Trp(7-F), Trp(6-F), Trp(5-F), Trp(4-F), 3-Quin-Ala, or 2-Quin-Ala; and Xaa⁸ is Sta or Leu. In some embodiments, Xaa¹ is an N-terminal amino acid residue D-Phe; Xaa² is Gln or Cit; Xaa³ is Trp or αMe-Trp; Xaa⁴ is Ala; Xaa⁵ is Val or Tle; Xaa⁶ is Gly or NMe-Gly; Xaa² is His, Trp, Trp(7-F), Trp(6-F), Trp(5-F), Trp(4-F), 3-Quin-Ala, or 2-Quin-Ala; Xaa⁸ is Sta or Leu; and Xaa⁹ is Pro or Leu. In some embodiments, Xaa¹ is an N-terminal amino acid residue D-Phe; Xaa² is Gln or Cit; Xaa³ is Trp or αMe-Trp; Xaa⁴ is Ala; Xaa⁵ is Val or Tle; Xaa⁶ is Gly or NMe-Gly; Xaa⁷ is His, Trp, Trp(5-F), or 3-Quin-Ala; and Xaa⁸ is Sta or Leu. In some embodiments, Xaa¹ is an N-terminal amino acid residue D-Phe; Xaa² is Gln or Cit; Xaa³ is Trp or αMe-Trp; Xaa⁴ is Ala; Xaa⁵ is Val or Tle; Xaa⁶ is Gly or NMe-Gly; Xaa² is His, Trp, Trp(5-F), or 3-Quin-Ala; Xaa⁸ is Sta or Leu; and Xaa⁹ is Pro or Leu. In some embodiments, the present disclosure relates to a peptidic compound of Formula (IF), or a salt or solvate of Formula (IF),

(IF)
R^{rad}ₙ₆-R^{L}-[linker]-R^{L}-Xaa¹-Xaa²-Xaa³-Xaa⁴-Xaa⁵-Xaa⁶-
Xaa⁷-Xaa⁸--R

wherein:
Xaa¹ is an N-terminal amino acid residue selected from D-Phe, 4-chlorophenylalanine (Cpa), D-Cpa, 3-(1-naphthyl)alanine (Nal), D-Nal, 3-(2-naphthyl)alanine (2-Nal), D-2-Nal, 2,3,4,9-tetrahydro-1H-pyrido[3,4-b]indol-3-carboxylic acid (Tpi), or D-Tpi;
Xaa² is Gly, Arg, Asp, Asn, Gln, Glu, homoserine (Hse), citrulline (Cit), 2-amino-4-ureidobutanoic acid (Aub), 2-amino-4-guanidinobutyric acid (Agb), or His;
Xaa³ is Trp, β-(3-benzothienyl)alanine (Bta), Trp(Me), Trp(7-Me), Trp(6-Me), Trp(5-Me), Trp(4-Me), Trp(2-Me), Trp(7-F), Trp(6-F), Trp(5-F), Trp(4-F), Trp(7-Cl), Trp(6-Cl), Trp(5-Cl), Trp(4-Cl), Trp(7-Br), Trp(6-Br), Trp(5-Br), Trp(4-Br), Trp(7-1), Trp(6-1), Trp(5-1), Trp(4-1), Trp(5-OH), αMe-Trp, Tpi, 7-azatryptophan (7-Aza), Nal, 2-pyridylalanine (2-Pal), 3-pyridylalanine (3-Pal), 4-pyridylalanine (4-Pal), 2-quinoylalanine, or 3-quinoylalanine;
Xaa⁴ is Ala, D-Ala, or Ser;
Xaa⁵ is Val, 2,3-dehydro-Val, cyclopentylglycine (Cpg), L-cyclopropylglycine, L-cyclobutylglycine, or tert-leucine (Tle);
Xaa⁶ is Gly or D-Ala;
Xaa⁷ is His, Trp, Trp(Me), Trp(7-Me), Trp(6-Me), Trp(5-Me), Trp(4-Me), Trp(2-Me), Trp(7-F), Trp(6-F), Trp(5-F), Trp(4-F), Trp(5-OH), Trp(7-Cl), Trp(6-Cl), Trp(5-Cl), Trp(4-Cl), Trp(7-Br), Trp(6-Br), Trp(5-Br), Trp(4-Br), Trp(7-1), Trp(6-1), Trp(5-1), Trp(4-1), αMe-Trp, Nal, 2-pyridylalanine (2-Pal), 3-pyridylalanine (3-Pal), 4-pyridylalanine (4-Pal), 2-quinoylalanine, or 3-quinoylalanine;
R is —NH₂ or —NHOH;
Xaa⁸-R is a C-terminally amidated (R is —NH₂) or a C-terminally hydroxylamidated (R is —NHOH) amino acid residue selected from statine (Sta), Leu, D-Pro, Phe, cis-4-NHR^{a}-D-Pro or cis-4-OR^{a}-D-Pro, wherein R^{a} is alkyl, cycloalkyl, or aryl;
each R^{L} is independently absent, —(C₁₋₆ alkyl)-, —(C₁₋₆ alkyl)-C(O)—, —C(O)—, —NH—C(O)—, or —NH—C(S)—;
the linker is a linear or branched chain of n1 units of -Xaa¹¹-, and/or -L¹R¹— and/or -(L¹)₂R¹—, wherein:
   n1 is 1-20;
   each Xaa¹¹ is independently an amino acid;
   each R¹ is, independently, a linear, branched, and/or cyclic C₁₋₂₀ alkylenyl, alkenylenyl and/or alkynylenyl, wherein any carbon bonded to two other carbons is optionally independently replaced by N, S, or O, and carbons are optionally independently substituted with oxo, hydroxyl, sulfhydryl, —SeH, halogen, guanidino, amine, amide, urea, carboxylic acid, sulfonic acid, sulfinic acid, or phosphoric acid;
   L¹ bonds to carbon, wherein each L¹ is independently —S—, —N(R²)C(O)—, —C(O)N(R²)—, —NH—C(O)—NH—, —NH—C(S)—NH—,

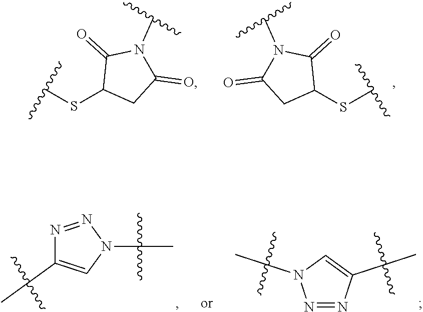

and
R² is H, methyl or ethyl;
optionally, the linker comprises an albumin binder (R^{alb}), wherein the albumin binder is:
   —(CH₂)ₙ₃—CH₃ wherein n3 is 8-20;
   —(CH₂)ₙ₄—C(O)OH wherein n4 is 8-20;

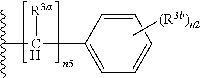

wherein n2 is 1-5, n5 is 1-4, R^{3a} is H or methyl, and R^{3b} is I, Br, F, Cl, H, OH, OCH₃, NH₂, NO₂ or C₁-C₆ alkyl;

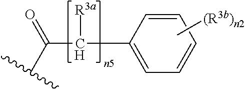

wherein n2 is 1-5, n5 is 1-4, R^{3a} is H or methyl, and R^{3b} is I, Br, F, Cl, H, OH, OCH₃, NH₂, NO₂ or C₁-C₆ alkyl; or

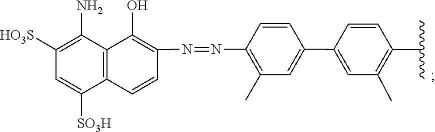 [SMILES: *c1ccc(-c2ccc(N=Nc3ccc4c(S(=O)(=O)O)cc(S(=O)(=O)O)c(N)c4c3O)c(C)c2)cc1C]

n6 is 1-5;
each R^{rad} is a radiolabeling group independently selected from a radiometal chelator; an aryl or heteroaryl substituted with at least one radiohalogen; a prosthetic group containing a trifluoroborate; an aryl or heteroaryl substituted with at least one fluorine precursor leaving group; a prosthetic group containing a silicon-fluorine-acceptor moiety; or a prosthetic group containing a fluorophosphate, fluorosulfate, sulfonyl fluoride, or a combination thereof; and
wherein Xaa¹, Xaa², Xaa³, Xaa⁴, Xaa⁵, Xaa⁶, Xaa⁷, or Xaa⁸ is each optionally methylated.

In some embodiments, the present disclosure relates to a peptidic compound of Formula (IF-1), or a salt or solvate of Formula (IF-1),

(IF-1)
R^{rad}-R^{L}-[linker]-Xaa¹-Xaa²-Xaa³-Xaa⁴-Xaa⁵-Xaa⁶-Xaa⁷-
Xaa⁸--R

wherein:
Xaa¹ is an N-terminal amino acid residue selected from D-Phe, 4-chlorophenylalanine (Cpa), D-Cpa, 3-(1-naphthyl)alanine (Nal), D-Nal, 3-(2-naphthyl)alanine (2-Nal), D-2-Nal, 2,3,4,9-tetrahydro-1H-pyrido[3,4-b]indol-3-carboxylic acid (Tpi), or D-Tpi;
Xaa² is Gly, Arg, Asp, Asn, Gln, Glu, homoserine (Hse), citrulline (Cit), 2-amino-4-ureidobutanoic acid (Aub), 2-amino-4-guanidinobutyric acid (Agb), or His;
Xaa³ is Trp, β-(3-benzothienyl)alanine (Bta), Trp(Me), Trp(7-Me), Trp(6-Me), Trp(5-Me), Trp(4-Me), Trp(2-Me), Trp(7-F), Trp(6-F), Trp(5-F), Trp(4-F), Trp(7-Cl), Trp(6-Cl), Trp(5-Cl), Trp(4-Cl), Trp(7-Br), Trp(6-Br), Trp(5-Br), Trp(4-Br), Trp(7-1), Trp(6-1), Trp(5-1), Trp(4-1), Trp(5-OH), αMe-Trp, Tpi, 7-azatryptophan (7-Aza), Nal, 2-pyridylalanine (2-Pal), 3-pyridylalanine (3-Pal), 4-pyridylalanine (4-Pal), 2-quinoylalanine, or 3-quinoylalanine;
Xaa⁴ is Ala, D-Ala, or Ser;
Xaa⁵ is Val, 2,3-dehydro-Val, cyclopentylglycine (Cpg), L-cyclopropylglycine, L-cyclobutylglycine, or tert-leucine (Tle);
Xaa⁶ is Gly or D-Ala;
Xaa⁷ is His, Trp, Trp(Me), Trp(7-Me), Trp(6-Me), Trp(5-Me), Trp(4-Me), Trp(2-Me), Trp(7-F), Trp(6-F), Trp(5-F), Trp(4-F), Trp(5-OH), Trp(7-Cl), Trp(6-Cl), Trp(5-Cl), Trp(4-Cl), Trp(7-Br), Trp(6-Br), Trp(5-Br), Trp(4-Br), Trp(7-1), Trp(6-1), Trp(5-1), Trp(4-1), αMe-Trp, Nal, 2-pyridylalanine (2-Pal), 3-pyridylalanine (3-Pal), 4-pyridylalanine (4-Pal), 2-quinoylalanine, or 3-quinoylalanine;
R is —NH₂ or —NHOH;
Xaa⁸-R is a C-terminally amidated (R is —NH₂) or a C-terminally hydroxylamidated (R is —NHOH) amino acid residue selected from is statine (Sta), Leu, D-Pro, Phe, cis-4-NHR^{a}-D-Pro or cis-4-OR^{a}-D-Pro, wherein R^{a} is alkyl, cycloalkyl, or aryl;
   the linker and R^{L} together form a p-aminomethylaniline-diglycolic acid (pABzA-DIG) linker, a 4-amino-(1-carboxymethyl)piperidine (Pip) linker, a 9-amino-4,7-dioxanonanoic acid (dPEG2) linker, or a 4-(2-aminoethyl)-1-carboxymethyl-piperazine (Acp) linker;
R^{rad} is a radiometal chelator; and
wherein Xaa¹, Xaa², Xaa³, Xaa⁴, Xaa⁵, Xaa⁶, Xaa⁷, or Xaa⁸, is each optionally N-methylated.

In some embodiments of the compounds of formula (IF) or (IF-1), Xaa⁸-R is a C-terminally amidated (R is —NH₂) or a C-terminally hydroxylamidated (R is —NHOH) amino acid residue selected from D-Pro, cis-4-NHR^{a}-D-Pro or cis-4-OR^{a}-D-Pro, wherein R^{a} is alkyl, cycloalkyl, or aryl. In some embodiments, R^{a} is C₁-C₆ alkyl, C₃-C₇ cycloalkyl, or phenyl. In some embodiments, R^{a} is C₃-C₇ cycloalkyl. In some embodiments, R^{a} is C₆ cycloalkyl.

In some embodiments of the compounds of formula (IF) or (IF-1), Xaa⁸-R is a C-terminally amidated (R is —NH₂) amino acid residue selected from cis-4-cyclohexyloxy-D-Pro or cis-4-cyclohexylamino-D-Pro.

In some embodiments of the compounds of formula (IF) or (IF-1), Xaa¹ is an N-terminal amino acid residue D-Phe. In some embodiments, Xaa² is Gln or Cit. In some embodiments, Xaa³ is Trp or αMe-Trp. In some embodiments, Xaa⁴ is Ala. In some embodiments, Xaa⁵ is Val or Tle. In some embodiments, Xaa⁶ is Gly or NMe-Gly. In some embodiments, Xaa² is His, Trp, Trp(7-F), Trp(6-F), Trp(5-F), Trp(4-F), 3-Quin-Ala, or 2-Quin-Ala. In some embodiments, Xaa² is His, Trp, Trp(5-F), or 3-Quin-Ala.

In some embodiments of the compounds of formula (IF) or (IF-1), Xaa¹ is an N-terminal amino acid residue D-Phe and Xaa² is Gln or Cit. In some embodiments, Xaa¹ is an N-terminal amino acid residue D-Phe; Xaa² is Gln or Cit; and Xaa³ is Trp or αMe-Trp. In some embodiments, Xaa¹ is an N-terminal amino acid residue D-Phe; Xaa² is Gln or Cit; Xaa³ is Trp or αMe-Trp; and Xaa⁴ is Ala. In some embodiments, Xaa¹ is an N-terminal amino acid residue D-Phe; Xaa² is Gln or Cit; Xaa³ is Trp or αMe-Trp; Xaa⁴ is Ala; and Xaa⁵ is Val or Tle. In some embodiments, Xaa¹ is an N-terminal amino acid residue D-Phe; Xaa² is Gln or Cit; Xaa³ is Trp or αMe-Trp; Xaa⁴ is Ala; Xaa⁵ is Val or Tle; and Xaa⁶ is Gly or NMe-Gly. In some embodiments, Xaa¹ is an N-terminal amino acid residue D-Phe; Xaa² is Gln or Cit; Xaa³ is Trp or αMe-Trp; Xaa⁴ is Ala; Xaa⁵ is Val or Tle; Xaa⁶ is Gly or NMe-Gly; and Xaa² is His, Trp, Trp(5-F), or 3-Quin-Ala. In some embodiments, Xaa¹ is an N-terminal amino acid residue D-Phe; Xaa² is Gln or Cit; Xaa³ is Trp or αMe-Trp; Xaa⁴ is Ala; Xaa⁵ is Val or Tle; Xaa⁶ is Gly or NMe-Gly; Xaa² is His, Trp, Trp(5-F), or 3-Quin-Ala; and Xaa⁸-R is a C-terminally amidated (R is —NH₂) amino acid residue selected from cis-4-cyclohexyloxy-D-Pro or cis-4-cyclohexylamino-D-Pro.

In some embodiments, the present disclosure relates to a peptidic compound of Formula (II), or a salt or solvate of Formula (II),

(II)
R^{rad}ₙ₆-[linker]-Xaa¹-Xaa²-Xaa³-Xaa⁴-Xaa⁵-Xaa⁶-Xaa⁷-
Xaa⁸-[ψ-Xaa⁹-(Xaa¹⁰)ₘ₂]ₘ₁-R

wherein:
Xaa¹ is an N-terminal amino acid residue selected from D-Phe, 4-chlorophenylalanine (Cpa), D-Cpa, 3-(1-naphthyl)alanine (Nal), D-Nal, 3-(2-naphthyl)alanine (2-Nal), D-2-Nal, 2,3,4,9-tetrahydro-1H-pyrido[3,4-b]indol-3-carboxylic acid (Tpi), or D-Tpi;
Xaa² is Gly, Arg, Asp, Asn, Gln, Glu, homoserine (Hse), citrulline (Cit), 2-amino-4-ureidobutanoic acid (Aub), 2-amino-4-guanidinobutyric acid (Agb), or His;
Xaa³ is Trp, β-(3-benzothienyl)alanine (Bta), Trp(Me), Trp(7-Me), Trp(6-Me), Trp(5-Me), Trp(4-Me), Trp(2-Me), Trp(7-F), Trp(6-F), Trp(5-F), Trp(4-F), Trp(7-Cl), Trp(6-Cl), Trp(5-Cl), Trp(4-Cl), Trp(7-Br), Trp(6-Br), Trp(5-Br), Trp(4-Br), Trp(7-1), Trp(6-1), Trp(5-1), Trp(4-1), Trp(5-OH), αMe-Trp, Tpi, 7-azatryptophan (7-Aza), Nal, 2-pyridylalanine (2-Pal), 3-pyridylalanine (3-Pal), 4-pyridylalanine (4-Pal), 2-quinoylalanine (2-Quin-Ala), or 3-quinoylalanine (3-Quin-Ala);
Xaa⁴ is Ala, D-Ala, or Ser;
Xaa⁵ is Val, 2,3-dehydro-Val, cyclopentylglycine (Cpg), L-cyclopropylglycine, L-cyclobutylglycine, or tert-leucine (Tle);
Xaa⁶ is Gly or D-Ala;
Xaa⁷ is His, Trp, Trp(Me), Trp(7-Me), Trp(6-Me), Trp(5-Me), Trp(4-Me), Trp(2-Me), Trp(7-F), Trp(6-F), Trp(5-F), Trp(4-F), Trp(7-Cl), Trp(6-Cl), Trp(5-Cl), Trp(4-Cl), Trp(7-Br), Trp(6-Br), Trp(5-Br), Trp(4-Br), Trp(7-1), Trp(6-1), Trp(5-1), Trp(4-1), Trp(5-OH), αMe-Trp, Nal, 2-pyridylalanine (2-Pal), 3-pyridylalanine (3-Pal), 4-pyridylalanine (4-Pal), 2-quinoylalanine (2-Quin-Ala), or 3-quinoylalanine (3-Quin-Ala);
Xaa⁸ is statine (Sta), Leu, D-Pro, Phe, cis-4-NHR^{a}-D-Pro or cis-4-OR^{a}-D-Pro, wherein R^{a} is alkyl, cycloalkyl, or aryl;
R is —NH₂ or —NHOH;
m1 is 0 or 1;
m2 is 0 or 1;
   when m1 is 1 and m2 is 0, Xaa⁹-R is a C-terminally amidated (R is —NH₂) or a C-terminally hydroxylamidated (R is —NHOH) amino acid residue selected from Pro, Phe, oxazolidine-4-carboxylic acid (4-oxa-L-Pro), 4,4-difluoroproline (difluoroPro), cis-4-NHR^{a}-D-Pro, cis-4-OR^{a}-D-Pro, Leu, 5,5-dimethyl-1,3-thiazolidine-4-carboxylic acid (Me₂Thz), or thiazoline-4-carboxylic acid (Thz), wherein R^{a} is alkyl, cycloalkyl, or aryl;
   when m1 is 1 and m2 is 1, Xaa⁹ is Pro, Phe, oxazolidine-4-carboxylic acid (4-oxa-L-Pro), 4,4-difluoroproline (difluoroPro), Leu, 5,5-dimethyl-1,3-thiazolidine-4-carboxylic acid (Me₂Thz), or thiazoline-4-carboxylic acid (Thz); and Xaa¹⁰-R is a C-terminally amidated (R is —NH₂) or a C-terminally hydroxylamidated (R is —NHOH) amino acid residue selected from Val, Leu, Tle, Ile, Nle, Ala, Gly, or Met;
ψ represents a peptide bond or reduced peptide bond joining Xaa⁸ to Xaa⁹;
the linker is a linear or branched chain of n1 units of -Xaa¹¹-, wherein at least one Xaa¹¹ is bonded to albumin binder (R^{ab});
   n1 is 1-20;
   each Xaa¹¹ is independently an amino acid;
   R^{alb} is:
      —(CH₂)ₙ₃—CH₃ wherein n3 is 8-20;
      —(CH₂)ₙ₄—C(O)OH wherein n4 is 8-20;

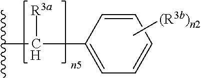

wherein n2 is 1-5, n5 is 1-4, R^{3a} is H or methyl, and R^{3b} is I, Br, F, Cl, H, OH, OCH₃, NH₂, NO₂ or C₁-C₆ alkyl;

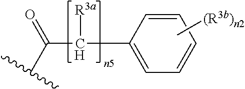

wherein n2 is 1-5, n5 is 1-4, R^{3a} is H or methyl, and R^{3b} is I, Br, F, Cl, H, OH, OCH₃, NH₂, NO₂ or C₁-C₆ alkyl; or

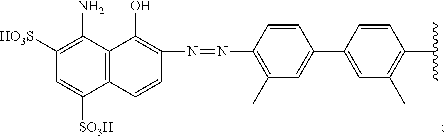 [SMILES: *c1ccc(-c2ccc(N=Nc3ccc4c(S(=O)(=O)O)cc(S(=O)(=O)O)c(N)c4c3O)c(C)c2)cc1C]

n6 is 1-5;
each R^{rad} is a radiolabeling group independently selected from a radiometal chelator; an aryl or heteroaryl substituted with at least one radiohalogen; a prosthetic group containing a trifluoroborate; an aryl or heteroaryl substituted with at least one fluorine precursor leaving group; a prosthetic group containing a silicon-fluorine-acceptor moiety; or a prosthetic group containing a fluorophosphate, fluorosulfate, sulfonyl fluoride, or a combination thereof; and
wherein Xaa¹, Xaa², Xaa³, Xaa⁴, Xaa⁵, Xaa⁶, Xaa⁷, Xaa⁸, or Xaa¹⁰ is each optionally methylated.

In some embodiments of the compounds of formula (II) the linker is a linear peptide of 3 to 6 amino acid residues, -Xaa^{11A}-Xaa^{11B}-(Xaa^{11C})₁₋₄-, wherein Xaa^{11A} Xaa^{11B}, and Xaa^{11C} are each independently an amino acid and wherein the radiolabeling group and the albumin-binding group are bonded to the free N-terminus of Xaa^{11A} and the side chain of Xaa^{11B}, respectively, or are bonded to the side chain of Xaa^{11B} and the free N-terminus of Xaa^{11A}, respectively. In some embodiments, Xaa^{11A} is Gly, Glu, D-Glu, Asp, D-Asp, 2-Aad, D-2-Aad, 3-Aad, or D-3-Aad. In some embodiments, Xaa^{11A} is Gly, Glu or D-Glu. In some embodiments, Xaa^{11A} is Gly. In some embodiments, Xaa^{11B} is Lys, Orn, D-Orn, Dab, D-Dab, Dap or D-Dap, any of which can form an amide bond with a carboxylate of a radiolabeling group or an albumin binding group. In some embodiments, Xaa^{11B} is Lys or D-Lys. In some such embodiments, (Xaa^{11C})₁₋₄ is diglycine. In some such embodiments, (Xaa^{11C})₁₋₄ is p-aminomethylaniline-diglycolic acid (pABzA-DIG), a 4-amino-(1-carboxymethyl)piperidine (Pip), a 9-amino-4,7-dioxanonanoic acid (dPEG2), or a 4-(2-aminoethyl)-1-carboxymethyl-piperazine (Acp). In some such embodiments, (Xaa^{11C})₁₋₄ is Pip. In some embodiments, the C-terminus of Xaa^{11C} and the N-terminus of Xaa¹ are bonded.

In some embodiments of the compounds of formula (II) the linker is a branched peptide of 3 to 6 amino acid residues, -Xaa^{11B}(Xaa^{11A})-(Xaa^{11C})₁₋₄-, wherein Xaa¹¹A, Xaa^{11B}, and Xaa^{11C} are each independently an amino acid and wherein the C-terminus of Xaa^{11A} forms an amide with the side chain of Xaa^{11B}, and wherein the radiolabeling group and the albumin-binding group are bonded to the free N-termini of Xaa^{11A} and Xaa^{11B}, respectively, or are bonded to the free N-termini of Xaa^{11B} and Xaa^{11A}, respectively. In some embodiments, Xaa^{11A} is Gly, Glu, D-Glu, Asp, D-Asp, 2-Aad, D-2-Aad, 3-Aad, or D-3-Aad. In some embodiments, Xaa^{11A} is Glu or D-Glu. In some embodiments, Xaa^{11B} is Lys, D-Lys, Orn, D-Orn, Dab, D-Dab, Dap or D-Dap. In some embodiments, Xaa^{11B} is Lys or D-Lys. In some such embodiments, (Xaa^{11C})_{1.4} is diglycine. In some such embodiments, (Xaa^{11C})₁₄ is p-aminomethylaniline-diglycolic acid (pABzA-DIG), a 4-amino-(1-carboxymethyl)piperidine (Pip), a 9-amino-4,7-dioxanonanoic acid (dPEG2), or a 4-(2-aminoethyl)-1-carboxymethyl-piperazine (Acp). In some such embodiments, (Xaa^{11C})₁₄ is Pip. In some embodiments, the C-terminus of Xaa^{11C} and the N-terminus of Xaa¹ are bonded.

In some embodiments, the present disclosure relates to a peptidic compound of Formula (IIA), or a salt or solvate of Formula (IIA),

(IIA)
R^{rad}-[linker]-Xaa¹-Xaa²-Xaa³-Xaa⁴-Xaa⁵-Xaa⁶-Xaa⁷-
Xaa⁸-ψ-Xaa⁹-(Xaa¹⁰)ₘ₂-R

wherein:
Xaa¹ is an N-terminal amino acid residue selected from D-Phe, 4-chlorophenylalanine (Cpa), D-Cpa, 3-(1-naphthyl)alanine (Nal), D-Nal, 3-(2-naphthyl)alanine (2-Nal), D-2-Nal, 2,3,4,9-tetrahydro-1H-pyrido[3,4-b]indol-3-carboxylic acid (Tpi), or D-Tpi;
Xaa² is Gly, Arg, Asp, Asn, Gln, Glu, homoserine (Hse), citrulline (Cit), 2-amino-4-ureidobutanoic acid (Aub), 2-amino-4-guanidinobutyric acid (Agb), or His;
Xaa³ is Trp, β-(3-benzothienyl)alanine (Bta), Trp(Me), Trp(7-Me), Trp(6-Me), Trp(5-Me), Trp(4-Me), Trp(2-Me), Trp(7-F), Trp(6-F), Trp(5-F), Trp(4-F), Trp(7-Cl), Trp(6-Cl), Trp(5-Cl), Trp(4-Cl), Trp(7-Br), Trp(6-Br), Trp(5-Br), Trp(4-Br), Trp(7-1), Trp(6-1), Trp(5-1), Trp(4-1), Trp(5-OH), αMe-Trp, Tpi, 7-azatryptophan (7-Aza), Nal, 2-pyridylalanine (2-Pal), 3-pyridylalanine (3-Pal), 4-pyridylalanine (4-Pal), 2-quinoylalanine (2-Quin-Ala), or 3-quinoylalanine (3-Quin-Ala);
Xaa⁴ is Ala, D-Ala, or Ser;
Xaa⁵ is Val, 2,3-dehydro-Val, cyclopentylglycine (Cpg), L-cyclopropylglycine, L-cyclobutylglycine, or tert-leucine (Tle);
Xaa⁶ is Gly or D-Ala;
Xaa⁷ is His, Trp, Trp(Me), Trp(7-Me), Trp(6-Me), Trp(5-Me), Trp(4-Me), Trp(2-Me), Trp(7-F), Trp(6-F), Trp(5-F), Trp(4-F), Trp(7-Cl), Trp(6-Cl), Trp(5-Cl), Trp(4-Cl), Trp(7-Br), Trp(6-Br), Trp(5-Br), Trp(4-Br), Trp(7-1), Trp(6-1), Trp(5-1), Trp(4-1), Trp(5-OH), αMe-Trp, Nal, 2-pyridylalanine (2-Pal), 3-pyridylalanine (3-Pal), 4-pyridylalanine (4-Pal), 2-quinoylalanine (2-Quin-Ala), or 3-quinoylalanine (3-Quin-Ala);
Xaa⁸ is statine (Sta), Leu, D-Pro, or Phe;
R is —NH₂ or —NHOH;
m2 is 0 or 1;
   when m2 is 0, Xaa⁹-R is a C-terminally amidated (R is —NH₂) or a C-terminally hydroxylamidated (R is —NHOH) amino acid residue selected from Pro, Phe, oxazolidine-4-carboxylic acid (4-oxa-L-Pro), 4,4-difluoroproline (difluoroPro), cis-4-NHR^{a}-D-Pro, cis-4-OR^{a}-D-Pro, Leu, Me₂Thz (5,5-dimethyl-1,3-thiazolidine-4-carboxylic acid), or thiazoline-4-carboxylic acid (Thz), wherein R^{a} is alkyl, cycloalkyl, or aryl;
   when m2 is 1, Xaa⁹ is Pro, Phe, oxazolidine-4-carboxylic acid (4-oxa-L-Pro), 4,4-difluoroproline (difluoroPro), Leu, Me₂Thz (5,5-dimethyl-1,3-thiazolidine-4-carboxylic acid), or thiazoline-4-carboxylic acid (Thz); and Xaa¹⁰-R is a C-terminally amidated (R is —NH₂) or a C-terminally hydroxylamidated (R is —NHOH) amino acid residue selected from Val, Leu, Tle, Ile, Nle, Ala, Gly, or Met;
ψ represents a peptide bond or reduced peptide bond joining Xaa⁸ to Xaa⁹;
the linker is:
   a linear peptide of 3 to 6 amino acid residues, -Xaa^{11A}-Xaa^{11B}-(Xaa^{11C})₁₋₄-, wherein Xaa^{11A} Xaa^{11B}, and Xaa^{11C} are each independently an amino acid and wherein the radiolabeling group and the albumin-binding group are bonded to the free N-terminus of Xaa^{11A} and the side chain of Xaa^{11B}, respectively, or are bonded to the side chain of Xaa^{11B} and the free N-terminus of Xaa^{11A}, respectively; or
   a branched peptide of 3 to 6 amino acid residues, -Xaa^{11B}(Xaa^{11A})-(Xaa^{11C})₁₋₄-, wherein Xaa^{11A} Xaa^{11B}, and Xaa^{11C} are each independently an amino acid and wherein the C-terminus of Xaa^{11A} forms an amide with the side chain of Xaa^{11B}, and wherein the radiolabeling group and the albumin-binding group are bonded to the free N-termini of Xaa^{11A} and Xaa^{11B} respectively, or are bonded to the free N-termini of Xaa^{11B} and Xaa^{11A}, respectively;
   R^{alb} is

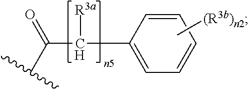

n2 is 1-5;
n5 is 1-4;
each R^{3a} is independently H or methyl;
each R^{3b} is independently I, Br, F, Cl, H, OH, OCH₃, NH₂, NO₂ or C₁-C₆ alkyl;
R^{rad} is a radiolabeling group independently selected from a radiometal chelator; an aryl or heteroaryl substituted with at least one radiohalogen; a prosthetic group containing a trifluoroborate; an aryl or heteroaryl substituted with at least one fluorine precursor leaving group; a prosthetic group containing a silicon-fluorine-acceptor moiety; or a prosthetic group containing a fluorophosphate, fluorosulfate, sulfonyl fluoride, or a combination thereof; and
wherein Xaa¹, Xaa², Xaa³, Xaa⁴, Xaa⁵, Xaa⁶, Xaa⁷, Xaa⁸, or Xaa¹⁰ is each optionally methylated.

In some embodiments of the compounds of formula (II) or (IIA), n2 is 1. In some embodiments, R^{alb} is

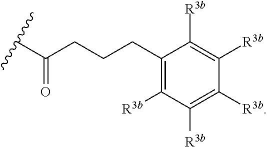

In some embodiments, R^{alb} is

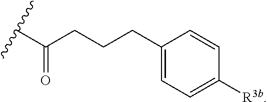

In some embodiments of the compounds of formula (II) or (IIA) the linker is a linear peptide of 3 amino acid residues, -Xaa^{11A}-Xaa^{11B}-Xaa^{11C}-, wherein Xaa^{1A}, Xaa^{11B}, and Xaa^{11C} are each independently an amino acid and wherein the radiolabeling group and the albumin-binding group are bonded to the free N-terminus of Xaa^{11A} and the side chain of Xaa^{11B}, respectively, or are bonded to the side chain of Xaa^{11B} and the free N-terminus of Xaa^{11A}, respectively. In some embodiments, Xaa^{11A} is Gly, Glu, D-Glu, Asp, D-Asp, 2-Aad, D-2-Aad, 3-Aad, or D-3-Aad. In some embodiments, Xaa^{11A} is Gly, Glu or D-Glu. In some embodiments, Xaa^{11A} is Gly. In some embodiments, Xaa^{11B} is Lys, D-Lys, Orn, D-Orn, Dab, D-Dab, Dap or D-Dap, any of which can form an amide bond with a carboxylate of a radiolabeling group or an albumin binding group. In some embodiments, Xaa^{11B} is Lys or D-Lys. In some such embodiments, Xaa^{11C} is p-aminomethylaniline-diglycolic acid (pABzA-DIG), a 4-amino-(1-carboxymethyl)piperidine (Pip), a 9-amino-4,7-dioxanonanoic acid (dPEG2), or a 4-(2-aminoethyl)-1-carboxymethyl-piperazine (Acp). In some such embodiments, Xaa^{11C} is Pip. In some embodiments, the C-terminus of Xaa^{11C} and the N-terminus of Xaa¹ are bonded.

In some embodiments of the compounds of formula (II) or (IIA) the linker is -Gly-Lys-Pip-. In some embodiments, the radiolabeling group is bonded to the free N-terminus of Xaa^{11A} and the albumin-binding group is bonded to the side chain of Xaa¹B. In some embodiments, the radiolabeling group is bonded to the free N-terminus of Gly and the albumin-binding group is bonded to the side chain of Lys.

In some embodiments of the compounds of formula (II) or (IIA) the linker is a branched peptide of 3 amino acid residues, -Xaa^{1B}(Xaa^{11A})-Xaa^{11C}-, wherein Xaa^{1A}, Xaa^{11B}, and Xaa^{11C} are each independently an amino acid and wherein the C-terminus of Xaa^{11A} forms an amide with the side chain of Xaa^{11B}, and wherein the radiolabeling group and the albumin-binding group are bonded to the free N-termini of Xaa^{11A} and Xaa^{11B}, respectively, or are bonded to the free N-termini of Xaa^{11B} and Xaa^{11A}, respectively. In some embodiments, Xaa^{11A} is Gly, Glu, D-Glu, Asp, D-Asp, 2-Aad, D-2-Aad, 3-Aad, or D-3-Aad. In some embodiments, Xaa^{11A} is Glu or D-Glu. In some embodiments, Xaa^{11B} is Lys, D-Lys, Orn, D-Orn, Dab, D-Dab, Dap or D-Dap. In some embodiments, Xaa^{11B} is Lys or D-Lys. In some such embodiments, Xaa^{11C} is p-aminomethylaniline-diglycolic acid (pABzA-DIG), a 4-amino-(1-carboxymethyl)piperidine (Pip), a 9-amino-4,7-dioxanonanoic acid (dPEG2), or a 4-(2-aminoethyl)-1-carboxymethyl-piperazine (Acp). In some such embodiments, Xaa^{11C} is Pip. In some embodiments, the C-terminus of Xaa^{11C} and the N-terminus of Xaa¹ are bonded.

In some embodiments of the compounds of formula (II) or (IIA), Xaa¹ is an N-terminal amino acid residue D-Phe. In some embodiments, Xaa² is Gln or Cit. In some embodiments, Xaa² is Gln. In some embodiments, Xaa³ is Trp or αMe-Trp. In some embodiments, Xaa⁴ is Ala. In some embodiments, Xaa⁵ is Val or Tle. In some embodiments, Xaa⁶ is Gly or NMe-Gly. In some embodiments, Xaa⁷ is His, Trp, Trp(7-F), Trp(6-F), Trp(5-F), or Trp(4-F). In some embodiments, Xaa⁸ is Sta or Leu. In some embodiments, m2 is 0 and Xaa⁹-R is a C-terminally amidated (R is —NH₂) amino acid residue Pro or Leu. In some embodiments, m2 is 1 and Xaa⁹ is Pro or Leu.

In some embodiments of the compounds of formula (II) or (IIA), Xaa¹ is an N-terminal amino acid residue D-Phe and Xaa² is Gln or Cit. In some embodiments, Xaa¹ is an N-terminal amino acid residue D-Phe; Xaa² is Gln or Cit; and Xaa³ is Trp or αMe-Trp. In some embodiments, Xaa¹ is an N-terminal amino acid residue D-Phe; Xaa² is Gln or Cit; Xaa³ is Trp or αMe-Trp; and Xaa⁴ is Ala. In some embodiments, Xaa¹ is an N-terminal amino acid residue D-Phe; Xaa² is Gln or Cit; Xaa³ is Trp or αMe-Trp; Xaa⁴ is Ala; and Xaa⁵ is Val or Tle. In some embodiments, Xaa¹ is an N-terminal amino acid residue D-Phe; Xaa² is Gln or Cit; Xaa³ is Trp or αMe-Trp; Xaa⁴ is Ala; Xaa⁵ is Val or Tle; and Xaa⁶ is Gly or NMe-Gly. In some embodiments, Xaa¹ is an N-terminal amino acid residue D-Phe; Xaa² is Gln or Cit; Xaa³ is Trp or αMe-Trp; Xaa⁴ is Ala; Xaa⁵ is Val or Tle; Xaa⁶ is Gly or NMe-Gly; and Xaa² is His, Trp, Trp(5-F), or 3-Quin-Ala. In some embodiments, Xaa¹ is an N-terminal amino acid residue D-Phe; Xaa² is Gln or Cit; Xaa³ is Trp or αMe-Trp; Xaa⁴ is Ala; Xaa⁵ is Val or Tle; Xaa⁶ is Gly or NMe-Gly; Xaa² is His, Trp, Trp(5-F), or 3-Quin-Ala; and Xaa⁸ is Sta or Leu. In some embodiments, Xaa¹ is an N-terminal amino acid residue D-Phe; Xaa² is Gln or Cit; Xaa³ is Trp or αMe-Trp; Xaa⁴ is Ala; Xaa⁵ is Val or Tle; Xaa⁶ is Gly or NMe-Gly; Xaa² is His, Trp, Trp(7-F), Trp(6-F), Trp(5-F), Trp(4-F), 3-Quin-Ala, or 2-Quin-Ala; and Xaa⁸ is Sta or Leu.

In some embodiments, the present disclosure relates to a peptidic compound of Formula (B), or a salt or solvate of Formula (B),

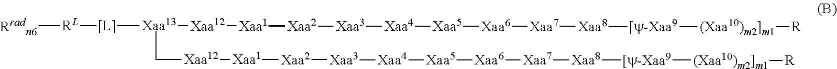

wherein:
each Xaa¹² is independently an N-terminal amino acid residue selected from p-aminomethylaniline-diglycolic acid (pABzA-DIG), 4-amino-(1-carboxymethyl)piperidine (Pip), 9-amino-4,7-dioxanonanoic acid (dPEG2), or 4-(2-aminoethyl)-1-carboxymethyl-piperazine (Acp);
each Xaa¹ is independently D-Phe, 4-chlorophenylalanine (Cpa), D-Cpa, 3-(1-naphthyl)alanine (Nal), D-Nal, 3-(2-naphthyl)alanine (2-Nal), D-2-Nal, 2,3,4,9-tetrahydro-1H-pyrido[3,4-b]indol-3-carboxylic acid (Tpi), or D-Tpi;
each Xaa² is independently Gly, Arg, Asp, Asn, Gln, Glu, homoserine (Hse), citrulline (Cit), 2-amino-4-ureidobutanoic acid (Aub), 2-amino-4-guanidinobutyric acid (Agb), or His;
each Xaa³ is independently Trp, β-(3-benzothienyl)alanine (Bta), Trp(Me), Trp(7-Me), Trp(6-Me), Trp(5-Me), Trp(4-Me), Trp(2-Me), Trp(7-F), Trp(6-F), Trp(5-F), Trp(4-F), Trp(7-Cl), Trp(6-Cl), Trp(5-Cl), Trp(4-Cl), Trp(7-Br), Trp(6-Br), Trp(5-Br), Trp(4-Br), Trp(7-1), Trp(6-1), Trp(5-1), Trp(4-1), Trp(5-OH), αMe-Trp, Tpi, 7-azatryptophan (7-Aza), Nal, 2-pyridylalanine (2-Pal), 3-pyridylalanine (3-Pal), 4-pyridylalanine (4-Pal), 2-quinoylalanine (2-Quin-Ala), or 3-quinoylalanine (3-Quin-Ala);
each Xaa⁴ is independently Ala, D-Ala, or Ser;
each Xaa⁵ is independently Val, 2,3-dehydro-Val, cyclopentylglycine (Cpg), L-cyclopropylglycine, L-cyclobutylglycine, or tert-leucine (Tle);
each Xaa⁶ is independently Gly or D-Ala;
each Xaa⁷ is independently His, Trp, Trp(Me), Trp(7-Me), Trp(6-Me), Trp(5-Me), Trp(4-Me), Trp(2-Me), Trp(7-F), Trp(6-F), Trp(5-F), Trp(4-F), Trp(7-Cl), Trp(6-Cl), Trp(5-Cl), Trp(4-Cl), Trp(7-Br), Trp(6-Br), Trp(5-Br), Trp(4-Br), Trp(7-1), Trp(6-1), Trp(5-1), Trp(4-1), Trp(5-OH), αMe-Trp, Nal, 2-pyridylalanine (2-Pal), 3-pyridylalanine (3-Pal), 4-pyridylalanine (4-Pal), 2-quinoylalanine (2-Quin-Ala), or 3-quinoylalanine (3-Quin-Ala);
each Xaa⁸ is independently statine (Sta), Leu, D-Pro, Phe, cis-4-NHR^{a}-D-Pro or cis-4-OR^{a}-D-Pro, wherein R^{a} is alkyl, cycloalkyl, or aryl;
each R is independently —NH₂ or —NHOH;
each m1 is independently 0 or 1;
each m2 is independently 0 or 1;
   when m1 is 1 and m2 is 0, each Xaa⁹-R is independently a C-terminally amidated (R is —NH₂) or a C-terminally hydroxylamidated (R is —NHOH) amino acid residue selected from Pro, Phe, oxazolidine-4-carboxylic acid (4-oxa-L-Pro), 4,4-difluoroproline (difluoroPro), cis-4-NHR^{a}-D-Pro, cis-4-OR^{a}-D-Pro, Leu, 5,5-dimethyl-1,3-thiazolidine-4-carboxylic acid (Me₂Thz), or thiazoline-4-carboxylic acid (Thz), wherein R^{a} is alkyl, cycloalkyl, or aryl;
   when m1 is 1 and m2 is 1, each Xaa⁹ is independently Pro, Phe, oxazolidine-4-carboxylic acid (4-oxa-L-Pro), 4,4-difluoroproline (difluoroPro), Leu, 5,5-dimethyl-1,3-thiazolidine-4-carboxylic acid (Me₂Thz), or thiazoline-4-carboxylic acid (Thz); and Xaa¹⁰-R is a C-terminally amidated (R is —NH₂) or a C-terminally hydroxylamidated (R is —NHOH) amino acid residue selected from Val, Leu, Tle, Ile, Nle, Ala, Gly, or Met;
Xaa¹³ is an amino acid with a sidechain comprising a carboxylic acid, wherein one Xaa¹² is bonded to the C-terminus of Xaa¹³ and the other Xaa¹² is bonded to the sidechain of Xaa¹³;
ψ represents a peptide bond or reduced peptide bond joining Xaa⁸ to Xaa⁹;
R^{L} is absent, —(C₁₋₆ alkyl)-, —(C₁₋₆ alkyl)-C(O)—, —C(O)—, —NH—C(O)—, or —NH—C(S)—;
L is a linker and the linker absent or is a linear or branched chain of n1 units of -Xaa¹¹-, and/or -L¹R¹— and/or -(L¹)₂R¹—, wherein:
   n1 is 1-20;
   each Xaa¹¹ is independently an amino acid;
   each R¹ is, independently, a linear, branched, and/or cyclic C₁₋₂₀ alkylenyl, alkenylenyl and/or alkynylenyl, wherein any carbon bonded to two other carbons is optionally independently replaced by N, S, or O, and carbons are optionally independently substituted with oxo, hydroxyl, sulfhydryl, —SeH, halogen, guanidino, amine, amide, urea, carboxylic acid, sulfonic acid, sulfinic acid, or phosphoric acid;
   L¹ bonds to carbon, wherein each L¹ is independently —S—, —N(R²)C(O)—, —C(O)N(R²)—, —NH—C(O)—NH—, —NH—C(S)—NH—,

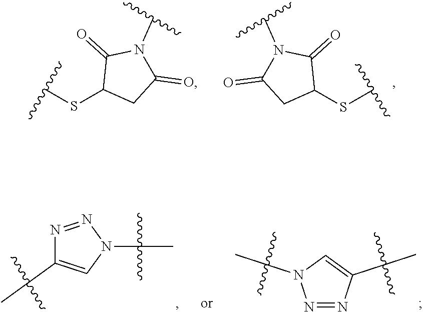

and
R² is H, methyl or ethyl;
optionally, the linker comprises an albumin binder (R^{alb}), wherein the albumin binder is:
   —(CH₂)ₙ₃—CH₃ wherein n3 is 8-20;
   —(CH₂)ₙ₄—C(O)OH wherein n4 is 8-20;

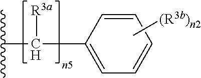

wherein n2 is 1-5, n5 is 1-4, R^{3a} is H or methyl, and R^{3b} is I, Br, F, Cl, H, OH, OCH₃, NH₂, NO₂ or C₁-C₆ alkyl;

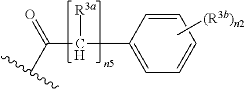

wherein n2 is 1-5, n5 is 1-4, R^{3a} is H or methyl, and R^{3b} is I, Br, F, Cl, H, OH, OCH₃, NH₂, NO₂ or C₁-C₆ alkyl; or

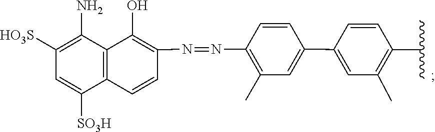 [SMILES: *c1ccc(-c2ccc(N=Nc3ccc4c(S(=O)(=O)O)cc(S(=O)(=O)O)c(N)c4c3O)c(C)c2)cc1C]

n6 is 1-5;
each R^{rad} is a radiolabeling group independently selected from a radiometal chelator; an aryl or heteroaryl substituted with at least one radiohalogen; a prosthetic group containing a trifluoroborate; an aryl or heteroaryl substituted with at least one fluorine precursor leaving group; a prosthetic group containing a silicon-fluorine-acceptor moiety; or a prosthetic group containing a fluorophosphate, fluorosulfate, sulfonyl fluoride, or a combination thereof; and
wherein Xaa¹, Xaa², Xaa³, Xaa⁴, Xaa⁵, Xaa⁶, Xaa⁷, Xaa^{B}, Xaa⁹ or Xaa¹⁰ is each optionally N-methylated.

In some embodiments, the present disclosure relates to a peptidic compound of Formula (B-1), or a salt or solvate of formula (B-1),

(B-1)
R^{rad}-[linker]-Xaa¹³-[Xaa¹²-Xaa¹-Xaa²-Xaa³-Xaa⁴-Xaa⁵-
Xaa⁶-Xaa⁷-Xaa⁸-ψ-Xaa⁹-(Xaa¹⁰)ₘ₂-R]₂

wherein:
Xaa¹² is an N-terminal amino acid residue selected from p-aminomethylaniline-diglycolic acid (pABzA-DIG), 4-amino-(1-carboxymethyl)piperidine (Pip), 9-amino-4,7-dioxanonanoic acid (dPEG2), or 4-(2-aminoethyl)-1-carboxymethyl-piperazine (Acp);
Xaa¹ is D-Phe, 4-chlorophenylalanine (Cpa), D-Cpa, 3-(1-naphthyl)alanine (Nal), D-Nal, 3-(2-naphthyl)alanine (2-Nal), D-2-Nal, 2,3,4,9-tetrahydro-1H-pyrido[3,4-b]indol-3-carboxylic acid (Tpi), or D-Tpi;
Xaa² is Gly, Arg, Asp, Asn, Gln, Glu, homoserine (Hse), citrulline (Cit), 2-amino-4-ureidobutanoic acid (Aub), 2-amino-4-guanidinobutyric acid (Agb), or His;
Xaa³ is Trp, β-(3-benzothienyl)alanine (Bta), Trp(Me), Trp(7-Me), Trp(6-Me), Trp(5-Me), Trp(4-Me), Trp(2-Me), Trp(7-F), Trp(6-F), Trp(5-F), Trp(4-F), Trp(7-Cl), Trp(6-Cl), Trp(5-Cl), Trp(4-Cl), Trp(7-Br), Trp(6-Br), Trp(5-Br), Trp(4-Br), Trp(7-1), Trp(6-1), Trp(5-1), Trp(4-1), Trp(5-OH), αMe-Trp, Tpi, 7-azatryptophan (7-Aza), Nal, 2-pyridylalanine (2-Pal), 3-pyridylalanine (3-Pal), 4-pyridylalanine (4-Pal), 2-quinoylalanine (2-Quin-Ala), or 3-quinoylalanine (3-Quin-Ala);
Xaa⁴ is Ala, D-Ala, or Ser;
Xaa⁵ is Val, 2,3-dehydro-Val, cyclopentylglycine (Cpg), L-cyclopropylglycine, L-cyclobutylglycine, or tert-leucine (Tle);
Xaa⁶ is Gly or D-Ala;
Xaa⁷ is His, Trp, Trp(Me), Trp(7-Me), Trp(6-Me), Trp(5-Me), Trp(4-Me), Trp(2-Me), Trp(7-F), Trp(6-F), Trp(5-F), Trp(4-F), Trp(7-Cl), Trp(6-Cl), Trp(5-Cl), Trp(4-Cl), Trp(7-Br), Trp(6-Br), Trp(5-Br), Trp(4-Br), Trp(7-1), Trp(6-1), Trp(5-1), Trp(4-1), Trp(5-OH), αMe-Trp, Nal, 2-pyridylalanine (2-Pal), 3-pyridylalanine (3-Pal), 4-pyridylalanine (4-Pal), 2-quinoylalanine (2-Quin-Ala), or 3-quinoylalanine (3-Quin-Ala);
Xaa⁸ is statine (Sta), Leu, D-Pro, or Phe;
R is —NH₂ or —NHOH;
m2 is 0 or 1;
   when m2 is 0, Xaa⁹-R is a C-terminally amidated (R is —NH₂) or a C-terminally hydroxylamidated (R is —NHOH) amino acid residue selected from Pro, Phe, oxazolidine-4-carboxylic acid (4-oxa-L-Pro), 4,4-difluoroproline (difluoroPro), cis-4-NHR^{a}-D-Pro, cis-4-OR^{a}-D-Pro, Leu, 5,5-dimethyl-1,3-thiazolidine-4-carboxylic acid (Me₂Thz), or thiazoline-4-carboxylic acid (Thz), wherein R^{a} is alkyl, cycloalkyl, or aryl;
   when m2 is 1, Xaa⁹ is Pro, Phe, oxazolidine-4-carboxylic acid (4-oxa-L-Pro), 4,4-difluoroproline (difluoroPro), Leu, 5,5-dimethyl-1,3-thiazolidine-4-carboxylic acid (Me₂Thz), or thiazoline-4-carboxylic acid (Thz); and Xaa¹⁰-R is a C-terminally amidated (R is —NH₂) or a C-terminally hydroxylamidated (R is —NHOH) amino acid residue selected from Val, Leu, Tle, Ile, Nle, Ala, Gly, or Met;
Xaa¹³ is an amino acid with a sidechain comprising a carboxylic acid, wherein one Xaa¹² is bonded to the C-terminus of Xaa¹³ and the other Xaa¹² is bonded to the sidechain of Xaa¹³;
ψ represents a peptide bond or reduced peptide bond joining Xaa⁸ to Xaa⁹;
the linker absent or is a linear or branched chain of n1 units of -Xaa¹¹-, wherein:
   n1 is 1-6;
   each Xaa¹¹ is independently an amino acid;
R^{rad} is a radiolabeling group independently selected from a radiometal chelator; an aryl or heteroaryl substituted with at least one radiohalogen; a prosthetic group containing a trifluoroborate; an aryl or heteroaryl substituted with at least one fluorine precursor leaving group; a prosthetic group containing a silicon-fluorine-acceptor moiety; or a prosthetic group containing a fluorophosphate, fluorosulfate, sulfonyl fluoride, or a combination thereof; and
wherein Xaa¹, Xaa², Xaa³, Xaa⁴, Xaa⁵, Xaa⁶, Xaa⁷, Xaa⁸, Xaa⁹ or Xaa¹⁰ is each optionally N-methylated.

In some embodiments of the peptidic compound of formula (B), the compound has the structure:

R^{rad}ₙ₆-R^{L}-[linker]-Xaa¹³-[Xaa¹²-Xaa¹-Xaa²-Xaa³-Xaa⁴-Xaa⁵-Xaa⁶-Xaa⁷-Xaa⁸-ψ-Xaa⁹-(Xaa¹⁰)ₘ-R]₂

In some embodiments of the peptidic compound of formula (B) or (B-1), both Xaa¹² is the same amino acid. In some embodiments, each Xaa¹² is the same or different amino acids.

In some embodiments of the peptidic compound of formula (B) or (B-1), both Xaa¹ is the same amino acid. In some embodiments, each Xaa¹ is the same or different amino acids.

In some embodiments of the peptidic compound of formula (B) or (B-1), both Xaa² is the same amino acid. In some embodiments, each Xaa² is the same or different amino acids.

In some embodiments of the peptidic compound of formula (B) or (B-1), both Xaa³ is the same amino acid. In some embodiments, each Xaa³ is the same or different amino acids.

In some embodiments of the peptidic compound of formula (B) or (B-1), both Xaa⁴ is the same amino acid. In some embodiments, each Xaa⁴ is the same or different amino acids.

In some embodiments of the peptidic compound of formula (B) or (B-1), both Xaa⁵ is the same amino acid. In some embodiments, each Xaa⁵ is the same or different amino acids.

In some embodiments of the peptidic compound of formula (B) or (B-1), both Xaa⁶ is the same amino acid. In some embodiments, each Xaa⁶ is the same or different amino acids.

In some embodiments of the peptidic compound of formula (B) or (B-1), both Xaa⁷ is the same amino acid. In some embodiments, each Xaa⁷ is the same or different amino acids.

In some embodiments of the peptidic compound of formula (B) or (B-1), both Xaa⁸ is the same amino acid. In some embodiments, each Xaa⁸ is the same or different amino acids.

In some embodiments of the peptidic compound of formula (B) or (B-1), both Xaa⁹ is the same amino acid. In some embodiments, each Xaa⁹ is the same or different amino acids.

In some embodiments of the peptidic compound of formula (B) or (B-1), both Xaa¹⁰ is the same amino acid. In some embodiments, each Xaa¹⁰ is the same or different amino acids.

In some embodiments of the compounds of formula (B) or (B-1), Xaa¹ is D-Phe. In some embodiments, Xaa¹ is Cpa or D-Cpa. In some embodiments, Xaa¹ is Nal, D-Nal, 2-Nal, D-2-Nal. In some embodiments, Xaa¹ is D-Cpa, Tpi, D-Tpi or D-Nal.

In some embodiments of the compounds of formula (B) or (B-1), Xaa¹² is Pip.

In some embodiments of the compounds of formula (B) or (B-1), Xaa¹³ is Glu, D-Glu, Asp, or D-Asp. In some embodiments, Xaa¹³ is Glu.

In some embodiments of the compounds of formula (B) or (B-1), the linker is absent.

In some embodiments of the compounds of formula (B) or (B-1), the linker is a linear or branched chain of n1 units of -Xaa¹¹-, wherein n1 is 1-3, and each Xaa¹¹ is independently 2-aminoadipic acid (2-Aad) or 3-aminoadipic acid (3-Aad). In some embodiments, each Xaa¹¹ is 2-Aad. In some embodiments, the linker is -2-Aad-2-Aad-.

In some embodiments of the compounds of formula (B) or (B-1), n1 is 1, 2, or 3.

In some embodiments of the compounds of formula (B) or (B-1), R^{rad} is a radiometal chelator. In some embodiments, the radiometal chelator is bound to a radiometal. As used herein, phrases that a radiometal is “bound to a chelator,” “chelated to a chelator,” “conjugated to a chelator,” “conjugated with a chelator,” and “associated with a chelator” are used interchangeably.

In some embodiments of the peptidic compound (e.g., compounds of formula (A), (I), (IA), (IA-1), (IB), (IB3-1), (IC), (IC-1), (ID), (ID-1), (IE), (IE-1), (IF), (IF-1), (II), (IIA), (B), or (B-1) or salt or solvate thereof), R is —NH₂.

In some embodiments of the peptidic compound, ψ represents a reduced peptide bond. In some embodiments, ψ represents a peptide bond.

In some embodiments of the peptidic compound, Xaa¹ is an N-terminal amino acid residue selected from D-Phe.

In some embodiments of the peptidic compound, Xaa² is Gln. In some embodiments, Xaa² is Cit. In some embodiments, Xaa² is Glu. In some embodiments, Xaa² is Agb. In some embodiments, Xaa² is Asn, Gln, or Hse. In some embodiments, Xaa² is Gln, Cit, Glu, or Agb. In some embodiments, Xaa² is Gln or Cit.

In some embodiments of the peptidic compound, Xaa³ is Trp. In some embodiments, Xaa³ is αMe-Trp. In some embodiments, Xaa³ is Trp, Trp(Me), Trp(7-Me), Trp(6-Me), Trp(5-Me), Trp(4-Me), Trp(2-Me), Trp(7-F), Trp(6-F), Trp(5-F), Trp(4-F), Trp(7-Cl), Trp(6-Cl), Trp(5-Cl), Trp(4-Cl), Trp(7-Br), Trp(6-Br), Trp(5-Br), Trp(4-Br), Trp(7-1), Trp(6-I), Trp(5-1), Trp(4-1), Trp(5-OH), αMe-Trp, Nal, 2-pyridylalanine (2-Pal), 3-pyridylalanine (3-Pal), 4-pyridylalanine (4-Pal), 2-quinoylalanine (2-Quin-Ala), or 3-quinoylalanine (3-Quin-Ala). In some embodiments, Xaa³ is Trp, Trp(Me), Trp(7-Me), Trp(6-Me), Trp(5-Me), Trp(4-Me), Trp(2-Me), Trp(7-F), Trp(6-F), Trp(5-F), Trp(4-F), Trp(5-OH), or αMe-Trp. In some embodiments, Xaa³ is Trp, Trp(7-F), or αMe-Trp.

In some embodiments of the peptidic compound, Xaa⁴ is Ala. In some embodiments, Xaa⁴ is Ser.

In some embodiments of the peptidic compound, Xaa⁵ is Val or Tie. In some embodiments, Xaa⁵ is Val. In some embodiments, Xaa⁵ is Tie. In some embodiments, Xaa⁵ is Cpg.

In some embodiments of the peptidic compound, Xaa⁶ is Gly or NMe-Gly. In some embodiments, Xaa⁶ is Gly. In some embodiments, Xaa⁶ is NMe-Gly. In some embodiments, Xaa⁶ is Gly, NMe-Gly, or D-Ala.

In some embodiments of the peptidic compound, Xaa⁷ is His. In some embodiments of the peptidic compound, Xaa⁷ is Trp. In some embodiments of the peptidic compound, Xaa⁷ is Trp(5-F). In some embodiments, Xaa⁷ is Trp, Trp(Me), Trp(7-Me), Trp(6-Me), Trp(5-Me), Trp(4-Me), Trp(2-Me), Trp(7-F), Trp(6-F), Trp(5-F), Trp(4-F), Trp(7-Cl), Trp(6-Cl), Trp(5-Cl), Trp(4-Cl), Trp(7-Br), Trp(6-Br), Trp(5-Br), Trp(4-Br), Trp(7-1), Trp(6-1), Trp(5-1), Trp(4-1), Trp(5-OH), or αMe-Trp. In some embodiments, Xaa⁷ is Nal, 2-pyridylalanine (2-Pal), 3-pyridylalanine (3-Pal), 4-pyridylalanine (4-Pal), 2-quinoylalanine (2-Quin-Ala), or 3-quinoylalanine (3-Quin-Ala). In some embodiments, Xaa⁷ is His, Trp, Trp(5-F), or 3-Quin-Ala.

In some embodiments of the peptidic compound, Xaa⁸ is Leu. In some embodiments, Xaa⁸ is Sta. In some embodiments, Xaa⁸ is D-Pro or Phe. In some embodiments, Xaa⁸ is cis-4-cyclohexyloxy-D-Pro or cis-4-cyclohexylamino-D-Pro.

In some embodiments of the peptidic compound, Xaa⁸-R is a C-terminally amidated cis-4-cyclohexyloxy-D-Pro or cis-4-cyclohexylamino-D-Pro.

In some embodiments of the peptidic compound, Xaa⁹-R is a C-terminally amidated Pro or Leu. In some embodiments of the peptidic compound, Xaa⁹-R is a C-terminally amidated Pro. In some embodiments of the peptidic compound, Xaa⁹-R is a C-terminally amidated Leu. In some embodiments of the peptidic compound, Xaa⁹-R is a C-terminally amidated Phe, diFPro, 4-oxa-Pro, Me₂Thz, or Thz. In some embodiments, Xaa⁹-R is a C-terminally amidated cis-4-cyclohexyloxy-D-Pro or cis-4-cyclohexylamino-D-Pro.

In some embodiments of the peptidic compound, Xaa⁹ is Pro or Leu. In some embodiments of the peptidic compound, Xaa⁹ is Pro. In some embodiments of the peptidic compound, Xaa⁹ is Leu.

In some embodiments of the peptidic compound, Xaa¹⁰-R is a C-terminally amidated Val or Tle. some embodiments of the peptidic compound, Xaa¹⁰-R is a C-terminally amidated Val. some embodiments of the peptidic compound, Xaa¹⁰-R is a C-terminally amidated Tle.

In some embodiments of the peptidic compound, Xaa¹ is an N-terminal amino acid residue selected from D-Phe; Xaa² is Gln, Cit, Glu, or Agb; Xaa³ is Trp or αMe-Trp; Xaa⁴ is Ala; Xaa⁵ is Val or Tle; Xaa⁶ is Gly or NMe-Gly; Xaa⁷ is His, Trp, Trp(5-F), or 3-Quin-Ala; Xaa⁸ is Leu or Sta; and Xaa⁹-R is a C-terminally amidated Pro or Leu.

In some embodiments of the peptidic compound, Xaa¹ is an N-terminal amino acid residue selected from D-Phe; Xaa² is Gln, Cit, Glu, or Agb; Xaa³ is Trp or αMe-Trp; Xaa⁴ is Ala; Xaa⁵ is Val or Tle; Xaa⁶ is Gly or NMe-Gly; Xaa⁷ is His, Trp, or Trp(5-F); Xaa⁸ is Leu or Sta; Xaa⁹ is Pro or Leu; and Xaa¹⁰-R is a C-terminally amidated Val or Tle.

In some embodiments of the peptidic compound, at least one of Xaa¹, Xaa², Xaa³, Xaa⁴, Xaa⁵, Xaa⁶, Xaa⁷, Xaa⁸, Xaa⁹ or Xaa¹⁰ is N-methylated. In some embodiments, at least one of Xaa¹, Xaa², Xaa³, Xaa⁴, Xaa⁵, Xaa⁶, Xaa⁷, or Xaa⁸ is N-methylated. For example, if the compound has a definition of Xaa⁴ as Ala, D-Ala, or Ser, then it also includes options for NMe-Ala, NMe-D-Ala, or NMe-Ser as Xaa⁴.

In some embodiments, Xaa⁶ is N-methylated.

In a specific embodiment, the compounds of formula (A), (I), (IA), (IB), (IC), (ID), (IE), (B), (B-1) do not comprise an albumin binder R^{alb} in the linker.

In some embodiments of the peptidic compound (e.g., compound of formula (A), (I), (IA), (IA-1), (IB), (IB3-1), (IC), (IC-1), (ID), (ID-1), (IE), (IE-1), (IF), (IF-1), (II), (IIA), (B), or (B-1)), ψ, is a reduced peptide bond joining Xaa⁸ to Xaa⁹, wherein Xaa⁸ is Leu and Xaa⁹ is Pro. In some embodiments, the reduced peptide bond joining Xaa⁸ and Xaa⁹ means that the main chain amide

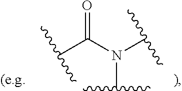 [SMILES: *C(=O)N(*)*]

formed between Xaa⁸ and Xaa⁹ is replaced by

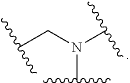 [SMILES: *CN(*)*]

In some embodiments of the peptidic compound, ψ, is a peptide bond joining Xaa⁸ and Xaa⁹.

In some embodiments of the peptidic compound, R^{L} is a linkage moiety joining the linker to the N-terminus of Xaa¹ or joining the linker and R^{rad} In some embodiments, R^{L} is —C(O)—. In other embodiments, R^{L} is —NH—C(O)—. In yet other embodiments, R^{L} is —NH—C(S)—. In some embodiments, R^{L} is —(C₁₋₃alkyl)-. In some embodiments, R^{L} is-CH₂—. In some embodiments, R^{L} is —(C₁₋₃ alkyl)-C(O)—. In some embodiments, R^{L} is —CH₂—C(O)—.

In some embodiments of the peptidic compound, the linker and R^{L} together form a Pip linker. In some embodiments, the linker and R^{L} together comprises a Pip linker.

In some embodiments of the peptidic compound, a non-limiting example of a suitable linker is a peptide linker. More generally, the linker is a linear or branched chain of n1 units of-L¹R¹— and/or -(L¹)₂R¹— (i.e. each unit is independently -L¹R¹— or -(L¹)₂R¹—), wherein n1 is 1-20. In alternative embodiments, n1 is 1, 2, 3, 4, 5, 6, 7, 8, 9, 10, 11, 12, 13, 14, 15, 16, 17, 18, 19, or 20. In some embodiments, n1 is 1-7. In some embodiments, n1 is 1. In other embodiments, n1 is 2. In other embodiments, n1 is 3. In other embodiments, n1 is 4. In other embodiments, n1 is 5. In other embodiments, n1 is 6. In other embodiments, n1 is 7.

In some embodiments of the peptidic compound, the linker is a linear or branched chain of n1 units of-Xaa¹¹-, wherein each Xaa¹¹ is independently an amino acid and n1 is 1-10. In some embodiments, the linker is a linear or branched chain of n1 units of -Xaa¹¹-, wherein each Xaa¹¹ is independently an amino acid and n1 is 1-6. In some embodiments, the linker comprises R^{alb} bonded to a free N-terminus of a Xaa¹¹. In some embodiments, the linker comprises R^{alb} bonded to a side chain of a Xaa¹¹. In some embodiments, at least one Xaa¹¹ is Lys. In some embodiments, at least one Xaa¹¹ is Lys and R^{alb} is bonded to the side chain the Lys.

In some embodiments of the peptidic compound, the linker is a linear peptide of 3 to 6 amino acid residues, -Xaa^{11A}-Xaa^{11B}-(Xaa^{11C})₄-, wherein Xaa^{11A} Xaa^{11B}, and Xaa^{11C} are each independently an amino acid and wherein the radiolabeling group and the albumin-binding group are bonded to the free N-terminus of Xaa^{11A} and the side chain of Xaa^{11B}, respectively, or are bonded to the side chain of Xaa^{11B} and the free N-terminus of Xaa^{11A}, respectively. In some embodiments, Xaa^{11A} is Gly, Glu, D-Glu, Asp, D-Asp, 2-Aad, D-2-Aad, 3-Aad, or D-3-Aad. In some embodiments, Xaa^{11A} is Gly, Glu or D-Glu. In some embodiments, Xaa^{11A} is Gly. In some embodiments, Xaa^{11B} is Lys, Orn, D-Orn, Dab, D-Dab, Dap or D-Dap, any of which can form an amide bond with a carboxylate of a radiolabeling group or an albumin binding group. In some embodiments, Xaa^{11B} is Lys or D-Lys. In some such embodiments, (Xaa^{11C})₁₋₄ is diglycine. In some such embodiments, (Xaa^{11C})₁₄ is p-aminomethylaniline-diglycolic acid (pABzA-DIG), a 4-amino-(1-carboxymethyl)piperidine (Pip), a 9-amino-4,7-dioxanonanoic acid (dPEG2), or a 4-(2-aminoethyl)-1-carboxymethyl-piperazine (Acp). In some such embodiments, (Xaa^{11C})₁₋₄ is Pip. In some embodiments, the C-terminus of Xaa^{11C} and the N-terminus of Xaa¹ are bonded.

In some embodiments of the peptidic compound, the linker is a branched peptide of 3 to 6 amino acid residues, -Xaa^{11B}(Xaa^{11A})-(Xaa^{11C})₁₋₄-, wherein Xaa^{11A} Xaa^{11B}, and Xaa^{11C} are each independently an amino acid and wherein the C-terminus of Xaa^{11A} forms an amide with the side chain of Xaa^{11B}, and wherein the radiolabeling group and the albumin-binding group are bonded to the free N-termini of Xaa^{11A} and Xaa^{11B}, respectively, or are bonded to the free N-termini of Xaa^{11B} and Xaa^{11A}, respectively. In some embodiments, Xaa^{11A} is Gly, Glu, D-Glu, Asp, D-Asp, 2-Aad, D-2-Aad, 3-Aad, or D-3-Aad. In some embodiments, Xaa^{11A} is Glu or D-Glu. In some embodiments, Xaa^{11B} is Lys, D-Lys, Orn, D-Orn, Dab, D-Dab, Dap or D-Dap. In some embodiments, Xaa^{11B} is Lys or D-Lys. In some such embodiments, (Xaa^{11C})₁₋₄ is diglycine. In some such embodiments, (Xaa^{11C})₁₋₄ is p-aminomethylaniline-diglycolic acid (pABzA-DIG), a 4-amino-(1-carboxymethyl)piperidine (Pip), a 9-amino-4,7-dioxanonanoic acid (dPEG2), or a 4-(2-aminoethyl)-1-carboxymethyl-piperazine (Acp). In some such embodiments, (Xaa^{11C})₁₋₄ is Pip. In some embodiments, the C-terminus of Xaa^{11C} and the N-terminus of Xaa¹ are bonded.

In some embodiments of the peptidic compound, the linker is a linear peptide of 3 amino acid residues, -Xaa^{11A}-Xaa^{11B}-Xaa^{11C}-, wherein Xaa^{11A}, Xaa^{11B}, and Xaa^{11C} are each independently an amino acid and wherein the radiolabeling group and the albumin-binding group are bonded to the free N-terminus of Xaa^{11A} and the side chain of Xaa¹B, respectively, or are bonded to the side chain of Xaa^{11B} and the free N-terminus of Xaa^{11A}, respectively. In some embodiments, Xaa^{11A} is Gly, Glu, D-Glu, Asp, D-Asp, 2-Aad, D-2-Aad, 3-Aad, or D-3-Aad. In some embodiments, Xaa^{11A} is Gly, Glu or D-Glu. In some embodiments, Xaa^{11A} is Gly. In some embodiments, Xaa^{11B} is Lys, D-Lys, Orn, D-Orn, Dab, D-Dab, Dap or D-Dap, any of which can form an amide bond with a carboxylate of a radiolabeling group or an albumin binding group. In some embodiments, Xaa^{11B} is Lys or D-Lys. In some such embodiments, Xaa^{11C} is p-aminomethylaniline-diglycolic acid (pABzA-DIG), a 4-amino-(1-carboxymethyl)piperidine (Pip), a 9-amino-4,7-dioxanonanoic acid (dPEG2), or a 4-(2-aminoethyl)-1-carboxymethyl-piperazine (Acp). In some such embodiments, Xaa^{11C} is Pip. In some embodiments, the C-terminus of Xaa^{11C} and the N-terminus of Xaa¹ are bonded.

In some embodiments of the peptidic compound, the linker is -Gly-Lys-Pip-. In some embodiments, the radiolabeling group is bonded to the free N-terminus of Xaa^{11A} and the albumin-binding group is bonded to the side chain of Xaa¹B. In some embodiments, the radiolabeling group is bonded to the free N-terminus of Gly and the albumin-binding group is bonded to the side chain of Lys. In some embodiments, the C-terminus of Pip and the N-terminus of Xaa¹ are bonded.

In some embodiments of the peptidic compound, the linker is a branched peptide of 3 amino acid residues, -Xaa^{1B}(Xaa^{11A})-Xaa^{11C}-, wherein Xaa^{1A}, Xaa^{11B}, and Xaa¹c are each independently an amino acid and wherein the C-terminus of Xaa^{11A} forms an amide with the side chain of Xaa^{11B}, and wherein the radiolabeling group and the albumin-binding group are bonded to the free N-termini of Xaa^{11A} and Xaa^{11B}, respectively, or are bonded to the free N-termini of Xaa^{11B} and Xaa^{11A}, respectively. In some embodiments, Xaa^{11A} is Gly, Glu, D-Glu, Asp, D-Asp, 2-Aad, D-2-Aad, 3-Aad, or D-3-Aad. In some embodiments, Xaa^{11A} is Glu or D-Glu. In some embodiments, Xaa^{11B} is Lys, D-Lys, Orn, D-Orn, Dab, D-Dab, Dap or D-Dap. In some embodiments, Xaa^{11B} is Lys or D-Lys. In some such embodiments, Xaa^{11C} is p-aminomethylaniline-diglycolic acid (pABzA-DIG), a 4-amino-(1-carboxymethyl)piperidine (Pip), a 9-amino-4,7-dioxanonanoic acid (dPEG2), or a 4-(2-aminoethyl)-1-carboxymethyl-piperazine (Acp). In some such embodiments, Xaa^{11C} is Pip. In some embodiments, the C-terminus of Xaa^{11C} and the N-terminus of Xaa¹ are bonded.

In some embodiments of the peptidic compound, R^{L} between Xaa¹¹ of the linker and Xaa¹ is absent and Xaa¹¹ of the linker joins with the N-terminus of Xaa¹. In some embodiments, R^{L} between Xaa¹¹ of the linker and Xaa¹ is absent and Xaa¹¹ of the linker joins with the N-terminus of Xaa¹ by forming an amide bond (i.e., peptide bond between Xaa¹¹-Xaa¹).

In some embodiments of the peptidic compound, the linker and R^{L} together form

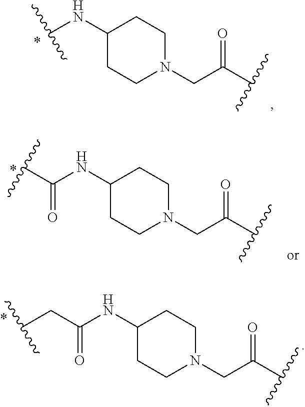 [SMILES: *NC1CCN(CC(*)=O)CC1 ; *C(=O)CN1CCC(NC(*)=O)CC1 ; *CC(=O)NC1CCN(CC(*)=O)CC1]

In some embodiments the linker is

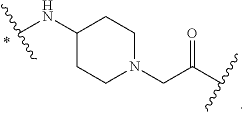 [SMILES: *NC1CCN(CC(*)=O)CC1]

In some embodiments of the peptidic compound, an albumin binder (R^{alb}) is bonded to the linker. In some embodiments of the peptidic compound, the linker does not comprise R^{alb}.

In some embodiments of the peptidic compound, n6 is 1-5. In some embodiments, n6 is 1. In other embodiments, n6 is 2. In other embodiments, n6 is 3. In other embodiments, n6 is 4. In other embodiments, n6 is 5.

In some embodiments of the peptidic compound, n6 is 1 and n1 is 1. In some embodiments, n6 is 1, n1 is 1, and L¹ is —C(O)NH—. In some embodiments, n6 is 1, n1 is 1, L¹ is —C(O)NH—, and R^{L} is —C(O)—. In some embodiments, n6 is 1, n1 is 1, L¹ is —C(O)NH—, R^{L} is —C(O)—, and R¹ is a linear C₁₋₅ alkylenyl or —(CH₂)₂—[O(CH₂)₂]₁-6—(CH₂)₀₋₂.

In some embodiments of the peptidic compound, R^{radn6}-[linker]- is configured as show n in Form ula (a):

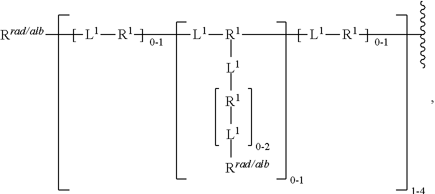

wherein L¹ and R¹ are as defined in the definition of the linker in Formula a, and R^{rad/alb} is either R^{rad} or R^{alb}, and wherein 0-1 R^{rad/alb} is R^{alb}.

In some embodiments of the peptidic compound, R¹ is, independently, a linear, branched, and/or cyclic C₁₋₂₀ alkylenyl, C₂₋₂₀ alkenylenyl and/or C₂₋₂₀ alkynylenyl, wherein any carbon bonded to two other carbons is optionally independently replaced by N, S, or O, and carbons are optionally independently substituted. In some embodiments, R¹ is, independently, a linear, branched, and/or cyclic C₁₋₁₅ alkylenyl, C₂₋₁₅ alkenylenyl and/or C₂₋₁₅ alkynylenyl, wherein any carbon bonded to two other carbons is optionally independently replaced by N, S, or O, and carbons are optionally independently substituted. In some embodiments, R¹ is, independently, a linear, branched, and/or cyclic C₁₋₁₀ alkylenyl, C₂₋₁₀ alkenylenyl and/or C₂₋₁₀ alkynylenyl, wherein any carbon bonded to two other carbons is optionally independently replaced by N, S, or O, and carbons are optionally independently substituted. In some embodiments, R¹ is, independently, a linear, branched, and/or cyclic C₁₋₁₀ alkylenyl. In some embodiments, each R¹ is independently a linear C₁₋₅ alkylenyl or —(CH₂)₂—[O(CH₂)₂]₁₋₆—(CH₂)₀₋₂—.

In some embodiments of the peptidic compound, n1 is 1-7. In some embodiments, each R¹ is independently —C(R^{aa})H—, wherein each R^{aa} is independently the sidechain of a proteinogenic amino acid or the sidechain of an alpha amino acid from Table A. In some embodiments, each R¹ is independently a proteinogenic amino acid or an amino acid from Table A omitting the backbone amino and carboxylic acid groups of the amino acid.

In some embodiments of the peptidic compound, L¹ is a linkage group. In some embodiments, at least one L¹ is —S—. In some embodiments, at least one L¹ is —N(R²)C(O)—; in some of these embodiments, at least one R² is hydrogen. In some embodiments, at least one L¹ is —C(O)N(R²)—; in some of these embodiments, at least one R² is hydrogen. In some embodiments, at least one L¹ is —NH—C(O)—NH—. In some embodiments, at least one L¹ is —NH—C(S)—NH—. In some embodiments, at least one L¹ is

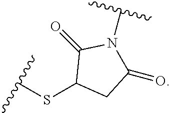 [SMILES: *SC1CC(=O)N(*)C1=O]

In some embodiments, at least one L¹ is

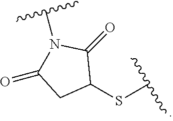 [SMILES: *SC1CC(=O)N(*)C1=O]

In some embodiments, at least one L¹ is

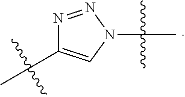 [SMILES: *c1cn(*)nn1]

In some embodiments, at least one L¹ is

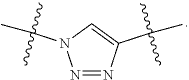 [SMILES: *c1cn(*)nn1]

In some embodiments of the peptidic compound, the linker has the configuration shown in Formula a, and each R¹ is independently a linear C₁₋₅ alkylenyl or —(CH₂)₂—[O(CH₂)₂]₁₋₆—(CH₂)₀₋₂—.

In some embodiments of the peptidic compound, n6 is 1, the linker is L¹R¹ and together with R^{L} forms —C(O)-Xaa¹¹- wherein Xaa¹¹ is a proteinogenic amino acid residue or an amino acid residue selected from Table A. In some embodiments, Xaa¹¹ is pABzA-DIG. In other embodiments, Xaa¹¹ is Pip. In other embodiments, Xaa¹¹ is dPEG2. In other embodiments, Xaa¹¹ is Acp.

In some embodiments of the peptidic compound, the linker together with R^{L} forms a peptide linker, wherein peptide (amide) bonds are independently optionally methylated, optionally replacing one or more amide bonds with 1,2,3-triazole linkages (product of a reaction between an azide and an alkyne). In some embodiments, the peptide linker is a linear peptide linker, optionally replacing one or more amide bonds with 1,2,3-triazole linkages. In some embodiments, the peptide linker is a branched peptide linker, where the amino acid residues may be connected through a combination of main chain amide (peptide) bonds and ‘side chain’-to-‘main chain’ or ‘side chain’-to-‘side chain’ bonds. For example, a branched peptide may be connected by one or more of: backbone (main chain) peptide (amide) bonds, ‘main chain’-to-side chain amide bonds (between an amino group and a carboxylic acid group), optionally replacing one or more amide bonds with 1,2,3-triazole linkages. In some such embodiments, the peptide linker is (Xaa^{A})₁₋₂₀, wherein each Xaa^{A} is independently a proteinogenic amino acid residue or a non-proteinogenic amino acid residue (e.g., selected from Table A) linked together as a linear or branched peptide linker. In some embodiments, (Xaa^{A})₁₋₂₀ is a linear peptide linker. In some embodiments, (Xaa^{A})₁₋₂₀ is a branched peptide linker. R^{rad} is bonded to the peptide linker through an amide bond or another L¹ linkage group; in some embodiments, R^{rad} is bonded to the peptide linker through an amide bond.

In some embodiments, each Xaa^{A} is independently —N(R^{a})R^{b}C(O)— wherein: R^{a} may be H or methyl; R^{b} may be a 1- to 30-atom alkylenyl, heteroalkylenyl, alkenylenyl, heteroalkenylenyl, alkynylenyl, or heteroalkynylenyl, including linear, branched, and/or cyclic (whether aromatic or nonaromatic as well as mono-cyclic, multicyclic or fused cyclic) structures; or N, R^{a} and R^{b} together may form a 5- to 7-atom heteroalkylenyl or heteroalkenylenyl.

In some embodiments, (Xaa^{A})₁₋₂₀ consists of a single amino acid or residue. In some embodiments, (Xaa^{A})₁₋₂₀ is a dipeptide, wherein each Xaa^{A} may be the same or different. In some embodiments, (Xaa^{A})₁₋₂₀ is a tripeptide, wherein each Xaa^{A} may be the same, different or a combination thereof. In some embodiments, (Xaa^{A})₁₋₂₀ consists of 4 amino acid residues connected by peptide bonds, wherein each Xaa^{A} may be the same, different or a combination thereof. In some embodiments, each Xaa^{A} is independently selected from proteinogenic amino acids and the non-proteinogenic amino acids listed in Table A, wherein each peptide backbone amino group of the peptide linker is independently optionally methylated. In some embodiments, all peptide backbone amino groups of the peptide linker are methylated. In other embodiments, only one peptide backbone amino group of the peptide linker is methylated. In other embodiments, only two peptide backbone amino groups of the peptide linker are methylated. In other embodiments, no peptide backbone amino groups of the peptide linker are methylated.

In some embodiments of the peptidic compound, R^{alb} is —(CH₂)ₙ₃—CH₃ wherein n3 is 8-20. In some embodiments, n3 is 8-15.

In some embodiments of the peptidic compound, R^{alb} is —(CH₂)ₙ₄—C(O)OH wherein n4 is 8-20. In some embodiments, n4 is 8-15.

In some embodiments of the peptidic compound, R^{alb} is

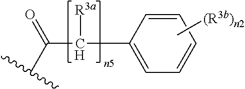

wherein n2 is 1-5, n5 is 1-4, and each R^{3a} is independently H or methyl, and each R^{3b} independently is I, Br, F, Cl, H, OH, OCH₃, NH₂, NO₂ or C₁-C₆ alkyl. In some embodiments, n2 is 1. In some embodiments, n5 is 1, 2, 3, or 4. In some embodiments, each R^{3a} is H. In some embodiments, each R^{3a} is methyl. In some embodiments, each R^{3b} is I, Br, F, or Cl, optionally in para position. In some embodiments, R^{3b} is H. In some embodiments, R^{3b} is OH, optionally in para position. In some embodiments, R^{3b} is OCH₃, optionally in para position. In certain embodiments, R^{3b} is NH₂, optionally in para position. In some embodiments, R^{3b} is NO₂, optionally in para position. In some embodiments, R^{3b} is C₁-C₆ alkyl, optionally in para position. In some embodiments, R^{3a} is H and R^{3b} is OCH₃ or NO₂.

In some embodiments of the peptidic compound, R^{alb} is

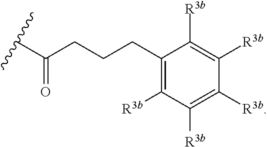

In some embodiments, R^{alb} is

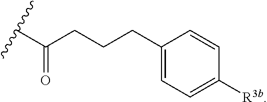

In some embodiments, R^{3b} is I, Br, F, Cl, H, OH, OCH₃, NH₂, or NO₂.

In some embodiments of the peptidic compound, R^{alb} is

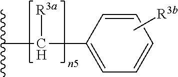

wherein n5 is 1-4 and each R^{3a} is independently H or methyl, and R^{3b} is I, Br, F, Cl, H, OH, OCH₃, NH₂, NO₂ or C₁-C₆ alkyl. In some embodiments, each R^{3a} is H. In some embodiments, each R^{3a} is methyl. In some embodiments, R^{3b} is I, Br, F, or Cl, optionally in para position. In some embodiments, R^{3b} is H. In some embodiments, R^{3b} is OH, optionally in para position. In some embodiments, R^{3b} is OCH₃, optionally in para position. In some embodiments, R^{3b} is NH₂, optionally in para position. In certain embodiments, R^{3b} is NO₂, optionally in para position. In some embodiments, R^{3b} is C₁-C₆ alkyl, optionally in para position. In some embodiments, R^{3a} is H and R^{3b} is OCH₃ or NO₂.

In some embodiments of the peptidic compound, R^{alb} is

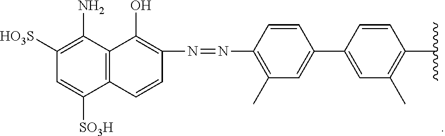 [SMILES: *c1ccc(-c2ccc(N=Nc3ccc4c(S(=O)(=O)O)cc(S(=O)(=O)O)c(N)c4c3O)c(C)c2)cc1C]

In some embodiments, at least one R^{rad} is or comprises a radiometal chelator. The radiometal chelator may be any chelator suitable for binding a radiometal, a radionuclide-bound metal, or a radionuclide-bound metal-containing prosthetic group, and which is attached to the linker by forming an amide bond (between an amino group and a carboxylic acid group) or a 1,2,3-triazole (reaction between an azide and an alkyne), or by reaction between a maleimide and a thiol group. Many suitable radiometal chelators are known, e.g., as summarized in Price and Orvig, Chem. Soc. Rev., 2014, 43, 260-290. In some embodiments, but without limitation, each radiometal chelator is independently selected from the group consisting of: DOTA and DOTA derivatives; DOTAGA (2-(4,7,10-tris(carboxymethyl)-1,4,7,10-tetraazacyclododecan-1-yl)pentanedioic acid); PSC (1,4,7,10-tetraazacyclododecane-1,4,7,10-tetriacetic acid); NOTA; NODAGA; NODASA; CB-DO2A; 3p-C-DEPA; TCMC (also known as DOTAM); DO3A; DTPA and DTPA analogues optionally selected from CHX-A″-DTPA and 1B4M-DTPA; TETA; NOPO; Me-3,2-HOPO; CB-TE1A1P; CB-TE2P; MM-TE2A; DM-TE2A; sarcophagine and sarcophagine derivatives optionally selected from SarAr, SarAr-NCS, diamSar, AmBaSar, and BaBaSar; TRAP; AAZTA; DATA and DATA derivatives; H2-macropa or a derivative thereof; H₂dedpa, H₄octapa, H₄py4pa, H₄Pypa, H₂azapa, H₅decapa, and other picolinic acid derivatives; CP256; PCTA; C-NETA; C-NE3TA; HBED; SHBED; BCPA; CP256; YM103; desferrioxamine (DFO) and DFO derivatives; H₆phospa; a trithiol chelate; mercaptoacetyl; hydrazinonicotinamide; dimercaptosuccinic acid; 1,2-ethylenediylbis-L-cysteine diethyl ester; methylenediphosphonate; hexamethylpropyleneamineoxime; and hexakis(methoxy isobutyl isonitrile). In some embodiments, at least one radiometal chelator is DOTA or a DOTA derivative.

In some embodiments, when R^{rad} is a radiometal chelator, an atom or a chemical group of the radiometal chelator is replaced to form a covalent bond to the linker. For example, a hydroxyl group (—OH) of a carboxylic acid of DOTA is replaced with a covalent bond to the linker. In another example, an amine group (—NH₂) of an acetamide of TCMC is replaced with a covalent bond to the linker.

In some embodiments, when R^{rad} is DOTA and —R^{L}-[linker]-R^{L}—, -[linker]-R^{L}—, or -[linker]- is Pip, and R^{rad}-[linker]-R^{L}— has the structure:

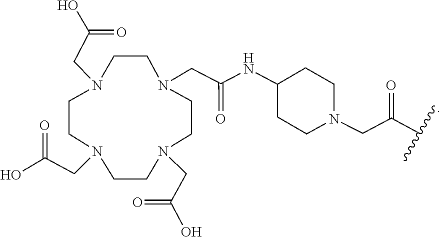 [SMILES: *C(=O)CN1CCC(NC(=O)CN2CCN(CC(=O)O)CCN(CC(=O)O)CCN(CC(=O)O)CC2)CC1]

In some embodiments, when R^{rad} is DOTA and R^{L}-[linker]-R^{L}—, -[linker]-R^{L}—, or -[linker]- is Pip, and R^{rad}—R^{L}-Xaa¹-R^{rad}-[linker]-R^{L}-Xaa¹- or R^{rad}-[linker]-Xaa¹- has the structure:

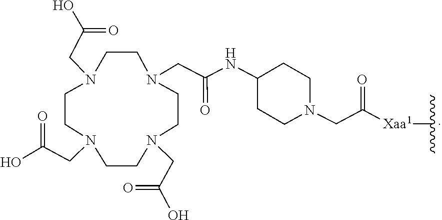

In some embodiments, when R^{rad} is DOTAGA and —R^{L}-[linker]-R^{L}—, -[inker]-R^{L}—, or -[linker]- is Pip, and R^{rad}-[linker]-R^{L}— has the structure:

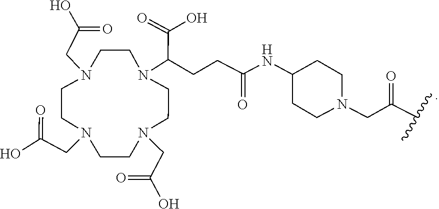 [SMILES: *C(=O)CN1CCC(NC(=O)CCC(C(=O)O)N2CCN(CC(=O)O)CCN(CC(=O)O)CCN(CC(=O)O)CC2)CC1]

In some embodiments, when R^{rad} is DOTAGA and R^{L}-[linker]-R^{L}—, -[linker]-R^{L}—, or -[linker]- is Pip, and R^{rad}—R^{L}-[linker]-R^{L}-Xaa¹-R^{rad}-[inker]-R^{L}-Xaa¹- or R^{rad}-[linker]-Xaa¹- has the structure:

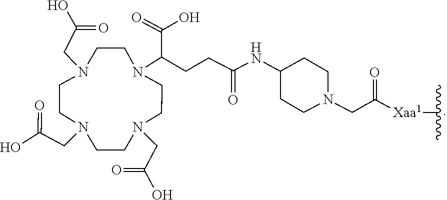

In some embodiments, when R^{rad} is CROWN and —R^{L}-[linker]-R^{L}—, -[inker]-R^{L}—, or -[linker]- is Pip, and R^{rad}—R^{L}-[linker]-R^{L}-Xaa¹- R^{rad}-[linker]-R^{L}-Xaa¹- or R^{rad}-[linker]-Xaa¹- has the structure:

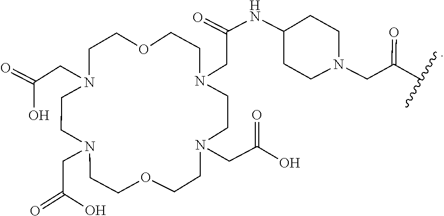 [SMILES: *C(=O)CN1CCC(NC(=O)CN2CCOCCN(CC(=O)O)CCN(CC(=O)O)CCOCCN(CC(=O)O)CC2)CC1]

In some embodiments, when R^{rad} is CROWN and —R^{L}-[linker]-R^{L}—, -[linker]-R^{L}—, or -[linker]- is Pip, and R^{rad}R^{rad}—R^{L}-[linker]-R^{L}-Xaa¹-R^{rad}-[linker]-R^{L}-Xaa¹- or R^{rad}-[linker]-Xaa¹- has the structure:

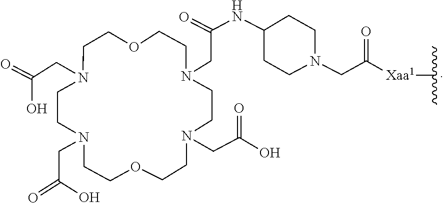

In some embodiments, when R^{rad} is CROWNGA and —R^{L}-[linker]-R^{L}—, -[linker]-R^{L}—, or -[linker]- is Pip, and R^{rad}—R^{L}-[inker]-R^{L}Xaa¹- R^{rad}-[inker]-Xaa¹- or R^{rad}-[linker]-Xaa¹- has the structure:

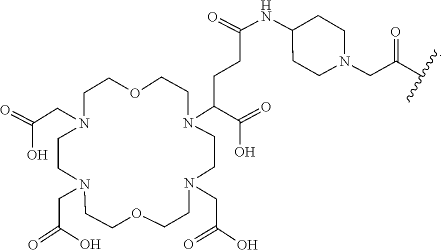 [SMILES: *C(=O)CN1CCC(NC(=O)CCC(C(=O)O)N2CCOCCN(CC(=O)O)CCN(CC(=O)O)CCOCCN(CC(=O)O)CC2)CC1]

In some embodiments, when R^{rad} is CROWNGA and —R-[linker]-R^{L}—, -[linker]-R^{L}—, or -[linker]- is Pip, and R^{rad} R^{rad}-R^{L}-[linker]-R^{L}-Xaa¹- R^{rad}-[linker]-R^{L}-Xaa¹- or R^{rad}-[linker]-Xaa¹- has the structure:

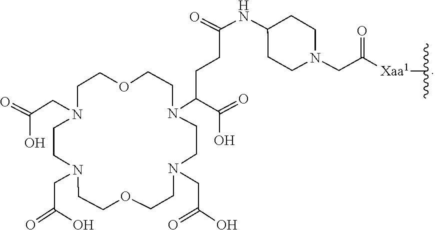

In some embodiments, when R^{rad} is TCMC and —R^{L}-[linker]-R^{L}—, -[linker]-R^{L}, or -[linker]- is Pip, and R^{rad}-[linker]-R^{L}— has the structure:

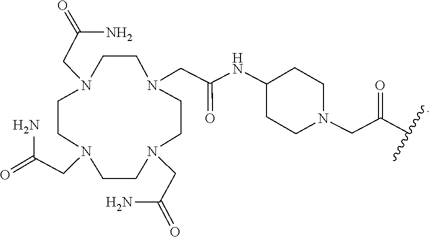 [SMILES: *C(=O)CN1CCC(NC(=O)CN2CCN(CC(N)=O)CCN(CC(N)=O)CCN(CC(N)=O)CC2)CC1]

In some embodiments, when R^{rad} is PSC and —R^{L}-[linker]-R^{L}—, -[inker]-R^{L}—, or -[linker]- is Pip, and R^{rad}—R^{L}-[linker]-R^{L}-Xaa¹- R^{rad}-[linker]R^{L}-Xaa¹- or R^{rad}-[linker]-Xaa¹- has the structure:

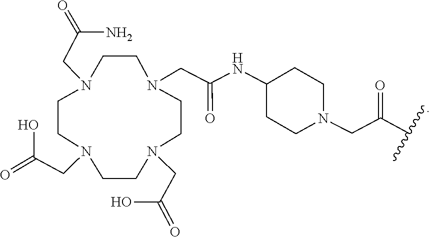 [SMILES: *C(=O)CN1CCC(NC(=O)CN2CCN(CC(N)=O)CCN(CC(=O)O)CCN(CC(=O)O)CC2)CC1]

TABLE B
Exemplary radiometal chelators and exemplary radiometals

| Chelator | Radiometal |
|---|---|
| 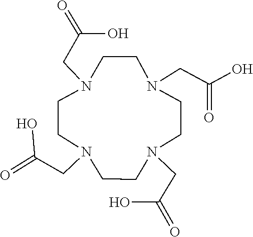 [SMILES: O=C(O)CN1CCN(CC(=O)O)CCN(CC(=O)O)CCN(CC(=O)O)CC1] DOTA, 1,4,7,10-tetraazacyclododecane- 1,4,7,10-tetraacetic acid 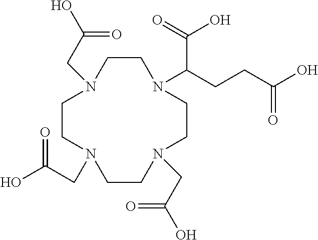 [SMILES: O=C(O)CCC(C(=O)O)N1CCN(CC(=O)O)CCN(CC(=O)O)CCN(CC(=O)O)CC1] DOTAGA, 2-(4,7,10-tris(carboxymethyl)-1,4,7,10-tetraazacyclododecan-1-yl) pantanedioic acid | Cu-64/67 Ga-67/68 In-111 Lu-177 Y-86/90 Bi-203/212/213 Pb-212 Ac-225 Gd-159 Yb-175 Ho-166 As-211 Sc-44/47 Pm-149 Pr-142 Sn-117m Sm-153 Tb-149/152/155/161 Er-165 Ra-223/224 Th-227 |
| 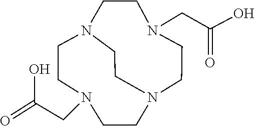 [SMILES: O=C(O)CN1CCN2CCN(CC1)CCN(CC(=O)O)CC2] | Cu-64/67 |
| CB-DO2A, 4,10-bis(carboxymethyl)-1,4,7,10- | |
| tetraazabicyclo[5.5.2]tetradecane | |
| 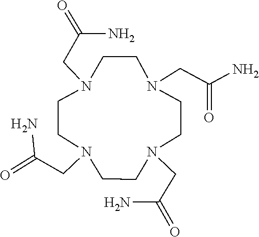 [SMILES: NC(=O)CN1CCN(CC(N)=O)CCN(CC(N)=O)CCN(CC(N)=O)CC1] | Pb-212 |
| TCMC, 1,4,7,10-tetrakis(carbamoylmethyl)- | |
| 1,4,7,10-tetraazacyclododecane | |
| 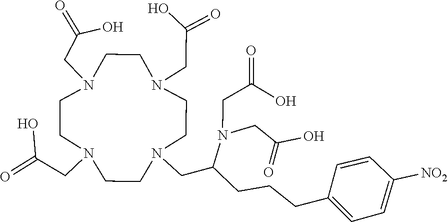 [SMILES: O=C(O)CN1CCN(CC(=O)O)CCN(CC(CCCc2ccc([N+](=O)[O-])cc2)N(CC(=O)O)CC(=O)O)CCN(CC(=O)O)CC1] | Bi-212/213 |
| 3p-C-DEPA | |
| 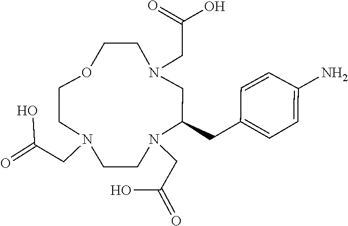 [SMILES: Nc1ccc(C[C@@H]2CN(CC(=O)O)CCOCCN(CC(=O)O)CCN2CC(=O)O)cc1] | Cu-64/67 |
| p-NH₂-Bn-Oxo-DO3A | |
| 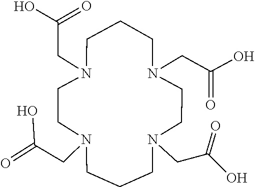 [SMILES: O=C(O)CN1CCCN(CC(=O)O)CCN(CC(=O)O)CCCN(CC(=O)O)CC1] | Cu-64/67 |
| TETA, 1,4,8,11-tetraazacyclotetradecane- | |
| 1,4,8,11-tetraacetic acid | |
| 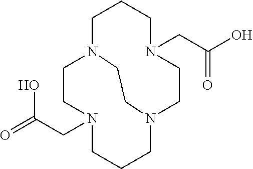 [SMILES: O=C(O)CN1CCCN2CCN(CCCN(CC(=O)O)CC2)CC1] | Cu-64/67 |
| CB-TE2A, 4,11-bis-(carboxymethyl)-1,4,8,11- | |
| tetraazabicyclo[6.6.2]-hexadecane | |
| 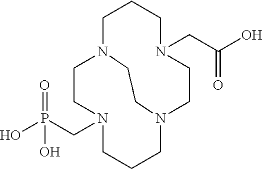 [SMILES: O=C(O)CN1CCCN2CCN(CCCN(CP(=O)(O)O)CC2)CC1] | Cu-64/67 |
| CB-TE1A1P | |
| 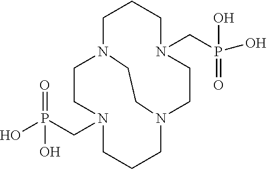 [SMILES: O=P(O)(O)CN1CCCN2CCN(CCCN(CP(=O)(O)O)CC2)CC1] | Cu-64/67 |
| CB-TE2P | |
| 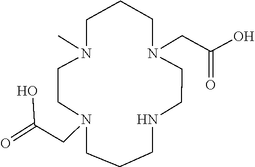 [SMILES: CN1CCCN(CC(=O)O)CCNCCCN(CC(=O)O)CC1] | Cu-64/67 |
| MM-TE2A | |
| 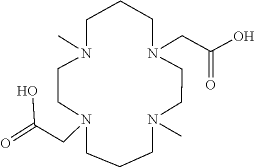 [SMILES: CN1CCCN(CC(=O)O)CCN(C)CCCN(CC(=O)O)CC1] | Cu-64/67 |
| DM-TE2A | |
| 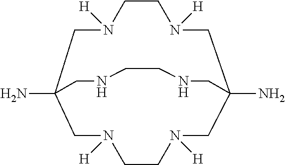 [SMILES: NC12CNCCNCC(N)(CNCCNC1)CNCCNC2] | Cu-64/67 |
| Diamsar | |
| 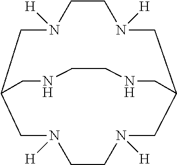 [SMILES: C1CNCC2CNCCNCC(CN1)CNCCNC2] | Cu-64/67 |
| Sarcophagine | |
| 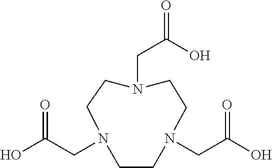 [SMILES: O=C(O)CN1CCN(CC(=O)O)CCN(CC(=O)O)CC1] | Cu-64/67 Ga-68 In-111 Sc-44/47 |
| NOTA, 1,4,7-triazacyclononane-1,4,7-triacetic acid | |
| 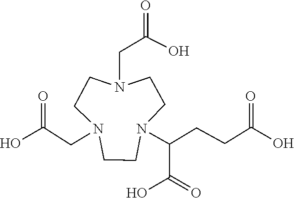 [SMILES: O=C(O)CCC(C(=O)O)N1CCN(CC(=O)O)CCN(CC(=O)O)CC1] | Cu-64/67 Ga-68 In-111 Sc-44/47 |
| NODAGA, 1,4,7-triazacyclononane, 1-glutaric acid-4,7-acetic acid | |
| 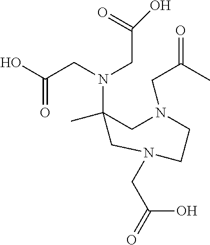 [SMILES: CC(=O)CN1CCN(CC(=O)O)CC(C)(N(CC(=O)O)CC(=O)O)C1] | Cu-64/67 Ga-68 In-111 Lu-177 |
| AAZTA | |
| 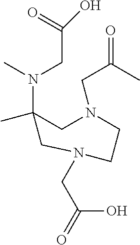 [SMILES: CC(=O)CN1CCN(CC(=O)O)CC(C)(N(C)CC(=O)O)C1] | Cu-64/67 Ga-68 In-111 |
| DATA | |
| 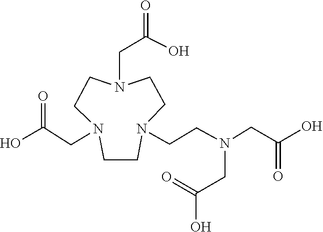 [SMILES: O=C(O)CN1CCN(CCN(CC(=O)O)CC(=O)O)CCN(CC(=O)O)CC1] | Cu-64/67 Ga-68 Lu-177 Y-86/90 Bi-213 Pb-212 |
| NETA, {4-[(2-(bis-carboxymethylamino)-ethyl]- | |
| 7-carboxymethyl-[1,4,7]triazonan-1-yl}-acetic acid | |
| 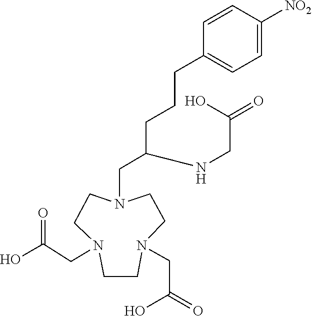 [SMILES: O=C(O)CNC(CCCc1ccc([N+](=O)[O-])cc1)CN1CCN(CC(=O)O)CCN(CC(=O)O)CC1] | Cu-64/67 Ga-68 Lu-177 Y-86/90 Bi-213 Pb-212 |
| 3p-C-NE3TA | |
| 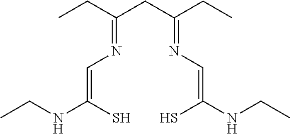 [SMILES: CCN/C(S)=C/N=C(\CC)C/C(CC)=N/C=C(\S)NCC] | Au-198/199 |
| HxTSE | |
| 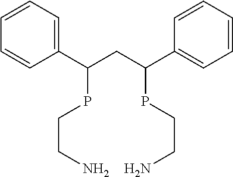 | Rh-105 |
| P₂N₂Ph₂ | |
| 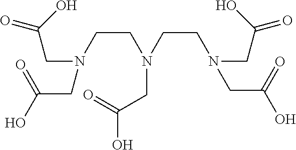 [SMILES: O=C(O)CN(CCN(CC(=O)O)CC(=O)O)CCN(CC(=O)O)CC(=O)O] | In-111 Sc-44/47 Lu-177 Y-86/90 Sn-117m Pd-109 |
| DTPA, diethylenetriaminepentaacetic acid | |
| 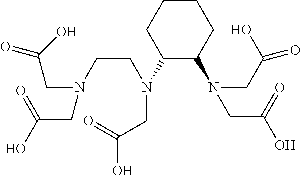 | In-111 Lu-177 Y-86/90 Bi-212/213 |
| CHX-A00-DTPA, 2-(p-isothiocyanatobenzyl)cyclohexyl- | |
| diethylenetriaminepentaacetic acid | |
| 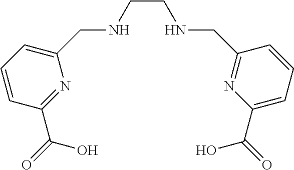 [SMILES: O=C(O)c1cccc(CNCCNCc2cccc(C(=O)O)n2)n1] | Cu-64/67 |
| H₂dedpa, 1,2-[[6-(carboxy)-pyridin-2-yl]- | |
| methylamino]ethane | |
| 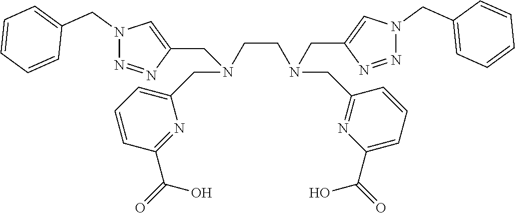 [SMILES: O=C(O)c1cccc(CN(CCN(Cc2cn(Cc3ccccc3)nn2)Cc2cccc(C(=O)O)n2)Cc2cn(Cc3ccccc3)nn2)n1] | Cu-64/67 |
| H₂azapa, N,N′-[1-benzyl-1,2,3-triazole-4-yl]methyl- | |
| N,N′-[6-(carboxy)pyridin-2-yl]-1,2-diaminoethane | |
| 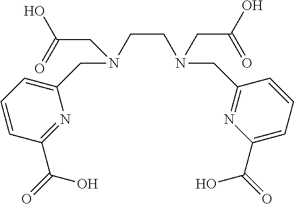 [SMILES: O=C(O)CN(CCN(CC(=O)O)Cc1cccc(C(=O)O)n1)Cc1cccc(C(=O)O)n1] | In-111 Lu-177 Y-86/90 Ac-225 |
| H₄octapa | |
| 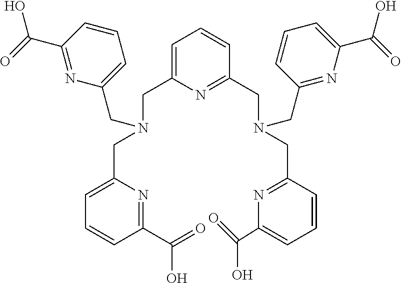 [SMILES: O=C(O)c1cccc(CN(Cc2cccc(CN(Cc3cccc(C(=O)O)n3)Cc3cccc(C(=O)O)n3)n2)Cc2cccc(C(=O)O)n2)n1] | Ac-225 |
| H₄py4pa | |
| 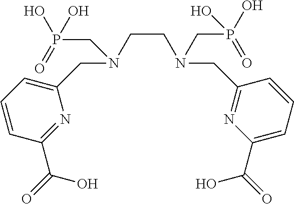 [SMILES: O=C(O)c1cccc(CN(CCN(Cc2cccc(C(=O)O)n2)CP(=O)(O)O)CP(=O)(O)O)n1] | Ac-225 |
| H₆phospa | |
| 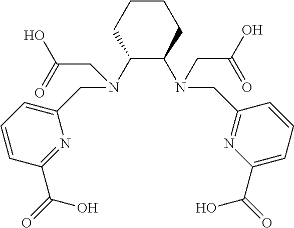 [SMILES: O=C(O)CN(Cc1cccc(C(=O)O)n1)[C@@H]1CCCC[C@H]1N(CC(=O)O)Cc1cccc(C(=O)O)n1] | In-111 Ac-225 |
| H₄CHXoctapa | |
| 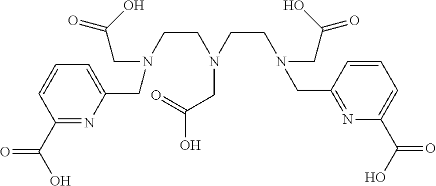 [SMILES: O=C(O)CN(CCN(CC(=O)O)Cc1cccc(C(=O)O)n1)CCN(CC(=O)O)Cc1cccc(C(=O)O)n1] | In-111 Lu-177 Ac-225 |
| H₅decapa | |
| 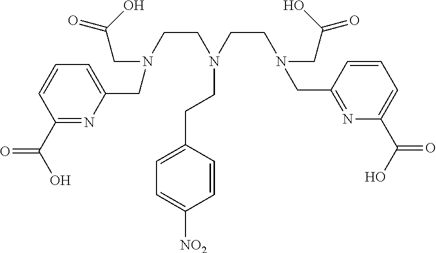 [SMILES: O=C(O)CN(CCN(CCc1ccc([N+](=O)[O-])cc1)CCN(CC(=O)O)Cc1cccc(C(=O)O)n1)Cc1cccc(C(=O)O)n1] | In-111 Lu-177 Ac-225 |
| H₄neunpa-p-Bn-NO₂ | |
| 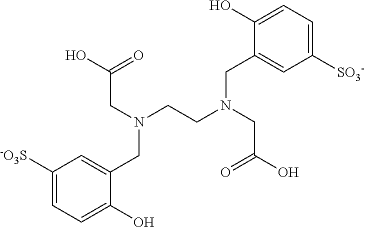 | In-111 Ga-68 |
| SHBED, N,N′-bis(2-hydroxy-5-sulfobenzyl)- | |
| ethylenediamine-N,N′-diacetic acid | |
| 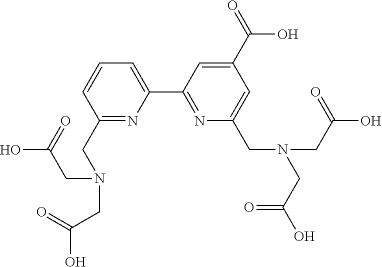 [SMILES: O=C(O)CN(CC(=O)O)Cc1cccc(-c2cc(C(=O)O)cc(CN(CC(=O)O)CC(=O)O)n2)n1] | In-111 |
| BPCA | |
| 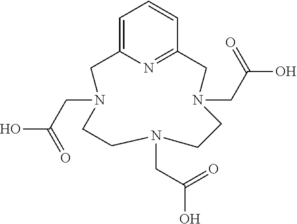 [SMILES: O=C(O)CN1CCN(CC(=O)O)Cc2cccc(n2)CN(CC(=O)O)CC1] | Cu-64/67 |
| PCTA, 3,6,9,15-tetraazabicyclo[9.3.1]- | |
| pentadeca-1(15),11,13-triene-3,6,9,-triacetic acid | |
| 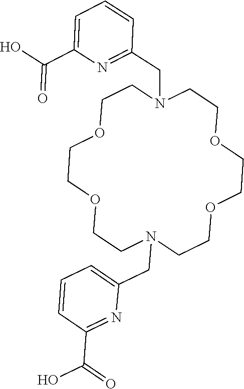 [SMILES: O=C(O)c1cccc(CN2CCOCCOCCN(Cc3cccc(C(=O)O)n3)CCOCCOCC2)n1] | Ac-225 |
| H2-MACROPA (N,N′-bis[(6-carboxy-2-pyridil)methyl]- | |
| 4,13-diaza-18-crown-6) | |
| 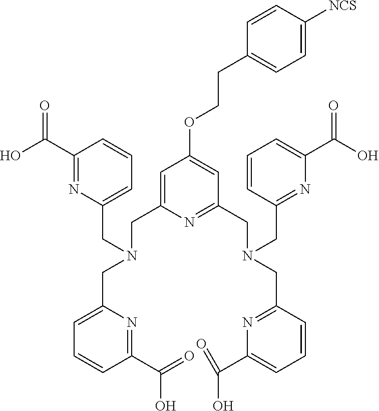 | Ac-225 |
| H4py4pa-phenyl-NCS | |
| 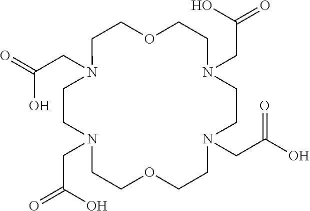 [SMILES: O=C(O)CN1CCOCCN(CC(=O)O)CCN(CC(=O)O)CCOCCN(CC(=O)O)CC1] | Ac-225 Tb-155 Tb-161 Th-227 |
| CROWN | |
| 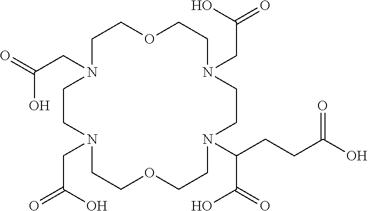 [SMILES: O=C(O)CCC(C(=O)O)N1CCOCCN(CC(=O)O)CCN(CC(=O)O)CCOCCN(CC(=O)O)CC1] | Ac-225 Tb-155 Tb-161 Th-227 |
| CROWNGA | |
| 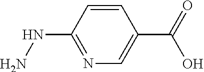 [SMILES: NNc1ccc(C(=O)O)cn1] | Tc-99m Tc-94m |
| HYNIC | |
| 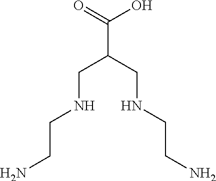 [SMILES: NCCNCC(CNCCN)C(=O)O] | Tc-99m Tc-94m |
| N4 (6-carboxy-1,4,7,11-tetraazaundecane) | |
| 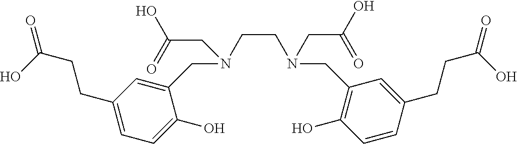 [SMILES: O=C(O)CCc1ccc(O)c(CN(CCN(CC(=O)O)Cc2cc(CCC(=O)O)ccc2O)CC(=O)O)c1] | Ga-68 |
| HBED-CC | |
| 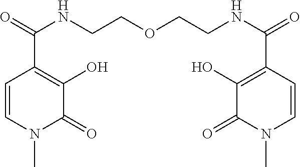 [SMILES: Cn1ccc(C(=O)NCCOCCNC(=O)c2ccn(C)c(=O)c2O)c(O)c1=O] | Th-227 |
| Me-3,2-HOPO (3-hydroxy-N-methyl-2-pyridinone) | |
| 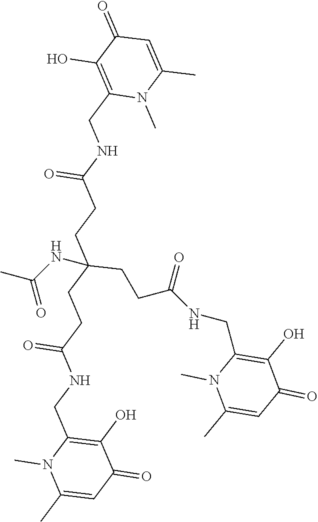 [SMILES: CC(=O)NC(CCC(=O)NCc1c(O)c(=O)cc(C)n1C)(CCC(=O)NCc1c(O)c(=O)cc(C)n1C)CCC(=O)NCc1c(O)c(=O)cc(C)n1C] | Ga-68 |
| CP256 | |
| 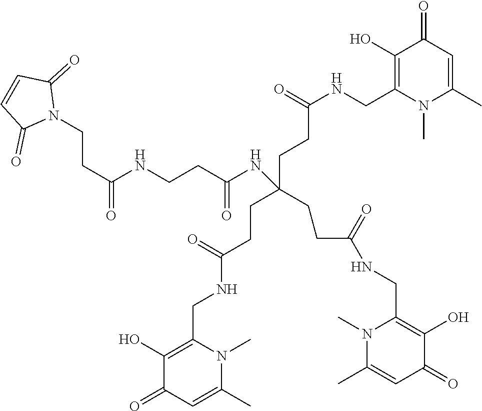 [SMILES: Cc1cc(=O)c(O)c(CNC(=O)CCC(CCC(=O)NCc2c(O)c(=O)cc(C)n2C)(CCC(=O)NCc2c(O)c(=O)cc(C)n2C)NC(=O)CCNC(=O)CCN2C(=O)C=CC2=O)n1C] | Ga-68 |
| YM103 | |
| 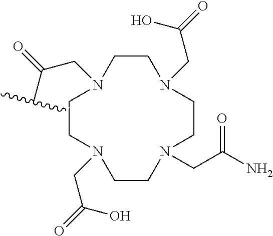 [SMILES: *C(=O)CN1CCN(CC(=O)O)CCN(CC(N)=O)CCN(CC(=O)O)CC1] | Pb-203 Pb-212 |
| Pb Specific Chelator (PSC) | |

In some embodiments of the peptidic compound, each radiometal chelator is independently selected from Table B, wherein each chelator is optionally bound by a radiometal. In some embodiments, each radiometal chelator is bound by one of the corresponding radiometal shown in Table B. In some embodiments, the radiometal chelator is bound by a radiometal, a radionuclide-bound metal, or a radionuclide-bound metal-containing prosthetic group.

In some embodiments of the peptidic compound, at least one R^{rad} is DOTA, or a derivative thereof, linked via an amide (e.g., formed from one of the carboxyl groups shown in Table B. In some embodiments, at least one R^{rad} is CB-DO2A, or a derivative thereof, linked via an amide (e.g., formed from one of the carboxyl groups shown in Table B). In some embodiments, at least one R^{rad} is TCMC, or a derivative thereof, linked via an amide (e.g., formed from one of the —CONH₂ groups shown in Table B). In some embodiments, the chelator at least one R^{rad} is 3p-C-DEPA, or a derivative thereof, linked via an amide (e.g., formed from one of the carboxyl groups shown in Table B). In some embodiments, at least one R^{rad} is p-NH₂—Bn-Oxo-DO3A or a derivative thereof, linked via an amide (e.g., formed from one of the carboxyl groups shown in Table B). In some embodiments, at least one R^{rad} is TETA, or a derivative thereof, linked via an amide (e.g., formed from one of the carboxyl groups shown in Table B). In some embodiments, at least one R^{rad} is CB-TE2A, or a derivative thereof, linked via an amide (e.g., formed from one of the carboxyl groups shown in Table B). In some embodiments, at least one R^{rad} is Diamsar, or a derivative thereof, linked via an amide (e.g., formed from one of the amino groups shown in Table B). In some embodiments, at least one R^{rad} is NOTA, or a derivative thereof, linked via an amide (e.g., formed from one of the carboxyl groups shown in Table B). In some embodiments, at least one R^{rad} is NETA, or a derivative thereof, linked via an amide (e.g., formed from one of the carboxyl groups shown in Table B). In some embodiments, at least one R^{rad} is HxTSE, or a derivative thereof, linked via an amide (e.g., formed from one of the amino groups shown in Table B). In some embodiments, at least one R^{rad} is P₂N₂Ph₂, or a derivative thereof, linked via an amide (e.g., formed from one of the amino groups shown in Table B). In some embodiments, at least one R^{rad} is DTPA, or a derivative thereof, linked via an amide (e.g., formed from one of the carboxyl groups shown in Table B). In some embodiments, at least one R^{rad} is CHX-A00-DTPA, or a derivative thereof, linked via an amide (e.g., formed from one of the carboxyl groups shown in Table B). In some embodiments, at least one R^{rad} is H₂dedpa, or a derivative thereof, linked via an amide (e.g., formed from one of the carboxyl groups shown in Table B). In some embodiments, at least one R^{rad} is H₂azapa, or a derivative thereof, linked via an amide (e.g., formed from one of the carboxyl groups shown in Table B). In some embodiments, at least one R^{rad} is H₄octapa, or a derivative thereof, linked via an amide (e.g., formed from one of the carboxyl groups shown in Table B). In some embodiments, at least one R^{rad} is H₆phospa, or a derivative thereof, linked via an amide (e.g., formed from one of the carboxyl groups shown in Table B). In some embodiments, at least one R^{rad} is H₄CHXoctapa, or a derivative thereof, linked via an amide (e.g., formed from one of the carboxyl groups shown in Table B). In some embodiments, at least one R^{rad} is H₅decapa, or a derivative thereof, linked via an amide (e.g., formed from one of the carboxyl groups shown in Table B). In some embodiments, at least one R^{rad} is H₄neunpa-p-Bn-NO2, or a derivative thereof, linked via an amide (e.g., formed from one of the carboxyl groups shown in Table B). In some embodiments, at least one R^{rad} is SHBED, or a derivative thereof, linked via an amide (e.g., formed from one of the carboxyl groups shown in Table B). In some embodiments, at least one R^{rad} is BPCA, or a derivative thereof, linked via an amide (e.g., formed from one of the carboxyl groups shown in Table B). In some embodiments, at least one R^{rad} is PCTA, or a derivative thereof, linked via an amide (e.g., formed from one of the carboxyl groups shown in Table B). In some embodiments, at least one R^{rad} is H2-MACROPA, or a derivative thereof, linked via an amide (e.g., formed from one of the carboxyl groups shown in Table B). In some embodiments, at least one R^{rad} is CROWN, or a derivative thereof, linked via an amide (e.g., formed from one of the carboxyl groups shown in Table B). In some embodiments, at least one R^{rad} is CROWNGA, or a derivative thereof, linked via an amide (e.g., formed from one of the carboxyl groups shown in Table B). In some embodiments, at least one R^{rad} is HYNIC, or a derivative thereof, linked via an amide (e.g., formed from the carboxyl group shown in Table B). In some embodiments, at least one R^{rad} is N₄, or a derivative thereof, linked via an amide (e.g., formed from the carboxyl group shown in Table B). In some embodiments, at least one R^{rad} is HBED-CC, or a derivative thereof, linked via an amide (e.g., formed from one of the carboxyl groups shown in Table B).

In some embodiments of the peptidic compound, the radiometal chelator (or one of the radiometal chelators) is a derivative of a radiometal chelator shown in Table B. A derivative may include, e.g., (1) modification of a functional group of the chelator (e.g., a carboxyl group, an amino group, etc.) or (2) attachment of a new functional group (e.g., attachment of an R-group to an ethylene carbon located between two nitrogen atoms, wherein the R-group is a functional group fused to a spacer). In some embodiments, a carboxyl functional group shown in Table B is replaced with azidopropyl ethylacetamide (e.g., azido-mono-amide-DOTA), butynylacetamide (e.g., butyne-DOTA), thioethylacetamide (e.g., DO3A-thiol), maleimidoethylacetamide (e.g., maleimido-mono-amide-DOTA), or N-hydroxysuccinimide ester (e.g., DOTA-NHS-ester). When linked, these derivative chelators can be linked either via an amide (formed from a remaining carboxyl group) or via —C(O)—NH—(CH₂)₂₋₃-(triazole) or —C(O)—NH—(CH₂)₂₋₃-(thiomaleimide). In other embodiments, a backbone carbon (e.g., in an ethylene positioned between two backbone nitrogen atoms) in the chelator ring is fused to an R-group containing a functional group, optionally wherein the R-group is —(CH₂)₁₋₃-(phenyl)-N═C═S or —(CH₂)₁₋₃-(phenyl)-N═C═O, optionally 1,4-isothiocyanatobenzyl; e.g., p-SCN-Bn-DOTA (S-2-(4-isothiocyanatobenzyl)-1,4,7,10-tetraazacyclododecane tetraacetic acid), p-SCN-Bn-NOTA (2-S-(4-isothiocyanatobenzyl)-1,4,7-triazacyclononane-1,4,7-triacetic acid), and the like. When linked, these derivatives can form a urea linkage (formed from isocyanate) or a thiourea linkage (formed from isothiocyanate).

In some embodiments of the peptidic compound, the radiometal chelator is 1,4,7,10-tetraazacyclotetradecane-1,4,7,10-tetraacetic acid (DOTA), 2-(4,7,10-tris(carboxymethyl)-1,4,7,10-tetraazacyclododecan-1-yl)pentanedioic acid (DOTAGA), 1,4,7-triazacyclononane-triacetic acid (NOTA), 1,4,7-triazacyclononane-1-glutaric acid-4,7-diacetic acid (NODAGA), 1,4,7-Triazacyclononane-1-succinic acid-4,7-diacetic acid (NODASA), 1,8-N,N′-bis-(carboxymethyl)-1,4,8,11- tetraazacyclotetradecane (TE2A), 3,6,9,15-tetraazabicyclo[9.3.1]pentadeca-1(15),11,13-triene-3,6,9-triacetic acid (PCTA), 1-substituted 1,4,7,-tricarboxymethyl-1,4,7,10-teraazacyclododecane triacetic acid (DO3A), DEDPA (6,6′-[1,2-ethanediylbis(iminomethylene)]bis(2-pyridinecarboxylic acid) and 1,4,8,11-tetraazacyclotetradecane-1,4,8,11-tetraacetic acid (TETA), ethylenediaminetetraacetic acid (EDTA), diethylenetriamine pentaacetic acid (DTPA), CB-DO2A, 3p-C-DEPA, TCMC, DO3A, DTPA, CHX-A″-DTPA, 1 B4M-DTPA, TETA, NOPO, Me-3,2-HOPO, CB-TE1A1P, CB-TE2P, MM-TE2A, DM-TE2A, sarcophagine, SarAr, SarAr-NCS, diamSar, AmBaSar, BaBaSar, TRAP, AAZTA, DATA, H2-macropa, H2dedpa, H4octapa, H4py4pa, H4Pypa, H2azapa, H5decapa, H4CHXoctapa, H4neunpa-p-Bn-NO2, CP256, PCTA, {4-[2-(bis-carboxymethylamino)-ethyl]-7-carboxymethyl-[1,4,7]triazonan-1-yl}-acetic acid (NETA), C-NETA, C-NE3TA, HBED, HBED-CC, BCPA, CP256, YM103, desferrioxamine (DFO), H6phospa, a trithiol chelate, mercaptoacetyl, hydrazinonicotinamide, dimercaptosuccinic acid, 1,2-ethylenediylbis-L-cysteine diethyl ester, methylenediphosphonate, N, N′-bis(2-hydroxy-5-sulfobenzyl)-ethylenediamine-N, N-diacetic acid (SHBED), hexamethylpropyleneamineoxime, hexakis(methoxy isobutyl isonitrile), 2,2′,2″,2′-(1,10-dioxa-4,7,13,16-tetraazacyclooctadecane-4,7,13,16-tetrayl) tetraacetic acid (CROWN), 2-(7,13,16-tris(carboxymethyl)-1,10-dioxa-4,7,13,16-tetraazacyclooctadecan-4-yl)pentanedio ic acid (CROWNGA), H4py4pa-phenyl-NCS, PSC, bis-aminoethanethiol (BAT), TE3A, TE2A, CB-TE2A, triazacyclononane (TACN), 1,4,7-Trimethyl-1,4,7-triazacyclononane (TM-TACN), BPCA, HEHA, PEPA, H6phospha, noneunpa, or DOTAM.

In some embodiments of the peptidic compound, the radiometal chelator is TETA (1,4,8,11-tetraazacyclotetradecane-1,4,8,11-tetraacetic acid), SarAr (1-N-(4-Aminobenzyl)-3,6,10,13,16,19-hexaazabicyclo[6.6.6]-eicosane-1,8-diamine), NOTA (1,4,7-triazacyclononane-1,4,7-triacetic acid), TRAP (1,4,7-triazacyclononane-1,4,7-tris[methyl(2-carboxyethyl)phosphinic acid), HBED (N,N′-bis(2-hydroxybenzyl)-ethylenediamine-N,N′-diacetic acid), 2,3-HOPO (3-hydroxypyridin-2-one), PCTA (3,6,9,15-tetraazabicyclo[9.3.1]-pentadeca-1 (15),11,13-triene-3,6,9,-triacetic acid), DFO (desferrioxamine), DTPA (diethylenetriaminepentaacetic acid), OCTAPA (N,N′-bis(6-carboxy-2-pyridylmethyl)-ethylenediamine-N,N′-diacetic acid) or another picolinic acid derivative.

In some embodiments of the peptidic compound, the radiometal chelator is DOTA, PSC (1,4,7,10-tetraazacyclododecane-1,4,7,10-tetriacetic acid), TCMC, or CROWN. In some embodiments, the radiometal chelator is DOTA, DOTAGA, PSC, TCMC, CROWN or CROWNGA. In some embodiments, the radiometal chelator is DOTA or CROWN. In some embodiments, the radiometal chelator is DOTA, DOTAGA, CROWN, or CROWNGA.

In some embodiments of the peptidic compound, a R^{rad} is a radiometal chelator for radiolabelling with ^{99m}Tc, ^{94m}Tc, ¹⁸⁶Re, or ¹⁸⁸Re, such as mercaptoacetyl, hydrazinonicotinamide, dimercaptosuccinic acid, 1,2-ethylenediylbis-L-cysteine diethyl ester, methylenediphosphonate, hexamethylpropyleneamineoxime and hexakis(methoxy isobutyl isonitrile), and the like. In some embodiments, a R^{rad} is mercaptoacetyl, hydrazinonicotinamide, dimercaptosuccinic acid, 1,2-ethylenediylbis-L-cysteine diethyl ester, methylenediphosphonate, hexamethylpropyleneamineoxime or hexakis(methoxy isobutyl isonitrile). In some of these embodiments, the chelator is bound by a radiometal selected from ^{99m}Tc, ^{94m}Tc, ¹⁸⁶Re, or ¹⁸⁸Re.

In some embodiments of the peptidic compound, R^{rad} is a chelator that can bind ¹⁸F-aluminum fluoride ([¹⁸F]AIF), such as 1,4,7-triazacyclononane-1,4-diacetate (NODA) and the like. In some embodiments, the chelator is NODA. In some embodiments, the chelator is bound by [¹⁸F]AIF.

In some embodiments of the peptidic compound, R^{rad} is a chelator that can bind ⁷²As or ⁷⁷As, such as a trithiol chelate and the like. In some embodiments, the chelator is a trithiol chelate. In some embodiments, the chelator is conjugated to ⁷²As. In some embodiments, the chelator is conjugated to ⁷⁷As.

In some embodiments, the radiometal chelator is DOTA or a derivative thereof conjugated with ⁴⁴Sc, ⁴⁷Sc, ⁶⁴Cu, ⁶⁷Cu, ⁶⁸Ga, ⁸⁶Y, ⁹⁰Y, ¹¹¹In, ^{117m}Sn, ¹⁴⁹Tb, ¹⁵²Tb, ¹⁵³Sm, ¹⁵⁵Tb, ¹⁶¹Tb, ¹⁶⁵Er, ¹⁷⁷Lu, ²⁰³Pb, ²¹²Pb, ²¹²Bi, ²¹³Bi, ²²⁴Ra, ²²⁵Ac, ²²⁷Th, or ²²³Ra. In some embodiments, the radiometal chelator is DOTA or a derivative thereof conjugated with ⁶⁸Ga or ¹⁷⁷Lu. In some embodiments, the radiometal chelator is DOTAGA or a derivative thereof conjugated with ⁴⁴Sc, ⁴⁷Sc, ⁶⁴Cu, ⁶⁷Cu, ⁶⁸Ga, ⁸⁶Y, ⁹⁰Y, ¹¹¹In, ^{117m}Sn, ¹⁴⁹Tb, ¹⁵²Tb, ¹⁵³Sm, ¹⁵⁵Tb, ¹⁶¹Tb, ¹⁶⁵Er, ¹⁷⁷Lu, ²⁰³Pb, ²¹²Pb, ²¹²Bi, ²¹³Bi, ²²⁴Ra, ²²⁵Ac, ²²⁷Th, or ²²³Ra. In some embodiments, the radiometal chelator is DOTAGA or a derivative thereof conjugated with ⁶⁸Ga or ¹⁷⁷Lu.

In some embodiments, the radiometal chelator is CROWN conjugated with ²²⁵Ac, 17Lu or ⁶⁸Ga. In some embodiments, the radiometal chelator is CROWNGA conjugated with ²²⁵Ac, ¹⁷⁷Lu or ⁶⁸Ga.

In some embodiments, the radiometal chelator is TCMC conjugated with ²⁰³Pb or ²¹²Pb. In some embodiments, the radiometal chelator is PSC conjugated with ²⁰³Pb or ²¹²Pb.

In some embodiments, the radiometal chelator is H2-MACROPA conjugated with ²²⁵Ac. In some embodiments, the radiometal chelator is Me-3,2-HOPO conjugated with ^{227Th}. In some embodiments, the radiometal chelator is H₄py4pa conjugated with ²²⁵Ac, ²²⁷Th or ¹⁷⁷Lu. In some embodiments, the radiometal chelator is H₄pypa conjugated with ¹⁷⁷Lu. In some embodiments, the radiometal chelator is NODAGA conjugated with ⁶⁸Ga. In some embodiments, the radiometal chelator is DTPA conjugated with ¹¹¹In. In some embodiments, the radiometal chelator is or DFO conjugated with ⁸⁹Zr.

In some embodiments, an R^{rad} is a chelator that can bind ¹⁸F-aluminum fluoride ([¹⁸F]AIF), such as 1,4,7-triazacyclononane-1,4-diacetate (NODA) and the like. In some embodiments, the chelator is NODA. In some embodiments, the chelator is bound by [¹⁸F]AIF.

In some embodiments, an R^{rad} is a chelator that can bind ⁷²As or ⁷⁷As, such as a trithiol chelate and the like. In some embodiments, the chelator is a trithiol chelate. In some embodiments, the chelator is conjugated to ⁷²As. In some embodiments, the chelator is conjugated to ⁷⁷As.

In some embodiments, R^{rad}n6-[linker]-R^{L}— is DOTA-Pip-, DOTAGA-Pip-, CROWN-Pip-, or CROWNGA-Pip-.

In some embodiments of the peptidic compound, at least one R^{rad} is a prosthetic group containing a trifluoroborate (BF₃), capable of ¹⁸F/¹⁹F exchange radiolabeling. In some embodiments, R^{rad} is BF₃—R⁵—R⁴—, wherein R⁴ is —(CH₂)₁₋₅— and wherein BF₃—R⁵-forms:

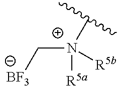

wherein R^{5a} and R^{5b} are each independently a C₁-C₅ linear or branched alkyl group. In some embodiments, R⁴ is —CH₂—. In some embodiments, R^{5a} is methyl. In some embodiments, R^{5a} is ethyl. In some embodiments, R^{5a} is propyl. In some embodiments, R^{5a} is isopropyl. In some embodiments, R^{5a} is butyl. In some embodiments, R^{5a} is n-butyl. In some embodiments, R^{5a} is pentyl. In some embodiments, R^{5b} is methyl. In some embodiments, R^{5b} is ethyl. In some embodiments, R^{5b} is propyl. In some embodiments, R^{5b} is isopropyl. In some embodiments, R^{5b} is butyl. In some embodiments, R^{5b} is n-butyl. In some embodiments, R^{5b} is pentyl. In some embodiments, R^{5a} and R^{5b} are both methyl. In some embodiments, BF₃—R⁵-is

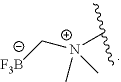

In some embodiments, at least one R^{rad} is

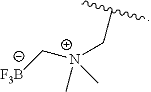

In some embodiments, one fluorine in BF₃ is ¹⁸F. In some embodiments, one, two, or three fluorines in BF₃ are ¹⁸F. In some embodiments, all three fluorines in BF₃ are ¹⁸F. In some embodiments, all three fluorines in BF₃ are ¹⁹F.

In some embodiments of the peptidic compound, at least one R^{rad} has the structure listed in Table C or Table D. in Tables C and D, each R in —OR, —SR, —NR—, —NHR or —NR₂ is independently a C₁-C₅ linear or branched alkyl. In some embodiments, one fluorine in BF₃ is ¹⁸F. In some embodiments, one, two, or three fluorines in BF₃ are ¹⁸F. In some embodiments, all three fluorines in BF₃ are ¹⁸F. In some embodiments, all three fluorines in BF₃ are ¹⁹F.

TABLE C
Exemplary BF₃—R⁵— groups.

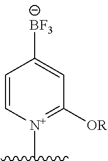

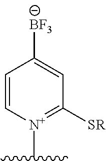

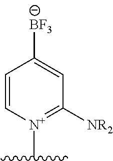

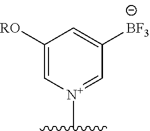

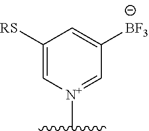

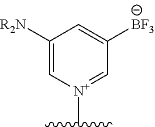

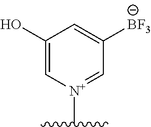

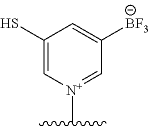

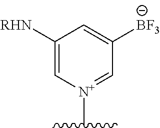

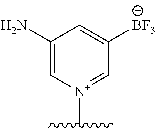

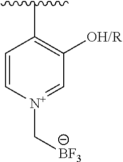

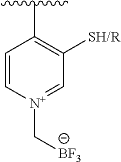

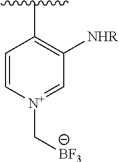

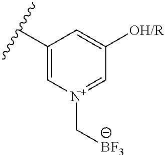

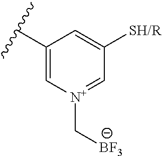

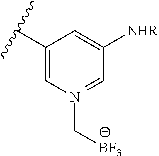

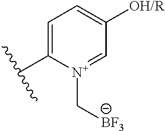

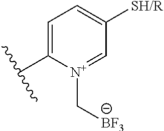

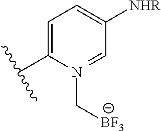

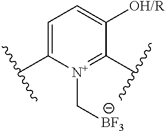

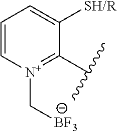

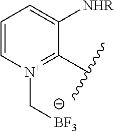

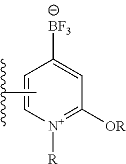

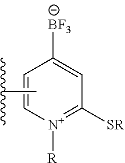

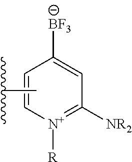

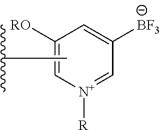

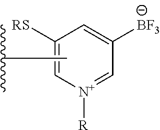

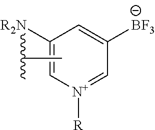

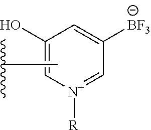

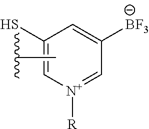

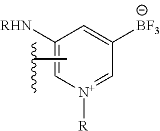

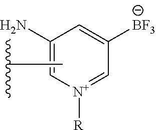

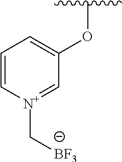

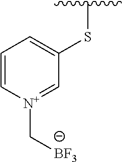

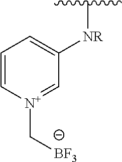

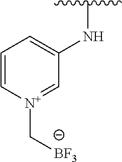

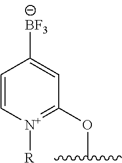

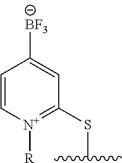

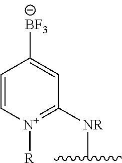

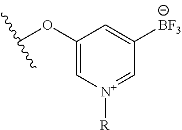

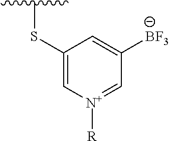

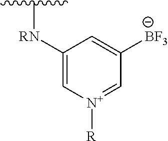

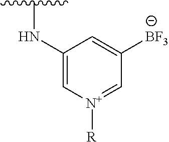

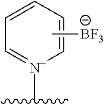

TABLE D
Exemplary BF₃—R⁵— groups.

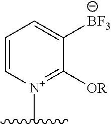

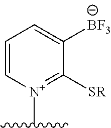

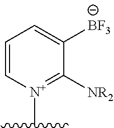

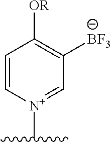

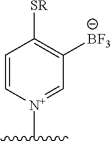

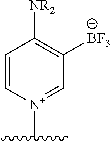

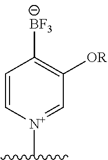

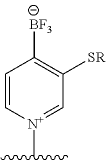

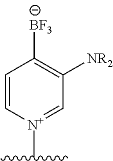

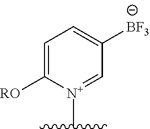

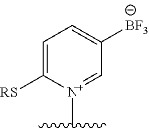

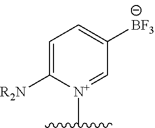

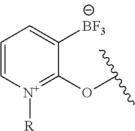

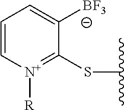

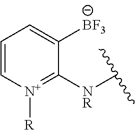

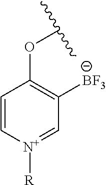

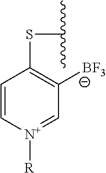

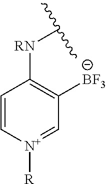

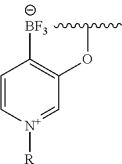

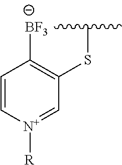

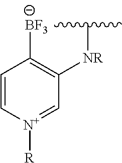

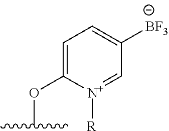

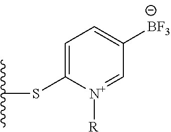

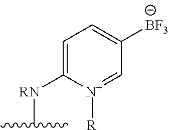

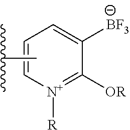

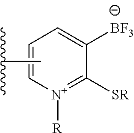

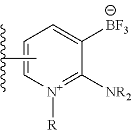

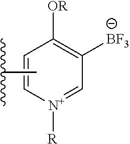

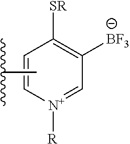

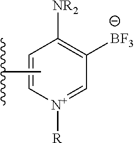

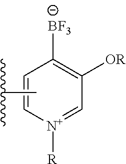

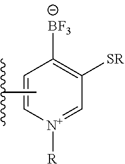

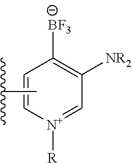

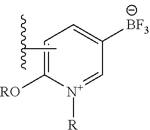

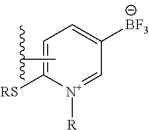

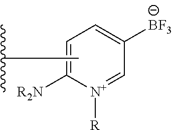

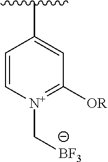

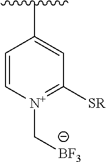

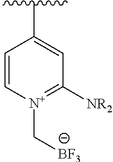

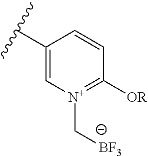

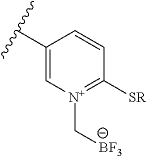

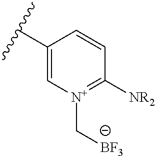

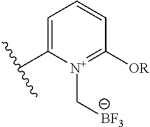

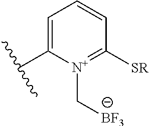

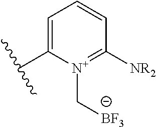

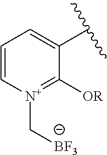

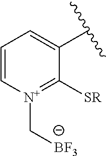

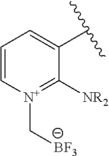

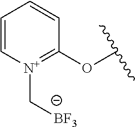

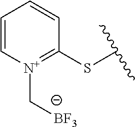

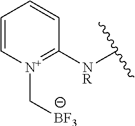

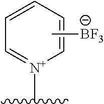

In some embodiments, a BF₃—R⁵— may independently selected from Table C, in which each R (when present) in the pyridine substituted —OR, —SR, —NR—, —NHR or —NR₂ is independently a linear or branched C₁-C₅ alkyl. In some embodiments, R is methyl. In some embodiments, R is ethyl. In some embodiments, R is propyl. In some embodiments, R is isopropyl. In some embodiments, R is n-butyl. The trifluoroborate-containing prosthetic group(s) may comprise ¹⁸F. In some embodiments, one fluorine is a BF₃—R⁵— is ¹⁸F. In some embodiments, all three fluorines in a BF₃—R⁵— are ¹⁸F. In some embodiments, all three fluorines in a BF₃—R⁵— are ¹⁹F.

In some embodiments, a BF₃—R⁵— may independently form selected from Table D, in which each R (when present) in the pyridine substituted —OR, —SR, —NR—, —NHR or —NR₂ is independently a linear or branched C₁-C₅ alkyl. In some embodiments, R is methyl. In some embodiments, R is ethyl. In some embodiments, R is propyl. In some embodiments, R is isopropyl. In some embodiments, R is n-butyl. In some embodiments, a BF₃—R⁵— is

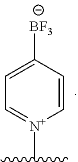

In some embodiments, all three fluorines in a BF₃—R⁵— are ¹⁸F. In some embodiments, one fluorine in a BF₃—R⁵— is ¹⁸F. In some embodiments, all three fluorines in a BF₃—R⁵— are ¹⁹F. In some embodiments, at least one R^{rad} is an aryl or heteroaryl substituted with at least one radiohalogen. In some embodiments, at least one R^{rad} is an aryl or an N-heteroaryl substituted with at least one radiohalogen. In some embodiments, at least one R^{rad} is an aryl or a 6-membered heteroaryl substituted with at least one radiohalogen. In some embodiments, at least one R^{rad} is a phenyl, a pyridyl, or a quinoyl substituted with at least one radiohalogen. In some embodiments, at least one R^{rad} is a pyridyl or a quinoyl substituted with at least one radiohalogen. In some embodiments, at least one R^{rad} is a pyridyl or a quinoyl substituted with at least one ¹⁸F. In some embodiments, at least one R^{rad} is a pyridyl or a quinoyl substituted with a ¹⁸F. In some embodiments, at least one R^{rad} is a pyridyl substituted with a ¹⁸F. In some embodiments, at least one R^{rad} is

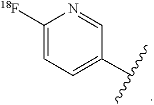 [SMILES: *c1ccc([18F])nc1]

In some embodiments, at least one R^{rad} is an aryl or heteroaryl substituted with at least one fluorine precursor leaving group. In some embodiments, the fluorine precursor leaving group is an electron-withdrawing leaving group. In some embodiments, the fluorine precursor leaving group is a sulfonate leaving group, aryl iodonium group, diazonium group, —NMe₃′, —NO₂, F, Cl, Br, I, —CN, or —CF₃. In some embodiments, the fluorine precursor leaving group is a -OTs (tosylate), —OMe (mesylate), -Ons (nosylate), —NMe₃′, —NO₂, F, Cl, Br, I, —CN, or —CF₃. In some embodiments, at least one R^{rad} is an aryl or heteroaryl substituted with at least one —NMe₃′, —NO₂, F, Cl, Br, I, —CN, or —CF₃. In some embodiments, at least one R^{rad} is an aryl or an N-heteroaryl substituted with at least one —NMe₃′, —NO₂, F, Cl, Br, I, —CN, or —CF₃. In some embodiments, at least one R^{rad} is an aryl or a 6-membered heteroaryl substituted with at least one —NMe₃′, —NO₂, F, Cl, Br, I, —CN, or —CF₃. In some embodiments, at least one R^{rad} is an aryl or a pyridyl substituted with at least one —NMe₃′, —NO₂, F, Cl, Br, I, —CN, or —CF₃. In some embodiments, at least one R^{rad} is an aryl or a pyridyl substituted with at least one —NMe₃⁺. In some embodiments, at least one R^{rad} is

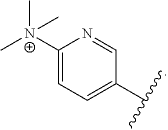 [SMILES: *c1ccc([N+](C)(C)C)nc1]

In some embodiments, -Xaa¹-Xaa²-Xaa³-Xaa⁴-Xaa⁵-Xaa⁶-Xaa⁷-Xaa⁸-ψ-Xaa⁹-R is: D-Phe-Gln-αMeTrp-Ala-Tle-Gly-His-LeuψPro-NH₂ (SEQ ID NO: 3); D-Phe-Gln-αMeTrp-Ala-Val-Gly-His-Sta-Leu-NH₂ (SEQ ID NO: 4); D-Phe-Gln-αMeTrp-Ala-Tle-Gly-His-Sta-Leu-NH₂ (SEQ ID NO: 5); D-Phe-Gln-Trp-Ala-Val-N-MeGly-Trp-LeuψPro-NH₂ (SEQ ID NO: 6); D-Phe-Gln-αMeTrp-Ala-Tle-Gly-Trp-LeuψPro-NH₂ (SEQ ID NO: 7); D-Phe-Gln-αMeTrp-Ala-Tle-Gly-Trp(5-F)-LeuψPro-NH₂ (SEQ ID NO: 8); D-Phe-Gln-αMeTrp-Ala-Tle-Gly-Trp-Sta-Leu-NH₂ (SEQ ID NO: 9); D-Phe-Gln-αMeTrp-Ala-Tle-Gly-Trp(5-F)-Sta-Leu-NH₂ (SEQ ID NO: 10); D-Phe-Cit-αMeTrp-Ala-Tle-Gly-His-LeuψPro-NH₂ (SEQ ID NO: 11); D-Phe-Glu-αMeTrp-Ala-Tle-Gly-His-LeuψPro-NH₂ (SEQ ID NO: 12); D-Phe-Agb-αMeTrp-Ala-Tle-Gly-His-LeuψPro-NH₂ (SEQ ID NO: 13); D-Phe-Gln-αMeTrp-N-MeGly-Tle-Gly-His-LeuψPro-NH₂ (SEQ ID NO: 14); or

D-Phe-Gln-αMeTrp-N-MeGly-Tle-Gly-Trp-LeuψPro-NH₂ (SEQ ID NO: 15), wherein ψ is a reduced peptide bond.

In some embodiments,

(SEQ ID NO: 16)
-Xaa¹-Xaa²-Xaa³-Xaa⁴-Xaa⁵-Xaa⁶-Xaa⁷-Xaa⁸-ψ-Xaa⁹-Xaa¹⁰-
R is D-Phe-Gln-Trp-Ala-Val-N-MeGly-His-LeuψPro-NH₂.

In some embodiments,

-Xaa¹-Xaa²-Xaa³-Xaa⁴-Xaa⁵-Xaa⁶-Xaa⁷-Xaa⁸-ψ-Xaa⁹-
Xaa¹⁰-R is
(SEQ ID NO: 17)
D-Phe-Gln-Trp-Ala-Val-N-MeGly-Trp-LeuψPro-
Val-NH₂;
(SEQ ID NO: 18)
D-Phe-Gln-Trp-Ala-Val-N-MeGly-His-LeuψPro-
Val-NH₂;
(SEQ ID NO: 19)
D-Phe-Gln-αMeTrp-Ala-Tle-Gly-Trp-LeuψPro-Tle-
Val-NH₂;
or
(SEQ ID NO: 20)
D-Phe-Gln-αMeTrp-Ala-Tle-Gly-His-LeuψPro-Tle-
Val-NH₂;
wherein ψ is a reduced peptide bond.

In some embodiments,

[Xaa¹²-Xaa¹-Xaa²-Xaa³-Xaa⁴-Xaa⁵-Xaa⁶-Xaa⁷-Xaa⁸-ψ-
Xaa⁹-R] is
(SEQ ID NO: 21)
Pip-D-Phe-Gln-7FTrp-Ala-Tle-Gly-His-LeuψPro-NH₂.

In some embodiments, the compounds of formula (A), (I), (IA), (IA-1), (IB), (IB-1), (IC), (IC-1), (ID), (ID-1), (IE), or (IE-1), excludes compounds disclosed in WO2009/109332. PCT application publication WO2009/109332 is incorporated by reference in its entirety herein.

In some embodiments, the compounds of formula (A), (I), (IA), (IA-1), (IB), (IB-1), (IC), (IC-1), (ID), (ID-1), (IE), or (IE-1), excludes compounds disclosed in WO2021/068051. PCT application publication WO2021/068051 is incorporated by reference in its entirety herein.

In some embodiments, the compounds of formula (A), (I), (IA), (IA-1), (IB), (IB-1), (IC), (IC-1), (ID), (ID-1), (IE), or (IE-1), excludes compounds described in Wang, L et al., Molecules, 2022 27, 3777, which is incorporated by reference in its entirety herein.

In some embodiments, the compounds of formula (A), (I), (IA), (IA-1), (IB), (IB-1), (IC), (IC-1), (ID), (ID-1), (IE), or (IE-1), excludes compounds described in Wang, L et al., Molecules, 2022 27, 3777, which is incorporated by reference in its entirety herein.

In some embodiments, the compounds of formula (A), (I), (IA), (IA-1), (IB), (IB-1), (IC), (IC-1), (ID), (ID-1), (IE), or (IE-1), excludes LW01186.

In some embodiments, the compounds of formula (A), (I), (IA), (IA-1), (IB), (IB-1), (IC), (IC-1), (ID), (ID-1), (IE), or (IE-1), excludes AMTG having the structure

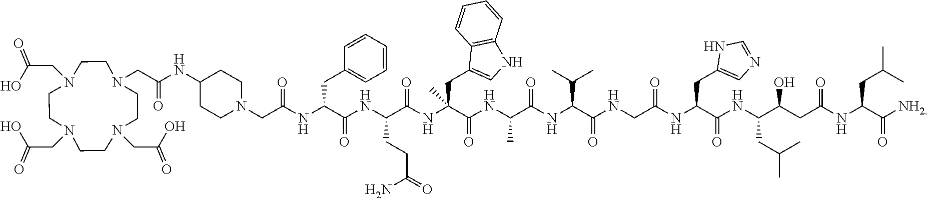 [SMILES: CC(C)C[C@H](NC(=O)C[C@H](O)[C@H](CC(C)C)NC(=O)[C@H](Cc1cnc[nH]1)NC(=O)CNC(=O)[C@@H](NC(=O)[C@H](C)NC(=O)[C@](C)(Cc1c[nH]c2ccccc12)NC(=O)[C@H](CCC(N)=O)NC(=O)[C@@H](Cc1ccccc1)NC(=O)CN1CCC(NC(=O)CN2CCN(CC(=O)O)CCN(CC(=O)O)CCN(CC(=O)O)CC2)CC1)C(C)C)C(N)=O]

In some embodiments, the compounds of formula (A), (I), (IA), (IA-1), (IB), (IB-1), (IC), (IC-1), (ID), (ID-1), (IE), or (IE-1), excludes AMTG2 having the structure

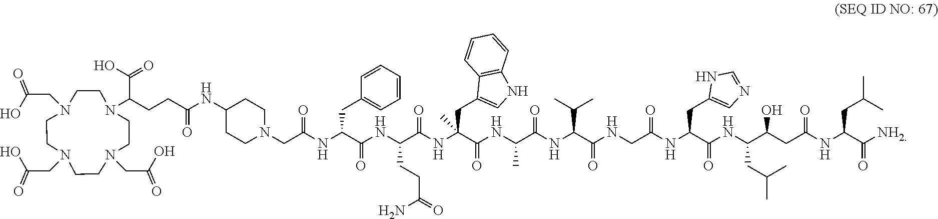 [SMILES: CC(C)C[C@H](NC(=O)C[C@H](O)[C@H](CC(C)C)NC(=O)[C@H](Cc1cnc[nH]1)NC(=O)CNC(=O)[C@@H](NC(=O)[C@H](C)NC(=O)[C@](C)(Cc1c[nH]c2ccccc12)NC(=O)[C@H](CCC(N)=O)NC(=O)[C@@H](Cc1ccccc1)NC(=O)CN1CCC(NC(=O)CCC(C(=O)O)N2CCN(CC(=O)O)CCN(CC(=O)O)CCN(CC(=O)O)CC2)CC1)C(C)C)C(N)=O]

In some embodiments, the peptidic compound described herein is:

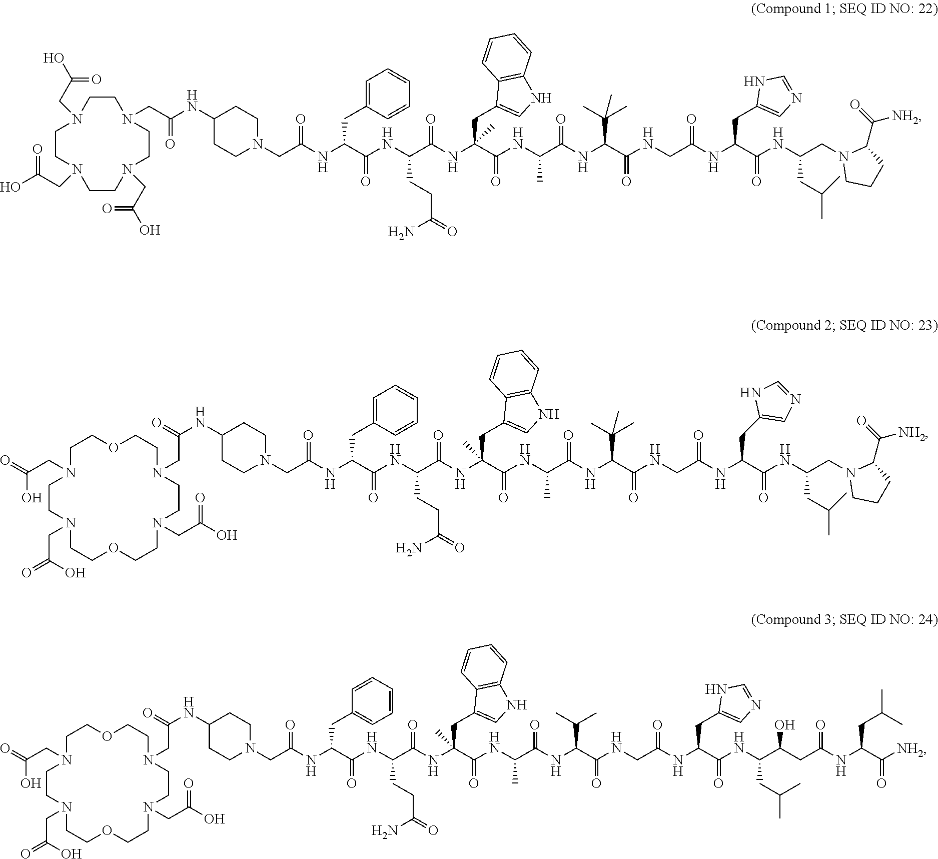

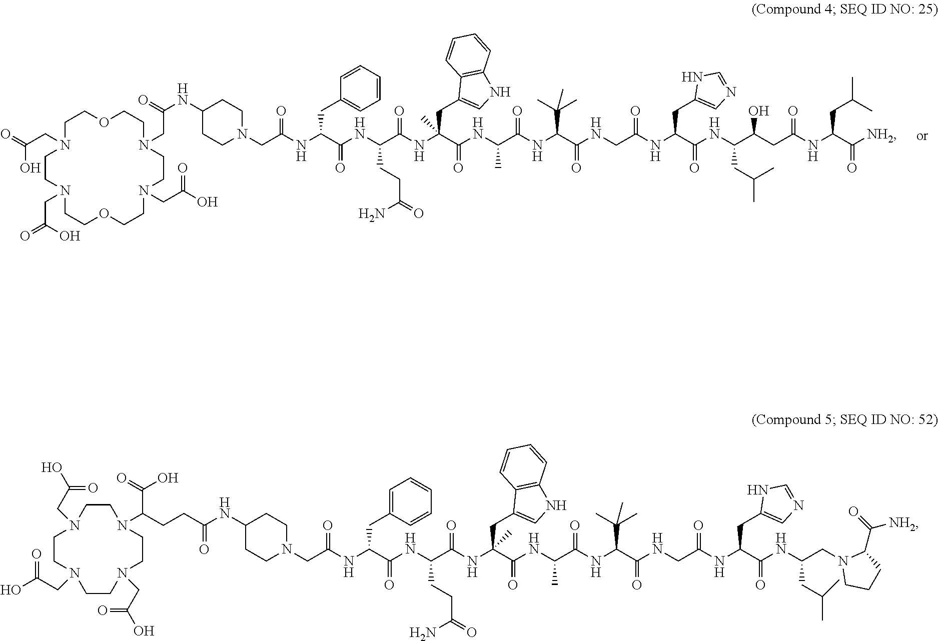

or a salt or a solvate thereof. In some embodiments, the compound is conjugated by a radiometal. In some embodiments, the radiometal is ⁶⁸Ga, ¹⁷⁷Lu, or ²²⁵Ac.

In some embodiments, the peptidic compound described herein is:

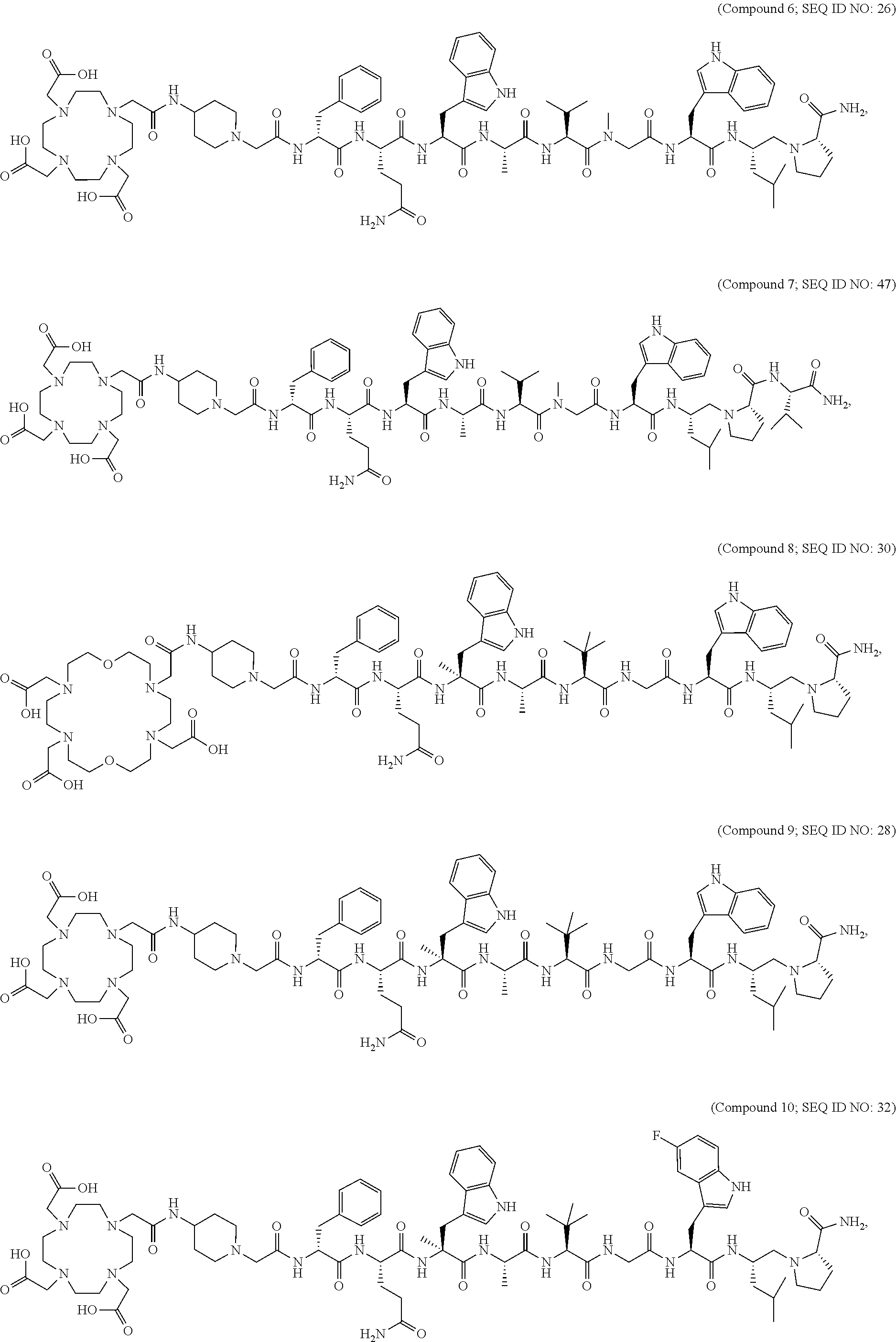

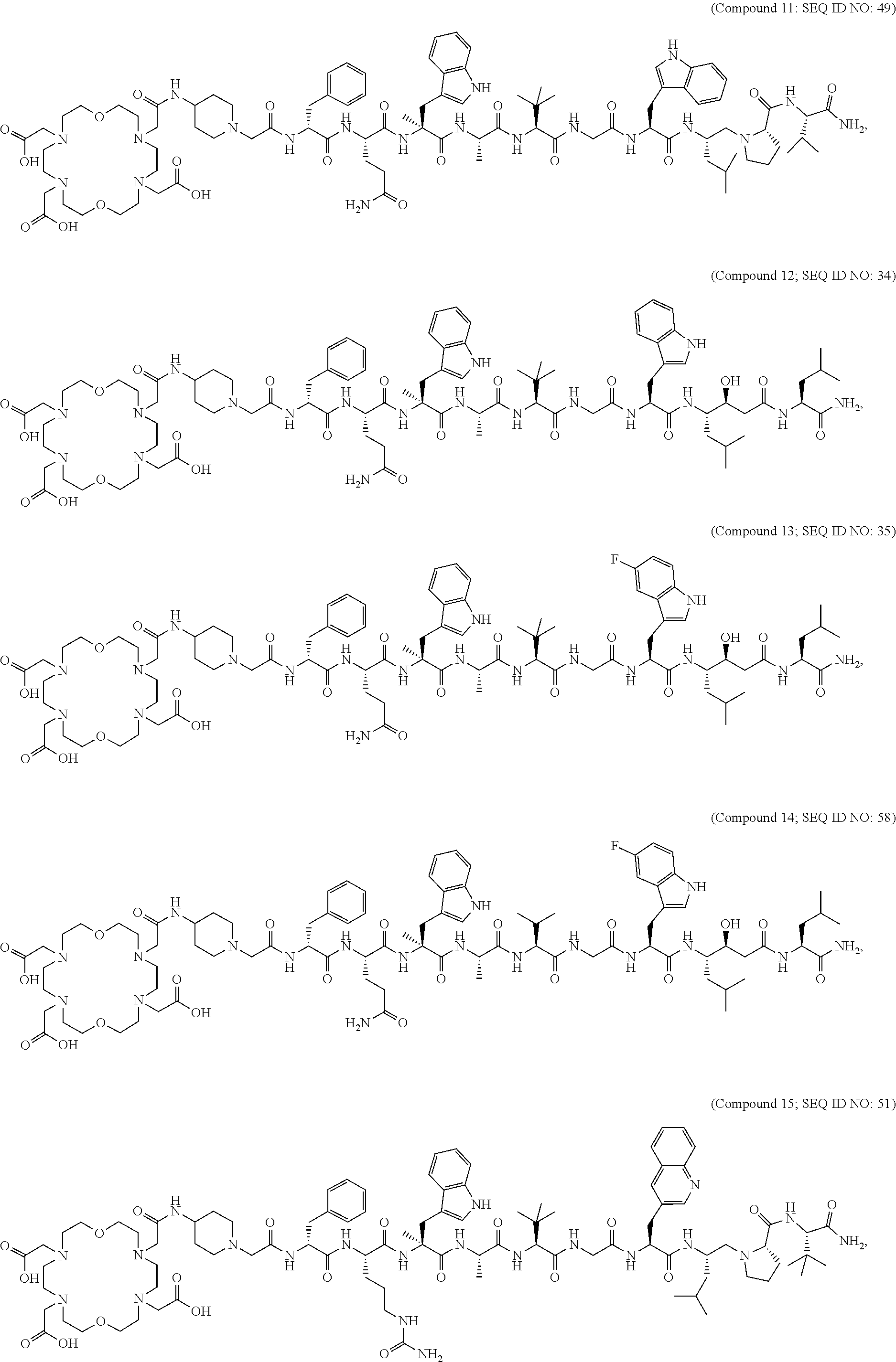

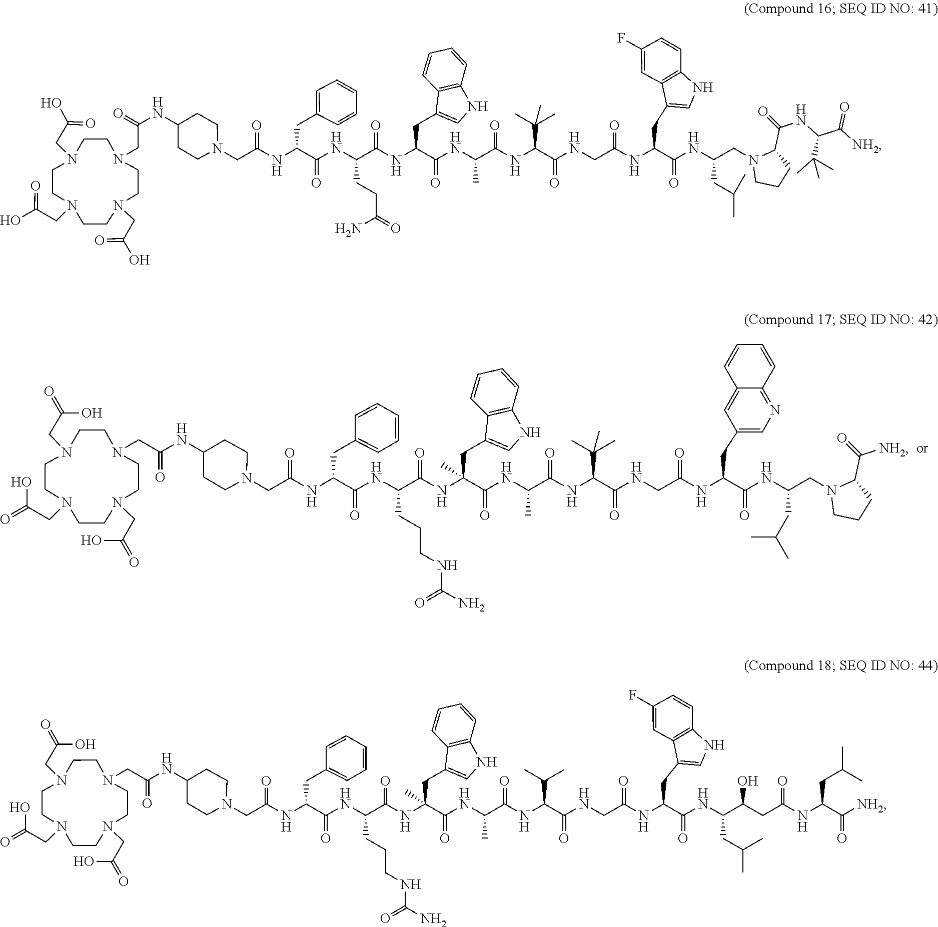

or a salt or a solvate thereof. In some embodiments, the compound is conjugated by a radiometal. In some embodiments, the radiometal is ⁶⁸Ga, ¹⁷⁷Lu, or ²²⁵Ac.

In some embodiments, the peptidic compound described herein is:

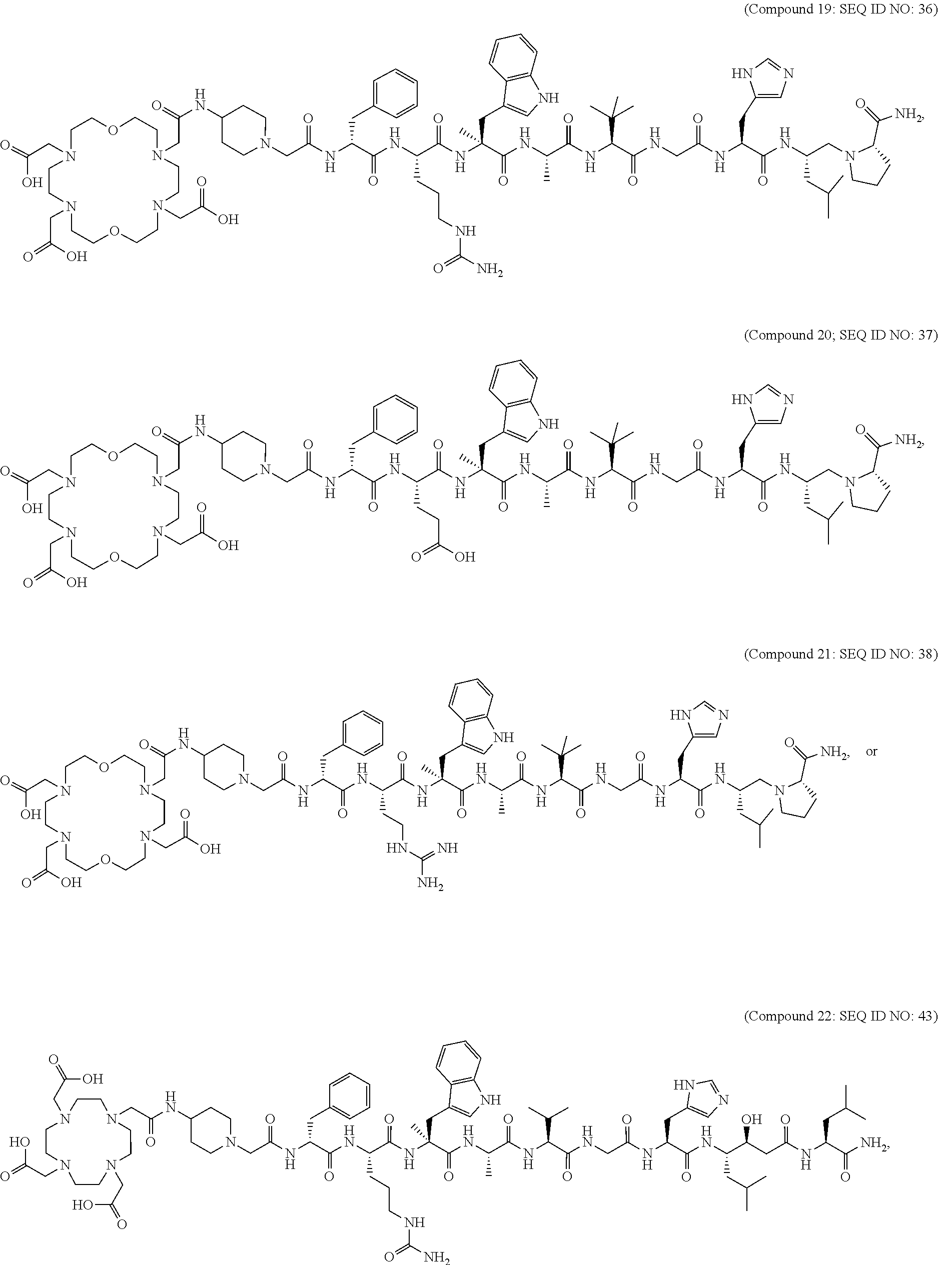

or a salt or a solvate thereof. In some embodiments, the compound is conjugated by a radiometal. In some embodiments, the radiometal is ⁶⁸Ga, ¹⁷⁷Lu, or ²²⁵Ac.

In some embodiments, the peptidic compound described herein is:

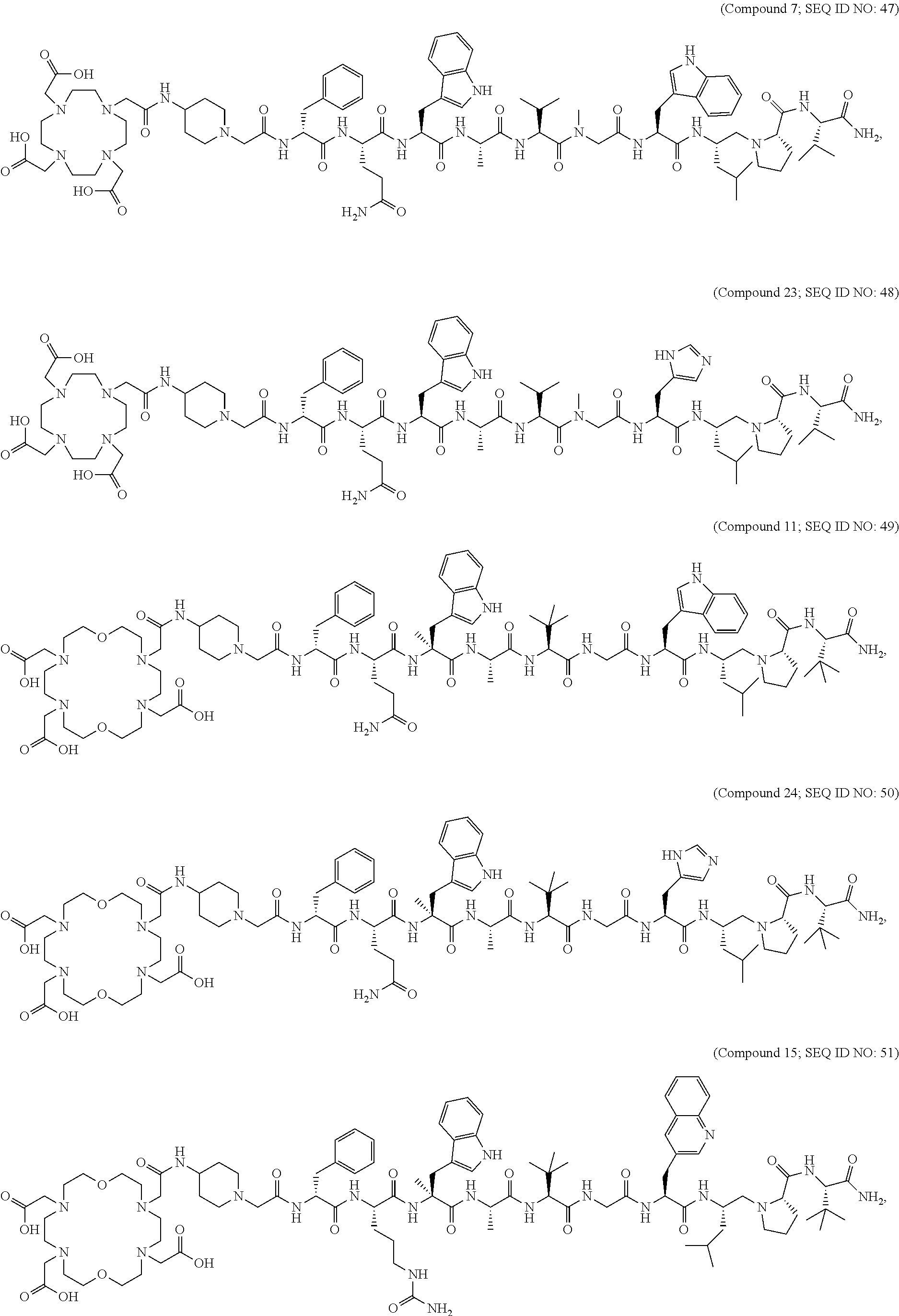

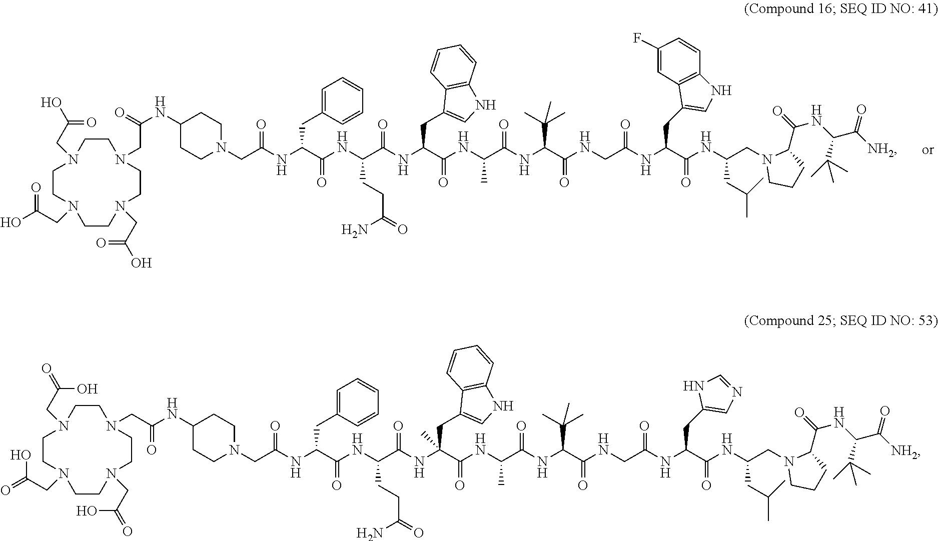

(Compound 25; SEQ ID NO: 53), or a salt or a solvate thereof. In some embodiments, the compound is conjugated by a radiometal. In some embodiments, the radiometal is ⁶⁸Ga, ¹⁷⁷Lu, or ²²⁵Ac.

In some embodiments, the peptidic compound described herein is:

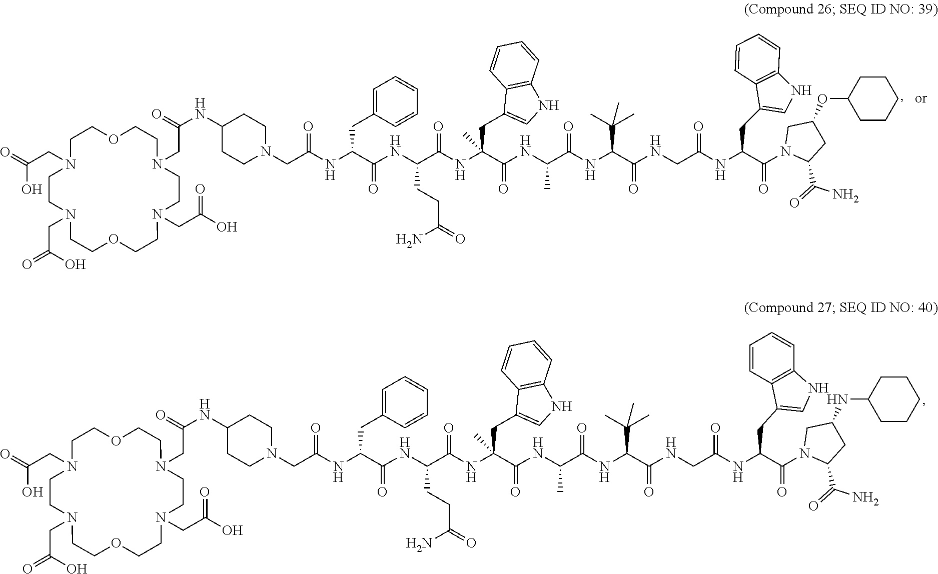

or a salt or a solvate thereof. In some embodiments, the compound is conjugated by a radiometal. In some embodiments, the radiometal is ⁶⁸Ga, ¹⁷⁷Lu, or ²²⁵Ac.

In some embodiments, the peptidic compound described herein is:

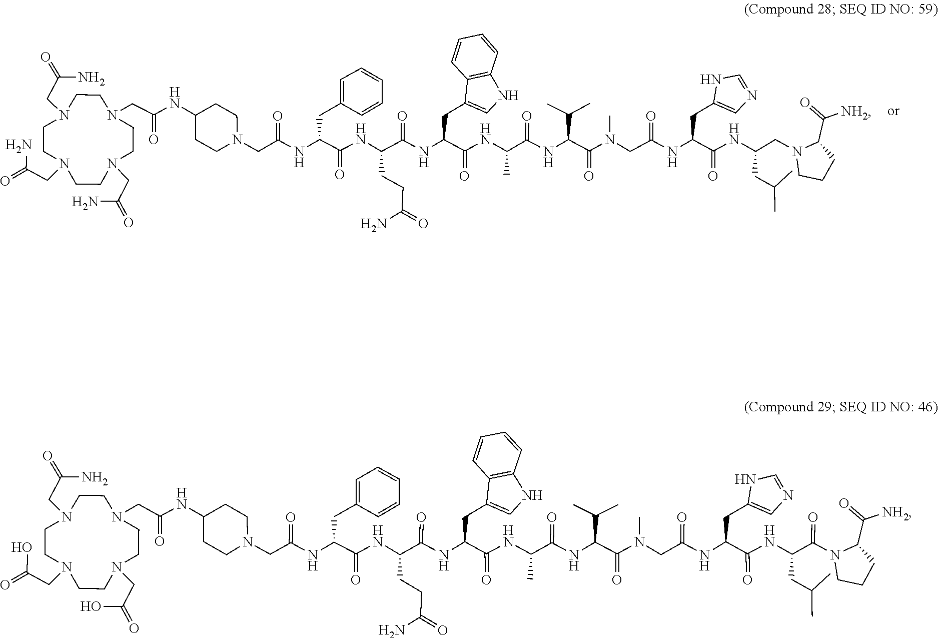 [SMILES: CC(C)C[C@@H](CN1CCC[C@H]1C(N)=O)NC(=O)[C@H](Cc1cnc[nH]1)NC(=O)CN(C)C(=O)[C@@H](NC(=O)[C@H](C)NC(=O)[C@H](Cc1c[nH]c2ccccc12)NC(=O)[C@H](CCC(N)=O)NC(=O)[C@@H](Cc1ccccc1)NC(=O)CN1CCC(NC(=O)CN2CCN(CC(N)=O)CCN(CC(N)=O)CCN(CC(N)=O)CC2)CC1)C(C)C ; CC(C)C[C@H](NC(=O)[C@H](Cc1cnc[nH]1)NC(=O)CN(C)C(=O)[C@@H](NC(=O)[C@H](C)NC(=O)[C@H](Cc1c[nH]c2ccccc12)NC(=O)[C@H](CCC(N)=O)NC(=O)[C@@H](Cc1ccccc1)NC(=O)CN1CCC(NC(=O)CN2CCN(CC(N)=O)CCN(CC(=O)O)CCN(CC(=O)O)CC2)CC1)C(C)C)C(=O)N1CCC[C@H]1C(N)=O]

or a salt or a solvate thereof. In some embodiments, the compound is conjugated by a radiometal. In some embodiments, the compound is conjugated with ²⁰³Pb or ²¹²Pb.

In some embodiments, the peptidic compound described herein is:

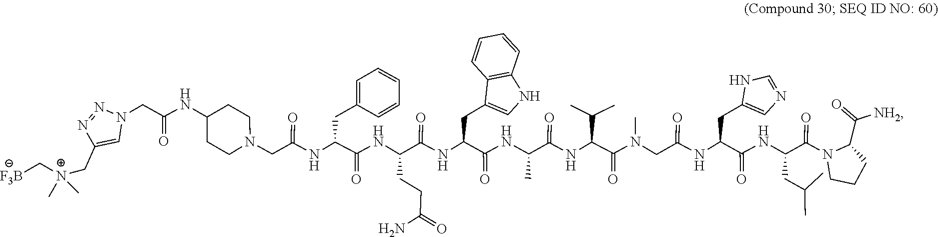

or a salt or a solvate thereof. In some embodiments, at least one fluorine in —BF₃ is ¹⁸F. In some embodiments, one or more fluorines in —BF₃ is ¹⁸F. In some embodiments, all fluorines in —BF₃ is ¹⁸F.

In some embodiments, the peptidic compound described herein is:

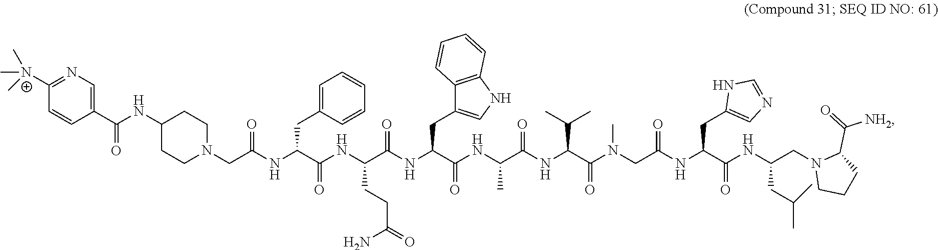 [SMILES: CC(C)C[C@@H](CN1CCC[C@H]1C(N)=O)NC(=O)[C@H](Cc1cnc[nH]1)NC(=O)CN(C)C(=O)[C@@H](NC(=O)[C@H](C)NC(=O)[C@H](Cc1c[nH]c2ccccc12)NC(=O)[C@H](CCC(N)=O)NC(=O)[C@@H](Cc1ccccc1)NC(=O)CN1CCC(NC(=O)c2ccc([N+](C)(C)C)nc2)CC1)C(C)C]

or a salt or a solvate thereof.

In some embodiments, the peptidic compound described herein is:

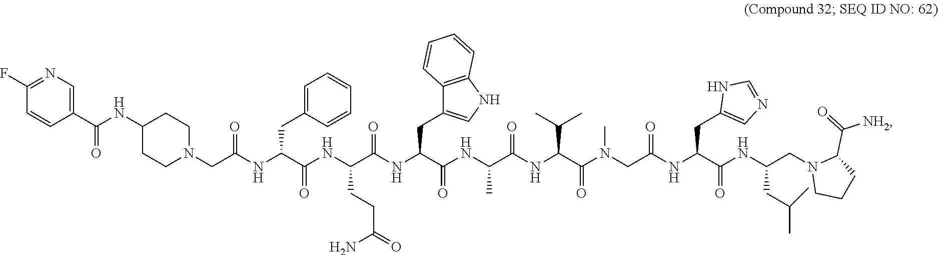 [SMILES: CC(C)C[C@@H](CN1CCC[C@H]1C(N)=O)NC(=O)[C@H](Cc1cnc[nH]1)NC(=O)CN(C)C(=O)[C@@H](NC(=O)[C@H](C)NC(=O)[C@H](Cc1c[nH]c2ccccc12)NC(=O)[C@H](CCC(N)=O)NC(=O)[C@@H](Cc1ccccc1)NC(=O)CN1CCC(NC(=O)c2ccc(F)nc2)CC1)C(C)C]

or a salt or a solvate thereof. In some embodiments, the fluorine (substituted on pyridine) is ¹⁸F.

In some embodiments, the peptidic compound described herein is:

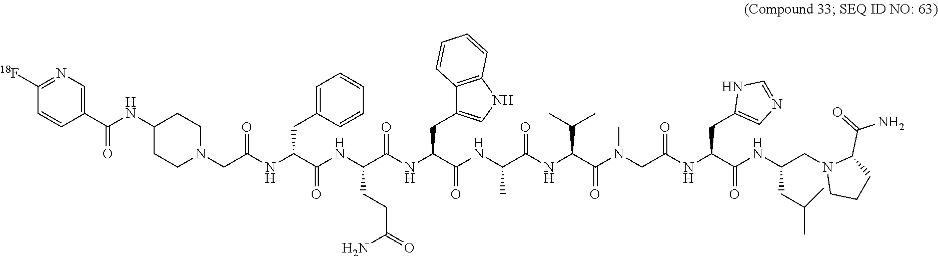 [SMILES: CC(C)C[C@@H](CN1CCC[C@H]1C(N)=O)NC(=O)[C@H](Cc1cnc[nH]1)NC(=O)CN(C)C(=O)[C@@H](NC(=O)[C@H](C)NC(=O)[C@H](Cc1c[nH]c2ccccc12)NC(=O)[C@H](CCC(N)=O)NC(=O)[C@@H](Cc1ccccc1)NC(=O)CN1CCC(NC(=O)c2ccc([18F])nc2)CC1)C(C)C]

or a salt or a solvate thereof.

In some embodiments, the peptidic compound described herein is:

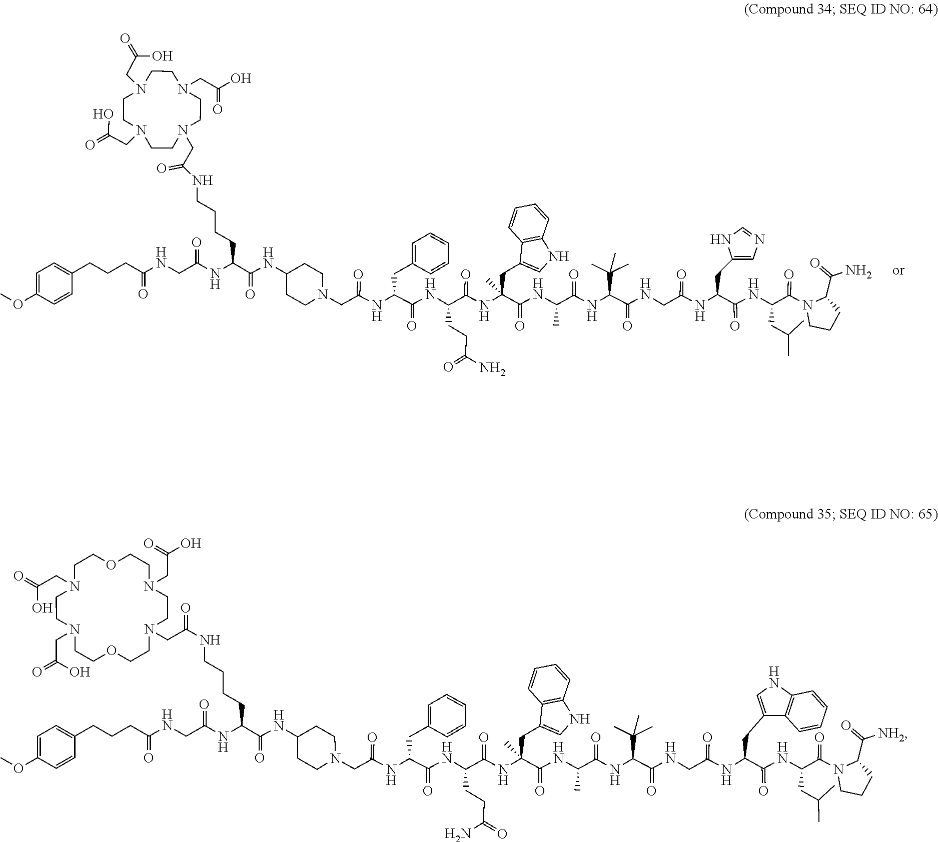

or a salt or a solvate thereof. In some embodiments, the compound is conjugated by a radiometal. In some embodiments, the radiometal is ⁶⁸Ga, ¹⁷⁷Lu, or ²²⁵Ac.

In some embodiments, the peptidic compound described herein is:

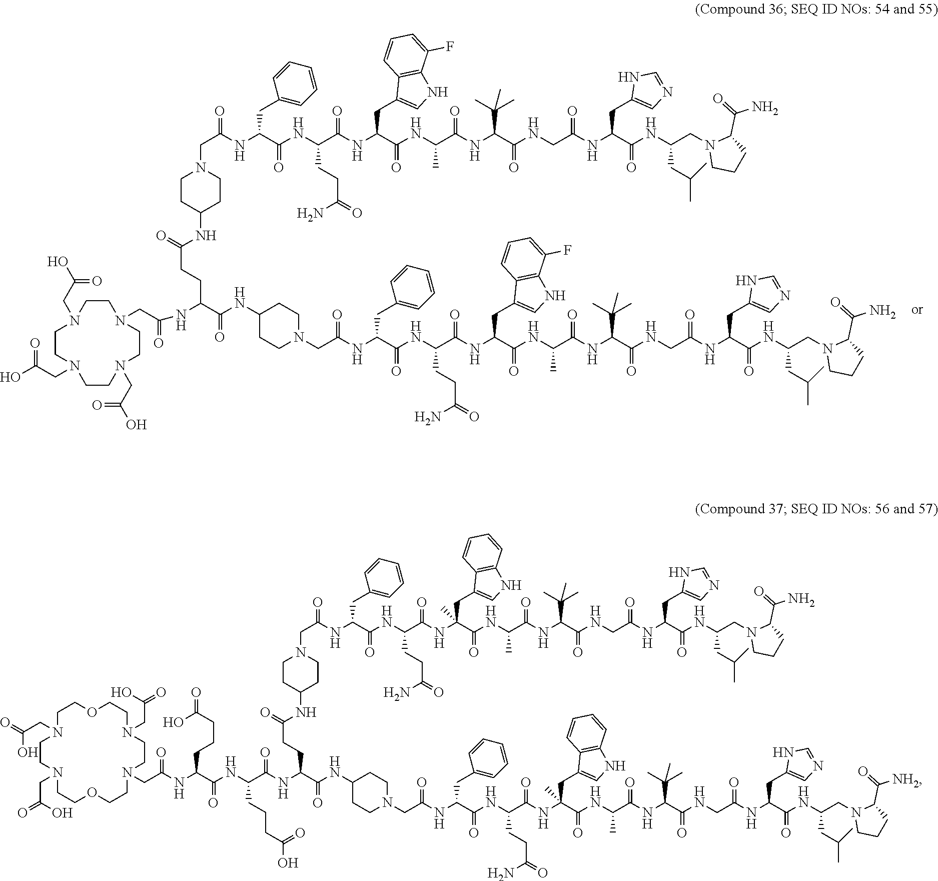

or a salt or a solvate thereof. In some embodiments, the compound is conjugated by a radiometal. In some embodiments, the radiometal is ⁶⁸Ga, 17Lu, or ²²⁵Ac.

In some embodiments, the peptidic compound described herein is selected from any one of Compounds 1-37 (including Compounds 6A, 8A, 9A, and 10A).

In some embodiments, the peptidic compound is

(SEQ ID NO: 22)
DOTA-Pip-D-Phe-Gln-αMeTrp-Ala-Tle-Gly-His-
LeuψPro-NH₂;
(SEQ ID NO: 23)
CROWN-Pip-D-Phe-Gln-αMeTrp-Ala-Tle-Gly-His-
LeuψPro-NH₂;
(SEQ ID NO: 24)
CROWN-Pip-D-Phe-Gln-αMeTrp-Ala-Val-Gly-His-
Sta-Leu-NH₂;
(SEQ ID NO: 25)
CROWN-Pip-D-Phe-Gln-αMeTrp-Ala-Tle-Gly-His-
Sta-Leu-NH₂;
(SEQ ID NO: 26)
DOTA-Pip-D-Phe-Gln-Trp-Ala-Val-N-MeGly-Trp-
LeuψPro-NH₂;
(SEQ ID NO: 27)
DOTAGA-Pip-D-Phe-Gln-Trp-Ala-Val-N-MeGly-Trp-
LeuψPro-NH₂;
(SEQ ID NO: 28)
DOTA-Pip-D-Phe-Gln-αMeTrp-Ala-Tle-Gly-Trp-
LeuψPro-NH₂;
(SEQ ID NO: 29)
DOTAGA-Pip-D-Phe-Gln-αMeTrp-Ala-Tle-Gly-Trp-
LeuψPro-NH₂;
(SEQ ID NO: 30)
CROWN-Pip-D-Phe-Gln-αMeTrp-Ala-Tle-Gly-Trp-
LeuψPro-NH₂;
(SEQ ID NO: 31)
CROWNGA-Pip-D-Phe-Gln-αMeTrp-Ala-Tle-Gly-
Trp-LeuψPro-NH₂;
(SEQ ID NO: 32)
DOTA-Pip-D-Phe-Gln-αMeTrp-Ala-Tle-Gly-Trp
(5-F)-LeuψPro-NH₂;
(SEQ ID NO: 33)
DOTAGA-Pip-D-Phe-Gln-αMeTrp-Ala-Tle-Gly-Trp
(5-F)-LeuψPro-NH₂;
(SEQ ID NO: 34)
CROWN-Pip-D-Phe-Gln-αMe Trp-Ala-Tle-Gly-
Trp-Sta-Leu-NH₂;
(SEQ ID NO: 35)
CROWN-Pip-D-Phe-Gln-αMeTrp-Ala-Tle-Gly-Trp
(5-F)-Sta-Leu-NH₂;
(SEQ ID NO: 36)
CROWN-Pip-D-Phe-Cit-αMeTrp-Ala-Tle-Gly-His-
LeuψPro-NH₂;
(SEQ ID NO: 37)
CROWN-Pip-D-Phe-Glu-αMeTrp-Ala-Tle-Gly-His-
LeuψPro-NH₂;
(SEQ ID NO: 38)
CROWN-Pip-D-Phe-Agb-αMeTrp-Ala-Tle-Gly-His-
LeuψPro-NH₂;
(SEQ ID NO: 39)
CROWN-Pip-D-Phe-Gln-αMeTrp-Ala-Tle-Gly-Trp-
(cis-4-OCy-D-Pro)-NH₂;
(SEQ ID NO: 40)
CROWN-Pip-D-Phe-Gln-αMeTrp-Ala-Tle-Gly-Trp-
(cis-4-NHCy-D-Pro)-NH₂;
(SEQ ID NO: 41)
DOTA-Pip-D-Phe-Gln-αMeTrp-Ala-Tle-Gly-5FTrp-
LeuψPro-Tle-NH₂;
(SEQ ID NO: 42)
DOTA-Pip-D-Phe-Cit-αMeTrp-Ala-Tle-Gly-(3-Quin-
Ala)-LeuψPro-NH₂;
(SEQ ID NO: 43)
DOTA-Pip-D-Phe-Cit-αMeTrp-Ala-Val-Gly-His-Sta-
Leu-NH₂;
or
(SEQ ID NO: 44)
DOTA-Pip-D-Phe-Cit-αMeTrp-Ala-Val-Gly-5FTrp-
Sta-Leu-NH₂;

wherein ψ is a reduced peptide bond. In some embodiments, the compound is conjugated by a radiometal. In some embodiments, the radiometal is ⁶⁸Ga, ¹⁷⁷Lu, or ²²⁵Ac.

In some embodiments, the peptidic compound is TCMC-Pip-D-Phe-Gln-Trp-Ala-Val-N-MeGly-His-LeuψPro-NH2 (SEQ ID NO: 45) or PSC-Pip-D-Phe-Gln-Trp-Ala-Val-N-MeGly-His-LeuψPro-NH2 (SEQ ID NO: 46), wherein ψ is a reduced peptide bond. In some embodiments, the compound is conjugated by a radiometal. In some embodiments, the radiometal is ⁶⁸Ga, ¹⁷⁷Lu, ²⁰³Pb, ²¹²Pb, or ²²⁵Ac. In some embodiments, the radiometal is ²⁰³Pb or ²¹²Pb.

In some embodiments, the peptidic compound is DOTA-Pip-D-Phe-Gln-Trp-Ala-Val-N-MeGly-Trp-LeuψPro-Val-NH₂ (SEQ ID NO: 47); DOTA-Pip-D-Phe-Gln-Trp-Ala-Val-N-MeGly-His-LeuψPro-Val-NH₂ (SEQ ID NO: 48); CROWN-Pip-D-Phe-Gln-αMeTrp-Ala-Tle-Gly-Trp-LeuψPro-Tle-NH₂ (SEQ ID NO: 49); or CROWN-Pip-D-Phe-Gln-αMeTrp-Ala-Tle-Gly-His-LeuψPro-Tle-NH₂ (SEQ ID NO: 50); CROWN-Pip-D-Phe-Cit-αMeTrp-Ala-Tle-Gly-(3-Quin-Ala)-LeuψPro-Tle-NH₂ (SEQ ID NO: 51); DOTAGA-Pip-D-Phe-Gln-αMeTrp-Ala-Tle-Gly-His-LeuψPro-NH₂ (SEQ ID NO: 52); or DOTA-Pip-D-Phe-Gln-αMeTrp-Ala-Tle-Gly-His-LeuψPro-Tle-NH₂ (SEQ ID NO: 53); wherein Lp is a reduced peptide bond. In some embodiments, the compound is conjugated by a radiometal. In some embodiments, the radiometal is ⁶⁸Ga, ¹⁷⁷Lu, or ²²⁵Ac.

In some embodiments, the peptidic compound is DOTA-GlU(Pip-^{D}Phe-Gln-7FTrp-Ala-Tle-Gly-His-LeupPro-NH2)₂ (SEQ ID NOs: 54 and 55) or CROWN-Aad-Aad-GlU(Pip-^{D}Phe-Gln-αMeTrp-Ala-Tle-Gly-His-LeuLPPro-NH₂)₂(SEQ ID NOs: 56 and 57), wherein ψ is a reduced peptide bond. In some embodiments, the compound is conjugated by a radiometal. In some embodiments, the radiometal is ⁶⁸Ga, ¹⁷⁷Lu, or ²²⁵Ac.

In some embodiments of the peptidic compound described herein (e.g., compounds of formula (A), (I), (IA), (IA-1), (IB), (IB-1), (IC), (IC-1), (ID), (ID-1), (IE), (IE-1), (IF), (IF-1), (II), (IIA), (B), or (B-1) or salt or solvate thereof), the compound is bound to (or conjugated with) or include a radiometal, a radiohalogen, or a radiohalogen containing metal. In some embodiments, the radiometal, the radiohalogen, or the radiohalogen containing metal is [¹⁸F]AIF, ⁴⁴Sc, ⁴⁷Sc, ⁶¹Cu, ⁶⁴Cu, ⁶⁷Cu, ⁶⁷Ga, ⁶⁸Ga, ⁷²As, ⁷⁷As, ⁸⁶Y, ⁸⁹Zr, ⁹⁰Y, ⁹⁰Nb, ^{94m}Tc, ^{99m}Tc ¹⁰⁵Rh, ¹⁰⁹Pd, ¹¹⁰In, ¹¹¹In, ¹¹⁴In, ^{117m}Sn, ¹²³I, ¹²⁴I, ¹³¹I, ¹³²La, ¹³³La, ¹³⁴La, ¹³⁴Ce, ¹³⁵La, ¹⁴²Pr, ¹⁴⁹Tb, ¹⁴⁹Pm ¹⁵²Tb, ¹⁵³Sm, ¹⁵⁵Tb, ¹⁵⁹Gd, ¹⁶¹Tb, ¹⁶⁵Er, ¹⁶⁶Ho, ¹⁷⁵Yb, ¹⁷⁷Lu ¹⁸⁶Re, ¹⁸⁸Re, ¹⁹⁸Au, ¹⁹⁹Au, ²⁰³Pb, ^{211A}t, ²¹²Pb, ²¹²Bi, ²¹³Bi, ²²³Ra, ²²⁴Ra, ²²⁵Ac, or ²²⁷Th. In some embodiments, the radiometal, the radiohalogen, or the radiohalogen containing metal is [¹⁸F]AIF, ⁴⁴Sc, ⁶¹Cu, ⁶⁴CU ⁶⁷Ga, ⁶⁸Ga, ⁷²As, ⁸⁶Y, ⁸⁹Zr, ⁹⁰Nb, ^{99m}Tc, ^{110m}In, ¹¹¹In, ¹²³I, ¹²⁴I, ¹³¹I, ¹⁵²Tb, ¹⁵⁵Tb, ¹⁷⁷Lu ²⁰³Pb, ²¹²Pb, or ²²⁵Ac. In some embodiments, the radiometal is ⁴⁷Sc, ⁶⁴Cu, ⁶⁷Cu, ⁷⁷As, ⁹⁰Y, ¹⁰⁵Rh, ¹⁰⁹Pd, ¹¹¹In, ^{117m}Sn, ¹⁴²Pr, ¹⁴⁹Pm, ¹⁴⁹Tb, ¹⁵³Sm, ¹⁵⁹Gd, ¹⁶¹Tb, ¹⁶⁵Er, ¹⁶⁶Ho, ¹⁷⁵Yb, ¹⁷⁷Lu ¹⁸⁶Re, ¹⁸⁸Re, ¹⁹⁸Au, ¹⁹⁹Au, ^{211A}t, ²¹²Pb, ²¹²Bi, ²¹³Bi, ²²³Ra, ²²⁴Ra, ²²⁵Ac, or ²²⁷Th. In some embodiments, the radiometal is ⁴⁴Sc, ⁴⁷Sc, ⁶⁴Cu, ⁶⁷Ga, ⁶⁷Cu, ⁶⁸Ga, ⁸⁶Y, ⁹⁰Y, ¹¹¹In, ^{117m}Sn, ¹⁴⁹Tb, ¹⁵²Tb, ¹⁵³Sm, ¹⁵⁵Tb, ¹⁶¹Tb, ¹⁶⁵Er, ¹⁷⁷Lu ²⁰³Pb, ²¹²Pb, ²¹²Bi, ²¹³Bi, ²²³Ra, ²²⁴Ra, ²²⁵Ac, or ²²⁷Th. In some embodiments, the radiometal is ⁴⁴Sc, ⁶¹Cu, ⁶⁴Cu, ⁶⁷Cu, ⁶⁷Ga, ⁶⁸Ga, ⁸⁶Y, ⁹⁰Y, ¹¹¹In, ¹⁷⁷Lu, ²¹³Bi, 212Bi, or ²²⁵Ac. In some embodiments, the radiometal is ⁶⁸Ga, ¹⁷⁷Lu, ²⁰³Pb, ²¹²Pb, or ²²⁵Ac. In some embodiments, the radiometal is ⁶⁸Ga, ¹⁷⁷Lu, or ²²⁵Ac.

In some embodiments, the peptidic compounds described herein are gastrin-releasing peptide receptor (GRPR) antagonists.

In some embodiments, the peptidic compounds described herein the compound is conjugated with a radiometal, a radiohalogen, or a radiohalogen for positron emission tomography (PET) or single photon emission computed tomography (SPECT) imaging of GRPR expressing tumors, wherein the compound is conjugated with a radiometal, a radiohalogen, or a radiohalogen that is a positron emitter or a gamma emitter. In some embodiments, the positron or gamma emitter is ¹⁸F, ⁴⁴Sc, ⁶¹Cu, ⁶⁴Cu, ⁶⁷Ga, ⁶⁸Ga, ⁷²As, ⁸⁶Y, ⁸⁹Zr, ⁹⁰Nb, ^{99m}Tc, ¹¹⁰In, ¹¹¹In, ¹²³I, ¹²⁴I, ¹³¹I, ¹³²La, ¹³³La ¹³⁴La, ¹³⁴Ce, ¹⁵²Tb, ¹⁵⁵Tb, or ²⁰³Pb.

In some embodiments, the peptidic compounds described herein the compound is conjugated with a radiometal that is used for therapy. In some embodiments, the radiometal is ⁴⁷Sc ⁶⁴Cu ⁶⁷Cu ⁷⁷As, ⁹⁰Y, ¹⁰⁵Rh, ¹⁰⁹Pd, ¹¹¹In, ^{117m}Sn, ¹³⁵La, ¹⁴²Pr, ¹⁴⁹Pm ¹⁴⁹Tb, ¹⁵³Sm, ¹⁵⁹Gd, ¹⁶¹Tb, ¹⁶⁵Er, ¹⁶⁶Ho, ¹⁷⁵Yb, ¹⁷⁷Lu ¹⁸⁶Re, ¹⁸⁸Re, ¹⁹⁸Au, ¹⁹⁹Au, ^{211A}t, ²¹²Pb, ²¹²Bi, ²¹³Bi, ²²³Ra, ²²⁴Ra, ²²⁵Ac, or ²²⁷Th.

The peptidic compounds described herein can be prepared by known peptide synthesis methods, such as methods employing 9-fluorenylmethoxycarbonyl (Fmoc) and/or t-butyloxycarbonyl (Boc) protecting groups. In some embodiments, peptide synthesis is solid-phase peptide synthesis. Fmoc protecting groups can be removed from the amino acid on the solid support under basic conditions. The amino acid to be added can be activated for coupling using activating reagents such as 2-(1H-benzotriazol-1-yl)-1,1,3,3-tetramethyluronium hexafluorophosphate (HBTU), 2-(1H-benzotriazol-1-yl)-1,1,3,3-tetramethyluronium tetrafluoroborate (TBTU), 2-(7-Aza-1H-benzotriazole-1-yl)-1,1,3,3-tetramethyluronium hexafluorophosphate (HATU), benzotriazole-1-yl-oxy-tris(dimethylamino)phosphoniumhexafluorophosphate (BOP), benzotriazole-1-yl-oxy-tris(pyrrolidino)phosphoniumhexafluorophosphate (PyBOP). Racemization is minimized by using triazoles, such as 1-hydroxy-benzotriazole (HOBt) and 1-hydroxy-7-aza-benzotriazole (HOAt). In some embodiments, the amino acid coupling reaction is performed in the presence of a suitable base to form a typical peptide bond.

Other than the formation of a typical amino acid peptide bond, amino acids can be coupled at their sidechain functional groups including carboxylic acid and amino groups. Amino acids with carboxylic acid or an amino group in their side chain can be used to form branched peptide chain. In some embodiments, an amino acid with a side chain with a carboxylic acid is Asp, D-Asp, Glu, or D-Glu. In some embodiments, an amino acid with a side chain with an amine is Lys, D-Lys, Orn, D-Orn, Dab, D-Dab, Dap, or D-Dap.

An amino acid with an azide side chain can be used to form a triazole with another amino acid or an input with an alkyne group. In some embodiments, an amino acid with a side chain with an azide is Lys(N₃) or D-Lys(N₃). In some embodiments, an amino acid with a side chain with an alkyne group is Pra or D-Pra.

In some embodiments of the peptidic compounds, Xaa¹, Xaa², Xaa³, Xaa⁴, Xaa⁵, Xaa⁶, Xaa⁷, Xaa⁸, Xaa⁹ or Xaa¹⁰ is each optionally N-methylated. In some embodiments, N-methylation can be achieved by directly using Fmoc-N-methylated amino acids during peptide synthesis.

In some embodiments, the radiolabeling group or the albumin binder is coupled to the peptide N-terminus while the peptide is attached to the solid support.

A prosthetic group containing a trifluoroborate, such as BF₃—R⁵—R⁴— component, can be prepared according to reported procedures (Liu et al. Angew Chem Int Ed 2014 53:11876-11880; Liu et al. J Nucl Med 2015 55:1499-1505; Liu et al. Nat Protoc 2015 10:1423-1432; Kuo et al. J Nucl Med, 2019 60:1160-1166; each of which is incorporated by reference in its entirety). [00239]¹⁸F-labeling of the BF₃-containing compounds via ¹⁸F-¹⁹F isotope exchange reaction can be performed following reported procedures (Liu et al. Nat Protoc 2015 10:1423-1432, incorporated by reference in its entirety).

### Binding and Function

A person having ordinary skill in the art would appreciate that the compounds disclosed herein are bombesin derivatives and therefore function in a similar manner to bombesin. Bombesin and its derivatives are known to bind to GRPR by inserting their C-termini into the binding pocket of the receptor (Peng, et al. 2023 Proc Nat/Acad Sci USA 120). Bombesin derivatives comprising a chelated radionuclide also bind to GRPR in a similar fashion while delivering a cytotoxic radiation dose to a cell (e.g., a tumor cell). A skilled person also appreciates that the disclosed compounds selectively bind to tumors that overexpress GRPR over healthy tissues and organs (e.g., pancreas and kidneys) based on the Examples disclosed herein.

In an aspect, the peptidic compounds of the invention comprise a means for binding to a GRPR-expressing tumor in a subject, wherein the peptidic compound exhibits a tumor/pancreas uptake ratio of at least 1. In some embodiments, the peptidic compound exhibits a tumor/pancreas uptake ratio of about 1.5 or higher. In some embodiments, the peptidic compound exhibits a tumor/pancreas uptake ratio of about 2 or higher. In some embodiments, the peptidic compound exhibits a tumor/pancreas uptake ratio of about 4 or higher. In some embodiments, the peptidic compound exhibits a tumor/pancreas uptake ratio of about 5 or higher. In some embodiments, the peptidic compound exhibits a tumor/pancreas uptake ratio of about 6 or higher. In some embodiments, the peptidic compound exhibits a tumor/pancreas uptake ratio of about 7 or higher. In some embodiments, the peptidic compound exhibits a tumor/pancreas uptake ratio of about 10 or higher. In some embodiments, the peptidic compound exhibits a tumor/pancreas uptake ratio of about 16. In some embodiments, the peptidic compound comprises a radiolabeling group. In some embodiments, the radiolabeling group is a radiometal chelator; an aryl or heteroaryl substituted with at least one radiohalogen; a prosthetic group containing a trifluoroborate; an aryl or heteroaryl substituted with at least one fluorine precursor leaving group; a prosthetic group containing a silicon-fluorine-acceptor moiety; or a prosthetic group containing a fluorophosphate, fluorosulfate, sulfonyl fluoride, or a combination thereof. In some embodiments, the peptidic compound comprises a radiometal chelator optionally comprising or complexed with a radionuclide.

In another aspect, the peptidic compounds of the invention comprise a means for binding to a GRPR-expressing tumor in a subject, wherein the peptidic compound exhibits a tumor comparative time-activity integration (AUCₜᵤₘₒᵣ) of at least 1000% ID/g·h. In some embodiments, the peptidic compound exhibits an AUCₜᵤₘₒᵣ of at least 1100% ID/g·h. In some embodiments, the peptidic compound exhibits an AUCₜᵤₘₒᵣ of at least 1200% ID/g·h. In some embodiments, the peptidic compound exhibits an AUCₜᵤₘₒᵣ of at least 1300% ID/g·h. In some embodiments, the peptidic compound exhibits an AUCₜᵤₘₒᵣ of at least 1400% ID/g·h. In some embodiments, the peptidic compound exhibits an AUCₜᵤₘₒᵣ of at least 1500% ID/g·h. In some embodiments, the peptidic compound exhibits an AUCₜᵤₘₒᵣ of at least 1600% ID/g·h. In some embodiments, the peptidic compound exhibits an AUCₜᵤₘₒᵣ of at least 1700% ID/g·h. In some embodiments, the peptidic compound exhibits an AUCₜᵤₘₒᵣ of at least 1800% ID/g·h. In some embodiments, the peptidic compound exhibits an AUCₜᵤₘₒᵣ of at least 1900% ID/g·h. In some embodiments, the peptidic compound exhibits an AUCₜᵤₘₒᵣ of at least 2000% ID/g·h. In some embodiments, the peptidic compound exhibits an AUCₜᵤₘₒᵣ of at least 2100% ID/g·h. In some embodiments, the peptidic compound comprises a radiolabeling group. In some embodiments, the radiolabeling group is a radiometal chelator; an aryl or heteroaryl substituted with at least one radiohalogen; a prosthetic group containing a trifluoroborate; an aryl or heteroaryl substituted with at least one fluorine precursor leaving group; a prosthetic group containing a silicon-fluorine-acceptor moiety; or a prosthetic group containing a fluorophosphate, fluorosulfate, sulfonyl fluoride, or a combination thereof. In some embodiments, the peptidic compound comprises a radiometal chelator optionally comprising or complexed with a radionuclide.

In yet another aspect, the peptidic compounds of the invention comprise a means for binding to a GRPR-expressing tumor in a subject, wherein the peptidic compound exhibits a tumor/kidney uptake ratio of at least 10. In some embodiments, the peptidic compound exhibits a tumor/kidney uptake ratio of at least 11. In some embodiments, the peptidic compound exhibits a tumor/kidney uptake ratio of at least 12. In some embodiments, the peptidic compound exhibits a tumor/kidney uptake ratio of at least 13. In some embodiments, the peptidic compound exhibits a tumor/kidney uptake ratio of at least 14. In some embodiments, the peptidic compound exhibits a tumor/kidney uptake ratio of at least 15. In some embodiments, the peptidic compound exhibits a tumor/kidney uptake ratio of at least 16. In some embodiments, the peptidic compound exhibits a tumor/kidney uptake ratio of at least 17. In some embodiments, the peptidic compound exhibits a tumor/kidney uptake ratio of at least 18. In some embodiments, the peptidic compound exhibits a tumor/kidney uptake ratio of at least 19. In some embodiments, the peptidic compound exhibits a tumor/kidney uptake ratio of at least 20. In some embodiments, the peptidic compound comprises a radiolabeling group. In some embodiments, the radiolabeling group is a radiometal chelator; an aryl or heteroaryl substituted with at least one radiohalogen; a prosthetic group containing a trifluoroborate; an aryl or heteroaryl substituted with at least one fluorine precursor leaving group; a prosthetic group containing a silicon-fluorine-acceptor moiety; or a prosthetic group containing a fluorophosphate, fluorosulfate, sulfonyl fluoride, or a combination thereof. In some embodiments, the peptidic compound comprises a radiometal chelator optionally comprising or complexed with a radionuclide.

In an additional aspect, the peptidic compounds of the invention comprise a means for binding to a GRPR-expressing tumor in a subject, wherein the peptidic compound comprises a chelator optionally complexed with a radionuclide and wherein the peptidic compound exhibits a tumor/pancreas uptake ratio of at least 1. In some embodiments, the peptidic compound exhibits a tumor/pancreas uptake ratio of 1.5 or higher. In some embodiments, the peptidic compound exhibits a tumor/pancreas uptake ratio of 2 or higher. In some embodiments, the peptidic compound exhibits a tumor/pancreas uptake ratio of 4 or higher. In some embodiments, the peptidic compound exhibits a tumor/pancreas uptake ratio of 5 or higher. In some embodiments, the peptidic compound exhibits a tumor/pancreas uptake ratio of 6 or higher. In some embodiments, the peptidic compound exhibits a tumor/pancreas uptake ratio of about 7 or higher. In some embodiments, the peptidic compound exhibits a tumor/pancreas uptake ratio of about 10 or higher. In some embodiments, the peptidic compound exhibits a tumor/pancreas uptake ratio of about 16.

In a further aspect, the peptidic compounds of the invention comprise a means for binding to a GRPR-expressing tumor in a subject, wherein the peptidic compound comprises a chelator optionally complexed with a radionuclide and wherein the peptidic compound exhibits a tumor comparative time-activity integration (AUCₜᵤₘₒᵣ) of at least 1000% ID/g·h. In some embodiments, the peptidic compound exhibits an AUCₜᵤₘₒᵣ of at least 1100% ID/g·h. In some embodiments, the peptidic compound exhibits an AUCₜᵤₘₒᵣ of at least 1200% ID/g·h. In some embodiments, the peptidic compound exhibits an AUCₜᵤₘₒᵣ of at least 1300% ID/g·h. In some embodiments, the peptidic compound exhibits an AUCₜᵤₘₒᵣ of at least 1400% ID/g·h. In some embodiments, the peptidic compound exhibits an AUCₜᵤₘₒᵣ of at least 1500% ID/g·h. In some embodiments, the peptidic compound exhibits an AUCₜᵤₘₒᵣ of at least 1600% ID/g·h. In some embodiments, the peptidic compound exhibits an AUCₜᵤₘₒᵣ of at least 1700% ID/g·h. In some embodiments, the peptidic compound exhibits an AUCₜᵤₘₒᵣ of at least 1800% ID/g·h. In some embodiments, the peptidic compound exhibits an AUCₜᵤₘₒᵣ of at least 1900% ID/g·h. In some embodiments, the peptidic compound exhibits an AUCₜᵤₘₒᵣ of at least 2000% ID/g·h. In some embodiments, the peptidic compound exhibits an AUCₜᵤₘₒᵣ of at least 2100% ID/g·h.

In yet another aspect, the peptidic compounds of the invention comprise a means for binding to a GRPR-expressing tumor in a subject, wherein the peptidic compound comprises a chelator optionally complexed with a radionuclide and wherein the peptidic compound exhibits a tumor/kidney uptake ratio of at least 10. In some embodiments, In some embodiments, the peptidic compound exhibits a tumor/kidney uptake ratio of at least 11. In some embodiments, the peptidic compound exhibits a tumor/kidney uptake ratio of at least 12. In some embodiments, the peptidic compound exhibits a tumor/kidney uptake ratio of at least 13. In some embodiments, the peptidic compound exhibits a tumor/kidney uptake ratio of at least 14. In some embodiments, the peptidic compound exhibits a tumor/kidney uptake ratio of at least 15. In some embodiments, the peptidic compound exhibits a tumor/kidney uptake ratio of at least 16. In some embodiments, the peptidic compound exhibits a tumor/kidney uptake ratio of at least 17. In some embodiments, the peptidic compound exhibits a tumor/kidney uptake ratio of at least 18. In some embodiments, the peptidic compound exhibits a tumor/kidney uptake ratio of at least 19. In some embodiments, the peptidic compound exhibits a tumor/kidney uptake ratio of at least 20.

In an additional aspect, the peptidic compounds of the invention comprise a means for binding to a GRPR-expressing tumor in a subject, wherein the peptidic compound comprises a chelator optionally complexed with a radionuclide and wherein the peptidic compound exhibits a tumor/pancreas uptake ratio of at least 1 (e.g., at least 1.5, at least 2, at least 4, at least 5, at least 6, at least 8, at least 10) and a tumor comparative time-activity integration (AUCₜᵤₘₒᵣ) of at least 1000% ID/g·h (e.g., at least 1100, at least 1200, at least 1300, at least 1400, at least 1500, at least 1600, at least 1700, at least 1800, at least 1900, or at least 2000% ID/g·h).

In another aspect, a peptidic compound of the invention comprises a means for binding to a GRPR-expressing tumor in a subject, wherein the peptidic compound is a compound of Formula (A), or a salt or solvate of Formula (A):

(A)
R^{rad}ₙ₆-R^{L}-[linker]-R^{L}-Xaa¹-Xaa²-Xaa³-Xaa⁴-Xaa⁵-
Xaa⁶-Xaa⁷-Xaa⁸-[ψ-Xaa⁹-(Xaa¹⁰)ₘ₂]ₘ₁-R

wherein:
Xaa¹ is an N-terminal amino acid residue selected from D-Phe, 4-chlorophenylalanine (Cpa), D-Cpa, 3-(1-naphthyl)alanine (Nal), D-Nal, 3-(2-naphthyl)alanine (2-Nal), D-2-Nal, 2,3,4,9-tetrahydro-1H-pyrido[3,4-b]indol-3-carboxylic acid (Tpi), or D-Tpi;
Xaa² is Gly, Arg, Asp, Asn, Gln, Glu, homoserine (Hse), citrulline (Cit), 2-amino-4-ureidobutanoic acid (Aub), 2-amino-4-guanidinobutyric acid (Agb), or His;
Xaa³ is Trp, β-(3-benzothienyl)alanine (Bta), Trp(Me), Trp(7-Me), Trp(6-Me), Trp(5-Me), Trp(4-Me), Trp(2-Me), Trp(7-F), Trp(6-F), Trp(5-F), Trp(4-F), Trp(7-Cl), Trp(6-Cl), Trp(5-Cl), Trp(4-Cl), Trp(7-Br), Trp(6-Br), Trp(5-Br), Trp(4-Br), Trp(7-1), Trp(6-1), Trp(5-1), Trp(4-1), Trp(5-OH), αMe-Trp, Tpi, 7-azatryptophan (7-Aza), Nal, 2-pyridylalanine (2-Pal), 3-pyridylalanine (3-Pal), 4-pyridylalanine (4-Pal), 2-quinoylalanine (2-Quin-Ala), or 3-quinoylalanine (3-Quin-Ala);
Xaa⁴ is Ala, D-Ala, or Ser;
Xaa⁵ is Val, 2,3-dehydro-Val, cyclopentylglycine (Cpg), L-cyclopropylglycine, L-cyclobutylglycine, or tert-leucine (Tle);
Xaa⁶ is Gly or D-Ala;
Xaa⁷ is His, Trp, Trp(Me), Trp(7-Me), Trp(6-Me), Trp(5-Me), Trp(4-Me), Trp(2-Me), Trp(7-F), Trp(6-F), Trp(5-F), Trp(4-F), Trp(7-Cl), Trp(6-Cl), Trp(5-Cl), Trp(4-Cl), Trp(7-Br), Trp(6-Br), Trp(5-Br), Trp(4-Br), Trp(7-1), Trp(6-1), Trp(5-1), Trp(4-1), Trp(5-OH), αMe-Trp, Nal, 2-pyridylalanine (2-Pal), 3-pyridylalanine (3-Pal), 4-pyridylalanine (4-Pal), 2-quinoylalanine (2-Quin-Ala), or 3-quinoylalanine (3-Quin-Ala);
Xaa⁸ is statine (Sta), Leu, D-Pro, Phe, cis-4-NHR^{a}-D-Pro or cis-4-OR^{a}-D-Pro, wherein R^{a} is alkyl, cycloalkyl, or aryl;
R is —NH₂ or —NHOH;
m1 is 0 or 1;
m2 is 0 or 1;
   when m1 is 1 and m2 is 0, Xaa⁹-R is a C-terminally amidated (R is —NH₂) or a C-terminally hydroxylamidated (R is —NHOH) amino acid residue selected from Pro, Phe, oxazolidine-4-carboxylic acid (4-oxa-L-Pro), 4,4-difluoroproline (difluoroPro), cis-4-NHR^{a}-D-Pro, cis-4-OR^{a}-D-Pro, Leu, 5,5-dimethyl-1,3-thiazolidine-4-carboxylic acid (Me₂Thz), or thiazoline-4-carboxylic acid (Thz), wherein R^{a} is alkyl, cycloalkyl, or aryl;
   when m1 is 1 and m2 is 1, Xaa⁹ is Pro, Phe, oxazolidine-4-carboxylic acid (4-oxa-L-Pro), 4,4-difluoroproline (difluoroPro), Leu, 5,5-dimethyl-1,3-thiazolidine-4-carboxylic acid (Me₂Thz), or thiazoline-4-carboxylic acid (Thz); and Xaa¹⁰-R is a C-terminally amidated (R is —NH₂) or a C-terminally hydroxylamidated (R is —NHOH) amino acid residue selected from Val, Leu, Tle, Ile, Nle, Ala, Gly, or Met;
ψ represents a peptide bond or reduced peptide bond joining Xaa⁸ to Xaa⁹;
each R^{L} is independently absent, —(C₁₋₆ alkyl)-, —(C₁₋₆ alkyl)-C(O)—, —C(O)—, —NH—C(O)—, or —NH—C(S)—;
the linker is a linear or branched chain of n1 units of -Xaa¹¹-, and/or -L¹R¹— and/or -(L¹)₂R¹—, wherein:
   n1 is 1-20;
   each Xaa¹¹ is independently an amino acid;
   each R¹ is, independently, a linear, branched, and/or cyclic C₁₋₂₀ alkylenyl, alkenylenyl and/or alkynylenyl, wherein any carbon bonded to two other carbons is optionally independently replaced by N, S, or O, and carbons are optionally independently substituted with oxo, hydroxyl, sulfhydryl, —SeH, halogen, guanidino, amine, amide, urea, carboxylic acid, sulfonic acid, sulfinic acid, or phosphoric acid;
   L¹ bonds to carbon, wherein each L¹ is independently —S—, —N(R²)C(O)—, —C(O)N(R²)—, —NH—C(O)—NH—, —NH—C(S)—NH—,

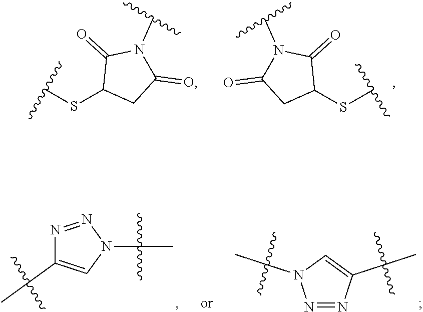

and
R² is H, methyl or ethyl;
optionally, the linker comprises an albumin binder (R^{alb}), wherein the albumin binder is:
   —(CH₂)ₙ₃—CH₃ wherein n3 is 8-20;
   —(CH₂)ₙ₄—C(O)OH wherein n4 is 8-20;

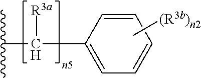

wherein n2 is 1-5, n5 is 1-4, R^{3a} is H or methyl, and R^{3b} is I, Br, F, Cl, H, OH, OCH₃, NH₂, NO₂ or C₁-C₆ alkyl;

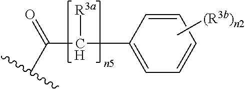

wherein n2 is 1-5, n5 is 1-4, R^{3a} is H or methyl, and R^{3b} is I, Br, F, Cl, H, OH, OCH₃, NH₂, NO₂ or C₁-C₆ alkyl; or

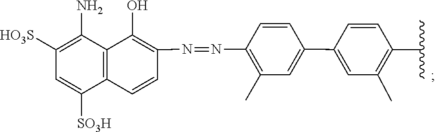 [SMILES: *c1ccc(-c2ccc(N=Nc3ccc4c(S(=O)(=O)O)cc(S(=O)(=O)O)c(N)c4c3O)c(C)c2)cc1C]

n6 is 1-5;
each R^{rad} is a radiolabeling group independently selected from a radiometal chelator; an aryl or heteroaryl substituted with at least one radiohalogen; a prosthetic group containing a trifluoroborate; an aryl or heteroaryl substituted with at least one fluorine precursor leaving group; a prosthetic group containing a silicon-fluorine-acceptor moiety; or a prosthetic group containing a fluorophosphate, fluorosulfate, sulfonyl fluoride, or a combination thereof; and
wherein Xaa¹, Xaa², Xaa³, Xaa⁴, Xaa⁵, Xaa⁶, Xaa⁷, Xaa⁸, Xaa⁹ or Xaa¹⁰ is each optionally N-methylated; wherein the above peptidic compound of Formula (A) exhibits a tumor/pancreas uptake ratio of at least 1 (e.g., at least 1.5 or higher, about 2 or higher, about 4 or higher, about 5 or higher, about 6 or higher, about 7 or higher, about 10 or higher, or about 16).

In an aspect, a peptidic compound of the invention comprises a means for binding to a GRPR-expressing tumor in a subject, wherein the peptidic compound is a compound of Formula (A), or a salt or solvate of Formula (A):

(A)
R^{rad}ₙ₆-R^{L}-[linker]-R^{L}-Xaa¹-Xaa²-Xaa³-Xaa⁴-Xaa⁵-
Xaa⁶-Xaa⁷-Xaa⁸-[ψ-Xaa⁹-(Xaa¹⁰)ₘ₂]ₘ₁-R

wherein:
Xaa¹ is an N-terminal amino acid residue selected from D-Phe, 4-chlorophenylalanine (Cpa), D-Cpa, 3-(1-naphthyl)alanine (Nal), D-Nal, 3-(2-naphthyl)alanine (2-Nal), D-2-Nal, 2,3,4,9-tetrahydro-1H-pyrido[3,4-b]indol-3-carboxylic acid (Tpi), or D-Tpi;
Xaa² is Gly, Arg, Asp, Asn, Gln, Glu, homoserine (Hse), citrulline (Cit), 2-amino-4-ureidobutanoic acid (Aub), 2-amino-4-guanidinobutyric acid (Agb), or His;
Xaa³ is Trp, β-(3-benzothienyl)alanine (Bta), Trp(Me), Trp(7-Me), Trp(6-Me), Trp(5-Me), Trp(4-Me), Trp(2-Me), Trp(7-F), Trp(6-F), Trp(5-F), Trp(4-F), Trp(7-Cl), Trp(6-Cl), Trp(5-Cl), Trp(4-Cl), Trp(7-Br), Trp(6-Br), Trp(5-Br), Trp(4-Br), Trp(7-1), Trp(6-1), Trp(5-1), Trp(4-1), Trp(5-OH), αMe-Trp, Tpi, 7-azatryptophan (7-Aza), Nal, 2-pyridylalanine (2-Pal), 3-pyridylalanine (3-Pal), 4-pyridylalanine (4-Pal), 2-quinoylalanine (2-Quin-Ala), or 3-quinoylalanine (3-Quin-Ala);
Xaa⁴ is Ala, D-Ala, or Ser;
Xaa⁵ is Val, 2,3-dehydro-Val, cyclopentylglycine (Cpg), L-cyclopropylglycine, L-cyclobutylglycine, or tert-leucine (Tle);
Xaa⁶ is Gly or D-Ala;
Xaa⁷ is His, Trp, Trp(Me), Trp(7-Me), Trp(6-Me), Trp(5-Me), Trp(4-Me), Trp(2-Me), Trp(7-F), Trp(6-F), Trp(5-F), Trp(4-F), Trp(7-Cl), Trp(6-Cl), Trp(5-Cl), Trp(4-Cl), Trp(7-Br), Trp(6-Br), Trp(5-Br), Trp(4-Br), Trp(7-1), Trp(6-1), Trp(5-1), Trp(4-1), Trp(5-OH), αMe-Trp, Nal, 2-pyridylalanine (2-Pal), 3-pyridylalanine (3-Pal), 4-pyridylalanine (4-Pal), 2-quinoylalanine (2-Quin-Ala), or 3-quinoylalanine (3-Quin-Ala);
Xaa⁸ is statine (Sta), Leu, D-Pro, Phe, cis-4-NHR^{a}-D-Pro or cis-4-OR^{a}-D-Pro, wherein R^{a} is alkyl, cycloalkyl, or aryl;
R is —NH₂ or —NHOH;
m1 is 0 or 1;
m2 is 0 or 1;
   when m1 is 1 and m2 is 0, Xaa⁹-R is a C-terminally amidated (R is —NH₂) or a C-terminally hydroxylamidated (R is —NHOH) amino acid residue selected from Pro, Phe, oxazolidine-4-carboxylic acid (4-oxa-L-Pro), 4,4-difluoroproline (difluoroPro), cis-4-NHR^{a}-D-Pro, cis-4-OR^{a}-D-Pro, Leu, 5,5-dimethyl-1,3-thiazolidine-4-carboxylic acid (Me₂Thz), or thiazoline-4-carboxylic acid (Thz), wherein R^{a} is alkyl, cycloalkyl, or aryl;
   when m1 is 1 and m2 is 1, Xaa⁹ is Pro, Phe, oxazolidine-4-carboxylic acid (4-oxa-L-Pro), 4,4-difluoroproline (difluoroPro), Leu, 5,5-dimethyl-1,3-thiazolidine-4-carboxylic acid (Me₂Thz), or thiazoline-4-carboxylic acid (Thz); and Xaa¹⁰-R is a C-terminally amidated (R is —NH₂) or a C-terminally hydroxylamidated (R is —NHOH) amino acid residue selected from Val, Leu, Tle, Ile, Nle, Ala, Gly, or Met;
ψ represents a peptide bond or reduced peptide bond joining Xaa⁸ to Xaa⁹;
each R^{L} is independently absent, —(C₁₋₆ alkyl)-, —(C₁₋₆ alkyl)-C(O)—, —C(O)—, —NH—C(O)—, or —NH—C(S)—;
the linker is a linear or branched chain of n1 units of -Xaa¹¹-, and/or -L¹R¹— and/or -(L¹)₂R¹—, wherein:
   n1 is 1-20;
   each Xaa¹¹ is independently an amino acid;
   each R¹ is, independently, a linear, branched, and/or cyclic C₁₋₂₀ alkylenyl, alkenylenyl and/or alkynylenyl, wherein any carbon bonded to two other carbons is optionally independently replaced by N, S, or O, and carbons are optionally independently substituted with oxo, hydroxyl, sulfhydryl, —SeH, halogen, guanidino, amine, amide, urea, carboxylic acid, sulfonic acid, sulfinic acid, or phosphoric acid;
   L¹ bonds to carbon, wherein each L¹ is independently —S—, —N(R²)C(O)—, —C(O)N(R²)—, —NH—C(O)—NH—, —NH—C(S)—NH—,

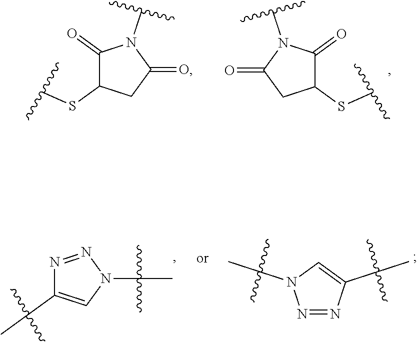

and
R² is H, methyl or ethyl;
optionally, the linker comprises an albumin binder (R^{alb}), wherein the albumin binder is:
   —(CH₂)ₙ₃—CH₃ wherein n3 is 8-20;
   —(CH₂)ₙ₄—C(O)OH wherein n4 is 8-20;

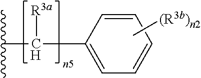

wherein n2 is 1-5, n5 is 1-4, R^{3a} is H or methyl, and R^{3b} is I, Br, F, Cl, H, OH, OCH₃, NH₂, NO₂ or C₁-C₆ alkyl;

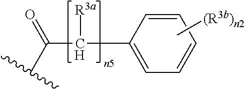

wherein n2 is 1-5, n5 is 1-4, R^{3a} is H or methyl, and R^{3b} is I, Br, F, Cl, H, OH, OCH₃, NH₂, NO₂ or C₁-C₆ alkyl; or

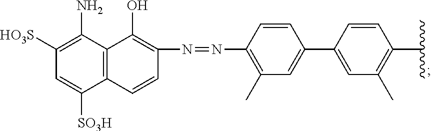 [SMILES: *c1ccc(-c2ccc(N=Nc3ccc4c(S(=O)(=O)O)cc(S(=O)(=O)O)c(N)c4c3O)c(C)c2)cc1C]

n6 is 1-5;
each R^{rad} is a radiolabeling group independently selected from a radiometal chelator; an aryl or heteroaryl substituted with at least one radiohalogen; a prosthetic group containing a trifluoroborate; an aryl or heteroaryl substituted with at least one fluorine precursor leaving group; a prosthetic group containing a silicon-fluorine-acceptor moiety; or a prosthetic group containing a fluorophosphate, fluorosulfate, sulfonyl fluoride, or a combination thereof; and
wherein Xaa¹, Xaa², Xaa³, Xaa⁴, Xaa⁵, Xaa⁶, Xaa⁷, Xaa⁸, Xaa⁹ or Xaa¹⁰ is each optionally N-methylated; wherein the above peptidic compound of Formula (A) exhibits a tumor comparative time-activity integration (AUCₜᵤₘₒᵣ) of at least 1000% ID/g·h (e.g., at least 1100% ID/g·h, at least 1200% ID/g·h, at least 1300% ID/g·h, at least 1400% ID/g·h, at least 1500% ID/g·h, at least 1600% ID/g·h, at least 1700% ID/g·h, at least 1800% ID/g·h, at least 1900% ID/g·h, at least 2000% ID/g·h, or at least 2100% ID/g·h).

In yet another aspect, a peptidic compound of the invention comprises a means for binding to a GRPR-expressing tumor in a subject, wherein the peptidic compound is a compound of Formula (A), or a salt or solvate of Formula (A):

(A)
R^{rad}ₙ₆-R^{L}-[linker]-R^{L}-Xaa¹-Xaa²-Xaa³-Xaa⁴-Xaa⁵-
Xaa⁶-Xaa⁷-Xaa⁸-[ψ-Xaa⁹-(Xaa¹⁰)ₘ₂]ₘ₁-R

wherein:
Xaa¹ is an N-terminal amino acid residue selected from D-Phe, 4-chlorophenylalanine (Cpa), D-Cpa, 3-(1-naphthyl)alanine (Nal), D-Nal, 3-(2-naphthyl)alanine (2-Nal), D-2-Nal, 2,3,4,9-tetrahydro-1H-pyrido[3,4-b]indol-3-carboxylic acid (Tpi), or D-Tpi;
Xaa² is Gly, Arg, Asp, Asn, Gln, Glu, homoserine (Hse), citrulline (Cit), 2-amino-4-ureidobutanoic acid (Aub), 2-amino-4-guanidinobutyric acid (Agb), or His;
Xaa³ is Trp, β-(3-benzothienyl)alanine (Bta), Trp(Me), Trp(7-Me), Trp(6-Me), Trp(5-Me), Trp(4-Me), Trp(2-Me), Trp(7-F), Trp(6-F), Trp(5-F), Trp(4-F), Trp(7-Cl), Trp(6-Cl), Trp(5-Cl), Trp(4-Cl), Trp(7-Br), Trp(6-Br), Trp(5-Br), Trp(4-Br), Trp(7-1), Trp(6-1), Trp(5-1), Trp(4-1), Trp(5-OH), αMe-Trp, Tpi, 7-azatryptophan (7-Aza), Nal, 2-pyridylalanine (2-Pal), 3-pyridylalanine (3-Pal), 4-pyridylalanine (4-Pal), 2-quinoylalanine (2-Quin-Ala), or 3-quinoylalanine (3-Quin-Ala);
Xaa⁴ is Ala, D-Ala, or Ser;
Xaa⁵ is Val, 2,3-dehydro-Val, cyclopentylglycine (Cpg), L-cyclopropylglycine, L-cyclobutylglycine, or tert-leucine (Tle);
Xaa⁶ is Gly or D-Ala;
Xaa⁷ is His, Trp, Trp(Me), Trp(7-Me), Trp(6-Me), Trp(5-Me), Trp(4-Me), Trp(2-Me), Trp(7-F), Trp(6-F), Trp(5-F), Trp(4-F), Trp(7-Cl), Trp(6-Cl), Trp(5-Cl), Trp(4-Cl), Trp(7-Br), Trp(6-Br), Trp(5-Br), Trp(4-Br), Trp(7-1), Trp(6-1), Trp(5-1), Trp(4-1), Trp(5-OH), αMe-Trp, Nal, 2-pyridylalanine (2-Pal), 3-pyridylalanine (3-Pal), 4-pyridylalanine (4-Pal), 2-quinoylalanine (2-Quin-Ala), or 3-quinoylalanine (3-Quin-Ala);
Xaa⁸ is statine (Sta), Leu, D-Pro, Phe, cis-4-NHR^{a}-D-Pro or cis-4-OR^{a}-D-Pro, wherein R^{a} is alkyl, cycloalkyl, or aryl;
R is —NH₂ or —NHOH;
m1 is 0 or 1;
m2 is 0 or 1;
   when m1 is 1 and m2 is 0, Xaa⁹-R is a C-terminally amidated (R is —NH₂) or a C-terminally hydroxylamidated (R is —NHOH) amino acid residue selected from Pro, Phe, oxazolidine-4-carboxylic acid (4-oxa-L-Pro), 4,4-difluoroproline (difluoroPro), cis-4-NHR^{a}-D-Pro, cis-4-OR^{a}-D-Pro, Leu, 5,5-dimethyl-1,3-thiazolidine-4-carboxylic acid (Me₂Thz), or thiazoline-4-carboxylic acid (Thz), wherein R^{a} is alkyl, cycloalkyl, or aryl;
   when m1 is 1 and m2 is 1, Xaa⁹ is Pro, Phe, oxazolidine-4-carboxylic acid (4-oxa-L-Pro), 4,4-difluoroproline (difluoroPro), Leu, 5,5-dimethyl-1,3-thiazolidine-4-carboxylic acid (Me₂Thz), or thiazoline-4-carboxylic acid (Thz); and Xaa¹⁰-R is a C-terminally amidated (R is —NH₂) or a C-terminally hydroxylamidated (R is —NHOH) amino acid residue selected from Val, Leu, Tle, Ile, Nle, Ala, Gly, or Met;
ψ represents a peptide bond or reduced peptide bond joining Xaa⁸ to Xaa⁹;
each R^{L} is independently absent, —(C₁₋₆ alkyl)-, —(C₁₋₆ alkyl)-C(O)—, —C(O)—, —NH—C(O)—, or —NH—C(S)—;
the linker is a linear or branched chain of n1 units of -Xaa¹¹-, and/or -L¹R¹— and/or -(L¹)₂R¹—, wherein:
   n1 is 1-20;
   each Xaa¹¹ is independently an amino acid;
   each R¹ is, independently, a linear, branched, and/or cyclic C₁₋₂₀ alkylenyl, alkenylenyl and/or alkynylenyl, wherein any carbon bonded to two other carbons is optionally independently replaced by N, S, or O, and carbons are optionally independently substituted with oxo, hydroxyl, sulfhydryl, —SeH, halogen, guanidino, amine, amide, urea, carboxylic acid, sulfonic acid, sulfinic acid, or phosphoric acid;
   L¹ bonds to carbon, wherein each L¹ is independently —S—, —N(R²)C(O)—, —C(O)N(R²)—, —NH—C(O)—NH—, —NH—C(S)—NH—,

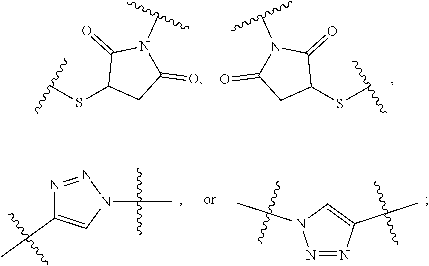

and
R² is H, methyl or ethyl;
optionally, the linker comprises an albumin binder (R^{alb}), wherein the albumin binder is:
   —(CH₂)ₙ₃—CH₃ wherein n3 is 8-20;
—(CH₂)ₙ₄—C(O)OH wherein n4 is 8-20;

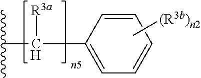

wherein n2 is 1-5, n5 is 1-4, R^{3a} is H or methyl, and R^{3b} is I, Br, F, Cl, H, OH, OCH₃, NH₂, NO₂ or C₁-C₆ alkyl;

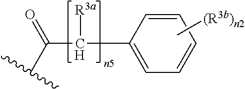

wherein n2 is 1-5, n5 is 1-4, R^{3a} is H or methyl, and R^{3b} is I, Br, F, Cl, H, OH, OCH₃, NH₂, NO₂ or C₁-C₆ alkyl; or

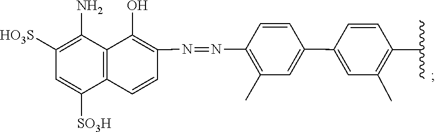 [SMILES: *c1ccc(-c2ccc(N=Nc3ccc4c(S(=O)(=O)O)cc(S(=O)(=O)O)c(N)c4c3O)c(C)c2)cc1C]

n6 is 1-5;
each R^{rad} is a radiolabeling group independently selected from a radiometal chelator; an aryl or heteroaryl substituted with at least one radiohalogen; a prosthetic group containing a trifluoroborate; an aryl or heteroaryl substituted with at least one fluorine precursor leaving group; a prosthetic group containing a silicon-fluorine-acceptor moiety; or a prosthetic group containing a fluorophosphate, fluorosulfate, sulfonyl fluoride, or a combination thereof; and
wherein Xaa¹, Xaa², Xaa³, Xaa⁴, Xaa⁵, Xaa⁶, Xaa⁷, Xaa^{B}, Xaa⁹ or Xaa¹⁰ is each optionally N-methylated; wherein the above peptidic compound exhibits a tumor/pancreas uptake ratio of at least 1 (e.g., at least 1.5, at least 2, at least 4, at least 5, at least 6, at least 8, at least 10) and a tumor comparative time-activity integration (AUCₜᵤₘₒᵣ) of at least 1000 (e.g., at least 1100, at least 1200, at least 1300, at least 1400, at least 1500, at least 1600, at least 1700, at least 1800, at least 1900, or at least 2000) % ID/g·h.

In another aspect, a peptidic compound of the invention comprises a means for binding to a GRPR-expressing tumor in a subject, wherein the peptidic compound is a compound of Formula (I), or a salt or solvate of Formula (I),

(I)
R^{rad}ₙ₆-[linker]-Xaa¹-Xaa²-Xaa³-Xaa⁴-Xaa⁵-Xaa⁶-
Xaa⁷-Xaa⁸-[ψ-Xaa⁹-(Xaa¹⁰)ₘ₂]ₘ₁-R

wherein:
Xaa¹ is an N-terminal amino acid residue selected from D-Phe, 4-chlorophenylalanine (Cpa), D-Cpa, 3-(1-naphthyl)alanine (Nal), D-Nal, 3-(2-naphthyl)alanine (2-Nal), D-2-Nal, 2,3,4,9-tetrahydro-1H-pyrido[3,4-b]indol-3-carboxylic acid (Tpi), or D-Tpi;
Xaa² is Gly, Arg, Asp, Asn, Gln, Glu, homoserine (Hse), citrulline (Cit), 2-amino-4-ureidobutanoic acid (Aub), 22-amino-4-guanidinobutyric acid (Agb), or His;
Xaa³ is Trp, β-(3-benzothienyl)alanine (Bta), Trp(Me), Trp(7-Me), Trp(6-Me), Trp(5-Me), Trp(4-Me), Trp(2-Me), Trp(7-F), Trp(6-F), Trp(5-F), Trp(4-F), Trp(7-Cl), Trp(6-Cl), Trp(5-Cl), Trp(4-Cl), Trp(7-Br), Trp(6-Br), Trp(5-Br), Trp(4-Br), Trp(7-1), Trp(6-1), Trp(5-1), Trp(4-1), Trp(5-OH), αMe-Trp, Tpi, 7-azatryptophan (7-Aza), Nal, 2-pyridylalanine (2-Pal), 3-pyridylalanine (3-Pal), 4-pyridylalanine (4-Pal), 2-quinoylalanine, or 3-quinoylalanine;
Xaa⁴ is Ala, D-Ala, or Ser;
Xaa⁵ is Val, 2,3-dehydro-Val, cyclopentylglycine (Cpg), L-cyclopropylglycine, L-cyclobutylglycine, or tert-leucine (Tle);
Xaa⁶ is Gly or D-Ala;
Xaa⁷ is His, Trp, Trp(Me), Trp(7-Me), Trp(6-Me), Trp(5-Me), Trp(4-Me), Trp(2-Me), Trp(7-F), Trp(6-F), Trp(5-F), Trp(4-F), Trp(7-Cl), Trp(6-Cl), Trp(5-Cl), Trp(4-Cl), Trp(7-Br), Trp(6-Br), Trp(5-Br), Trp(4-Br), Trp(7-1), Trp(6-1), Trp(5-1), Trp(4-1),Trp(5-OH), αMe-Trp, Nal, 2-pyridylalanine (2-Pal), 3-pyridylalanine (3-Pal), 4-pyridylalanine (4-Pal), 2-quinoylalanine, or 3-quinoylalanine;
Xaa⁸ is statine (Sta), Leu, D-Pro, Phe, cis-4-NHR^{a}-D-Pro or cis-4-OR^{a}-D-Pro, wherein R^{a} is alkyl, cycloalkyl, or aryl;
R is —NH₂ or —NHOH;
m1 is 0 or 1;
m2 is 0 or 1;
   when m1 is 1 and m2 is 0, Xaa⁹-R is a C-terminally amidated (R is —NH₂) or a C-terminally hydroxylamidated (R is —NHOH) amino acid residue selected from Pro, Phe, oxazolidine-4-carboxylic acid (4-oxa-L-Pro), 4,4-difluoroproline (difluoroPro), cis-4-NHR^{a}-D-Pro, cis-4-OR^{a}-D-Pro, Leu, 5,5-dimethyl-1,3-thiazolidine-4-carboxylic acid (Me₂Thz), or thiazoline-4-carboxylic acid (Thz), wherein R^{a} is alkyl, cycloalkyl, or aryl;
   when m1 is 1 and m2 is 1, Xaa⁹ is Pro, Phe, oxazolidine-4-carboxylic acid (4-oxa-L-Pro), 4,4-difluoroproline (difluoroPro), Leu, Me₂Thz (5,5-dimethyl-1,3-thiazolidine-4-carboxylic acid), or thiazoline-4-carboxylic acid (Thz); and
      Xaa¹⁰-R is a C-terminally amidated (R is —NH₂) or a C-terminally hydroxylamidated (R is —NHOH) amino acid residue selected from Val, Leu, Tle, Ile, Nle, Ala, Gly, or Met;
ψ represents a peptide bond or reduced peptide bond joining Xaa⁸ to Xaa⁹;
the linker is a linear or branched chain of n1 units of -Xaa¹¹-;
   n1 is 1-20;
   each Xaa¹¹ is independently an amino acid;
   optionally, the linker comprises an albumin binder (R^{alb}), wherein the albumin binder is:
   —(CH₂)ₙ₃—CH₃ wherein n3 is 8-20;
   —(CH₂)ₙ₄—C(O)OH wherein n4 is 8-20;

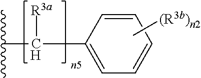

wherein n2 is 1-5, n5 is 1-4, R^{3a} is H or methyl, and R^{3b} is I, Br, F, Cl, H, OH, OCH₃, NH₂, NO₂ or C₁-C₆ alkyl;

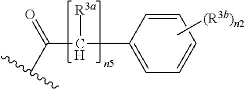

wherein n2 is 1-5, n5 is 1-4, R^{3a} is H or methyl, and R^{3b} is I, Br, F, Cl, H, OH, OCH₃, NH₂, NO₂ or C₁-C₆ alkyl; or

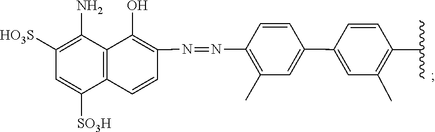 [SMILES: *c1ccc(-c2ccc(N=Nc3ccc4c(S(=O)(=O)O)cc(S(=O)(=O)O)c(N)c4c3O)c(C)c2)cc1C]

n6 is 1-5;
each R^{rad} is a radiolabeling group independently selected from a radiometal chelator; an aryl or heteroaryl substituted with at least one radiohalogen; a prosthetic group containing a trifluoroborate; an aryl or heteroaryl substituted with at least one fluorine precursor leaving group; a prosthetic group containing a silicon-fluorine-acceptor moiety; or a prosthetic group containing a fluorophosphate, fluorosulfate, sulfonyl fluoride, or a combination thereof; and
wherein Xaa¹, Xaa², Xaa³, Xaa⁴, Xaa⁵, Xaa⁶, Xaa⁷, Xaa⁸, Xaa⁹ or Xaa¹⁰ is each optionally N-methylated; wherein the above peptidic compound exhibits a tumor/pancreas uptake ratio of at least 1 (e.g., at least 1.5 or higher, about 2 or higher, about 4 or higher, about 5 or higher, about 6 or higher, about 7 or higher, about 10 or higher, or about 16).

In another aspect, a peptidic compound of the invention comprises a means for binding to a GRPR-expressing tumor in a subject, wherein the peptidic compound is a compound of Formula (I), or a salt or solvate of Formula (I),

(I)
R^{rad}ₙ₆-[linker]-Xaa¹-Xaa²-Xaa³-Xaa⁴-Xaa⁵-Xaa⁶-
Xaa⁷-Xaa⁸-[ψ-Xaa⁹-(Xaa¹⁰)ₘ₂]ₘ₁-R

wherein:
Xaa¹ is an N-terminal amino acid residue selected from D-Phe, 4-chlorophenylalanine (Cpa), D-Cpa, 3-(1-naphthyl)alanine (Nal), D-Nal, 3-(2-naphthyl)alanine (2-Nal), D-2-Nal, 2,3,4,9-tetrahydro-1H-pyrido[3,4-b]indol-3-carboxylic acid (Tpi), or D-Tpi;
Xaa² is Gly, Arg, Asp, Asn, Gln, Glu, homoserine (Hse), citrulline (Cit), 2-amino-4-ureidobutanoic acid (Aub), 22-amino-4-guanidinobutyric acid (Agb), or His;
Xaa³ is Trp, β-(3-benzothienyl)alanine (Bta), Trp(Me), Trp(7-Me), Trp(6-Me), Trp(5-Me), Trp(4-Me), Trp(2-Me), Trp(7-F), Trp(6-F), Trp(5-F), Trp(4-F), Trp(7-Cl), Trp(6-Cl), Trp(5-Cl), Trp(4-Cl), Trp(7-Br), Trp(6-Br), Trp(5-Br), Trp(4-Br), Trp(7-1), Trp(6-1), Trp(5-1), Trp(4-1), Trp(5-OH), αMe-Trp, Tpi, 7-azatryptophan (7-Aza), Nal, 2-pyridylalanine (2-Pal), 3-pyridylalanine (3-Pal), 4-pyridylalanine (4-Pal), 2-quinoylalanine, or 3-quinoylalanine;
Xaa⁴ is Ala, D-Ala, or Ser;
Xaa⁵ is Val, 2,3-dehydro-Val, cyclopentylglycine (Cpg), L-cyclopropylglycine, L-cyclobutylglycine, or tert-leucine (Tle);
Xaa⁶ is Gly or D-Ala;
Xaa⁷ is His, Trp, Trp(Me), Trp(7-Me), Trp(6-Me), Trp(5-Me), Trp(4-Me), Trp(2-Me), Trp(7-F), Trp(6-F), Trp(5-F), Trp(4-F), Trp(7-Cl), Trp(6-Cl), Trp(5-Cl), Trp(4-Cl), Trp(7-Br), Trp(6-Br), Trp(5-Br), Trp(4-Br), Trp(7-1), Trp(6-1), Trp(5-1), Trp(4-1),Trp(5-OH), αMe-Trp, Nal, 2-pyridylalanine (2-Pal), 3-pyridylalanine (3-Pal), 4-pyridylalanine (4-Pal), 2-quinoylalanine, or 3-quinoylalanine;
Xaa⁸ is statine (Sta), Leu, D-Pro, Phe, cis-4-NHR^{a}-D-Pro or cis-4-OR^{a}-D-Pro, wherein R^{a} is alkyl, cycloalkyl, or aryl;
R is —NH₂ or —NHOH;
m1 is 0 or 1;
m2 is 0 or 1;
   when m1 is 1 and m2 is 0, Xaa⁹-R is a C-terminally amidated (R is —NH₂) or a C-terminally hydroxylamidated (R is —NHOH) amino acid residue selected from Pro, Phe, oxazolidine-4-carboxylic acid (4-oxa-L-Pro), 4,4-difluoroproline (difluoroPro), cis-4-NHR^{a}-D-Pro, cis-4-OR^{a}-D-Pro, Leu, 5,5-dimethyl-1,3-thiazolidine-4-carboxylic acid (Me₂Thz), or thiazoline-4-carboxylic acid (Thz), wherein R^{a} is alkyl, cycloalkyl, or aryl;
   when m1 is 1 and m2 is 1, Xaa⁹ is Pro, Phe, oxazolidine-4-carboxylic acid (4-oxa-L-Pro), 4,4-difluoroproline (difluoroPro), Leu, Me₂Thz (5,5-dimethyl-1,3-thiazolidine-4-carboxylic acid), or thiazoline-4-carboxylic acid (Thz); and
      Xaa¹⁰-R is a C-terminally amidated (R is —NH₂) or a C-terminally hydroxylamidated (R is —NHOH) amino acid residue selected from Val, Leu, Tle, Ile, Nle, Ala, Gly, or Met;
ψ represents a peptide bond or reduced peptide bond joining Xaa⁸ to Xaa⁹;
the linker is a linear or branched chain of n1 units of -Xaa¹¹-;
   n1 is 1-20;
   each Xaa¹¹ is independently an amino acid;
   optionally, the linker comprises an albumin binder (R^{alb}), wherein the albumin binder is:
   —(CH₂)ₙ₃—CH₃ wherein n3 is 8-20;
   —(CH₂)ₙ₄—C(O)OH wherein n4 is 8-20;

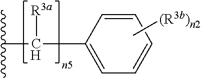

wherein n2 is 1-5, n5 is 1-4, R^{3a} is H or methyl, and R^{3b} is I, Br, F, Cl, H, OH, OCH₃, NH₂, NO₂ or C₁-C₆ alkyl;

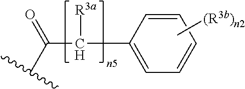

wherein n2 is 1-5, n5 is 1-4, R^{3a} is H or methyl, and R^{3b} is I, Br, F, Cl, H, OH, OCH₃, NH₂, NO₂ or C₁-C₆ alkyl; or

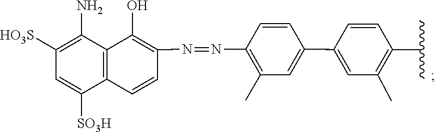 [SMILES: *c1ccc(-c2ccc(N=Nc3ccc4c(S(=O)(=O)O)cc(S(=O)(=O)O)c(N)c4c3O)c(C)c2)cc1C]

n6 is 1-5;
each R^{rad} is a radiolabeling group independently selected from a radiometal chelator; an aryl or heteroaryl substituted with at least one radiohalogen; a prosthetic group containing a trifluoroborate; an aryl or heteroaryl substituted with at least one fluorine precursor leaving group; a prosthetic group containing a silicon-fluorine-acceptor moiety; or a prosthetic group containing a fluorophosphate, fluorosulfate, sulfonyl fluoride, or a combination thereof; and
wherein Xaa¹, Xaa², Xaa³, Xaa⁴, Xaa⁵, Xaa⁶, Xaa⁷, Xaa⁸, Xaa⁹ or Xaa¹⁰ is each optionally N-methylated; wherein the above peptidic compound exhibits a tumor comparative time-activity integration (AUCₜᵤₘₒᵣ) of at least 1000% ID/g·h (e.g., at least 1100% ID/g·h, at least 1200% ID/g·h, at least 1300% ID/g·h, at least 1400% ID/g·h, at least 1500% ID/g·h, at least 1600% ID/g·h, at least 1700% ID/g·h, at least 1800% ID/g·h, at least 1900% ID/g·h, at least 2000% ID/g·h, or at least 2100% ID/g·h).

In another aspect, a peptidic compound of the invention comprises a means for binding to a GRPR-expressing tumor in a subject, wherein the peptidic compound is a compound of Formula (I), or a salt or solvate of Formula (I),

(I)
R^{rad}ₙ₆-[linker]-Xaa¹-Xaa²-Xaa³-Xaa⁴-Xaa⁵-Xaa⁶-
Xaa⁷-Xaa⁸⁻[ψ-Xaa⁹-(Xaa¹⁰)ₘ₂]ₘ₁-R

wherein:
Xaa¹ is an N-terminal amino acid residue selected from D-Phe, 4-chlorophenylalanine (Cpa), D-Cpa, 3-(1-naphthyl)alanine (Nal), D-Nal, 3-(2-naphthyl)alanine (2-Nal), D-2-Nal, 2,3,4,9-tetrahydro-1H-pyrido[3,4-b]indol-3-carboxylic acid (Tpi), or D-Tpi;
Xaa² is Gly, Arg, Asp, Asn, Gln, Glu, homoserine (Hse), citrulline (Cit), 2-amino-4-ureidobutanoic acid (Aub), 22-amino-4-guanidinobutyric acid (Agb), or His;
Xaa³ is Trp, β-(3-benzothienyl)alanine (Bta), Trp(Me), Trp(7-Me), Trp(6-Me), Trp(5-Me), Trp(4-Me), Trp(2-Me), Trp(7-F), Trp(6-F), Trp(5-F), Trp(4-F), Trp(7-Cl), Trp(6-Cl), Trp(5-Cl), Trp(4-Cl), Trp(7-Br), Trp(6-Br), Trp(5-Br), Trp(4-Br), Trp(7-1), Trp(6-1), Trp(5-1), Trp(4-1), Trp(5-OH), αMe-Trp, Tpi, 7-azatryptophan (7-Aza), Nal, 2-pyridylalanine (2-Pal), 3-pyridylalanine (3-Pal), 4-pyridylalanine (4-Pal), 2-quinoylalanine, or 3-quinoylalanine;
Xaa⁴ is Ala, D-Ala, or Ser;
Xaa⁵ is Val, 2,3-dehydro-Val, cyclopentylglycine (Cpg), L-cyclopropylglycine, L-cyclobutylglycine, or tert-leucine (Tle);
Xaa⁶ is Gly or D-Ala;
Xaa⁷ is His, Trp, Trp(Me), Trp(7-Me), Trp(6-Me), Trp(5-Me), Trp(4-Me), Trp(2-Me), Trp(7-F), Trp(6-F), Trp(5-F), Trp(4-F), Trp(7-Cl), Trp(6-Cl), Trp(5-Cl), Trp(4-Cl), Trp(7-Br), Trp(6-Br), Trp(5-Br), Trp(4-Br), Trp(7-1), Trp(6-1), Trp(5-1), Trp(4-1),Trp(5-OH), αMe-Trp, Nal, 2-pyridylalanine (2-Pal), 3-pyridylalanine (3-Pal), 4-pyridylalanine (4-Pal), 2-quinoylalanine, or 3-quinoylalanine;
Xaa⁸ is statine (Sta), Leu, D-Pro, Phe, cis-4-NHR^{a}-D-Pro or cis-4-OR^{a}-D-Pro, wherein R^{a} is alkyl, cycloalkyl, or aryl;
R is —NH₂ or —NHOH;
m1 is 0 or 1;
m2 is 0 or 1;
   when m1 is 1 and m2 is 0, Xaa⁹-R is a C-terminally amidated (R is —NH₂) or a C-terminally hydroxylamidated (R is —NHOH) amino acid residue selected from Pro, Phe, oxazolidine-4-carboxylic acid (4-oxa-L-Pro), 4,4-difluoroproline (difluoroPro), cis-4-NHR^{a}-D-Pro, cis-4-OR^{a}-D-Pro, Leu, 5,5-dimethyl-1,3-thiazolidine-4-carboxylic acid (Me₂Thz), or thiazoline-4-carboxylic acid (Thz), wherein R^{a} is alkyl, cycloalkyl, or aryl;
   when m1 is 1 and m2 is 1, Xaa⁹ is Pro, Phe, oxazolidine-4-carboxylic acid (4-oxa-L-Pro), 4,4-difluoroproline (difluoroPro), Leu, Me₂Thz (5,5-dimethyl-1,3-thiazolidine-4-carboxylic acid), or thiazoline-4-carboxylic acid (Thz); and
      Xaa¹⁰-R is a C-terminally amidated (R is —NH₂) or a C-terminally hydroxylamidated (R is —NHOH) amino acid residue selected from Val, Leu, Tle, Ile, Nle, Ala, Gly, or Met;
ψ represents a peptide bond or reduced peptide bond joining Xaa⁸ to Xaa⁹;
the linker is a linear or branched chain of n1 units of -Xaa¹¹-;
   n1 is 1-20;
   each Xaa¹¹ is independently an amino acid;
   optionally, the linker comprises an albumin binder (R^{alb}), wherein the albumin binder is:
   —(CH₂)ₙ₃—CH₃ wherein n3 is 8-20;
   —(CH₂)ₙ₄—C(O)OH wherein n4 is 8-20;

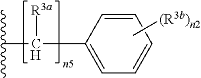

wherein n2 is 1-5, n5 is 1-4, R^{3a} is H or methyl, and R^{3b} is I, Br, F, Cl, H, OH, OCH₃, NH₂, NO₂ or C₁-C₆ alkyl;

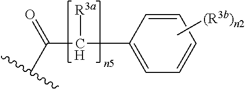

wherein n2 is 1-5, n5 is 1-4, R^{3a} is H or methyl, and R^{3b} is I, Br, F, Cl, H, OH, OCH₃, NH₂, NO₂ or C₁-C₆ alkyl; or

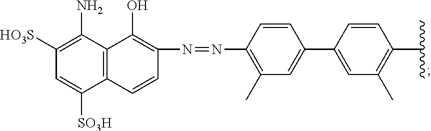 [SMILES: *c1ccc(-c2ccc(N=Nc3ccc4c(S(=O)(=O)O)cc(S(=O)(=O)O)c(N)c4c3O)c(C)c2)cc1C]

n6 is 1-5;
each R^{rad} is a radiolabeling group independently selected from a radiometal chelator; an aryl or heteroaryl substituted with at least one radiohalogen; a prosthetic group containing a trifluoroborate; an aryl or heteroaryl substituted with at least one fluorine precursor leaving group; a prosthetic group containing a silicon-fluorine-acceptor moiety; or a prosthetic group containing a fluorophosphate, fluorosulfate, sulfonyl fluoride, or a combination thereof; and
wherein Xaa¹, Xaa², Xaa³, Xaa⁴, Xaa⁵, Xaa⁶, Xaa⁷, Xaa⁸, Xaa⁹ or Xaa¹⁰ is each optionally N-methylated; wherein the above peptidic compound exhibits a tumor/pancreas uptake ratio of at least 1 (e.g., at least 1.5, at least 2, at least 4, at least 5, at least 6, at least 8, at least 10) and a tumor comparative time-activity integration (AUCₜᵤₘₒᵣ) of at least 1000 (e.g., at least 1100, at least 1200, at least 1300, at least 1400, at least 1500, at least 1600, at least 1700, at least 1800, at least 1900, or at least 2000) % ID/g·h.

In yet another aspect, a peptidic compound of the invention comprises a means for binding to a GRPR-expressing tumor in a subject, wherein the peptidic compound is a compound of Formula (A), or a salt or solvate of Formula (A): R^{rad}n6-R^{L}-[linker]-R^{L}-Xaa¹-Xaa²-Xaa³-Xaa⁴-Xaa⁵-Xaa⁶-Xaa⁷-Xaa⁸-[y,-Xaa⁹-(Xaa¹⁰)ₘ₂]ₘ₁-R (A) wherein:
Xaa¹ is an N-terminal residue comprising any proteinogenic or nonproteinogenic amino acid;
Xaa² is any proteinogenic or nonproteinogenic amino acid;
Xaa³ is Trp, β-(3-benzothienyl)alanine (Bta), Trp(Me), Trp(7-Me), Trp(6-Me), Trp(5-Me), Trp(4-Me), Trp(2-Me), Trp(7-F), Trp(6-F), Trp(5-F), Trp(4-F), Trp(7-Cl), Trp(6-Cl), Trp(5-Cl), Trp(4-Cl), Trp(7-Br), Trp(6-Br), Trp(5-Br), Trp(4-Br), Trp(7-1), Trp(6-1), Trp(5-1), Trp(4-1), Trp(5-OH), αMe-Trp, Tpi, 7-azatryptophan (7-Aza), Nal, 2-pyridylalanine (2-Pal), 3-pyridylalanine (3-Pal), 4-pyridylalanine (4-Pal), 2-quinoylalanine (2-Quin-Ala), or 3-quinoylalanine (3-Quin-Ala);
Xaa⁴ is any proteinogenic or nonproteinogenic amino acid;
Xaa⁵ is any proteinogenic or nonproteinogenic amino acid;
Xaa⁶ is any proteinogenic or nonproteinogenic amino acid;
Xaa⁷ is Trp, Trp(Me), Trp(7-Me), Trp(6-Me), Trp(5-Me), Trp(4-Me), Trp(2-Me), Trp(7-F), Trp(6-F), Trp(5-F), Trp(4-F), Trp(7-Cl), Trp(6-Cl), Trp(5-Cl), Trp(4-Cl), Trp(7-Br), Trp(6-Br), Trp(5-Br), Trp(4-Br), Trp(7-1), Trp(6-1), Trp(5-1), Trp(4-1), Trp(5-OH), αMe-Trp, Nal, 2-pyridylalanine (2-Pal), 3-pyridylalanine (3-Pal), 4-pyridylalanine (4-Pal), 2-quinoylalanine (2-Quin-Ala), or 3-quinoylalanine (3-Quin-Ala);
Xaa⁸ is any proteinogenic or nonproteinogenic amino acid;
R is —NH₂ or —NHOH;
m1 is 0 or 1;
m2 is 0 or 1;
when m1 is 1 and m2 is 0, Xaa⁹-R is a C-terminally amidated (R is —NH₂) or a C-terminally hydroxylamidated (R is —NHOH) amino acid residue;
when m1 is 1 and m2 is 1, Xaa⁹ is any proteinogenic or nonproteinogenic amino acid; and Xaa¹⁰-R is a C-terminally amidated (R is —NH₂) or a C-terminally hydroxylamidated (R is —NHOH) amino acid residue;
ψ represents a peptide bond or reduced peptide bond joining Xaa⁸ to Xaa⁹;
each R^{L} is independently absent, —(C₁₋₆ alkyl)-, —(C₁₋₆ alkyl)-C(O)—, —C(O)—, —NH—C(O)—, or —NH—C(S)—;
the linker is a linear or branched chain optionally comprising an albumin binder; n6 is 1-5;
each R^{rad} is a radiolabeling group independently selected from a radiometal chelator; an aryl or heteroaryl substituted with at least one radiohalogen; a prosthetic group containing a trifluoroborate; an aryl or heteroaryl substituted with at least one fluorine precursor leaving group; a prosthetic group containing a silicon-fluorine-acceptor moiety; or a prosthetic group containing a fluorophosphate, fluorosulfate, sulfonyl fluoride, or a combination thereof; and
wherein Xaa¹, Xaa², Xaa³, Xaa⁴, Xaa⁵, Xaa⁶, Xaa⁷, Xaa⁸, Xaa⁹ or Xaa¹⁰ is each optionally N-methylated; wherein the above peptidic compound of Formula (A) exhibits a tumor/pancreas uptake ratio of at least 1 (e.g., at least 1.5 or higher, about 2 or higher, about 4 or higher, about 5 or higher, about 6 or higher, about 7 or higher, about 10 or higher, or about 16).

In an aspect, a peptidic compound of the invention comprises a means for binding to a GRPR-expressing tumor in a subject, wherein the peptidic compound is a compound of Formula (A), or a salt or solvate of Formula (A):

(A)
R^{rad}ₙ₆-R^{L}-[linker]-R^{L}-Xaa¹-Xaa²-Xaa³-Xaa⁴-Xaa⁵-
Xaa⁶-Xaa⁷-Xaa⁸-[ψ-Xaa⁹-(Xaa¹⁰)ₘ₂]ₘ₁-R

wherein:
Xaa¹ is an N-terminal residue comprising any proteinogenic or nonproteinogenic amino acid;
Xaa² is any proteinogenic or nonproteinogenic amino acid;
Xaa³ is Trp, β-(3-benzothienyl)alanine (Bta), Trp(Me), Trp(7-Me), Trp(6-Me), Trp(5-Me), Trp(4-Me), Trp(2-Me), Trp(7-F), Trp(6-F), Trp(5-F), Trp(4-F), Trp(7-Cl), Trp(6-Cl), Trp(5-Cl), Trp(4-Cl), Trp(7-Br), Trp(6-Br), Trp(5-Br), Trp(4-Br), Trp(7-1), Trp(6-1), Trp(5-1), Trp(4-1), Trp(5-OH), αMe-Trp, Tpi, 7-azatryptophan (7-Aza), Nal, 2-pyridylalanine (2-Pal), 3-pyridylalanine (3-Pal), 4-pyridylalanine (4-Pal), 2-quinoylalanine (2-Quin-Ala), or 3-quinoylalanine (3-Quin-Ala);
Xaa⁴ is any proteinogenic or nonproteinogenic amino acid;
Xaa⁵ is any proteinogenic or nonproteinogenic amino acid;
Xaa⁶ is any proteinogenic or nonproteinogenic amino acid;
Xaa⁷ is Trp, Trp(Me), Trp(7-Me), Trp(6-Me), Trp(5-Me), Trp(4-Me), Trp(2-Me), Trp(7-F), Trp(6-F), Trp(5-F), Trp(4-F), Trp(7-Cl), Trp(6-Cl), Trp(5-Cl), Trp(4-Cl), Trp(7-Br), Trp(6-Br), Trp(5-Br), Trp(4-Br), Trp(7-1), Trp(6-1), Trp(5-1), Trp(4-1), Trp(5-OH), αMe-Trp, Nal, 2-pyridylalanine (2-Pal), 3-pyridylalanine (3-Pal), 4-pyridylalanine (4-Pal), 2-quinoylalanine (2-Quin-Ala), or 3-quinoylalanine (3-Quin-Ala);
Xaa⁸ is any proteinogenic or nonproteinogenic amino acid;
R is —NH₂ or —NHOH;
m1 is 0 or 1;
m2 is 0 or 1;
   when m1 is 1 and m2 is 0, Xaa⁹-R is a C-terminally amidated (R is —NH₂) or a C-terminally hydroxylamidated (R is —NHOH) amino acid residue;
   when m1 is 1 and m2 is 1, Xaa⁹ is any proteinogenic or nonproteinogenic amino acid; and Xaa¹⁰-R is a C-terminally amidated (R is —NH₂) or a C-terminally hydroxylamidated (R is —NHOH) amino acid residue;
ψ represents a peptide bond or reduced peptide bond joining Xaa⁸ to Xaa⁹;
each R^{L} is independently absent, —(C₁₋₆ alkyl)-, —(C₁₋₆ alkyl)-C(O)—, —C(O)—, —NH—C(O)—, or —NH—C(S)—;
the linker is a linear or branched chain optionally comprising an albumin binder;
n6 is 1-5;
each R^{rad} is a radiolabeling group independently selected from a radiometal chelator; an aryl or heteroaryl substituted with at least one radiohalogen; a prosthetic group containing a trifluoroborate; an aryl or heteroaryl substituted with at least one fluorine precursor leaving group; a prosthetic group containing a silicon-fluorine-acceptor moiety; or a prosthetic group containing a fluorophosphate, fluorosulfate, sulfonyl fluoride, or a combination thereof; and
wherein Xaa¹, Xaa², Xaa³, Xaa⁴, Xaa⁵, Xaa⁶, Xaa⁷, Xaa⁸, Xaa⁹ or Xaa¹⁰ is each optionally N-methylated; wherein the above peptidic compound of Formula (A) exhibits a tumor comparative time-activity integration (AUCₜᵤₘₒᵣ) of at least 1000% ID/g·h (e.g., at least 1100% ID/g·h, at least 1200% ID/g·h, at least 1300% ID/g·h, at least 1400% ID/g·h, at least 1500% ID/g·h, at least 1600% ID/g·h, at least 1700% ID/g·h, at least 1800% ID/g·h, at least 1900% ID/g·h, at least 2000% ID/g·h, or at least 2100% ID/g·h).

In yet another aspect, a peptidic compound of the invention comprises a means for binding to a GRPR-expressing tumor in a subject, wherein the peptidic compound is a compound of Formula (A), or a salt or solvate of Formula (A):

(A)
R^{rad}ₙ₆-R^{L}-[linker]-R^{L}-Xaa¹-Xaa²-Xaa³-Xaa⁴-Xaa⁵-
Xaa⁶-Xaa⁷-Xaa⁸-[ψ-Xaa⁹-(Xaa¹⁰)ₘ₂]ₘ₁-R

wherein:
Xaa¹ is an N-terminal amino acid residue comprising any proteinogenic or nonproteinogenic amino acid;
Xaa² is any proteinogenic or nonproteinogenic amino acid;
Xaa³ is Trp, β-(3-benzothienyl)alanine (Bta), Trp(Me), Trp(7-Me), Trp(6-Me), Trp(5-Me), Trp(4-Me), Trp(2-Me), Trp(7-F), Trp(6-F), Trp(5-F), Trp(4-F), Trp(7-Cl), Trp(6-Cl), Trp(5-Cl), Trp(4-Cl), Trp(7-Br), Trp(6-Br), Trp(5-Br), Trp(4-Br), Trp(7-1), Trp(6-1), Trp(5-1), Trp(4-1), Trp(5-OH), αMe-Trp, Tpi, 7-azatryptophan (7-Aza), Nal, 2-pyridylalanine (2-Pal), 3-pyridylalanine (3-Pal), 4-pyridylalanine (4-Pal), 2-quinoylalanine (2-Quin-Ala), or 3-quinoylalanine (3-Quin-Ala);
Xaa⁴ is any proteinogenic or nonproteinogenic amino acid;
Xaa⁵ is any proteinogenic or nonproteinogenic amino acid;
Xaa⁶ is any proteinogenic or nonproteinogenic amino acid;
Xaa⁷ is Trp, Trp(Me), Trp(7-Me), Trp(6-Me), Trp(5-Me), Trp(4-Me), Trp(2-Me), Trp(7-F), Trp(6-F), Trp(5-F), Trp(4-F), Trp(7-Cl), Trp(6-Cl), Trp(5-Cl), Trp(4-Cl), Trp(7-Br), Trp(6-Br), Trp(5-Br), Trp(4-Br), Trp(7-1), Trp(6-1), Trp(5-1), Trp(4-1), Trp(5-OH), αMe-Trp, Nal, 2-pyridylalanine (2-Pal), 3-pyridylalanine (3-Pal), 4-pyridylalanine (4-Pal), 2-quinoylalanine (2-Quin-Ala), or 3-quinoylalanine (3-Quin-Ala);
Xaa⁸ is any proteinogenic or nonproteinogenic amino acid;
R is —NH₂ or —NHOH;
m1 is 0 or 1;
m2 is 0 or 1;
   when m1 is 1 and m2 is 0, Xaa⁹-R is a C-terminally amidated (R is —NH₂) or a C-terminally hydroxylamidated (R is —NHOH) amino acid residue;
   when m1 is 1 and m2 is 1, Xaa⁹ is any proteinogenic or nonproteinogenic amino acid; and Xaa¹⁰-R is a C-terminally amidated (R is —NH₂) or a C-terminally hydroxylamidated (R is —NHOH) amino acid residue;
ψ represents a peptide bond or reduced peptide bond joining Xaa⁸ to Xaa⁹;
each R^{L} is independently absent, —(C₁₋₆ alkyl)-, —(C₁₋₆ alkyl)-C(O)—, —C(O)—, —NH—C(O)—, or —NH—C(S)—;
the linker is a linear or branched chain optionally comprising an albumin binder; n6 is 1-5;
each R^{rad} is a radiolabeling group independently selected from a radiometal chelator; an aryl or heteroaryl substituted with at least one radiohalogen; a prosthetic group containing a trifluoroborate; an aryl or heteroaryl substituted with at least one fluorine precursor leaving group; a prosthetic group containing a silicon-fluorine-acceptor moiety; or a prosthetic group containing a fluorophosphate, fluorosulfate, sulfonyl fluoride, or a combination thereof; and
wherein Xaa¹, Xaa², Xaa³, Xaa⁴, Xaa⁵, Xaa⁶, Xaa⁷, Xaa⁸, Xaa⁹ or Xaa¹⁰ is each optionally N-methylated; wherein the above peptidic compound exhibits a tumor/pancreas uptake ratio of at least 1 (e.g., at least 1.5, at least 2, at least 4, at least 5, at least 6, at least 8, at least 10) and a tumor comparative time-activity integration (AUCₜᵤₘₒᵣ) of at least 1000 (e.g., at least 1100, at least 1200, at least 1300, at least 1400, at least 1500, at least 1600, at least 1700, at least 1800, at least 1900, or at least 2000) % ID/g·h.

In another aspect, a peptidic compound of the invention comprises a means for binding to a GRPR-expressing tumor in a subject, wherein the peptidic compound is a compound of Formula (I), or a salt or solvate of Formula (I),

(I)
R^{rad}ₙ₆-[linker]-Xaa¹-Xaa²-Xaa³-Xaa⁴-Xaa⁵-Xaa⁶-
Xaa⁷-Xaa⁸-[ψ-Xaa⁹-(Xaa¹⁰)ₘ₂]ₘ₁-R

wherein:
Xaa¹ is an N-terminal amino acid residue comprising any proteinogenic or nonproteinogenic amino acid;
Xaa² is any proteinogenic or nonproteinogenic amino acid;
Xaa³ is Trp, β-(3-benzothienyl)alanine (Bta), Trp(Me), Trp(7-Me), Trp(6-Me), Trp(5-Me), Trp(4-Me), Trp(2-Me), Trp(7-F), Trp(6-F), Trp(5-F), Trp(4-F), Trp(7-Cl), Trp(6-Cl), Trp(5-Cl), Trp(4-Cl), Trp(7-Br), Trp(6-Br), Trp(5-Br), Trp(4-Br), Trp(7-1), Trp(6-1), Trp(5-1), Trp(4-1), Trp(5-OH), αMe-Trp, Tpi, 7-azatryptophan (7-Aza), Nal, 2-pyridylalanine (2-Pal), 3-pyridylalanine (3-Pal), 4-pyridylalanine (4-Pal), 2-quinoylalanine, or 3-quinoylalanine;
Xaa⁴ is any proteinogenic or nonproteinogenic amino acid;
Xaa⁵ is any proteinogenic or nonproteinogenic amino acid;
Xaa⁶ is any proteinogenic or nonproteinogenic amino acid;
Xaa⁷ is Trp, Trp(Me), Trp(7-Me), Trp(6-Me), Trp(5-Me), Trp(4-Me), Trp(2-Me), Trp(7-F), Trp(6-F), Trp(5-F), Trp(4-F), Trp(7-Cl), Trp(6-Cl), Trp(5-Cl), Trp(4-Cl), Trp(7-Br), Trp(6-Br), Trp(5-Br), Trp(4-Br), Trp(7-1), Trp(6-1), Trp(5-1), Trp(4-1),Trp(5-OH), αMe-Trp, Nal, 2-pyridylalanine (2-Pal), 3-pyridylalanine (3-Pal), 4-pyridylalanine (4-Pal), 2-quinoylalanine, or 3-quinoylalanine;
Xaa⁸ is any proteinogenic or nonproteinogenic amino acid;
R is —NH₂ or —NHOH;
m1 is 0 or 1;
m2 is 0 or 1;
   when m1 is 1 and m2 is 0, Xaa⁹-R is a C-terminally amidated (R is —NH₂) or a C-terminally hydroxylamidated (R is —NHOH) amino acid residue;
   when m1 is 1 and m2 is 1, Xaa⁹ is any proteinogenic or nonproteinogenic amino acid; and Xaa¹⁰-R is a C-terminally amidated (R is —NH₂) or a C-terminally hydroxylamidated (R is —NHOH) amino acid residue;
ψ represents a peptide bond or reduced peptide bond joining Xaa⁸ to Xaa⁹;
the linker is a linear or branched chain optionally comprising an albumin binder; n6 is 1-5;
each R^{rad} is a radiolabeling group independently selected from a radiometal chelator; an aryl or heteroaryl substituted with at least one radiohalogen; a prosthetic group containing a trifluoroborate; an aryl or heteroaryl substituted with at least one fluorine precursor leaving group; a prosthetic group containing a silicon-fluorine-acceptor moiety; or a prosthetic group containing a fluorophosphate, fluorosulfate, sulfonyl fluoride, or a combination thereof; and
wherein Xaa¹, Xaa², Xaa³, Xaa⁴, Xaa⁵, Xaa⁶, Xaa⁷, Xaa⁸, Xaa⁹ or Xaa¹⁰ is each optionally N-methylated; wherein the above peptidic compound exhibits a tumor/pancreas uptake ratio of at least 1 (e.g., at least 1.5 or higher, about 2 or higher, about 4 or higher, about 5 or higher, about 6 or higher, about 7 or higher, about 10 or higher, or about 16).

In another aspect, a peptidic compound of the invention comprises a means for binding to a GRPR-expressing tumor in a subject, wherein the peptidic compound is a compound of Formula (I), or a salt or solvate of Formula (I),

(I)
R^{rad}ₙ₆-[linker]-Xaa¹-Xaa²-Xaa³-Xaa⁴-Xaa⁵-Xaa⁶-
Xaa⁷-Xaa⁸-[ψ-Xaa⁹-(Xaa¹⁰)ₘ₂]ₘ₁-R

wherein:
Xaa¹ is an N-terminal amino acid residue comprising any proteinogenic or nonproteinogenic amino acid;
Xaa² is any proteinogenic or nonproteinogenic amino acid;
Xaa³ is Trp, β-(3-benzothienyl)alanine (Bta), Trp(Me), Trp(7-Me), Trp(6-Me), Trp(5-Me), Trp(4-Me), Trp(2-Me), Trp(7-F), Trp(6-F), Trp(5-F), Trp(4-F), Trp(7-Cl), Trp(6-Cl), Trp(5-Cl), Trp(4-Cl), Trp(7-Br), Trp(6-Br), Trp(5-Br), Trp(4-Br), Trp(7-1), Trp(6-1), Trp(5-1), Trp(4-1), Trp(5-OH), αMe-Trp, Tpi, 7-azatryptophan (7-Aza), Nal, 2-pyridylalanine (2-Pal), 3-pyridylalanine (3-Pal), 4-pyridylalanine (4-Pal), 2-quinoylalanine, or 3-quinoylalanine;
Xaa⁴ is any proteinogenic or nonproteinogenic amino acid;
Xaa⁵ is any proteinogenic or nonproteinogenic amino acid;
Xaa⁶ is any proteinogenic or nonproteinogenic amino acid;
Xaa⁷ is Trp, Trp(Me), Trp(7-Me), Trp(6-Me), Trp(5-Me), Trp(4-Me), Trp(2-Me), Trp(7-F), Trp(6-F), Trp(5-F), Trp(4-F), Trp(7-Cl), Trp(6-Cl), Trp(5-Cl), Trp(4-Cl), Trp(7-Br), Trp(6-Br), Trp(5-Br), Trp(4-Br), Trp(7-1), Trp(6-1), Trp(5-1), Trp(4-1),Trp(5-OH), αMe-Trp, Nal, 2-pyridylalanine (2-Pal), 3-pyridylalanine (3-Pal), 4-pyridylalanine (4-Pal), 2-quinoylalanine, or 3-quinoylalanine;
Xaa⁸ is any proteinogenic or nonproteinogenic amino acid;
R is —NH₂ or —NHOH;
m1 is 0 or 1;
m2 is 0 or 1;
   when m1 is 1 and m2 is 0, Xaa⁹-R is a C-terminally amidated (R is —NH₂) or a C-terminally hydroxylamidated (R is —NHOH) amino acid residue;
   when m1 is 1 and m2 is 1, Xaa⁹ is any proteinogenic or nonproteinogenic amino acid; and Xaa¹⁰-R is a C-terminally amidated (R is —NH₂) or a C-terminally hydroxylamidated (R is —NHOH) amino acid residue;
ψ represents a peptide bond or reduced peptide bond joining Xaa⁸ to Xaa⁹;
the linker is a linear or branched chain optionally comprising an albumin linker;
n6 is 1-5;
each R^{rad} is a radiolabeling group independently selected from a radiometal chelator; an aryl or heteroaryl substituted with at least one radiohalogen; a prosthetic group containing a trifluoroborate; an aryl or heteroaryl substituted with at least one fluorine precursor leaving group; a prosthetic group containing a silicon-fluorine-acceptor moiety; or a prosthetic group containing a fluorophosphate, fluorosulfate, sulfonyl fluoride, or a combination thereof; and
wherein Xaa¹, Xaa², Xaa³, Xaa⁴, Xaa⁵, Xaa⁶, Xaa⁷, Xaa⁸, Xaa⁹ or Xaa¹⁰ is each optionally N-methylated; wherein the above peptidic compound exhibits a tumor comparative time-activity integration (AUCₜᵤₘₒᵣ) of at least 1000% ID/g·h (e.g., at least 1100% ID/g·h, at least 1200% ID/g·h, at least 1300% ID/g·h, at least 1400% ID/g·h, at least 1500% ID/g·h, at least 1600% ID/g·h, at least 1700% ID/g·h, at least 1800% ID/g·h, at least 1900% ID/g·h, at least 2000% ID/g·h, or at least 2100% ID/g·h).

In another aspect, a peptidic compound of the invention comprises a means for binding to a GRPR-expressing tumor in a subject, wherein the peptidic compound is a compound of Formula (I), or a salt or solvate of Formula (I),

(I)
R^{rad}ₙ₆-[linker]-Xaa¹-Xaa²-Xaa³-Xaa⁴-Xaa⁵-Xaa⁶-
Xaa⁷-Xaa⁸-[ψ-Xaa⁹-(Xaa¹⁰)ₘ₂]ₘ₁-R

wherein:
Xaa¹ is an N-terminal amino acid residue comprising any proteinogenic or nonproteinogenic amino acid;
Xaa² is any proteinogenic or nonproteinogenic amino acid;
Xaa³ is Trp, β-(3-benzothienyl)alanine (Bta), Trp(Me), Trp(7-Me), Trp(6-Me), Trp(5-Me), Trp(4-Me), Trp(2-Me), Trp(7-F), Trp(6-F), Trp(5-F), Trp(4-F), Trp(7-Cl), Trp(6-Cl), Trp(5-Cl), Trp(4-Cl), Trp(7-Br), Trp(6-Br), Trp(5-Br), Trp(4-Br), Trp(7-1), Trp(6-1), Trp(5-1), Trp(4-1), Trp(5-OH), αMe-Trp, Tpi, 7-azatryptophan (7-Aza), Nal, 2-pyridylalanine (2-Pal), 3-pyridylalanine (3-Pal), 4-pyridylalanine (4-Pal), 2-quinoylalanine, or 3-quinoylalanine;
Xaa⁴ is any proteinogenic or nonproteinogenic amino acid;
Xaa⁵ is any proteinogenic or nonproteinogenic amino acid;
Xaa⁶ is any proteinogenic or nonproteinogenic amino acid;
Xaa⁷ is Trp, Trp(Me), Trp(7-Me), Trp(6-Me), Trp(5-Me), Trp(4-Me), Trp(2-Me), Trp(7-F), Trp(6-F), Trp(5-F), Trp(4-F), Trp(7-Cl), Trp(6-Cl), Trp(5-Cl), Trp(4-Cl), Trp(7-Br), Trp(6-Br), Trp(5-Br), Trp(4-Br), Trp(7-1), Trp(6-1), Trp(5-1), Trp(4-I),Trp(5-OH), αMe-Trp, Nal, 2-pyridylalanine (2-Pal), 3-pyridylalanine (3-Pal), 4-pyridylalanine (4-Pal), 2-quinoylalanine, or 3-quinoylalanine;
Xaa⁸ is any proteinogenic or nonproteinogenic amino acid;
R is —NH₂ or —NHOH;
m1 is 0 or 1;
m2 is 0 or 1;
   when m1 is 1 and m2 is 0, Xaa⁹-R is a C-terminally amidated (R is —NH₂) or a C-terminally hydroxylamidated (R is —NHOH) amino acid residue;
   when m1 is 1 and m2 is 1, Xaa⁹ is any proteinogenic or nonproteinogenic amino acid; and Xaa¹⁰-R is a C-terminally amidated (R is —NH₂) or a C-terminally hydroxylamidated (R is —NHOH) amino acid residue;
ψ represents a peptide bond or reduced peptide bond joining Xaa⁸ to Xaa⁹;
the linker is a linear or branched chain optionally comprising an albumin binder; n6 is 1-5;
each R^{rad} is a radiolabeling group independently selected from a radiometal chelator; an aryl or heteroaryl substituted with at least one radiohalogen; a prosthetic group containing a trifluoroborate; an aryl or heteroaryl substituted with at least one fluorine precursor leaving group; a prosthetic group containing a silicon-fluorine-acceptor moiety; or a prosthetic group containing a fluorophosphate, fluorosulfate, sulfonyl fluoride, or a combination thereof; and
wherein Xaa¹, Xaa², Xaa³, Xaa⁴, Xaa⁵, Xaa⁶, Xaa⁷, Xaa⁸, Xaa⁹ or Xaa¹⁰ is each optionally N-methylated; wherein the above peptidic compound exhibits a tumor/pancreas uptake ratio of at least 1 (e.g., at least 1.5, at least 2, at least 4, at least 5, at least 6, at least 8, at least 10) and a tumor comparative time-activity integration (AUCₜᵤₘₒᵣ) of at least 1000 (e.g., at least 1100, at least 1200, at least 1300, at least 1400, at least 1500, at least 1600, at least 1700, at least 1800, at least 1900, or at least 2000) % ID/g·h.

In some embodiments, the means for binding to a GRPR-expressing tumor in a subject is achieved by a portion of the peptidic compound having any one of SEQ ID NOs: 3-21. In some embodiments, the means for binding to a GRPR-expressing tumor in a subject is achieved by a peptidic compound having SEQ ID NO: 2. In some embodiments, the means for binding to a GRPR-expressing tumor in a subject is achieved by a peptidic compound having any one of SEQ ID NOs: 22-65.

### Compositions

In another aspect, a composition comprising any one of the peptidic compounds as described herein is disclosed. In some embodiments, the composition is a pharmaceutical composition. In some embodiments, the composition comprises a pharmaceutically acceptable excipient or carrier.

The GRPR targeting compounds disclosed herein are typically formulated for administration to a patient, either before or after incorporation of a radiolabel. Certain embodiments of the invention thus relate to pharmaceutical compositions comprising one or more of the radio-labelled GRPR targeting compounds, or unlabeled GRPR targeting compounds, and a pharmaceutically acceptable carrier, diluent, or excipient. The pharmaceutical compositions are prepared by known procedures using well-known and readily available ingredients.

The pharmaceutical compositions comprising the GRPR targeting compounds are typically formulated for parenteral administration. The term parenteral as used herein includes subcutaneous, intradermal, intra-articular, intravenous, intraperitoneal, intramuscular, intravascular, intrasternal, intrathecal injection or infusion techniques.

In certain embodiments, the pharmaceutical compositions may be in the form of a sterile injectable aqueous or oleaginous suspension. This suspension may be formulated according to known art using those suitable dispersing or wetting agents and suspending agents that have been mentioned above. The sterile injectable preparation may also be a sterile injectable solution or a suspension in a non-toxic parentally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or diglycerides. In addition, fatty acids such as oleic acid find use in the preparation of injectables. Adjuvants such as local anesthetics, preservatives and buffering agents can also be included in the injectable solution or suspension.

Other pharmaceutical compositions and methods of preparing pharmaceutical compositions are known in the art and are described, for example, in “Remington: The Science and Practice of Pharmacy” (formerly “Remingtons Pharmaceutical Sciences”); Gennaro, A., Lippincott, Williams & Wilkins, Philadelphia, PA (2000).

### Diagnostic Applications

In a further aspect, this disclosure relates to diagnostic applications of the radio-labelled GRPR targeting compounds for imaging a cancer or tissue in which GRPR is expressed, aberrantly expressed, ectopically expressed, or overexpressed, for example, in oncology.

Overexpression of GRPR has been demonstrated in many malignancies, including psychiatric disorder, neurological disorder, inflammatory disease, prostate cancer, lung cancer, head and neck cancer, colon cancer, kidney cancer, ovarian cancer, liver cancer, pancreatic cancer, breast cancer, glioma (e.g., glioblastoma), or neuroblastoma. GRPR activation has been shown to potentiate malignant behaviors by inducing cell proliferation, migration, and angiogenesis. Certain embodiments of the invention thus contemplate that the radio-labelled GRPR targeting compounds could be used as imaging probes for cancers of prostate cancer, lung cancer, head and neck cancer, colon cancer, kidney cancer, ovarian cancer, liver cancer, pancreatic cancer, breast cancer, glioma (e.g., glioblastoma), or neuroblastoma, including for use in early diagnosis of solid malignancies that express GRPR, as imaging agents to confirm the diagnostic of malignancy, or to guide focal ablative treatment if the disease is localized. According to certain embodiments, the GRPR targeting compounds could also be used to monitor response to therapy, by providing an independent assessment of the residual cellular content of a tumor known to overexpress GRPR.

Radio-labelled GRPR targeting compounds of this disclosure can show high contrast, rapid renal clearance, minimal non-target organ uptake, and high tumour to normal tissue ratios, which properties make these compounds well-suited for use as imaging agents for cancer diagnosis, including diagnosis of early-stage cancer. In particular, certain GRPR targeting compounds according to embodiments of the invention exhibit relatively low renal retention, and relatively high ratio of tumor:kidney and/or tumor/pancreas uptake, facilitating safe and effective use of the compounds in diagnostic and radiotherapeutic treatments.

In some embodiments of the invention, it is contemplated that the radio-labelled GRPR targeting compounds may be used as PET/SPECT imaging probes to assist with precise localization of primary or recurrent cancer in order to guide and assist with focal ablative therapies.

In some embodiments, the invention contemplates that the radio-labelled GRPR targeting compounds could be used to monitor response to therapy, by providing an independent assessment of the residual cellular content of a tumour known to overexpress GRPR. Overexpression of GRPR may be an indicator of angiogenesis in tumours, as blocking of GRPR activation is known to have antiangiogenic activity.

There is some evidence that GRPR antagonists might cause growth inhibition in some cancers. In certain embodiments, GRPR expression and receptor blockage could be detected by imaging with the radio-labelled GRPR targeting compounds, which could then act as a predictive biomarker for treatment success.

In some embodiments, the use of the radio-labelled GRPR targeting compounds in multimodality imaging of cancers is contemplated, for example, combined functional imaging and anatomical imaging, such as PET/CT or SPECT/CT. Multimodality imaging may be useful in situations in which a cancer is present, but the uptake of imaging agent is low.

### Therapeutic Uses

In an additional aspect, the present disclosure provides a method for treating a disease or condition. In some embodiments, the disease or the condition is a GRPR-expressing condition or disease.

In an additional aspect, the present disclosure provides a method for treating a GRPR-expressing condition or disease comprises administering to a subject in need of imaging any one of the peptidic compounds or compositions comprising any one of the peptidic compounds disclosed herein. In some embodiments, the GRPR-expressing condition or disease is cancer. In some embodiments, the GRPR-expressing condition or disease is a psychiatric disorder, neurological disorder, inflammatory disease, prostate cancer, lung cancer, head and neck cancer, colon cancer, kidney cancer, ovarian cancer, liver cancer, pancreatic cancer, breast cancer, glioma (e.g., glioblastoma), or neuroblastoma. In some embodiments, the cancer is prostate cancer. In some embodiments, the cancer is breast cancer. In some embodiments, the cancer is glioblastoma. According to further embodiments, the GRPR targeting compounds can be used for endoradiotherapy targeting cells expressing GRPR, by radiolabelling the compound with a radioisotope, usually a p- or a-particle emitter, to deliver a high local dose of radiation to lesions, to inflict DNA damage and inducing cellular death. Unlike external beam radiation therapy, in certain embodiments this systemic treatment can be effective even in the treatment of metastases.

### Pharmaceutical Packs or Kits

Certain embodiments of the invention relate to pharmaceutical packs or kits containing one or more GRPR targeting compounds, for example, therapeutic or diagnostic packs or kits. The compounds may be provided radio-labelled or as precursors suitable for radio-labelling, in which case the kit may optionally include additional reagents for radio-labelling the compounds.

In certain embodiments, one or more of the components of the kit can be lyophilized and the kit can additionally contain a suitable solvent for reconstitution of the lyophilized components. Individual components of the kit would typically be packaged in separate containers and, associated with such containers, can be a notice in the form prescribed by a governmental agency regulating the manufacture, use or sale of pharmaceuticals or biological products, which notice reflects approval by the agency of manufacture, for use or sale for human or animal administration.

In certain embodiments, the compound(s) are provided in the kit in the form of pharmaceutical compositions suitable for administration to a subject. In this case, if desired, the container may itself be an inhalant, syringe, pipette, eye dropper, or other such like apparatus, from which the composition may be administered to the subject.

GRPR targeting compounds described herein can be synthesized according to conventional methods known to those of ordinary skill in the art. Exemplary, non-limiting procedures are provided in the Examples below.

It is contemplated that any embodiment discussed herein can be implemented with respect to any method, use or composition of the invention, and vice versa. Furthermore, compositions and kits of the invention can be used to achieve methods and uses of the invention.

To gain a better understanding of the invention described herein, the following examples are set forth. It will be understood that these examples are intended to describe illustrative embodiments of the invention and are not intended to limit the scope of the invention in any way.

### EXAMPLES

### General Methods

Chemicals were procured from commercial sources and used without further purification. All peptides were synthesized on a peptide synthesizer (Liberty Blue 2.0). Purification and quality control of peptide precursor and nonradioactive Lu-complexed and La-complexed standards were performed on Agilent HPLC and LC/MS systems. HPLC columns used were preparative columns (Luna 5 μm C18(2) 100 Å, LC Column 150×21.2 mm), (Luna 5 μm C18(2) 100 Å, LC Column 50×30 mm), semipreparative columns (Luna C18, 5 μm particle size, 100 A pore size, 250×10 mm), (Aeris 3.6 μm PEPTIDE XB—C18 100 Å, LC Column 250×4.6 mm) and analytical columns (Luna C18, 5 μm particle size, 100 A pore size, 250×4.6 mm), (Kinetex 2.6 μm PS C18 100 Å, LC Column 100×3 mm) and (Kinetex 2.6 μm C18 100 Å, LC Column 100×3 mm) from Phenomenex. The collected HPLC eluates containing the desired peptides were lyophilized using a Labconco FreeZone 4.5 Plus freeze drier. Mass analyses were performed using Agilent mass spectrometer.

### General Peptide Synthesis

At a 0.1 mmol scale, Sieber resin or Rink Amide Resin was deprotected by treating the resin with 20% piperidine in DMF (3×8 min). Fmoc-Leu-ψ-Pro-OH (or the first amino acid) was coupled to the resin via solid-phase peptide synthesis using Fmoc-based chemistry. The coupling was carried out in DMF using Fmoc-Leu-ψ-Pro-OH (4 eq.), HATU (4 eq.), HOAt (4 eq.) and DIEPA (11 eq.) at room temperature for overnight (16 hours). The preloaded Fmoc-Leu-ψ-Pro-Sieber resin (or Rink Amide resin) was then loaded onto the Liberty Blue, and the Fmoc was deprotected with 10% Piperidine in DMF at 45° C. (3 min×3) or 20% piperidine in DMF at room temperature (3×8 min). According to the peptide sequence, each Fmoc-protected amino acid (4 eq., 0.4 mmol, 0.2 M in DMF) was subsequently coupled at 45° C. (20 min) using 1 M DIC/1 M Oxyma in DMF or coupled at room temperature (1-3h) using HATU (4 eq.), HOAt (4 eq.) in DMF, followed by Fmoc-deprotection as described above. Fmoc-Ala-OH was double-coupled at 45° C. (20 min×2) or room temperature (1 h×2). The synthesis continued with the addition of the linker, Fmoc-4-amino-(1-carboxymethyl) piperidine (4 eq.), using double coupling at 45° C. for 1.5 h or couple at room temperature for 3 h and then followed by the chelator DOTA(tBu)₃ (4 eq) or Crown(tBu)₃ (4 eq) at 25° C. for 18 h. The coupling of the linker and the chelator were carried out using HATU/HOAt/DIPEA (4/4/11 eq). The peptide was then deprotected and simultaneously cleaved from the resin by treating with 95/5 trifluoroacetic acid (TFA)/triisopropylsilane (TIS) for 2 h at room temperature. After filtration, the crude peptide was precipitated by the addition of cold diethyl ether to the TFA solution and lyophilized in a H2O/MeCN mixture.

### Cold Lutetium or Lanthanum Cold Complexations Synthesis:

The precursor (1 eq., 5-10 mg) and LuCl₃ (5 eq.) or LaCl₃ (5 eq.) were dissolved in 0.1 M aq. NaOAc and MeCN (2.5:1, DMSO was added when solubility is low). The reaction mixture was heated to 90° C. for 30 minutes, at which point most reactions were complete (monitored by MS). The reaction mixture was then diluted with H₂O/MeCN and directly purified by semi-prep HPLC (0.1% TFA in H₂O/MeCN) to afford the Lu or La-cold standard. Isolated amounts ranged from 3-8 mg, and purities (HPLC) were between 95% and 99%.

General Methods of Radiolabeling with ¹⁷⁷Lu:

Generally, a small aliquot of the precursor (1-5 μL, 1-3 nmol) was dissolved in the reaction buffer/solution with a known pH. To the solution was added a known amount/activity of ¹⁷⁷LuCl₃. Then, the reaction was heated to 85° C. for 20-30 minutes. At this point, the reaction was cooled down and further diluted with the suitable formulation or dilution buffer. For analysis, the reaction mixtures and/or diluted solutions were analyzed using radio-TLC (iTLC) and radio-HPLC. Generally, iTLCs were run on SG paper plates, with 0.1 M sodium citrate as the eluent and other optional solvents as needed. The developed plates were then read using a radio-TLC reading instrument. HPLC analyses were performed using a suitable C18 column, with different combinations of H₂O and MeCN (both with 0.1% TFA) as the eluent. The eluting components were visualized by UV (DAD) and radio-detection flow monitors. In all cases, the radiochemical purity of the radiolabeled compound was assessed at the end of synthesis (EOS) and after 3 h (estimated time between end of synthesis and in vivo experiments).

General Methods of Radiolabeling with ²²⁵Ac:

Generally, a small aliquot of the precursor (1-10 μL, 1-10 nmol) was dissolved in the reaction buffer/solution with a known pH. To the solution was added a known amount/activity of ²²⁵AcCl₃ or ²²⁵Ac(NO₃)₃ Then, the reaction was heated to 95° C. for 15-30 minutes. At this point, the reaction was cooled down and further diluted with the suitable formulation or dilution buffer. For analysis, the reaction mixtures and/or diluted solutions were analyzed using radio-TLC (iTLC) and radio-HPLC. Generally, iTLCs were run on SG paper plates, with 0.1 M sodium citrate as the eluent and other optional solvents as needed. The developed plates were then read using a radio-TLC reading instrument. HPLC analyses were performed using a suitable C18 column, with different combinations of H₂O and MeCN (both with 0.1% TFA) as the eluent. The eluting components were visualized by UV (DAD) and radio-detection flow monitors. To detect the emissions resulting from the decay chain of ²²⁵Ac, all collected fractions from HPLC were allowed to equilibrate for 6 h, then were placed in a Gamma counting instrument for subsequent measurements. In all cases, the radiochemical purity of the radiolabeled compound was assessed at the end of synthesis (EOS) and after 3-5 h (estimated time between end of synthesis and in vivo experiments).

### Example 1: Synthesis

### Synthesis of Compound 1

Sieber resin was deprotected by treating the resin with 20% piperidine in DMF (3×8 min). Fmoc-Leu-ψ-Pro-OH was coupled to the resin via solid-phase peptide synthesis using Fmoc-based chemistry. The coupling was carried out in DMF using Fmoc-Leu-ψ-Pro-OH (4 eq.), HATU (4 eq.), HOAt (4 eq.) and DIEPA (11 eq.) at room temperature for overnight (16 hours). At a 0.15 mmol scale, the preloaded Fmoc-Leu-ψ-Pro-Sieber resin was then loaded onto the peptide synthesizer, and the Fmoc was deprotected with 10% Piperidine in DMF at 45° C. (3 min×3). Then, Fmoc-His(Boc)-OH (4 eq., 0.4 mmol, 0.2 M in DMF) was coupled at 45° C. (10 min) using 1 M DIC/1 M Oxyma in DMF (1 mL/0.5 mL), followed by Fmoc-deprotection as described above. Fmoc-Gly-OH, Fmoc-Tle-OH, Fmoc-Ala-OH (x2), Fmoc-αMeTrp(Boc)-OH, Fmoc-Gln(Trt)-OH (x2), and Fmoc-D-Phe-OH were coupled consecutively in a similar manner, except Fmoc-Gln(Trt)-OH was using HATU/HOAt/DIEPA(4/4/11 eq.) as coupling reagents. The synthesis continued with the addition of the linker, Fmoc-4-amino-(1-carboxymethyl) piperidine (4 eq.), at 45° C. for 1.5 h x2 and then followed by the chelator DOTA(tBu)₃ (4 eq) at 25° C. for 18 h. The coupling of the linker and the chelator were carried out by using HATU/HOAt/DIPEA (4/4/11 eq.). The peptide was then deprotected and simultaneously cleaved from the resin by treating with 92.5/2.5/2.5/2.5 TFA/TIS/DODT/H2O for 50 min at 40° C. After filtration, the crude peptide was precipitated by the addition of cold diethyl ether to the TFA solution and lyophilized in a H₂O/MeCN mixture. The crude peptide was purified with prep HPLC using solvents A: H₂O/0.1% TFA and B: MeCN/0.1% TFA, 20 mL/min (21%-31% B over 7 min, tR=6.5 min). The sample was lyophilized to a white solid. Calc mass [M+2H]²⁺/2: 797.45 m/z; found [M+2H]²⁺/2: 797.50 m/z.

Other compounds were synthesized in similar manner. Table 1 lists the LC-MS data for the synthesized compounds.

TABLE 1
Mass values obtained from LC-MS spectrum
of illustrative compounds

| | Calculated mass | Found |
|---|---|---|
| Compound | ([M + 2H]²⁺, m/z) | ([M + 2H]²⁺, m/z) |
| 1 | 797.45 | 797.5 |
| 2 | 841.45 | 841.7 |
| 3 | 871.49 | 871.8 |
| 4 | 878.5 | 878.8 |
| 5 | 833.45 | 833.8 |
| 6 | 814.94 | 815.2 |
| 7 | 864.48 | 864.8 |
| 23 | 839.95 | 840.3 |
| 8 | 865.98 | 886.1 |
| 9 | 821.95 | 822.3 |
| 10 | 830.95 | 831.3 |
| 11 | 922.52 | 922.8 |
| 14 | 905.00 | 905.5 |
| 17 | 842.45 | 842.7 |
| 24 | 898.01 | 898.3 |
| 19 | 855.98 | 856.3 |
| 20 | 841.96 | 842.3 |
| 22 | 804.45 | 804.7 |
| 35 | 1046.6 | 1046.9 |
| | Calculated mass | Found |
| Compound | ([M + 3H]³⁺, m/z) | ([M + 3H]³⁺, m/z) |
| 34 | 652.37 | 652.7 |
| 36 | 973.86 | 974.1 |
| | Calculated mass | Found |
| Compound | ([M + 4H]⁴⁺, m/z) | ([M + 4H]⁴⁺, m/z) |
| 37 | 822.2 | 822.7 |

### Synthesis of Lu-Compound 1

The precursor of compound 1 (7.4 mg, 4.6 μmol) was dissolved in 600 μL 0.2 NaOAc buffer (4.5 pH) and to it was added 5 eq of LuCl₃ (23 μmol). The solution was heated to 90° C. for 30 minutes and the reaction mixture was then purified by HPLC using the semipreparative column. The HPLC conditions were 24% acetonitrile in water with 0.1% TFA at a flow rate of 4 mL/min (t_{R}=8.3 min). The HPLC eluates containing the desired peptide were collected, pooled, and lyophilized. The yield was 65% with 95% purity. ESI-MS: calculated [M+2H]²⁺ for Lu-Compound 1 C₇₇H₂₁₅N₂₀O₁₇[Lu]883.4; found [M+2H]²+883.7.

### Synthesis of La-Compound 1

The precursor of compound 1 (5.6 mg, 3.5 μmol) was dissolved in 500 μL 0.2 NaOAc buffer (4.5 pH) and to it was added 5 eq of LaCl₃ (23 μmol). The solution was heated to 90° C. for 30 minutes and the reaction mixture was then purified by HPLC using the analytic column. The HPLC conditions were 23% acetonitrile in water with 0.1% TFA at a flow rate of 1 mL/min (t_{R}=10.6 min). The HPLC eluates containing the desired peptide were collected, pooled, and lyophilized. The yield was 29% with 97% purity. ESI-MS: calculated [M+2H]²⁺ for La-Compound 1 C₇₇H₂₁₅N₂₀O₁₇[La]865.4; found [M+2H]²⁺865.7.

### Radiolabelling of ¹⁷⁷Lu-Compound 1

2.4 μL aliquot of compound 1 precursor (1 mM, 2.4 nmol) was dissolved in 205.3 μL 0.2 M NaOAc (pH 5.5), followed by 8.3 μL, 126.9 MBq of ¹⁷⁷LuCl₃. Then, the reaction was heated to 95° C. for 15 minutes. After cooling down, the reaction mixture was further diluted with the formulation buffer. For quality control, the formulated solutions were analyzed using radio-TLC (iTLC) and radio-HPLC. iTLCs were run on SG paper plates, with 0.1 M sodium citrate (5% MeOH) as the eluent. The developed plates were then read using a radio-TLC reading instrument. HPLC analyses were performed using an analytical Luna column, the mobile phase condition was using gradient from 5 to 85% acetonitrile in water with 0.1% TFA in 15 minutes at a flow rate of 1 mL/min (t_{R}=10.8 min). The eluting components were visualized by UV (DAD) and radio-detection flow monitors. The radiochemical purity of ¹⁷⁷Lu-compound 1 at EOS (end of synthesis) and after 3 h were >99% by iTLC and HPLC with molar activity 44.57 MBq/nmol.

### Radiolabelling of ²²⁵Ac-Compound 1

5 μL aliquot of compound 1 precursor (1 mM, 5 nmol) was dissolved in 138 μL 0.2 M NaOAc (pH 5.5) and 20 μL EtOH (10%), followed by 37.3 μL, 1.06 MBq of ²²⁵AcCl₃. Then, the reaction was heated to 95° C. for 15 minutes. After cooling down, the reaction mixture was diluted with 3800 μL formulation buffer. For quality control, the formulated solutions were analyzed using radio-TLC (iTLC) and radio-HPLC. iTLCs were run on SG paper plates, with 0.1 M sodium citrate (5% MeOH) as the eluent. The developed plates were then read using a radio-TLC reading instrument. HPLC analyses were performed using an analytical Luna column with fraction collection. The mobile phase condition was using gradient from 5 to 85% acetonitrile in water with 0.1% TFA in 15 minutes at a flow rate of 1 mL/min (t_{R}=10.8 min). The eluting components were visualized by UV (DAD) and radio-detection flow monitors. To detect the emissions resulting from the decay chain of ²²⁵Ac, all collected fractions from HPLC were allowed to equilibrate for 6 h, then were placed in a Gamma counting instrument for subsequent measurements. The radiochemical purity of ²²⁵Ac-compound 1 at EOS (end of synthesis) and after 3 h was >97% by iTLC and >92% by HPLC with molar activity of 0.17 MBq/nmol.

### Example 2: In Vitro Competition Binding Assay

PC-3 cells were seeded at 2×10⁵ cells/well in 24-well poly-D-lysine plates 24-48 hours prior to the experiment. The growth medium was replaced by 400 μL of reaction medium (RPMI 1640 containing 2 mg/mL BSA, 4.8 mg/mL HEPES, 1 U/mL penicillin G and 1 μg/mL streptomycin). Cells were incubated for 30-60 min at 37° C. Peptides as provided in Table 9 below provided in 50 μL of decreasing concentrations (10 μM to 1 pM) and 50 μL of 0.011 nM [¹²⁵1-Tyr⁴]bombesin were added to wells. The cells were incubated with moderate agitation for 1 h at 27° C., washed twice with ice-cold PBS, harvested by trypsinization, and measured for radioactivity on the gamma counter. Data were analyzed using nonlinear regression (one binding site model for competition assay) with GraphPad Prism 8.

TABLE 2
Binding affinities (Ki, nM) of GRPR-targeting peptides

| Compound | Binding affinities Ki (n = 1-4) |
|---|---|
| La-Compound 1 | 7.80 ± 3.30 |
| Lu-Compound 1 | 11.64 ± 6.69 |
| La-Compound 2 | 62.47 ± 31.47 |
| La-Compound 3 | 8.70 ± 0.39 |
| La-Compound 4 | 15.83 ± 7.09 |
| La-Compound 5 | 46.02 ± 16.08 |
| Lu-Compound 5 | 20.28 ± 4.36 |
| La-Compound 6 | 4.54 ± 0.78 |
| La-Compound 7 | 1.71 ± 0.46 |
| La-Compound 23 | 18.31 ± 10.05 |
| La-Compound 8 | 1.05 ± 0.52 |
| La-Compound 19 | 9.25 ± 3.27 |
| La-Compound 9 | 1.71 ± 0.95 |
| Lu-Compound 9 | 3.38 ± 0.55 |
| La-Compound 10 | 1.78 ± 1.19 |
| Lu-Compound 10 | 5.03 ± 0.27 |
| La-Compound 11 | 1.12 ± 0.26 |
| La-Compound 14 | >100 |
| La-Compound 17 | >100 |
| La-Compound 20 | 94.45 ± 49.12 |
| La-Compound 24 | 9.26 ± 6.39 |
| La-Compound 34 | 2.00 ± 0.68 |
| Lu-Compound 34 | 7.62 ± 4.45 |
| La-Compound 35 | 3.81 ± 1.93 |
| La-Compound 36 | 12.39 ± 2.21 |
| Lu-Compound 36 | 6.51 ± 2.20 |

### Example 3: Ex Vivo Biodistribution in PC-3 Tumor-Bearing Mice

Biodistribution studies were performed using male NOD.Cg-Rag1^{tm1/Mom}II2rg^{tm1Wjl}/SzJ (NRG) mice and conducted according to the guidelines established by the Canadian Council on Animal Care and approved by Animal Ethics Committee of the University of British Columbia. For tumor inoculations, mice were anesthetized by inhalation with 2% isoflurane in oxygen and implanted subcutaneously with 5×10⁶ PC-3 cells below the left shoulder. Imaging and biodistribution studies were performed only after tumors grew to 5-8 mm in diameter.

For ex vivo biodistribution studies, mice were injected with ~20 kBq of the ²²⁵Ac-labeled tracer. Mice were euthanized at various times (1h, 4 h, 24h, 48h, 72h, 96h, and/or 120 h p.i.), blood was drawn from heart, and organs/tissues of interest were collected, rinsed with PBS, blotted dry, weighed, and counted using an automated gamma counter. The uptake in each organ/tissue was normalized to the injected dose and expressed as the percentage of the injected dose per gram of tissue (% ID/g).

Biodistribution data is shown in Tables 3-11.

TABLE 3
Biodistribution data (average % ID/g with ±SD at 1 h, 4 h, 24 h, 48 h, and 120 h
post-injection) of ²²⁵Ac-Compound 1 in mice bearing PC-3 tumor xenografts (8.15 ± 0.29 kBq).

| | Time (h) | | | | | | | | | | | | | | |
|---|---|---|---|---|---|---|---|---|---|---|---|---|---|---|---|
| | 1 | | | 4 | | | 24 | | | 48 | | | 120 | | |
| Organs | AVG | SD | n | AVG | SD | n | AVG | SD | n | AVG | SD | n | AVG | SD | n |
| Blood | 0.37 | 0.06 | 4 | 0.11 | 0.03 | 4 | — | — | — | — | — | — | — | — | — |
| Tumor | 10.87 | 1.01 | 4 | 14.23 | 3.90 | 4 | 17.09 | 2.41 | 4 | 10.71 | 1.98 | 4 | 3.41 | 0.70 | 4 |
| Pancreas | 57.60 | 9.16 | 4 | 38.62 | 7.54 | 4 | 4.86 | 0.80 | 4 | 3.29 | 0.69 | 4 | 2.05 | 0.41 | 4 |
| Kidneys | 2.11 | 0.45 | 4 | 1.74 | 0.19 | 4 | 0.85 | 0.10 | 4 | 0.51 | 0.17 | 4 | 0.22 | 0.08 | 4 |
| Liver | 0.41 | 0.04 | 4 | 0.42 | 0.10 | 4 | 0.37 | 0.06 | 4 | 0.36 | 0.07 | 4 | 0.43 | 0.08 | 4 |
| Small intestine | 3.57 | 1.64 | 4 | 2.74 | 0.63 | 4 | 0.32 | 0.09 | 4 | 0.12 | 0.04 | 4 | 0.06 | 0.01 | 3 |
| Large intestine | 3.41 | 0.15 | 4 | 2.31 | 0.54 | 4 | 0.76 | 0.20 | 4 | 0.29 | 0.08 | 4 | 0.10 | 0.03 | 4 |
| Tumor/Blood | 29.92 | 2.78 | 4 | 133.28 | 44.76 | 4 | — | — | — | — | — | — | — | — | — |
| Tumor/Kidneys | 5.27 | 0.74 | 4 | 8.09 | 1.53 | 4 | 20.00 | 1.93 | 4 | 21.53 | 2.93 | 4 | 16.40 | 2.99 | 4 |
| Tumor/liver | 26.74 | 3.16 | 4 | 33.59 | 6.29 | 4 | 46.39 | 2.78 | 4 | 30.33 | 2.85 | 4 | 7.91 | 0.25 | 4 |
| Tumor/pancreas | 0.19 | 0.02 | 4 | 0.37 | 0.05 | 4 | 3.54 | 0.34 | 4 | 3.27 | 0.15 | 4 | 1.66 | 0.03 | 4 |

TABLE 4
Biodistribution data (average % ID/g with ±SD at 1 h, 4 h, 24 h, 48 h, and 120 h
post-injection) of ²²⁵Ac-Compound 4 in mice bearing PC-3 tumor xenografts (8.10 ± 1.42 kBq).

| | Time (h) | | | | | | | | | | | | | | |
|---|---|---|---|---|---|---|---|---|---|---|---|---|---|---|---|
| | 1 | | | 4 | | | 24 | | | 48 | | | 120 | | |
| Organs | AVG | SD | n | AVG | SD | n | AVG | SD | n | AVG | SD | n | AVG | SD | n |
| Blood | 0.29 | 0.09 | 4 | 0.08 | 0.04 | 4 | — | — | — | — | — | — | — | — | — |
| Tumor | 9.60 | 2.84 | 4 | 12.15 | 2.92 | 4 | 11.90 | 2.85 | 4 | 6.64 | 2.69 | 4 | 2.67 | 0.80 | 4 |
| Pancreas | 24.51 | 5.58 | 4 | 8.62 | 1.54 | 4 | 0.83 | 0.15 | 4 | 1.00 | 0.33 | 4 | 0.79 | 0.22 | 4 |
| Kidneys | 1.80 | 0.44 | 4 | 2.05 | 0.52 | 4 | 0.93 | 0.14 | 4 | 0.80 | 0.25 | 4 | 0.48 | 0.17 | 4 |
| Liver | 0.28 | 0.05 | 4 | 0.31 | 0.08 | 4 | 0.48 | 0.06 | 4 | 0.60 | 0.24 | 4 | 0.84 | 0.22 | 4 |
| Small intestine | 1.90 | 0.47 | 4 | 1.05 | 0.17 | 4 | 0.13 | 0.04 | 4 | 0.08 | 0.02 | 4 | 0.06 | 0.03 | 4 |
| Large intestine | 1.81 | 0.40 | 4 | 1.02 | 0.28 | 3 | 0.41 | 0.01 | 3 | 0.12 | 0.04 | 4 | 0.15 | 0.10 | 4 |
| Tumor/Blood | 34.89 | 8.76 | 4 | 184.46 | 103.09 | 4 | — | — | — | — | — | — | — | — | — |
| Tumor/Kidneys | 5.32 | 0.52 | 4 | 5.99 | 0.76 | 4 | 12.64 | 1.71 | 4 | 8.26 | 1.75 | 4 | 5.68 | 0.43 | 4 |
| Tumor/liver | 34.57 | 8.51 | 4 | 38.99 | 2.44 | 4 | 24.60 | 5.08 | 4 | 11.17 | 2.18 | 4 | 3.17 | 0.15 | 4 |
| Tumor/pancreas | 0.39 | 0.05 | 4 | 1.40 | 0.10 | 4 | 14.25 | 2.14 | 4 | 6.58 | 0.46 | 4 | 3.41 | 0.45 | 4 |

TABLE 5
Biodistribution data (average % ID/g with ±SD at 1 h, 4 h, 24 h, 48 h, and 96 h post-
injection) of ²²⁵Ac-Compound 36 in mice bearing PC-3 tumor xenografts (8.59 ± 0.49 kBq).

| | Time (h) | | | | | | | | | | | | | | |
|---|---|---|---|---|---|---|---|---|---|---|---|---|---|---|---|
| | 1 | | | 4 | | | 24 | | | 48 | | | 96 | | |
| Organs | AVG | SD | n | AVG | SD | n | AVG | SD | n | AVG | SD | n | AVG | SD | n |
| Blood | 1.54 | 0.24 | 4 | 0.13 | 0.05 | 4 | — | — | — | — | — | — | — | — | — |
| Tumor | 7.61 | 1.11 | 4 | 8.07 | 0.77 | 4 | 4.51 | 0.51 | 4 | 2.70 | 0.27 | 4 | 0.98 | 0.05 | 4 |
| Pancreas | 14.51 | 1.92 | 4 | 2.05 | 0.22 | 4 | 0.59 | 0.07 | 4 | 0.60 | 0.13 | 4 | 0.37 | 0.06 | 4 |
| Kidneys | 6.82 | 1.10 | 4 | 5.21 | 0.33 | 4 | 2.51 | 0.35 | 4 | 1.26 | 0.13 | 4 | 0.69 | 0.11 | 4 |
| Liver | 11.85 | 2.18 | 4 | 9.42 | 0.68 | 4 | 7.44 | 0.42 | 4 | 5.94 | 0.68 | 4 | 6.56 | 0.33 | 4 |
| Small intestine | 2.14 | 0.61 | 4 | 0.60 | 0.15 | 4 | 0.16 | 0.02 | 4 | 0.14 | 0.05 | 4 | 0.04 | 0.00 | 3 |
| Large intestine | 1.52 | 0.38 | 4 | 0.99 | 0.25 | 4 | 0.46 | 0.10 | 4 | 0.29 | 0.11 | 4 | 0.12 | 0.03 | 4 |
| Tumor/Blood | 4.95 | 0.48 | 4 | 70.13 | 27.96 | 4 | — | — | — | — | — | — | — | — | — |
| Tumor/Kidneys | 1.12 | 0.12 | 4 | 1.55 | 0.12 | 4 | 1.80 | 0.09 | 4 | 2.16 | 0.31 | 4 | 1.46 | 0.22 | 4 |
| Tumor/liver | 0.65 | 0.05 | 4 | 0.86 | 0.03 | 4 | 0.60 | 0.05 | 4 | 0.46 | 0.07 | 4 | 0.15 | 0.01 | 4 |
| Tumor/pancreas | 0.53 | 0.06 | 4 | 3.95 | 0.22 | 4 | 7.71 | 0.86 | 4 | 4.71 | 1.28 | 4 | 2.73 | 0.42 | 4 |

TABLE 6
Biodistribution data (average % ID/g with ±SD at 1 h, 4 h, 23 h, 102 h, and 120 h
post-injection) of ²²⁵Ac-Compound 2 in mice bearing PC-3 tumor xenografts (19.71 ± 0.81 kBq).

| | Time (h) | | | | | | | | | | | | | | |
|---|---|---|---|---|---|---|---|---|---|---|---|---|---|---|---|
| | 1 | | | 4 | | | 23 | | | 102 | | | 120 | | |
| Organs | AVG | SD | n | AVG | SD | n | AVG | SD | n | AVG | SD | n | AVG | SD | n |
| Blood | 0.68 | 0.11 | 4 | — | — | — | — | — | — | — | — | — | — | — | — |
| Tumor | 9.45 | 1.52 | 4 | 7.79 | 0.95 | 4 | 2.32 | 0.11 | 4 | 0.56 | 0.08 | 4 | 0.54 | 0.11 | 4 |
| Muscle | 0.18 | 0.06 | 2 | — | — | — | — | — | — | — | — | — | — | — | — |
| Pancreas | 6.17 | 0.97 | 4 | 0.52 | 0.07 | 4 | 0.22 | 0.09 | 3 | 0.20 | 0.00 | 2 | — | — | — |
| Kidneys | 2.90 | 0.19 | 4 | 1.99 | 0.31 | 4 | 0.93 | 0.07 | 4 | 036 | 0.03 | 4 | 0.36 | 0.08 | 4 |
| Liver | 0.49 | 0.05 | 4 | 0.48 | 0.06 | 4 | 0.53 | 0.04 | 4 | 0.63 | 0.06 | 4 | 0.56 | 0.04 | 4 |
| Small intestine | 0.89 | 0.20 | 4 | 0.32 | 0.17 | 4 | 0.04 | 0.01 | 4 | — | — | — | 0.02 | 0.00 | 2 |
| Large intestine | 0.76 | 0.13 | 4 | 0.16 | 0.03 | 4 | 0.11 | 0.06 | 4 | — | — | — | — | — | — |
| Tumor/Muscle | 54.93 | 15.73 | 2 | — | — | — | — | — | — | — | — | — | — | — | — |
| Tumor/Blood | 14.30 | 3.59 | 4 | — | — | — | — | — | — | — | — | — | — | — | — |
| Tumor/Kidneys | 3.25 | 0.44 | 4 | 3.95 | 0.34 | 4 | 2.50 | 0.27 | 4 | 1.55 | 0.20 | 4 | 1.50 | 0.10 | 4 |
| Tumor/liver | 19.16 | 1.63 | 4 | 16.32 | 1.01 | 4 | 4.37 | 0.38 | 4 | 0.90 | 0.14 | 4 | 0.97 | 0.14 | 4 |
| Tumor/pancreas | 1.55 | 0.26 | 4 | 15.27 | 3.96 | 4 | 11.54 | 4.02 | 3 | 2.75 | 0.61 | 2 | — | — | — |

TABLE 7
Biodistribution data (average % ID/g with ±SD at 1 h, 4 h, 23 h, 46 h, and 166 h
post-injection) of ²²⁵Ac-Compound 3 in mice bearing PC-3 tumor xenografts (9.10 ± 0.36 kBq).

| | Time (h) | | | | | | | | | | | | | | |
|---|---|---|---|---|---|---|---|---|---|---|---|---|---|---|---|
| | 1 | | | 4 | | | 23 | | | 46 | | | 166 | | |
| Organs | AVG | SD | n | AVG | SD | n | AVG | SD | n | AVG | SD | n | AVG | SD | n |
| Blood | 0.51 | 0.05 | 4 | 0.09 | 0.01 | 4 | — | — | — | — | — | — | — | — | — |
| Tumor | 15.36 | 0.78 | 4 | 14.14 | 2.16 | 4 | 10.10 | 1.18 | 4 | 5.90 | 1.37 | 4 | 1.22 | 0.38 | 4 |
| Muscle | 0.21 | 0.02 | 3 | — | — | — | — | — | — | — | — | — | — | — | — |
| Spleen | 0.92 | 0.34 | 4 | — | — | — | — | — | — | — | — | — | — | — | — |
| Pancreas | 23.59 | 2.66 | 4 | 5.56 | 1.79 | 4 | 0.68 | 0.10 | 4 | 0.63 | 0.23 | 4 | 0.59 | 0.16 | 4 |
| Kidneys | 3.10 | 0.46 | 4 | 2.37 | 0.22 | 4 | 1.25 | 0.09 | 4 | 0.92 | 0.17 | 4 | 0.38 | 0.06 | 4 |
| Liver | 0.39 | 0.05 | 4 | 0.27 | 0.03 | 4 | 0.39 | 0.02 | 4 | 0.50 | 0.12 | 4 | 0.74 | 0.26 | 4 |
| Small intestine | 2.68 | 0.39 | 4 | 1.08 | 0.38 | 4 | 0.09 | 0.03 | 4 | 0.06 | 0.02 | 4 | 0.04 | 0.01 | 4 |
| Large intestine | 1.78 | 0.12 | 4 | 0.66 | 0.15 | 4 | 0.18 | 0.07 | 4 | 0.12 | 0.01 | 4 | 0.08 | 0.03 | 4 |
| Tumor/Muscle | 76.26 | 4.70 | 3 | — | — | — | — | — | — | — | — | — | — | — | — |
| Tumor/Blood | 30.26 | 2.87 | 4 | 163.79 | 33.44 | 4 | — | — | — | — | — | — | — | — | — |
| Tumor/Kidneys | 5.03 | 0.74 | 4 | 5.96 | 0.44 | 4 | 8.14 | 1.05 | 4 | 6.39 | 0.65 | 4 | 3.18 | 0.58 | 4 |
| Tumor/liver | 39.67 | 4.46 | 4 | 51.27 | 3.62 | 4 | 25.76 | 1.96 | 4 | 12.02 | 1.77 | 4 | 1.91 | 0.24 | 4 |
| Tumor/pancreas | 0.66 | 0.08 | 4 | 2.64 | 0.44 | 4 | 14.92 | 0.69 | 4 | 9.92 | 2.14 | 4 | 2.32 | 0.34 | 4 |

TABLE 8
Biodistribution data (average % ID/g with ±SD at 1 h, 4 h, 24 h, 95 h, and 117 h
post-injection) of ²²⁵Ac-Compound 8 in mice bearing PC-3 tumor xenografts (6.71 ± 0.31 kBq).

| | Time (h) | | | | | | | | | | | | | | |
|---|---|---|---|---|---|---|---|---|---|---|---|---|---|---|---|
| | 1 | | | 4 | | | 24 | | | 95 | | | 117 | | |
| Organs | AVG | SD | n | AVG | SD | n | AVG | SD | n | AVG | SD | n | AVG | SD | n |
| Blood | 2.98 | 0.22 | 4 | 0.41 | 0.04 | 4 | — | — | — | — | — | — | 0.06 | 0.01 | 2 |
| Tumor | 16.71 | 2.62 | 4 | 19.59 | 2.62 | 4 | 10.17 | 2.84 | 4 | 3.38 | 0.51 | 4 | 2.66 | 0.01 | 3 |
| Muscle | 0.21 | 0.02 | 4 | — | — | — | — | — | — | — | — | — | 0.30 | 0.12 | 2 |
| Spleen | 1.00 | 0.24 | 3 | — | — | — | — | — | — | — | — | — | 1.92 | 0.58 | 3 |
| Pancreas | 34.51 | 3.18 | 4 | 7.29 | 0.95 | 4 | 1.15 | 0.38 | 3 | 1.00 | 0.15 | 4 | 1.17 | 0.23 | 4 |
| Kidneys | 6.19 | 0.47 | 4 | 3.21 | 0.13 | 4 | 1.80 | 0.33 | 4 | 0.95 | 0.07 | 4 | 0.73 | 0.14 | 4 |
| Liver | 4.35 | 0.37 | 4 | 2.56 | 0.24 | 4 | 2.24 | 0.37 | 4 | 2.66 | 0.26 | 4 | 2.47 | 0.35 | 4 |
| Small intestine | 3.45 | 0.17 | 4 | 0.92 | 0.16 | 4 | 0.16 | 0.03 | 4 | 0.09 | 0.02 | 4 | 0.12 | 0.02 | 4 |
| Large intestine | 3.02 | 0.44 | 4 | 1.04 | 0.07 | 4 | 0.27 | 0.04 | 4 | 0.17 | 0.03 | 4 | 0.25 | 0.03 | 4 |
| Tumor/Muscle | 69.96 | 0.58 | 4 | — | — | — | — | — | — | — | — | — | 9.92 | 4.22 | 2 |
| Tumor/Blood | 5.60 | 0.72 | 4 | 48.12 | 8.10 | 4 | — | — | — | — | — | — | 41.37 | 13.80 | 2 |
| Tumor/Kidneys | 2.69 | 0.31 | 4 | 6.09 | 0.64 | 4 | 5.60 | 0.79 | 4 | 3.55 | 0.41 | 4 | 3.39 | 0.32 | 3 |
| Tumor/liver | 3.83 | 0.34 | 4 | 7.66 | 1.04 | 4 | 4.47 | 0.57 | 4 | 1.27 | 0.19 | 4 | 1.01 | 0.06 | 3 |
| Tumor/pancreas | 0.48 | 0.07 | 4 | 2.69 | 0.05 | 4 | 8.50 | 0.22 | 3 | 3.37 | 0.36 | 4 | 2.11 | 0.20 | 3 |

TABLE 9
Biodistribution data (average % ID/g with ±SD at 1 h, 4 h, 23 h, 101 h, and 137 h
post-injection) of ²²⁵Ac-Compound 10 in mice bearing PC-3 tumor xenografts (16.93 ± 0.35 kBq).

| | Time (h) | | | | | | | | | | | | | | |
|---|---|---|---|---|---|---|---|---|---|---|---|---|---|---|---|
| | 1 | | | 4 | | | 23 | | | 101 | | | 137 | | |
| Organs | AVG | SD | n | AVG | SD | n | AVG | SD | n | AVG | SD | n | AVG | SD | n |
| Blood | 2.93 | 0.24 | 4 | 1.00 | 0.17 | 4 | 0.06 | 0.01 | 3 | — | — | — | — | — | — |
| Tumor | 10.76 | 1.25 | 4 | 18.70 | 1.97 | 4 | 23.33 | 1.98 | 4 | 9.82 | 0.80 | 4 | 6.73 | 0.61 | 4 |
| Muscle | 0.28 | 0.09 | 4 | 0.10 | 0.02 | 3 | — | — | — | — | — | — | — | — | — |
| Spleen | 0.92 | 0.14 | 4 | 0.37 | 0.13 | 3 | 0.46 | 0.12 | 2 | — | — | — | 0.62 | 0.22 | 3 |
| Pancreas | 63.20 | 4.02 | 4 | 42.59 | 4.67 | 4 | 6.96 | 0.60 | 4 | 3.67 | 0.23 | 4 | 3.48 | 1.05 | 4 |
| Kidneys | 5.17 | 0.39 | 4 | 3.18 | 0.32 | 4 | 1.16 | 0.09 | 4 | 0.46 | 0.00 | 3 | 0.39 | 0.05 | 4 |
| Liver | 4.98 | 0.25 | 4 | 3.82 | 0.22 | 4 | 3.26 | 0.12 | 4 | 2.51 | 0.17 | 4 | 2.71 | 0.31 | 4 |
| Small intestine | 4.08 | 0.33 | 4 | 3.18 | 0.48 | 4 | 0.63 | 0.10 | 4 | 0.16 | 0.02 | 4 | 0.14 | 0.00 | 3 |
| Large intestine | 3.09 | 0.76 | 4 | 2.19 | 0.20 | 4 | 1.10 | 0.09 | 4 | 0.31 | 0.03 | 4 | 0.27 | 0.08 | 4 |
| Tumor/Muscle | 41.37 | 10.55 | 4 | 187.24 | 39.67 | 3 | — | — | — | — | — | — | — | — | — |
| Tumor/Blood | 3.69 | 0.44 | 4 | 18.89 | 1.91 | 4 | 390.35 | 40.65 | 3 | — | — | — | — | — | — |
| Tumor/Kidneys | 2.09 | 0.28 | 4 | 5.89 | 0.18 | 4 | 20.18 | 1.92 | 4 | 21.27 | 2.06 | 3 | 17.45 | 2.56 | 4 |
| Tumor/liver | 2.16 | 0.27 | 4 | 4.89 | 0.45 | 4 | 7.15 | 0.53 | 4 | 3.91 | 0.34 | 4 | 2.51 | 0.40 | 4 |
| Tumor/pancreas | 0.17 | 0.02 | 4 | 0.44 | 0.02 | 4 | 3.36 | 0.14 | 4 | 2.68 | 0.27 | 4 | 2.11 | 0.87 | 4 |

TABLE 10
Biodistribution data (average % ID/g with ±SD at 1 h, 4 h, 26 h, 48 h, and 118 h post-
injection) of ²²⁵Ac-Compound 19 in mice bearing PC-3 tumor xenografts (20.79 ± 0.34 kBq).

| | Time (h) | | | | | | | | | | | | | | |
|---|---|---|---|---|---|---|---|---|---|---|---|---|---|---|---|
| | 1 | | | 4 | | | 26 | | | 48 | | | 118 | | |
| Organs | AVG | SD | n | AVG | SD | n | AVG | SD | n | AVG | SD | n | AVG | SD | n |
| Blood | 0.62 | 0.08 | 4 | 0.07 | 0.01 | 4 | 0.02 | 0.00 | 2 | — | — | — | — | — | — |
| Tumor | 10.91 | 0.47 | 4 | 4.88 | 0.02 | 4 | 1.47 | 0.24 | 4 | 1.05 | 0.11 | 4 | 0.40 | 0.05 | 4 |
| Muscle | 0.16 | 0.03 | 4 | — | — | — | — | — | — | — | — | — | 0.10 | 0.00 | 2 |
| Spleen | 0.61 | 0.25 | 3 | 0.50 | 0.01 | 2 | — | — | — | — | — | — | 0.49 | 0.09 | 3 |
| Pancreas | 8.63 | 1.47 | 4 | 0.95 | 0.27 | 4 | 0.34 | 0.10 | 4 | 0.37 | 0.05 | 4 | 0.35 | 0.06 | 4 |
| Kidneys | 3.20 | 0.25 | 4 | 2.21 | 0.24 | 4 | 1.13 | 0.24 | 4 | 1.02 | 0.20 | 4 | 0.52 | 0.09 | 4 |
| Liver | 0.72 | 0.06 | 4 | 0.63 | 0.06 | 4 | 0.64 | 0.13 | 4 | 0.87 | 0.23 | 4 | 0.68 | 0.13 | 4 |
| Small intestine | 1.24 | 0.25 | 4 | 0.42 | 0.23 | 4 | 0.08 | 0.02 | 4 | 0.05 | 0.02 | 4 | 0.03 | 0.00 | 4 |
| Large intestine | 1.04 | 0.12 | 4 | 0.36 | 0.08 | 4 | 0.32 | 0.10 | 4 | 0.09 | 0.02 | 4 | 0.04 | 0.01 | 4 |
| Tumor/Muscle | 68.30 | 14.50 | 4 | — | — | — | — | — | — | — | — | — | 4.58 | 0.29 | 2 |
| Tumor/Blood | 17.84 | 1.91 | 4 | 75.14 | 16.60 | 3 | 90.54 | 24.52 | 2 | — | — | — | — | — | — |
| Tumor/Kidneys | 3.42 | 0.25 | 4 | 2.33 | 0.13 | 3 | 1.31 | 0.07 | 4 | 1.06 | 0.21 | 4 | 0.79 | 0.09 | 4 |
| Tumor/liver | 15.25 | 1.19 | 4 | 8.13 | 0.77 | 3 | 2.32 | 0.11 | 4 | 1.24 | 0.18 | 4 | 0.60 | 0.06 | 4 |
| Tumor/pancreas | 1.29 | 0.20 | 4 | 5.99 | 0.80 | 3 | 4.49 | 0.62 | 4 | 2.88 | 0.36 | 4 | 1.20 | 0.03 | 3 |

TABLE 11
Biodistribution data (average % ID/g with ± SD at 1 h, 4 h, 22 h, 45 h, and 165 h
post-injection) of ²²⁵Ac-Compound 34 in mice bearing PC-3 tumor xenografts (16.8 ± 0.18 kBq).

| | Time (h) | | | | | | | | | | | | | | |
|---|---|---|---|---|---|---|---|---|---|---|---|---|---|---|---|
| | 1 | | | 4 | | | 22 | | | 45 | | | 165 | | |
| Organs | AVG | SD | n | AVG | SD | n | AVG | SD | n | AVG | SD | n | AVG | SD | n |
| Blood | 6.13 | 0.49 | 4 | 2.95 | 0.49 | 4 | 0.16 | 0.09 | 4 | 0.02 | 0.00 | 3 | — | — | — |
| Tumor | 9.48 | 1.01 | 4 | 18.33 | 2.73 | 4 | 17.85 | 1.34 | 4 | 9.35 | 1.04 | 4 | 2.51 | 0.37 | 4 |
| Muscle | 0.36 | 0.13 | 4 | 0.25 | 0.08 | 4 | — | — | — | — | — | — | — | — | — |
| Spleen | 0.95 | 0.19 | 4 | 0.65 | 0.15 | 4 | 0.71 | 0.12 | 3 | 0.58 | 0.12 | 2 | — | — | — |
| Pancreas | 24.72 | 2.40 | 4 | 17.02 | 3.00 | 4 | 3.51 | 0.74 | 4 | 2.26 | 0.22 | 4 | 1.75 | 0.16 | 4 |
| Kidneys | 6.00 | 0.70 | 4 | 5.17 | 0.63 | 4 | 2.75 | 0.86 | 4 | 1.37 | 0.24 | 4 | 0.47 | 0.09 | 4 |
| Liver | 10.18 | 1.74 | 4 | 7.07 | 1.33 | 4 | 2.88 | 0.20 | 4 | 2.00 | 0.34 | 4 | 1.73 | 0.16 | 4 |
| Small intestine | 2.54 | 0.52 | 4 | 1.92 | 0.35 | 4 | 0.31 | 0.10 | 4 | 0.14 | 0.04 | 4 | 0.06 | 0.01 | 4 |
| Large intestine | 1.71 | 0.26 | 4 | 1.51 | 0.37 | 4 | 0.70 | 0.13 | 4 | 0.26 | 0.05 | 4 | 0.12 | 0.03 | 4 |
| Tumor/Muscle | 29.05 | 9.57 | 4 | 77.20 | 21.68 | 4 | — | — | — | — | — | — | — | — | — |
| Tumor/Blood | 1.56 | 0.26 | 4 | 6.21 | 0.27 | 4 | 142.03 | 77.19 | 4 | 495.84 | 40.31 | 3 | — | — | — |
| Tumor/Kidneys | 1.58 | 0.11 | 4 | 3.55 | 0.30 | 4 | 6.84 | 1.53 | 4 | 6.94 | 1.18 | 4 | 5.36 | 0.32 | 4 |
| Tumor/liver | 0.94 | 0.08 | 4 | 2.61 | 0.18 | 4 | 6.19 | 0.10 | 4 | 4.72 | 0.38 | 4 | 1.46 | 0.27 | 4 |
| Tumor/pancreas | 0.38 | 0.01 | 4 | 1.08 | 0.04 | 4 | 5.24 | 0.98 | 4 | 4.17 | 0.60 | 4 | 1.44 | 0.23 | 4 |

### Example 4: Ex Vivo Biodistribution in PC-3 Tumor-Bearing Mice

Biodistribution studies were performed using male NOD.Cg-Rag1^{tm1Mom}II2rg^{tm1Wgl}/SzJ (NRG) mice and conducted according to the guidelines established by the Canadian Council on Animal Care and approved by Animal Ethics Committee of the University of British Columbia. For tumor inoculations, mice were anesthetized by inhalation with 2% isoflurane in oxygen and implanted subcutaneously with 5×10⁶ PC-3 cells below the left shoulder. Imaging and biodistribution studies were performed only after tumors grew to 5-8 mm in diameter.

For ex vivo biodistribution studies, mice were injected with ~1 MBq of the ¹⁷⁷Lu-labeled tracer. Mice were euthanized at various times (1h, 4 h, 24h, 48h, 72h, and/or 120h, 144 h p.i.), blood was drawn from heart, and organs/tissues of interest were collected, rinsed with PBS, blotted dry, weighed, and counted using an automated gamma counter. The uptake in each organ/tissue was normalized to the injected dose and expressed as the percentage of the injected dose per gram of tissue (% ID/g).

Biodistribution data is shown in Tables 12-14.

TABLE 12
Biodistribution data (average % ID/g with ± SD at 1 h, 4 h, 24 h, 72 h, and 120 h post-
injection) of ¹⁷⁷Lu-Compound 1 in mice bearing PC-3 tumor xenografts (1077.22 ± 32.65 kBq).

| | Time (h) | | | | | | | | | | | | | | |
|---|---|---|---|---|---|---|---|---|---|---|---|---|---|---|---|
| | 1 | | | 4 | | | 24 | | | 72 | | | 120 | | |
| Organs | AVG | SD | n | AVG | SD | n | AVG | SD | n | AVG | SD | n | AVG | SD | n |
| Blood | 0.47 | 0.10 | 4 | 0.03 | 0.01 | 4 | — | — | — | — | — | — | — | — | — |
| Tumor | 12.70 | 2.75 | 4 | 8.36 | 1.14 | 4 | 5.09 | 0.72 | 4 | 1.93 | 0.35 | 4 | 1.00 | 0.23 | 4 |
| Muscle | 0.10 | 0.06 | 4 | 0.03 | 0.02 | 4 | — | — | — | — | — | — | — | — | — |
| Spleen | 0.28 | 0.08 | 4 | 0.11 | 0.05 | 4 | 0.08 | 0.01 | 3 | — | — | — | 0.10 | 0.03 | 2 |
| Pancreas | 11.55 | 1.62 | 4 | 1.18 | 0.42 | 4 | 0.38 | 0.07 | 4 | 0.21 | 0.05 | 3 | 0.12 | 0.00 | 3 |
| Kidneys | 2.63 | 0.53 | 4 | 1.64 | 0.20 | 4 | 0.59 | 0.06 | 4 | 0.20 | 0.03 | 4 | 0.11 | 0.02 | 4 |
| Liver | 0.38 | 0.06 | 4 | 0.21 | 0.03 | 4 | 0.14 | 0.00 | 3 | 0.08 | 0.01 | 4 | 0.07 | 0.01 | 4 |
| Small intestine | 1.51 | 0.24 | 4 | 0.40 | 0.14 | 4 | 0.03 | 0.00 | 3 | 0.04 | 0.01 | 4 | 0.01 | 0.00 | 4 |
| Large intestine | 0.94 | 0.21 | 4 | 0.33 | 0.07 | 4 | 0.12 | 0.10 | 4 | 0.12 | 0.03 | 4 | 0.02 | 0.00 | 4 |
| Tumor/Muscle | 145.30 | 52.22 | 4 | 410.76 | 213.84 | 4 | — | — | — | — | — | — | — | — | — |
| Tumor/Blood | 27.11 | 2.44 | 4 | 325.78 | 74.76 | 4 | — | — | — | — | — | — | — | — | — |
| Tumor/Kidneys | 4.83 | 0.45 | 4 | 5.11 | 0.52 | 4 | 8.63 | 0.62 | 4 | 9.39 | 0.64 | 4 | 9.26 | 1.19 | 4 |
| Tumor/liver | 33.54 | 5.89 | 4 | 40.62 | 4.43 | 4 | 37.31 | 6.44 | 3 | 23.49 | 3.68 | 4 | 13.12 | 2.12 | 4 |
| Tumor/pancreas | 1.09 | 0.12 | 4 | 7.59 | 1.83 | 4 | 13.57 | 0.78 | 4 | 9.12 | 0.95 | 3 | 7.80 | 1.90 | 3 |

TABLE 13
Biodistribution data (average % ID/g with ±SD at 1 h, 4 h, 26 h, 46 h, and 120 h
post-injection) of ¹⁷⁷Lu-Compound 9 in mice bearing PC-3 tumor xenografts (0.93 ± 0.04 MBq).

| | Time (h) | | | | | | | | | | | | | | |
|---|---|---|---|---|---|---|---|---|---|---|---|---|---|---|---|
| | 1 | | | 4 | | | 26 | | | 46 | | | 120 | | |
| Organs | AVG | SD | n | AVG | SD | n | AVG | SD | n | AVG | SD | n | AVG | SD | n |
| Blood | 2.28 | 0.24 | 5 | 0.51 | 0.06 | 5 | 0.01 | 0.00 | 4 | 0.01 | 0.00 | 3 | — | — | — |
| Tumor | 17.23 | 1.14 | 5 | 25.59 | 1.65 | 5 | 19.43 | 2.33 | 5 | 15.60 | 2.46 | 5 | 3.85 | 0.42 | 5 |
| Muscle | 0.22 | 0.05 | 5 | 0.08 | 0.02 | 5 | — | — | — | — | — | — | — | — | — |
| Spleen | 0.56 | 0.08 | 4 | 0.39 | 0.06 | 5 | 0.19 | 0.03 | 5 | 0.21 | 0.06 | 5 | — | — | — |
| Pancreas | 53.20 | 3.34 | 5 | 21.58 | 3.97 | 5 | 2.06 | 0.30 | 5 | 1.63 | 0.42 | 5 | 0.27 | 0.03 | 5 |
| Kidneys | 5.48 | 0.10 | 5 | 3.52 | 0.52 | 5 | 1.11 | 0.21 | 5 | 0.76 | 0.18 | 5 | 0.21 | 0.04 | 5 |
| Liver | 3.22 | 0.20 | 5 | 2.05 | 0.24 | 5 | 0.88 | 0.21 | 5 | 0.64 | 0.17 | 5 | 0.17 | 0.02 | 5 |
| Small intestine | 4.52 | 0.45 | 5 | 2.30 | 0.51 | 5 | 0.26 | 0.05 | 5 | 0.13 | 0.03 | 4 | 0.02 | 0.00 | 4 |
| Large intestine | 2.97 | 0.33 | 5 | 1.72 | 0.12 | 5 | 0.78 | 0.32 | 5 | 0.34 | 0.07 | 5 | 0.07 | 0.01 | 5 |
| Tumor/Muscle | 81.74 | 21.37 | 5 | 337.44 | 66.36 | 5 | — | — | — | — | — | — | — | — | — |
| Tumor/Blood | 7.64 | 1.20 | 5 | 50.02 | 3.81 | 5 | 1445.62 | 162.25 | 5 | 2241.28 | 381.41 | 3 | — | — | — |
| Tumor/Kidneys | 3.14 | 0.18 | 5 | 7.35 | 0.69 | 5 | 18.37 | 0.24 | 4 | 20.83 | 2.84 | 5 | 19.19 | 1.84 | 5 |
| Tumor/liver | 5.36 | 0.41 | 5 | 12.55 | 0.67 | 5 | 22.56 | 2.89 | 5 | 25.08 | 3.21 | 5 | 22.26 | 2.46 | 5 |
| Tumor/pancreas | 0.32 | 0.02 | 5 | 1.21 | 0.17 | 5 | 9.49 | 0.61 | 5 | 9.79 | 1.43 | 5 | 14.14 | 1.35 | 5 |

TABLE 14
Biodistribution data (average % ID/g with ±SD at 1 h, 4 h, 27 h, 46 h, and 118 h
post-injection) of ¹⁷⁷Lu-Compound 10 in mice bearing PC-3 tumor xenografts (1.09 ± 0.06 MBq).

| | Time (h) | | | | | | | | | | | | | | |
|---|---|---|---|---|---|---|---|---|---|---|---|---|---|---|---|
| | 1 | | | 4 | | | 27 | | | 46 | | | 118 | | |
| Organs | AVG | SD | n | AVG | SD | n | AVG | SD | n | AVG | SD | n | AVG | SD | n |
| Blood | 3.72 | 0.49 | 5 | 0.98 | 0.11 | 5 | 0.02 | 0.00 | 4 | 0.01 | 0.01 | 5 | — | — | — |
| Tumor | 21.55 | 0.97 | 5 | 25.88 | 3.30 | 5 | 10.87 | 1.09 | 5 | 8.23 | 0.91 | 5 | 2.71 | 0.68 | 5 |
| Muscle | 0.24 | 0.03 | 5 | 0.10 | 0.01 | 5 | 0.02 | 0.01 | 5 | — | — | — | — | — | — |
| Spleen | 0.78 | 0.06 | 5 | 0.47 | 0.06 | 5 | 0.26 | 0.04 | 5 | 0.35 | 0.07 | 5 | 0.17 | 0.02 | 4 |
| Pancreas | 17.97 | 1.84 | 5 | 6.94 | 1.56 | 5 | 0.83 | 0.07 | 5 | 0.79 | 0.11 | 5 | 0.19 | 0.06 | 5 |
| Kidneys | 6.68 | 0.50 | 5 | 4.15 | 0.41 | 5 | 1.25 | 0.17 | 5 | 1.02 | 0.16 | 5 | 0.32 | 0.14 | 5 |
| Liver | 5.78 | 0.60 | 5 | 4.05 | 0.41 | 5 | 1.51 | 0.38 | 5 | 1.02 | 0.22 | 5 | 0.34 | 0.10 | 5 |
| Small intestine | 2.44 | 0.23 | 5 | 1.26 | 0.13 | 5 | 0.28 | 0.03 | 5 | 0.15 | 0.03 | 5 | 0.03 | 0.00 | 4 |
| Large intestine | 1.45 | 0.13 | 5 | 0.98 | 0.09 | 5 | 0.51 | 0.10 | 5 | 0.33 | 0.05 | 4 | 0.08 | 0.04 | 5 |
| Tumor/Muscle | 88.90 | 9.18 | 5 | 265.92 | 19.21 | 5 | 481.09 | 98.94 | 5 | — | — | — | — | — | — |
| Tumor/Blood | 5.87 | 0.81 | 5 | 26.55 | 1.97 | 5 | 610.83 | 40.74 | 4 | 687.86 | 278.17 | 5 | — | — | — |
| Tumor/Kidneys | 3.24 | 0.25 | 5 | 6.23 | 0.46 | 5 | 8.76 | 0.48 | 5 | 8.13 | 0.72 | 5 | 9.85 | 2.01 | 5 |
| Tumor/liver | 3.76 | 0.42 | 5 | 6.38 | 0.23 | 5 | 7.37 | 0.95 | 5 | 8.24 | 1.36 | 5 | 8.15 | 1.22 | 5 |
| Tumor/pancreas | 1.21 | 0.10 | 5 | 3.80 | 0.45 | 5 | 13.05 | 0.40 | 5 | 10.57 | 1.73 | 5 | 15.29 | 2.02 | 5 |

### Example 5: Ex Vivo Biodistribution in PC-3 Tumor-Bearing Mice

Biodistribution studies were performed using male NOD.Cg-Rag1^{tm1Mom}II2rg^{tm1Wgl}/SzJ (NRG) mice and conducted according to the guidelines established by the Canadian Council on Animal Care and approved by Animal Ethics Committee of the University of British Columbia. For tumor inoculations, mice were anesthetized by inhalation with 2% isoflurane in oxygen and implanted subcutaneously with 5×10⁶ PC-3 cells below the left shoulder. Imaging and biodistribution studies were performed only after tumors grew to 5-8 mm in diameter.

For ex vivo biodistribution studies, mice were injected with ~1 MBq of the ¹⁷⁷Lu, or ~20 kBq of the ²²⁵Ac-labeled tracer. Mice were euthanized at various times (1h, 4 h, 24h, 48h, 72h, and/or 120h, 144 h p.i.), blood was drawn from heart, and organs/tissues of interest were collected, rinsed with PBS, blotted dry, weighed, and counted using an automated gamma counter. The uptake in each organ/tissue was normalized to the injected dose and expressed as the percentage of the injected dose per gram of tissue (% ID/g).

The biodistribution data was decayed from injection to its corresponding time point. Mono-exponential and/or bi-exponential fits were performed to generate the time-activity-curves for each organ. Activity under the curve (AUC) values were then integrated for each organ.

Comparative Time-Activity Integration (AUC) of various radiotracers is shown in Table 15.

TABLE 15
Comparative Time-Activity Integration (AUC) of Radiotracers
Uptake in Tumor, Pancreas, and Kidney.

| | | | | Tumor/ | |
|---|---|---|---|---|---|
| | | | | Pan- | Tumor/ |
| Compounds | AUCₜᵤₘₒᵣ | AUC_{Pancreas} | AUC_{Kidney} | creas | Kidney |
| ²²⁵Ac-AMTG | 1004.1 | 1698.2 | 69.3 | 0.59 | 14.49 |
| ²²⁵Ac-RM2 | 564.4 | 1575.7 | 89.0 | 0.36 | 6.34 |
| ²²⁵Ac- | 271.1 | 17.0 | 92.4 | 15.91 | 2.93 |
| Compound 2 | | | | | |
| ²²⁵Ac- | 939.5 | 478.8 | 200.8 | 1.96 | 4.68 |
| Compound 8 | | | | | |
| ²²⁵Ac- | 2041.6 | 1403.7 | 121.5 | 1.45 | 16.80 |
| Compound 10 | | | | | |
| ²²⁵Ac- | 1254.7 | 675.6 | 232.6 | 1.86 | 5.39 |
| Compound 34 | | | | | |
| ¹⁷⁷Lu-AMTG | 879.8 | 167.9 | 55.8 | 5.24 | 15.77 |
| ¹⁷⁷Lu- | 1346.4 | 351.0 | 99.8 | 3.84 | 13.50 |
| Compound 9 | | | | | |
| ¹⁷⁷Lu- | 860.88 | 138.85 | 127.12 | 6.20 | 6.77 |
| Compound 10 | | | | | |

AUC unit: % ID/g · h

All publications, patents and patent applications, including any drawings and appendices therein are incorporated by reference in their entirety for all purposes to the same extent as if each individual publication, patent or patent application, drawing, or appendix was specifically and individually indicated to be incorporated by reference in its entirety for all purposes. It will be apparent to persons skilled in the art that a number of variations and modifications can be made without departing from the scope of the invention as defined in the following claims. The scope of the invention should therefore not be limited by the preferred embodiments set forth in the above Examples, but should be given the broadest interpretation consistent with the description as a whole.

1.-85. (canceled)

86. A compound of Formula (I),
(I)
R^{rad}ₙ₆-[linker]-Xaa¹-Xaa²-Xaa³-Xaa⁴-Xaa⁵-Xaa⁶-
Xaa⁷-Xaa⁸-[ψ-Xaa⁹-(Xaa¹⁰)ₘ₂]ₘ₁-R
or a salt thereof, wherein:
Xaa¹ is an N-terminal amino acid residue selected from D-Phe, D-Cpa, D-Nal, or D-2-Nal;
Xaa² is Asp, Asn, Gln, or Glu;
Xaa³ is Trp, Trp(Me), αMe-Trp, Trp(7-F), Trp(6-F), Trp(5-F), or Trp(4-F);
Xaa⁴ is Ala or D-Ala;
Xaa⁵ is Val or tert-leucine (Tle);
Xaa⁶ is Gly or D-Ala;
Xaa⁷ is Trp, Trp(Me), Trp(7-Me), Trp(6-Me), Trp(5-Me), Trp(4-Me), Trp(2-Me), Trp(7-F), Trp(6-F), Trp(5-F), Trp(4-F), Trp(7-Cl), Trp(6-Cl), Trp(5-Cl), Trp(4-Cl), Trp(7-Br), Trp(6-Br), Trp(5-Br), Trp(4-Br), Trp(7-I), Trp(6-I), Trp(5-I), Trp(4-I), Trp(5-OH), or αMe-Trp;
Xaa⁸ is Leu;
m1 is 1 and m2 is 0;
Xaa⁹ is Pro, oxazolidine-4-carboxylic acid (4-oxa-L-Pro), or 4,4-difluoroproline (difluoroPro);
R is —NH₂;
ψ represents a peptide bond or reduced peptide bond joining Xaa⁸ to Xaa⁹;
the linker comprises p-aminomethylaniline-diglycolic acid (pABzA-DIG), 4-amino-(1-carboxymethyl)piperidine (Pip), 9-amino-4,7-dioxanonanoic acid (dPEG2), or 4-(2-aminoethyl)-1-carboxymethyl-piperazine (Acp), wherein the linker optionally comprises an albumin binder;
n6 is 1;
R^{rad} is a radiometal chelator;
wherein the compound is optionally bound by a radiometal; and
wherein Xaa¹, Xaa², Xaa³, Xaa⁴, Xaa⁵, Xaa⁶, Xaa⁷, Xaa⁸, or Xaa⁹ is each optionally N-methylated.

87. The compound of claim 86, or a salt thereof, wherein Xaa¹ is D-Phe, D-Nal, or D-2-Nal.

88. The compound of claim 86, or a salt thereof, wherein Xaa² is Gln or Glu.

89. The compound of claim 86, or a salt thereof, wherein Xaa³ is Trp or αMe-Trp.

90. The compound of claim 86, or a salt thereof, wherein Xaa⁵ is tert-leucine (Tle).

91. The compound of claim 86, or a salt thereof, wherein Xaa⁶ is Gly.

92. The compound of claim 86, or a salt thereof, wherein Xaa⁷ is Trp, Trp(5-Me), Trp(5-F), Trp(5-Cl), Trp(4-Cl), Trp(5-Br), Trp(5-I), or Trp(5-OH).

93. The compound of claim 86, or a salt thereof, wherein Xaa⁹ is Pro or 4,4-difluoroproline (difluoroPro).

94. The compound of claim 86, or a salt thereof, wherein W represents a reduced peptide bond joining Xaa⁸ to Xaa⁹.

95. The compound of claim 86, or a salt thereof, wherein the linker is 4-amino-(1-carboxymethyl)piperidine (Pip) or 4-(2-aminoethyl)-1-carboxymethyl-piperazine (Acp).

96. The compound of claim 86, or a salt thereof, wherein Xaa¹, Xaa², Xaa³, Xaa⁴, Xaa⁵, Xaa⁶, Xaa⁷, Xaa⁸, and Xaa⁹ are not N-methylated.

97. The compound of claim 86, or a salt thereof, wherein the radiometal chelator is selected from the group consisting of DOTA and derivatives; DOTAGA; NOTA; NODAGA; NODASA; CB-DO2A; 3p-C-DEPA; TCMC; DO3A; DTPA and DTPA analogues optionally selected from CHX-A″-DTPA and 1B4M-DTPA; TETA; NOPO; Me-3,2-HOPO; CB-TE1A1P; CB-TE2P; MM-TE2A; DM-TE2A; sarcophagine and sarcophagine derivatives optionally selected from SarAr, SarAr-NCS, diamSar, AmBaSar, and BaBaSar; TRAP; AAZTA; DATA and DATA derivatives; H2-macropa or a derivative thereof; H₂dedpa, H₄octapa, H₄py4pa, H4Pypa, H2azapa, H5decapa, and other picolinic acid derivatives; CP256; PCTA; C-NETA; C-NE3TA; HBED; SHBED; BCPA; CP256; YM103; desferrioxamine (DFO) and DFO derivatives; H₆phospa; a trithiol chelate; mercaptoacetyl; hydrazinonicotinamide; dimercaptosuccinic acid; 1,2-ethylenediylbis-L-cysteine diethyl ester; methylenediphosphonate; hexamethylpropyleneamineoxime; hexakis(methoxy isobutyl isonitrile), H4py4pa-phenyl-NCS, PSC, CROWN, and CROWNGA.

98. The compound of claim 97, or a salt thereof, wherein the radiometal chelator is DOTA, DOTAGA, CROWN, or CROWNGA.

99. The compound of claim 86, or a salt thereof, wherein the radiometal chelator is bound by or conjugated with a radiometal or a radiohalogen containing metal selected from [¹⁸F]AlF, ⁴⁴Sc, ⁴⁷Sc, ⁶¹Cu, ⁶⁴Cu, ⁶⁷Cu, ⁶⁷Ga, ⁶⁸Ga, ⁷²As, ⁷⁷As, ⁸⁶Y, ⁸⁹Zr, ⁹⁰Y, ⁹⁰Nb, ^{94m}Tc, ^{99m}Tc ¹⁰⁵Rh ¹⁰⁹Pd, ¹¹¹In, ¹¹⁴In, ¹¹⁷Sn, ¹³²La, ¹³³La, ¹³⁴La, ¹³⁵La, ¹³⁴Ce, ¹⁴²Pr, ¹⁴⁹Pm ¹⁴⁹Tb, ¹⁵²Tb, Tb, ¹⁵⁹Gd, 161Tb, 165Er 166Ho ¹⁷⁵Yb ¹⁷⁷Lu, ¹⁸⁶Re ¹⁸⁸Re, ¹⁹⁸Au, ¹⁹⁹Au, ²⁰³Pb, ²¹ At ²¹²Pb ²¹²Bi, ²¹³Bi, ²²⁵Ac, or ²²⁷Th.

100. The compound of claim 99, or a salt thereof, wherein the radiometal chelator is bound by a radiometal selected from ⁶⁸Ga, ¹⁷⁷Lu, or ²²⁵Ac.

101. A composition comprising a compound of claim 86, or a salt thereof, and a pharmaceutically acceptable carrier or excipient.

102. A method of imaging gastrin-releasing peptide receptor (GRPR)-expressing tissue in a subject, the method comprising: administering to the subject a compound of claim 86, or a salt thereof; and imaging tissue of the subject.

103. The method of claim 102, wherein the compound, or a salt thereof, is bound by a radiometal selected from ⁶⁸Ga or ¹⁷⁷Lu.

104. A method of treating cancer in a subject comprising, administering to the subject in need thereof a therapeutically effective amount of a compound of claim 86, or a salt thereof.

105. The method of claim 104, wherein the cancer is prostate cancer, lung cancer, head and neck cancer, colon cancer, kidney cancer, ovarian cancer, liver cancer, pancreatic cancer, breast cancer, glioma or neuroblastoma.

106. The method of claim 104, wherein the compound, or a salt thereof, is bound by a radiometal selected from ¹⁷⁷Lu or ²²⁵Ac.


SEQUENCE LISTING (67 sequences)

SEQ ID NO: 1 - protein, 10 aa, synthetic construct
  SITE 1: note = 4-amino-1-carboxymethyl-piperidine modified with 1,4,7,10- tetraazacyclododecane-1,4,7,10-tetraacetic acid
  SITE 2: note = D-phenylalanine
  SITE 4: note = N-methyltryptophan
  SITE 6: note = tert-leucine
  MOD_RES 9..10: note = linked via peptide or reduced peptide bond
  SITE 10: note = thiazoline-4-carboxylic acid
  SITE 10: note = C-terminus is amidated
XFQWAXGHLX

SEQ ID NO: 2 - protein, 10 aa, synthetic construct
  SITE 1: note = N-terminus linked to a radiometal chelator via 4-amino-(1-carboxymethyl)piperidine 4-amido-(1-carboxymethyl)piperidine or 4-acetamido-(1-carboxymethyl)piperidine
  SITE 1: note = D-phenylalanine
  SITE 1: note = may be N-methylated
  SITE 2: note = may be N-methylated
  SITE 3: note = N-methyltryptophan
  SITE 3: note = may be N-methylated
  SITE 4: note = may be N-methylated
  VARIANT 5: note = valine or tert-leucine
  SITE 5: note = may be N-methylated
  SITE 6: note = may be N-methylated
  SITE 7: note = may be N-methylated
  VARIANT 8: note = statine or leucine
  SITE 8: note = may be N-methylated
  MOD_RES 8..9: note = linked via peptide or reduced peptide bond
  VARIANT 9: note = amidated or hydroxylamidated proline, amidated or hydroxylamidated leucine or may be absent
  SITE 9: note = may be N-methylated
  VARIANT 10: note = amidated or hydroxylamidated valine, amidated or hydroxylamidated tert-leucine or may be absent
  SITE 10: note = may be N-methylated
FQWAXGHXXX

SEQ ID NO: 3 - protein, 9 aa, synthetic construct
  SITE 1: note = D-phenylalanine
  SITE 3: note = N-methyltryptophan
  SITE 5: note = tert-leucine
  MOD_RES 8..9: note = linked via reduced peptide bond
  SITE 9: note = C-terminus is amidated
FQWAXGHLP

SEQ ID NO: 4 - protein, 9 aa, synthetic construct
  SITE 1: note = D-phenylalanine
  SITE 3: note = N-methyltryptophan
  SITE 8: note = statine
  SITE 9: note = C-terminus is amidated
FQWAVGHXL

SEQ ID NO: 5 - protein, 9 aa, synthetic construct
  SITE 1: note = D-phenylalanine
  SITE 3: note = N-methyltryptophan
  SITE 5: note = tert-leucine
  SITE 8: note = statine
  SITE 9: note = C-terminus is amidated
FQWAXGHXL

SEQ ID NO: 6 - protein, 9 aa, synthetic construct
  SITE 1: note = D-phenylalanine
  SITE 6: note = N-methylated glycine
  SITE 5: note = tert-leucine
  SITE 8: note = statine
  MOD_RES 8..9: note = linked via reduced peptide bond
  SITE 9: note = C-terminus is amidated
FQWAVGWLP

SEQ ID NO: 7 - protein, 9 aa, synthetic construct
  SITE 1: note = D-phenylalanine
  SITE 3: note = N-methyltryptophan
  SITE 5: note = tert-leucine
  MOD_RES 8..9: note = linked via reduced peptide bond
  SITE 9: note = C-terminus is amidated
FQWAXGWLP

SEQ ID NO: 8 - protein, 9 aa, synthetic construct
  SITE 1: note = D-phenylalanine
  SITE 3: note = N-methyltryptophan
  SITE 5: note = tert-leucine
  SITE 7: note = 5-fluorotryptophan
  MOD_RES 8..9: note = linked via reduced peptide bond
  SITE 9: note = C-terminus is amidated
FQWAXGWLP

SEQ ID NO: 9 - protein, 9 aa, synthetic construct
  SITE 1: note = D-phenylalanine
  SITE 3: note = N-methyltryptophan
  SITE 5: note = tert-leucine
  SITE 8: note = statine
  SITE 9: note = C-terminus is amidated
FQWAXGWXL

SEQ ID NO: 10 - protein, 9 aa, synthetic construct
  SITE 1: note = D-phenylalanine
  SITE 3: note = N-methyltryptophan
  SITE 5: note = tert-leucine
  SITE 7: note = 5-fluorotryptophan
  SITE 8: note = statine
  SITE 9: note = C-terminus is amidated
FQWAXGWXL

SEQ ID NO: 11 - protein, 9 aa, synthetic construct
  SITE 1: note = D-phenylalanine
  SITE 2: note = citrulline
  SITE 3: note = N-methyltryptophan
  SITE 5: note = tert-leucine
  MOD_RES 8..9: note = linked via reduced peptide bond
  SITE 9: note = C-terminus is amidated
FXWAXGHLP

SEQ ID NO: 12 - protein, 9 aa, synthetic construct
  SITE 1: note = D-phenylalanine
  SITE 3: note = N-methyltryptophan
  SITE 5: note = tert-leucine
  MOD_RES 8..9: note = linked via reduced peptide bond
  SITE 9: note = C-terminus is amidated
FEWAXGHLP

SEQ ID NO: 13 - protein, 9 aa, synthetic construct
  SITE 1: note = D-phenylalanine
  SITE 2: note = 2-amino-4-guanidinobutyric acid
  SITE 3: note = N-methyltryptophan
  SITE 5: note = tert-leucine
  MOD_RES 8..9: note = linked via reduced peptide bond
  SITE 9: note = C-terminus is amidated
FXWAXGHLP

SEQ ID NO: 14 - protein, 9 aa, synthetic construct
  SITE 1: note = D-phenylalanine
  SITE 3: note = N-methyltryptophan
  SITE 4: note = N-methylated glycine
  SITE 5: note = tert-leucine
  MOD_RES 8..9: note = linked via reduced peptide bond
  SITE 9: note = C-terminus is amidated
FQWGXGHLP

SEQ ID NO: 15 - protein, 9 aa, synthetic construct
  SITE 1: note = D-phenylalanine
  SITE 3: note = N-methyltryptophan
  SITE 4: note = N-methylated glycine
  SITE 5: note = tert-leucine
  MOD_RES 8..9: note = linked via reduced peptide bond
  SITE 9: note = C-terminus is amidated
FQWGXGWLP

SEQ ID NO: 16 - protein, 9 aa, synthetic construct
  SITE 1: note = D-phenylalanine
  SITE 6: note = N-methylated glycine
  MOD_RES 8..9: note = linked via reduced peptide bond
  SITE 9: note = C-terminus is amidated
FQWAVGHLP

SEQ ID NO: 17 - protein, 10 aa, synthetic construct
  SITE 1: note = D-phenylalanine
  SITE 6: note = N-methylated glycine
  MOD_RES 8..9: note = linked via reduced peptide bond
  SITE 9: note = C-terminus is amidated
FQWAVGWLPV

SEQ ID NO: 18 - protein, 10 aa, synthetic construct
  SITE 1: note = D-phenylalanine
  SITE 6: note = N-methylated glycine
  MOD_RES 8..9: note = linked via reduced peptide bond
  SITE 9: note = C-terminus is amidated
FQWAVGHLPV

SEQ ID NO: 19 - protein, 10 aa, synthetic construct
  SITE 1: note = D-phenylalanine
  SITE 3: note = N-methyltryptophan
  SITE 5: note = tert-leucine
  MOD_RES 8..9: note = linked via reduced peptide bond
  SITE 10: note = tert-leucine
  SITE 10: note = C-terminus is amidated
FQWAXGWLPX

SEQ ID NO: 20 - protein, 10 aa, synthetic construct
  SITE 1: note = D-phenylalanine
  SITE 3: note = N-methyltryptophan
  SITE 5: note = tert-leucine
  MOD_RES 8..9: note = linked via reduced peptide bond
  SITE 10: note = tert-leucine
  SITE 10: note = C-terminus is amidated
FQWAXGHLPX

SEQ ID NO: 21 - protein, 10 aa, synthetic construct
  SITE 1: note = piperidine
  SITE 2: note = D-phenylalanine
  SITE 4: note = 7-fluorotryptophan
  SITE 6: note = tert-leucine
  MOD_RES 9..10: note = linked via reduced peptide bond
  SITE 10: note = C-terminus is amidated
XFQWAXGHLP

SEQ ID NO: 22 - protein, 10 aa, synthetic construct
  SITE 1: note = 4-amino-1-carboxymethyl-piperidine modified with 1,4,7,10- tetraazacyclododecane-1,4,7,10-tetraacetic acid
  SITE 2: note = D-phenylalanine
  SITE 4: note = N-methyltryptophan
  SITE 6: note = tert-leucine
  MOD_RES 9..10: note = linked via reduced peptide bond
  SITE 10: note = C-terminus is amidated
XFQWAXGHLP

SEQ ID NO: 23 - protein, 10 aa, synthetic construct
  SITE 1: note = 4-amino-1-carboxymethyl-piperidine modified with 2,2',2'',2'''-(1,10- dioxa-4,7,13,16-tetraazacyclooctadecane-4,7,13,16-tetrayl) tetraacetic acid
  SITE 2: note = D-phenylalanine
  SITE 4: note = N-methyltryptophan
  SITE 6: note = tert-leucine
  MOD_RES 9..10: note = linked via reduced peptide bond
  SITE 10: note = C-terminus is amidated
XFQWAXGHLP

SEQ ID NO: 24 - protein, 10 aa, synthetic construct
  SITE 1: note = 4-amino-1-carboxymethyl-piperidine modified with 2,2',2'',2'''-(1,10- dioxa-4,7,13,16-tetraazacyclooctadecane-4,7,13,16-tetrayl) tetraacetic acid
  SITE 2: note = D-phenylalanine
  SITE 4: note = N-methyltryptophan
  SITE 9: note = statine
  SITE 10: note = C-terminus is amidated
XFQWAVGHXL

SEQ ID NO: 25 - protein, 10 aa, synthetic construct
  SITE 1: note = 4-amino-1-carboxymethyl-piperidine modified with 2,2',2'',2'''-(1,10- dioxa-4,7,13,16-tetraazacyclooctadecane-4,7,13,16-tetrayl) tetraacetic acid
  SITE 2: note = D-phenylalanine
  SITE 4: note = N-methyltryptophan
  SITE 6: note = tert-leucine
  SITE 9: note = statine
  SITE 10: note = C-terminus is amidated
XFQWAXGHXL

SEQ ID NO: 26 - protein, 10 aa, synthetic construct
  SITE 1: note = 4-amino-1-carboxymethyl-piperidine modified with 1,4,7,10- tetraazacyclododecane-1,4,7,10-tetraacetic acid
  SITE 2: note = D-phenylalanine
  SITE 7: note = N-methylated glycine
  MOD_RES 9..10: note = linked via reduced peptide bond
  SITE 10: note = C-terminus is amidated
XFQWAVGWLP

SEQ ID NO: 27 - protein, 10 aa, synthetic construct
  SITE 1: note = 4-amino-1-carboxymethyl-piperidine modified with 1,4,7,10- tetrakis(carboxymethyl)-7-acetamide-1,4,10-triaz acyclododecane glutaric acid
  SITE 2: note = D-phenylalanine
  SITE 7: note = N-methylated glycine
  MOD_RES 9..10: note = linked via reduced peptide bond
  SITE 10: note = C-terminus is amidated
XFQWAVGWLP

SEQ ID NO: 28 - protein, 10 aa, synthetic construct
  SITE 1: note = 4-amino-1-carboxymethyl-piperidine modified with 1,4,7,10- tetraazacyclododecane-1,4,7,10-tetraacetic acid
  SITE 2: note = D-phenylalanine
  SITE 4: note = N-methyltryptophan
  SITE 6: note = tert-leucine
  MOD_RES 9..10: note = linked via reduced peptide bond
  SITE 10: note = C-terminus is amidated
XFQWAXGWLP

SEQ ID NO: 29 - protein, 10 aa, synthetic construct
  SITE 1: note = 4-amino-1-carboxymethyl-piperidine modified with 1,4,7,10- tetrakis(carboxymethyl)-7-acetamide-1,4,10-triaz acyclododecane glutaric acid
  SITE 2: note = D-phenylalanine
  SITE 4: note = N-methyltryptophan
  SITE 6: note = tert-leucine
  MOD_RES 9..10: note = linked via reduced peptide bond
  SITE 10: note = C-terminus is amidated
XFQWAXGWLP

SEQ ID NO: 30 - protein, 10 aa, synthetic construct
  SITE 1: note = 4-amino-1-carboxymethyl-piperidine modified with 2,2',2'',2'''-(1,10- dioxa-4,7,13,16-tetraazacyclooctadecane-4,7,13,16-tetrayl) tetraacetic acid
  SITE 2: note = D-phenylalanine
  SITE 4: note = N-methyltryptophan
  SITE 6: note = tert-leucine
  MOD_RES 9..10: note = linked via reduced peptide bond
  SITE 10: note = C-terminus is amidated
XFQWAXGWLP

SEQ ID NO: 31 - protein, 10 aa, synthetic construct
  SITE 1: note = 4-amino-1-carboxymethyl-piperidine modified with 2-(7,13,16-tris(carboxymethyl)- 1,10-dioxa-4,7,13,16-tetra azacyclooctadecan-4-yl)pentanedioic acid
  SITE 2: note = D-phenylalanine
  SITE 4: note = N-methyltryptophan
  SITE 6: note = tert-leucine
  MOD_RES 9..10: note = linked via reduced peptide bond
  SITE 10: note = C-terminus is amidated
XFQWAXGWLP

SEQ ID NO: 32 - protein, 10 aa, synthetic construct
  SITE 1: note = 4-amino-1-carboxymethyl-piperidine modified with 1,4,7,10- tetraazacyclododecane-1,4,7,10-tetraacetic acid
  SITE 2: note = D-phenylalanine
  SITE 4: note = N-methyltryptophan
  SITE 6: note = tert-leucine
  SITE 8: note = 5-fluorotryptophan
  MOD_RES 9..10: note = linked via reduced peptide bond
  SITE 10: note = C-terminus is amidated
XFQWAXGWLP

SEQ ID NO: 33 - protein, 10 aa, synthetic construct
  SITE 1: note = 4-amino-1-carboxymethyl-piperidine modified with 1,4,7,10- tetrakis(carboxymethyl)-7-acetamide-1,4,10-triaz acyclododecane glutaric acid
  SITE 2: note = D-phenylalanine
  SITE 4: note = N-methyltryptophan
  SITE 6: note = tert-leucine
  SITE 8: note = 5-fluorotryptophan
  MOD_RES 9..10: note = linked via reduced peptide bond
  SITE 10: note = C-terminus is amidated
XFQWAXGWLP

SEQ ID NO: 34 - protein, 10 aa, synthetic construct
  SITE 1: note = 4-amino-1-carboxymethyl-piperidine modified with 2,2',2'',2'''-(1,10- dioxa-4,7,13,16-tetraazacyclooctadecane-4,7,13,16-tetrayl) tetraacetic acid
  SITE 2: note = D-phenylalanine
  SITE 4: note = N-methyltryptophan
  SITE 6: note = tert-leucine
  SITE 9: note = statine
  SITE 10: note = C-terminus is amidated
XFQWAXGWXL

SEQ ID NO: 35 - protein, 10 aa, synthetic construct
  SITE 1: note = 4-amino-1-carboxymethyl-piperidine modified with 2,2',2'',2'''-(1,10- dioxa-4,7,13,16-tetraazacyclooctadecane-4,7,13,16-tetrayl) tetraacetic acid
  SITE 2: note = D-phenylalanine
  SITE 4: note = N-methyltryptophan
  SITE 6: note = tert-leucine
  SITE 8: note = 5-fluorotryptophan
  SITE 9: note = statine
  SITE 10: note = C-terminus is amidated
XFQWAXGWXL

SEQ ID NO: 36 - protein, 10 aa, synthetic construct
  SITE 1: note = 4-amino-1-carboxymethyl-piperidine modified with 2,2',2'',2'''-(1,10- dioxa-4,7,13,16-tetraazacyclooctadecane-4,7,13,16-tetrayl) tetraacetic acid
  SITE 2: note = D-phenylalanine
  SITE 3: note = citrulline
  SITE 4: note = N-methyltryptophan
  SITE 6: note = tert-leucine
  MOD_RES 9..10: note = linked via reduced peptide bond
  SITE 10: note = C-terminus is amidated
XFXWAXGHLP

SEQ ID NO: 37 - protein, 10 aa, synthetic construct
  SITE 1: note = 4-amino-1-carboxymethyl-piperidine modified with 2,2',2'',2'''-(1,10- dioxa-4,7,13,16-tetraazacyclooctadecane-4,7,13,16-tetrayl) tetraacetic acid
  SITE 2: note = D-phenylalanine
  SITE 4: note = N-methyltryptophan
  SITE 6: note = tert-leucine
  MOD_RES 9..10: note = linked via reduced peptide bond
  SITE 10: note = C-terminus is amidated
XFEWAXGHLP

SEQ ID NO: 38 - protein, 10 aa, synthetic construct
  SITE 1: note = 4-amino-1-carboxymethyl-piperidine modified with 2,2',2'',2'''-(1,10- dioxa-4,7,13,16-tetraazacyclooctadecane-4,7,13,16-tetrayl) tetraacetic acid
  SITE 2: note = D-phenylalanine
  SITE 3: note = 2-amino-4-guanidinobutyric acid
  SITE 4: note = N-methyltryptophan
  SITE 6: note = tert-leucine
  MOD_RES 9..10: note = linked via reduced peptide bond
  SITE 10: note = C-terminus is amidated
XFXWAXGHLP

SEQ ID NO: 39 - protein, 9 aa, synthetic construct
  SITE 1: note = 4-amino-1-carboxymethyl-piperidine modified with 2,2',2'',2'''-(1,10- dioxa-4,7,13,16-tetraazacyclooctadecane-4,7,13,16-tetrayl) tetraacetic acid
  SITE 2: note = D-phenylalanine
  SITE 4: note = N-methyltryptophan
  SITE 6: note = tert-leucine
  SITE 9: note = cis-4-cyclohexyloxy-D-proline
  SITE 9: note = C-terminus is amidated
XFQWAXGWX

SEQ ID NO: 40 - protein, 9 aa, synthetic construct
  SITE 1: note = 4-amino-1-carboxymethyl-piperidine modified with 2,2',2'',2'''-(1,10- dioxa-4,7,13,16-tetraazacyclooctadecane-4,7,13,16-tetrayl) tetraacetic acid
  SITE 2: note = D-phenylalanine
  SITE 4: note = N-methyltryptophan
  SITE 6: note = tert-leucine
  SITE 9: note = cis-4-cyclohexylamino-D-proline
  SITE 9: note = C-terminus is amidated
XFQWAXGWX

SEQ ID NO: 41 - protein, 11 aa, synthetic construct
  SITE 1: note = 4-amino-1-carboxymethyl-piperidine modified with 1,4,7,10- tetraazacyclododecane-1,4,7,10-tetraacetic acid
  SITE 2: note = D-phenylalanine
  SITE 4: note = N-methyltryptophan
  SITE 6: note = tert-leucine
  SITE 8: note = 5-fluorotryptophan
  MOD_RES 9..10: note = linked via reduced peptide bond
  SITE 11: note = tert-leucine
  SITE 11: note = C-terminus is amidated
XFQWAXGWLPX

SEQ ID NO: 42 - protein, 10 aa, synthetic construct
  SITE 1: note = 4-amino-1-carboxymethyl-piperidine modified with 1,4,7,10- tetraazacyclododecane-1,4,7,10-tetraacetic acid
  SITE 2: note = D-phenylalanine
  SITE 3: note = citrulline
  SITE 4: note = N-methyltryptophan
  SITE 6: note = tert-leucine
  SITE 8: note = 3-quinoylalanine
  MOD_RES 9..10: note = linked via reduced peptide bond
  SITE 10: note = C-terminus is amidated
XFXWAXGXLP

SEQ ID NO: 43 - protein, 10 aa, synthetic construct
  SITE 1: note = 4-amino-1-carboxymethyl-piperidine modified with 1,4,7,10- tetraazacyclododecane-1,4,7,10-tetraacetic acid
  SITE 2: note = D-phenylalanine
  SITE 3: note = citrulline
  SITE 4: note = N-methyltryptophan
  SITE 9: note = statine
  SITE 10: note = C-terminus is amidated
XFXWAVGHXL

SEQ ID NO: 44 - protein, 10 aa, synthetic construct
  SITE 1: note = 4-amino-1-carboxymethyl-piperidine modified with 1,4,7,10- tetraazacyclododecane-1,4,7,10-tetraacetic acid
  SITE 2: note = D-phenylalanine
  SITE 3: note = citrulline
  SITE 4: note = N-methyltryptophan
  SITE 8: note = 5-fluorotryptophan
  SITE 9: note = statine
  SITE 10: note = C-terminus is amidated
XFXWAVGWXL

SEQ ID NO: 45 - protein, 10 aa, synthetic construct
  SITE 1: note = 4-amino-1-carboxymethyl-piperidine modified with 1,4,7,10- tetrakis(carbamoylmethyl)-1,4,7,10-tetraazacyclododecane
  SITE 2: note = D-phenylalanine
  SITE 7: note = N-methylated glycine
  MOD_RES 9..10: note = linked via reduced peptide bond
  SITE 10: note = C-terminus is amidated
XFQWAVGHLP

SEQ ID NO: 46 - protein, 10 aa, synthetic construct
  SITE 1: note = 4-amino-1-carboxymethyl-piperidine modified with 1,4,7,10- tetraazacyclododecane-1,4,7,10-tetraacetic acid
  SITE 2: note = D-phenylalanine
  SITE 7: note = N-methylated glycine
  MOD_RES 9..10: note = linked via reduced peptide bond
  SITE 10: note = C-terminus is amidated
XFQWAVGHLP

SEQ ID NO: 47 - protein, 11 aa, synthetic construct
  SITE 1: note = 4-amino-1-carboxymethyl-piperidine modified with 1,4,7,10- tetraazacyclododecane-1,4,7,10-tetraacetic acid
  SITE 2: note = D-phenylalanine
  SITE 7: note = N-methylated glycine
  MOD_RES 9..10: note = linked via reduced peptide bond
  SITE 11: note = C-terminus is amidated
XFQWAVGWLPV

SEQ ID NO: 48 - protein, 11 aa, synthetic construct
  SITE 1: note = 4-amino-1-carboxymethyl-piperidine modified with 1,4,7,10- tetraazacyclododecane-1,4,7,10-tetraacetic acid
  SITE 2: note = D-phenylalanine
  SITE 7: note = N-methylated glycine
  MOD_RES 9..10: note = linked via reduced peptide bond
  SITE 11: note = C-terminus is amidated
XFQWAVGHLPV

SEQ ID NO: 49 - protein, 11 aa, synthetic construct
  SITE 1: note = 4-amino-1-carboxymethyl-piperidine modified with 2,2',2'',2'''-(1,10- dioxa-4,7,13,16-tetraazacyclooctadecane-4,7,13,16-tetrayl) tetraacetic acid
  SITE 2: note = D-phenylalanine
  SITE 4: note = N-methyltryptophan
  SITE 6: note = tert-leucine
  MOD_RES 9..10: note = linked via reduced peptide bond
  SITE 11: note = tert-leucine
  SITE 11: note = C-terminus is amidated
XFQWAXGWLPX

SEQ ID NO: 50 - protein, 11 aa, synthetic construct
  SITE 1: note = 4-amino-1-carboxymethyl-piperidine modified with 2,2',2'',2'''-(1,10- dioxa-4,7,13,16-tetraazacyclooctadecane-4,7,13,16-tetrayl) tetraacetic acid
  SITE 2: note = D-phenylalanine
  SITE 4: note = N-methyltryptophan
  SITE 6: note = tert-leucine
  MOD_RES 9..10: note = linked via reduced peptide bond
  SITE 11: note = tert-leucine
  SITE 11: note = C-terminus is amidated
XFQWAXGHLPX

SEQ ID NO: 51 - protein, 11 aa, synthetic construct
  SITE 1: note = 4-amino-1-carboxymethyl-piperidine modified with 2,2',2'',2'''-(1,10- dioxa-4,7,13,16-tetraazacyclooctadecane-4,7,13,16-tetrayl) tetraacetic acid
  SITE 2: note = D-phenylalanine
  SITE 3: note = citrulline
  SITE 4: note = N-methyltryptophan
  SITE 6: note = tert-leucine
  SITE 8: note = 3-quinoylalanine
  MOD_RES 9..10: note = linked via reduced peptide bond
  SITE 11: note = tert-leucine
  SITE 11: note = C-terminus is amidated
XFXWAXGXLPX

SEQ ID NO: 52 - protein, 10 aa, synthetic construct
  SITE 1: note = 4-amino-1-carboxymethyl-piperidine modified with 1,4,7,10- tetrakis(carboxymethyl)-7-acetamide-1,4,10-triaz acyclododecane glutaric acid
  SITE 2: note = D-phenylalanine
  SITE 4: note = N-methyltryptophan
  SITE 6: note = tert-leucine
  MOD_RES 9..10: note = linked via reduced peptide bond
  SITE 10: note = C-terminus is amidated
XFQWAXGHLP

SEQ ID NO: 53 - protein, 11 aa, synthetic construct
  SITE 1: note = 4-amino-1-carboxymethyl-piperidine modified with 1,4,7,10- tetraazacyclododecane-1,4,7,10-tetraacetic acid
  SITE 2: note = D-phenylalanine
  SITE 4: note = N-methyltryptophan
  SITE 6: note = tert-leucine
  MOD_RES 9..10: note = linked via reduced peptide bond
  SITE 11: note = tert-leucine
  SITE 11: note = C-terminus is amidated
XFQWAXGHLPX

SEQ ID NO: 54 - protein, 10 aa, synthetic construct
  SITE 1..10: note = This sequence is one part of a branched amino acid sequence
  SITE 1: note = 4-amino-1-carboxymethyl-piperidine modified with 1,4,7,10- tetraazacyclododecane-1,4,7,10-tetraacetic acid glutamate
  SITE 2: note = D-phenylalanine
  SITE 4: note = 7-fluorotryptophan
  SITE 6: note = tert-leucine
  MOD_RES 9..10: note = linked via reduced peptide bond
  SITE 10: note = C-terminus is amidated
XFQWAXGHLP

SEQ ID NO: 55 - protein, 10 aa, synthetic construct
  SITE 1..10: note = This sequence is one part of a branched amino acid sequence
  SITE 1: note = linked to another sequence
  SITE 1: note = piperidine
  SITE 2: note = D-phenylalanine
  SITE 4: note = 7-fluorotryptophan
  SITE 6: note = tert-leucine
  MOD_RES 9..10: note = linked via reduced peptide bond
  SITE 10: note = C-terminus is amidated
XFQWAXGHLP

SEQ ID NO: 56 - protein, 10 aa, synthetic construct
  SITE 1..10: note = This sequence is one part of a branched amino acid sequence
  SITE 1: note = 4-amino-1-carboxymethyl-piperidine modified with 2,2',2'',2'''-(1,10- dioxa-4,7,13,16-tetraazacyclooctadecane-4,7,13,16-tetrayl) tetraacetic acid- aminoadipic acid-aminoadipic acid-glutamate tripeptide
  SITE 2: note = D-phenylalanine
  SITE 4: note = N-methyltryptophan
  SITE 6: note = tert-leucine
  MOD_RES 9..10: note = linked via reduced peptide bond
  SITE 10: note = C-terminus is amidated
XFQWAXGHLP

SEQ ID NO: 57 - protein, 10 aa, synthetic construct
  SITE 1..10: note = This sequence is one part of a branched amino acid sequence
  SITE 1: note = linked to another sequence
  SITE 1: note = piperidine
  SITE 2: note = D-phenylalanine
  SITE 4: note = N-methyltryptophan
  SITE 6: note = tert-leucine
  MOD_RES 9..10: note = linked via reduced peptide bond
  SITE 10: note = C-terminus is amidated
XFQWAXGHLP

SEQ ID NO: 58 - protein, 10 aa, synthetic construct
  SITE 1: note = 4-amino-1-carboxymethyl-piperidine modified with 2,2',2'',2'''-(1,10- dioxa-4,7,13,16-tetraazacyclooctadecane-4,7,13,16-tetrayl) tetraacetic acid
  SITE 2: note = D-phenylalanine
  SITE 4: note = N-methyltryptophan
  SITE 8: note = 5-fluorotryptophan
  SITE 9: note = statine
  SITE 10: note = C-terminus is amidated
XFQWAVGWXL

SEQ ID NO: 59 - protein, 10 aa, synthetic construct
  SITE 1: note = 4-amino-1-carboxymethyl-piperidine modified with 1,4,7,10- tetrakis(carbamoylmethyl)-1,4,7,10-tetraazacyclododecane
  SITE 2: note = D-phenylalanine
  SITE 7: note = N-methylated glycine
  MOD_RES 9..10: note = linked via reduced peptide bond
  SITE 10: note = C-terminus is amidated
XFQWAVGHLP

SEQ ID NO: 60 - protein, 10 aa, synthetic construct
  SITE 1: note = 4-acetamido-(1-carboxymethyl)piperidine modified with amidated boron trifluoride triazole
  SITE 2: note = D-phenylalanine
  SITE 7: note = N-methylated glycine
  MOD_RES 9..10: note = linked via reduced peptide bond
  SITE 10: note = C-terminus is amidated
XFQWAVGHLP

SEQ ID NO: 61 - protein, 10 aa, synthetic construct
  SITE 1: note = 4-amido-(1-carboxymethyl)piperidine modified with trimethylated 2-aminopyridine
  SITE 2: note = D-phenylalanine
  SITE 7: note = N-methylated glycine
  MOD_RES 9..10: note = linked via reduced peptide bond
  SITE 10: note = C-terminus is amidated
XFQWAVGHLP

SEQ ID NO: 62 - protein, 10 aa, synthetic construct
  SITE 1: note = Fluoro-4-amido-(1-carboxymethyl)piperidine
  SITE 2: note = D-phenylalanine
  SITE 7: note = N-methylated glycine
  MOD_RES 9..10: note = linked via reduced peptide bond
  SITE 10: note = C-terminus is amidated
XFQWAVGHLP

SEQ ID NO: 63 - protein, 10 aa, synthetic construct
  SITE 1: note = 18-Fluoro-4-amido-(1-carboxymethyl)piperidine
  SITE 2: note = D-phenylalanine
  SITE 7: note = N-methylated glycine
  MOD_RES 9..10: note = linked via reduced peptide bond
  SITE 10: note = C-terminus is amidated
XFQWAVGHLP

SEQ ID NO: 64 - protein, 10 aa, synthetic construct
  SITE 1: note = 4-amido-(1-carboxymethyl)piperidine modified with 1,4,7,10- tetraazacyclododecane-1,4,7,10-tetraacetic acid-O-methyl pyridine glycine lysine
  SITE 2: note = D-phenylalanine
  SITE 4: note = N-methyltryptophan
  SITE 6: note = tert-leucine
  MOD_RES 9..10: note = linked via reduced peptide bond
  SITE 10: note = C-terminus is amidated
XFQWAXGHLP

SEQ ID NO: 65 - protein, 10 aa, synthetic construct
  SITE 1: note = 4-amido-(1-carboxymethyl)piperidine modified with 2,2',2'',2'''-(1,10- dioxa-4,7,13,16-tetraazacyclooctadecane-4,7,13,16-tetrayl) tetraacetic acid- O-methyl pyridine glycine lysine
  SITE 2: note = D-phenylalanine
  SITE 4: note = N-methyltryptophan
  SITE 6: note = tert-leucine
  MOD_RES 9..10: note = linked via reduced peptide bond
  SITE 10: note = C-terminus is amidated
XFQWAXGWLP

SEQ ID NO: 66 - protein, 10 aa, synthetic construct
  SITE 1: note = 4-amino-1-carboxymethyl-piperidine modified with 1,4,7,10- tetraazacyclododecane-1,4,7,10-tetraacetic acid
  SITE 2: note = D-phenylalanine
  SITE 4: note = N-methyltryptophan
  SITE 9: note = statine
  SITE 10: note = C-terminus is amidated
XFQWAVGHXL

SEQ ID NO: 67 - protein, 10 aa, synthetic construct
  SITE 1: note = 4-amino-1-carboxymethyl-piperidine modified with 1,4,7,10- tetrakis(carboxymethyl)-7-acetamide-1,4,10-triaz acyclododecane glutaric acid
  SITE 2: note = D-phenylalanine
  SITE 4: note = N-methyltryptophan
  SITE 9: note = statine
  SITE 10: note = C-terminus is amidated
XFQWAVGHXL